(19) United States
(12) Patent Application Publication
(10) Pub. No.: US 2026/0034246 A1
(43) Pub. Date: Feb. 5, 2026

(54) VECTORIZED ANTI-COMPLEMENT ANTIBODIES AND COMPLEMENT AGENTS AND ADMINISTRATION THEREOF

(71) Applicant: REGENXBIO INC., Rockville, MD (US)

(72) Inventors: Joseph BRUDER; Wei-Hua LEE; Mi SHI

(21) Appl. No.: 18/862,484

(22) PCT Filed: May 3, 2023
(86) PCT No.: PCT/US2023/066574

§ 371 Date: Nov. 1, 2024

Related U.S. Application Data
(60) Provisional application No. 63/338,009, filed on May 3, 2022.

(51) Int. Cl. A61K 48/00; A61K 9/00; A61P 27/02; C07K 14/005; C07K 16/18; C12N 15/86

(52) U.S. Cl. CPC A61K 48/0058; A61K 9/0043; A61K 48/0075; A61K 48/0083; A61P 27/02; C07K 14/005; C07K 16/18; C12N 15/86; C07K 2317/565; C07K 2317/622; C12N 2750/14122; C12N 2750/14143; C12N 2750/14152

(57) ABSTRACT

Compositions and methods are described for the delivery of a fully human post-translationally modified therapeutic monoclonal antibody, or an antigen binding fragment thereof, that binds to C3 or C5 to a human subject for treatment of an ocular indication, particularly AMD. Also provided are compositions and methods for the delivery hCHL1 to a human subject for treatment of an ocular indication, particularly AMD. The nucleotide sequence encoding the antibody is delivered in a rAAV vector that targets ocular tissue cells for expression of the transgene.

The contents of the electronic sequence listing submitted herewith as file 38013_0026P1.xml; Size: 441,363 bytes; and Date of Creation: May 3, 2023, is herein incorporated by reference in its entirety.

### 1. INTRODUCTION

Compositions and methods are described for the delivery of a fully human post-translationally modified (HuPTM) proteins, including therapeutic monoclonal antibodies (“mAbs”) that bind to C3 or C5. Also described are HuPTM antigen-binding fragments of a therapeutic mAb that bind to C3 or C5—e.g., a fully human-glycosylated (HuGly) Fab of the therapeutic mAb—to a human subject diagnosed with Age-Related Macular Degeneration (AMD). Compositions and methods are also described for the delivery of hCFHL1 to a human subject diagnosed with AMD.

### 2. BACKGROUND OF THE INVENTION

Therapeutic mAbs have been shown to be effective in treating a number of diseases and conditions. However, because these agents are effective for only a short period of time, repeated injections for long durations are often required, thereby creating considerable treatment burden for patients.

The complement system is a critical element of the immune system that enhances the clearing of microbes and damaged cells, promotes inflammation, and attacks a pathogen's cell membrane. Three biochemical pathways activate the complement system, 1) the classical complement system, 2) the alternative complement pathway and 3) the lectin pathway.

Age-Related Macular Degeneration (AMD) causes progressive and permanent vision impairment. There are two forms of AMD, dry AMD and wet AMD. Dry AMD accounts for about 85-90% of the 196 million global AMD cases. Overactivation of the complement system is an important driver of AMD. Over one million patients who also present with geographic atrophy (GA) secondary to age-related macular degeneration (AMD) may also benefit from intervention to counteract over-active complement in the eye.

There is a need for more effective treatments that reduce the treatment burden on patients suffering from AMD. Intravitreal medications have become a promising mode of drug administration in patients as they provide high volume of drug to the target tissues, eliminating the risk of systemic toxicity. Reducing or eliminating the need for periodic ocular administration would reduce patient burden and improve therapy.

### 3. SUMMARY OF THE INVENTION

Therapeutic antibodies and other proteins delivered by gene therapy have several advantages over injected or infused therapeutic antibodies that dissipate over time resulting in peak and trough levels. Sustained expression of the transgene product antibody or protein, as opposed to injecting an antibody or protein repeatedly, allows for a more consistent level of antibody or protein to be present at the site of action, and is less risky and more convenient for patients, since fewer injections need to be made. Furthermore, antibodies and other proteins expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies. In addition, factors such as anti-C3 or anti-C5 antibodies or complement factor H-like protein (see Clark et al., 2014, J. Immunol. 193:4962) may inhibit complement activation and drusen deposit in the eye to inhibit, reduce the progression of dry AMD. Accordingly, provided herein are compositions and methods for anti-C3 or anti-C5 gene therapy and complement factor H-like (CFHL) protein therapy, particularly recombinant AAV gene therapy, designed to target the eye and generate a depot of transgenes for expression of anti-C3 or anti-C5 antibodies, including crovalimab or eculizumab, or an antigen binding fragment thereof, or CFHL1 protein that result in a therapeutic or prophylactic levels (in ocular tissues and, also, in embodiments in serum) of the antibody within 20 days, 30 days, 40 days, 50 days, 60 days, or 90 days of administration of the rAAV composition for treatment or reduction in the progression of dry AMD and the geographic atrophy associated therewith.

Compositions and methods are described for the ocular or systemic delivery of an anti-C3 or anti-C5 or an anti-C3 or anti-C5 antigen-binding fragment of a therapeutic mAb (for example, a fully human-glycosylated Fab (HuGlyFab) of a therapeutic mAb) or a hCFHL-1 protein (for example, a fully human-glycosylated CFHL-1), to a patient (human subject) diagnosed with AMD or other condition indicated for treatment with the therapeutic anti-C3 or anti-C5 mAb or CFHL-1 protein. Such antigen-binding fragments of therapeutic mAbs include a Fab, F(ab′)₂, or scFv (single-chain variable fragment) (collectively referred to herein as “antigen-binding fragment”). “HuPTM Fab” as used herein may include other antigen binding fragments of a mAb. In an alternative embodiment, full-length mAbs can be used. In addition, scFv forms of antibodies may be used as well. Delivery may be advantageously accomplished via gene therapy—e.g., by administering a viral vector or other DNA expression construct encoding a therapeutic anti-C3 or anti-C5 mAb or its antigen-binding fragment or CFHL protein (or a hyperglycosylated derivative of the foregoing) diagnosed with a condition indicated for treatment with the therapeutic anti-C3 or anti-C5 mAb, or CFHL-1—to create a permanent depot in the eye, or in alternative embodiments, liver and/or muscle, of the patient that continuously supplies the HuPTM mAb or antigen-binding fragment of the therapeutic mAb, e.g., a human-glycosylated transgene product, or peptide to one or more ocular tissues where the mAb or antigen-binding fragment thereof or peptide exerts its therapeutic or prophylactic effect.

Provided are gene therapy vectors, particularly rAAV gene therapy vectors, which when administered to a human subject result in expression of an anti-C3 or anti-C5 antibody or CFHL-1 protein to achieve a maximum or steady states concentrations in ocular tissues, such as aqueous humor, vitreous humor, or in serum for example, 20, 30, 40, 50, 60 or 90 days after administration of the vector encoding the anti-C3 or anti-C5 antibody or CFHL-1 protein.

The recombinant vector used for delivering the transgene includes non-replicating recombinant adeno-associated virus vectors (“rAAV”). In embodiments, the AAV type has a tropism for ocular tissues, including, for example, retinal cells, RPE, choroid, Bruch's membrane (BrM) and epithelial cells thereof, choriocapillaris and epithelial cells thereof, photoreceptor cells (rods and cones) and retinal ganglion cells. The AAV type may be, for example, AAV8, AAV9, AAV3B, or AAVrh73 (or a variant thereof) subtype of AAV. However, other viral vectors may be used, including but not limited to lentiviral vectors; vaccinia viral vectors, or non-viral expression vectors referred to as “naked DNA” constructs. Expression of the transgene can be controlled by constitutive expression elements, such as a CAG promoter, or tissue-specific expression control elements, particularly elements that are ocular tissue, liver and/or muscle specific control elements, for example one or more elements of Tables 1 and 1a.

In certain embodiments, the HuPTM mAb or HuPTM antigen-binding fragment encoded by the transgene can include, but is not limited to, a full-length or an antigen-binding fragment of a therapeutic antibody that binds to C3, particularly NGM621, or therapeutic antibodies that bind to C5, including crovalimab, eculizumab, ravulizumab or tesidolumab, see, for example, FIGS. 1A-1C for structure and FIGS. 2A-2G and Table 7 for amino acid sequences. In other embodiments, provided are HuPTM of CFHL-1. In other embodiments, provided are recombinant AAV vectors comprising a transgene encoding a BB5.1 antibody which may be used as a surrogate for anti C5-binding antibodies, including eculizumab or ravulizumab, or anti-C3.105B9 for anti-C3-binding antibodies, such as NGM621, in non-human animal models, such as non-human primates (cynomolgus monkeys) rat or mouse models, for pre-clinical assessment for Dry AMD.

Gene therapy constructs for the therapeutic antibodies are designed such that both the heavy and light chains are expressed. The coding sequences for the heavy and light chains can be engineered in a single construct in which the heavy and light chains are separated by a cleavable linker or IRES so that separate heavy and light chain polypeptides are expressed. In particular embodiments, the linker is a Furin T2A linker (SEQ ID NOS: 143 or 144). In certain embodiments, the coding sequences encode for a Fab or F(ab′)₂ or an scFv. In certain embodiments the full length heavy and light chains or Fab fragments of the antibody are expressed. In other embodiments, the constructs express an scFv in which the heavy and light chain variable domains are connected via a flexible, non-cleavable linker, such as GGGGSGGGGSGGGGS (SEQ ID NO: 53). In certain embodiments, the construct expresses, from the N-terminus, NH₂-V_{L}-linker-V_{H}-COOH or NH₂-V_{H}-linker-V_{L}-COOH. In certain embodiments, the construct encodes, from the N-terminus, NH₂-signal or leader sequence-V_{L}-GGGGSGGGGSGGGGS-V_{H}-COOH or NH₂-signal or leader sequence-V_{H}-GGGGSGGGGSGGGGS-V_{L}-COOH.

In addition, antibodies or other proteins expressed from transgenes in vivo are not likely to contain degradation products associated with antibodies or other proteins produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

The production of HuPTM mAb, HuPTM Fab, or HuPTM scFv or HuPTM CFHL-1 in ocular tissue cells of the human subject should result in a “biobetter” molecule for the treatment of disease accomplished via gene therapy—e.g., by administering a viral vector or other DNA expression construct encoding a full-length HuPTM mAb or HuPTM Fab or HuPTM scFv of a therapeutic mAb or CFHL-1 to a patient (human subject) diagnosed with a disease indication for that mAb or CFHL-1 protein, to create a permanent depot in the subject that continuously supplies the human-glycosylated, sulfated transgene product produced by the subject's transduced cells. The cDNA construct for the HuPTMmAb or HuPTM Fab or HuPTM scFv should include a signal peptide (including signal peptides at the N-terminus of each separately expressed heavy and light chains or at the N-terminus of the scFv) that ensures proper co- and post-translational processing (glycosylation and protein sulfation) by the transduced human cells.

As an alternative, or an additional treatment to gene therapy, the full-length HuPTM mAb or HuPTM Fab or HuPTM scFv or CHFL-1 protein can be produced in human cell lines by recombinant DNA technology, and the glycoprotein can be administered to patients.

Combination therapies involving systemic delivery of the full-length HuPTM anti-C3 or anti-C5, mAb or HuPTM anti-C3 or anti-C5 Fab or scFv or CFHL-1 protein to the patient accompanied by administration of other available treatments are encompassed by the methods provided herein. The additional treatments may be administered before, concurrently or subsequent to the gene therapy treatment. Such additional treatments can include but are not limited to co-therapy with the therapeutic mAb.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

Methods of administration and manufacture are also provided.

### 3.1. EMBODIMENTS

Embodiment 1. A pharmaceutical composition for treating Age-Related Macular Degeneration (AMD) in a human subject in need thereof, comprising an adeno-associated virus (AAV) vector having: (a) a viral capsid that has a tropism for ocular tissue cells; and (b) an artificial genome comprising an expression cassette flanked by AAV inverted terminal repeats (ITRs), wherein the expression cassette comprises a transgene encoding a heavy chain and a light chain of a substantially full-length or full-length anti-C3 or anti-C5 antibody, or an antigen-binding fragment thereof, operably linked to one or more regulatory sequences that promote expression of the transgene in human ocular tissue cells; wherein said AAV vector is formulated for subretinal, intravitreal, intranasal, intracameral, suprachoroidal, or systemic administration to said human subject.

Embodiment 2. The pharmaceutical composition of embodiment 1, wherein the viral capsid is at least 95% identical to the amino acid sequence of AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).

Embodiment 3. The pharmaceutical composition of embodiment 1 or embodiment 2, wherein the AAV capsid is AAV9, AAV8, AAV3B, or AAVrh73, or a variant thereof.

Embodiment 4. The pharmaceutical composition of any one of embodiments 1 to 3, wherein the human ocular tissue cells are retinal cells, RPE-choroid tissue cells, BrM epithelial cells, choriocapillaris epithelial cells, or photoreceptor cells (rods, cones and/or retinal ganglion cells).

Embodiment 5. The pharmaceutical composition of any one of embodiments 1 to 4, wherein the regulatory sequence comprises a regulatory sequence from Table 1.

Embodiment 6. The pharmaceutical composition of embodiment 5, wherein the regulatory sequence is a CAG promoter (SEQ ID NO: 74), a mutated CAG promoter (SEQ ID NO: 304, SEQ ID NO: 305 or SEQ ID NO: 306), a CB promoter (SEQ ID NO: 222 or 223), a human rhodopsin kinase (GRK1) promoter (SEQ ID NOS: 77 or 217), a mouse cone arresting (CAR) promoter (SEQ ID NOS: 214-216), a human red opsin (RedO) promoter (SEQ ID NO: 212) or a Best1/GRK1 tandem promoter (SEQ ID NO: 224).

Embodiment 7. The pharmaceutical composition of embodiment 6, wherein the regulatory sequence is a CAG promoter (SEQ ID NO: 74) or a mutated CAG promoter (SEQ ID NO: 304, SEQ ID NO: 305 or SEQ ID NO: 306).

Embodiment 8. The pharmaceutical composition of any one of embodiments 1 to 7, wherein the transgene comprises a Furin/2A linker between the nucleotide sequences coding for the heavy chain and the nucleotide sequence coding for the light chain of said substantially full-length or full-length anti-C3 or anti-C5 antibody, or an antigen-binding fragment thereof.

Embodiment 9. The pharmaceutical composition of embodiment 8, wherein said Furin/2A linker is a Furin/T2A linker having the amino acid sequence RKRR(GSG)APVKQTLNFDLLKLAGDVESNPGP (SEQ ID NOS: 143 or 144).

Embodiment 10. The pharmaceutical composition of any one of embodiments 1 to 9, wherein the transgene encodes a signal sequence at the N-terminus of the heavy chain and the light chain of said antigen-binding fragment that directs secretion and post-translational modification in said human ocular tissue cells.

Embodiment 11. The pharmaceutical composition of embodiment 10, wherein said signal sequence is MYRMQLLLLIALSLALVINS (SEQ ID NO: 85) or a signal sequence from Table 2.

Embodiment 12. The pharmaceutical composition of any one of embodiments 1 to 7, wherein the transgene encodes an scFv with the structure: signal sequence-VH-linker-VL-poly A.

Embodiment 13. The pharmaceutical composition of any one of embodiments 1 to 7, wherein the transgene encodes an scFv with the structure: signal sequence-VL-linker-VH-poly A.

Embodiment 14. The pharmaceutical composition of embodiment 12 or 13, wherein the linker is GGGGS (SEQ ID NO: 51), GGGGSGGGGS (SEQ ID NO: 52), GGGGSGGGGSGGGGS (SEQ ID NO: 53), GGGGSGGGGSGGGGSGGGGS (SEQ ID NO: 54) or GGGGSGGGGGGGGSGGGGSGGGGS (SEQ ID NO: 55).

Embodiment 15. The pharmaceutical composition of embodiment 14, wherein the linker is GGGGSGGGGGGGGS (SEQ ID NO: 53).

Embodiment 16. The pharmaceutical composition of any one of embodiments 12-15, wherein the signal sequence is MYRMQLLLLIALSLALVINS (SEQ ID NO: 85) or a signal sequence from Table 2.

Embodiment 17. The pharmaceutical composition of embodiment 16, wherein the signal sequence is MYRMQLLLLIALSLALVINS (SEQ ID NO: 85).

Embodiment 18. The pharmaceutical composition of any one of embodiments 12-17, wherein VH is SEQ ID NO: 251 and VL is SEQ ID NO: 252, wherein VH is SEQ ID NO: 253 and VL is SEQ ID NO: 254, wherein VH is SEQ ID NO: 255 and the VL is SEQ ID NO: 256, wherein VH is SEQ ID NO: 257 and VL is SEQ ID NO: 258, or wherein VH is SEQ ID NO: 259 and VL is SEQ ID NO: 260.

Embodiment 19. The pharmaceutical composition of embodiment 18, wherein the transgene encodes a polypeptide having an amino acid sequence of SEQ ID NO: 261, SEQ ID NO: 262, SEQ ID NO: 263, SEQ ID NO: 264, SEQ ID NO: 265, SEQ ID NO: 266, SEQ ID NO: 270, SEQ ID NO: 271, SEQ ID NO: 272, SEQ ID NO: 273, SEQ ID NO: 274, SEQ ID NO: 275, or SEQ ID NO: 276 or wherein the artificial genome comprises Crovalimab.scFv (SEQ ID NO: 267 or SEQ ID NO: 268 or SEQ ID NO: 269 or SEQ ID NO: 277 or SEQ ID NO: 278 or SEQ ID NO: 279 or SEQ ID NO: 280 or SEQ ID NO: 281 or SEQ ID NO: 282 or SEQ ID NO: 283 or SEQ ID NO: 284 or SEQ ID NO: 285 or SEQ ID NO: 286 or SEQ ID NO: 287 or SEQ ID NO: 288 or SEQ ID NO: 289 or SEQ ID NO: 290 or SEQ ID NO: 291 or SEQ ID NO: 292).

Embodiment 20. The pharmaceutical composition of any one of embodiments 1 to 11, wherein transgene has the structure: signal sequence-Heavy chain-Furin site-2A site-signal sequence-Light chain-Poly A or signal sequence-light chain-Furin site-2A site-signal sequence-heavy chain-PolyA.

Embodiment 21. The pharmaceutical composition of any one of embodiments 1 to 11 or 20, wherein the anti-C5 antibody is crovalimab, eculizumab, ravulizumab or tesidolumab, or an antigen binding fragment thereof.

Embodiment 22. The pharmaceutical composition of any one of embodiments 1 to 11 or 20 to 21, wherein the substantially full-length or full-length anti-C5 antibody, or an antigen-binding fragment thereof comprises a heavy chain with an amino acid sequence of SEQ ID NO: 1 and optionally an Fc polypeptide with an amino acid sequence of SEQ ID NO: 64 and a light chain with an amino acid sequence of SEQ ID NO: 2; a heavy chain with an amino acid sequence of SEQ ID NO: 3 and optionally an Fc polypeptide with an amino acid sequence of SEQ ID NO: 65 and a light chain with an amino acid sequence of SEQ ID NO: 5; a heavy chain with an amino acid sequence of SEQ ID NO: 4 and optionally an Fc polypeptide with an amino acid sequence of SEQ ID NO: 65 and a light chain with an amino acid sequence of SEQ ID NO: 5; a heavy chain with an amino acid sequence of SEQ ID NO: 6 and optionally an Fc polypeptide with an amino acid sequence of SEQ ID NO: 66 and a light chain with an amino acid sequence of SEQ ID NO: 7; a heavy chain with an amino acid sequence of SEQ ID NO: 8 and optionally an Fc polypeptide with an amino acid sequence of SEQ ID NO: 67 and a light chain with an amino acid sequence of SEQ ID NO: 9.

Embodiment 23. The pharmaceutical composition of any one of embodiments 1 to 11 or 20 to 22, wherein the transgene comprises a nucleotide sequence of SEQ ID NO: 26 encoding the heavy chain and a nucleotide sequence of SEQ ID NO: 27 encoding the light chain; a nucleotide sequence of SEQ ID NO: 28 encoding the heavy chain and a nucleotide sequence of SEQ ID NO: 30 encoding the light chain; a nucleotide sequence of SEQ ID NO: 29 encoding the heavy chain and a nucleotide sequence of SEQ ID NO: 30 encoding the light chain; a nucleotide sequence of SEQ ID NO: 31 encoding the heavy chain and a nucleotide sequence of SEQ ID NO: 32 encoding the light chain; or a nucleotide sequence of SEQ ID NO: 33 encoding the heavy chain and a nucleotide sequence of SEQ ID NO: 34 encoding the light chain.

Embodiment 24. The pharmaceutical composition of any one of embodiments 1 to 11 or 20, wherein the anti-C3 antibody is NGM621, or an antigen binding fragment thereof.

Embodiment 25. The pharmaceutical composition of any one of embodiments 1 to 11 or 20 or 24, wherein the substantially full-length or full-length anti-C3, or an antigen-binding fragment thereof comprises a heavy chain with an amino acid sequence of SEQ ID NO: 10 and optionally an Fc polypeptide with an amino acid sequence of SEQ ID NO: 68 and a light chain with an amino acid sequence of SEQ ID NO: 13; or a heavy chain with an amino acid sequence of SEQ ID NO: 11 and optionally an Fc polypeptide with an amino acid sequence of SEQ ID NO: 68 and a light chain with an amino acid sequence of SEQ ID NO: 13.

Embodiment 26. The pharmaceutical composition of any one of embodiments 1 to 25, wherein the antigen-binding fragment is a Fab, a F(ab′) 2, or an scFv.

Embodiment 27. The pharmaceutical composition of any one of embodiments 1 to 11 or 20-23 or 26 wherein the artificial genome comprises CAG.Eculizumab.full (SEQ ID NO: 47), CAG.Crovalimab.full (SEQ ID NO: 44), CAG.Eculizumab.fab.IgG1 (SEQ ID NO: 45), CAG.Eculizumab.fab.IgG2 (SEQ ID NO: 46), or CAG.Crovalimab.fab (SEQ ID NO: 43).

Embodiment 28. The pharmaceutical composition of any one of the foregoing embodiments where the artificial genome is a self complementary genome.

Embodiment 29. A composition comprising an adeno-associated virus (AAV) vector having: a a viral AAV capsid, that is optionally at least 95% identical to the amino acid sequence of AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26); and b. an artificial genome comprising an expression cassette flanked by AAV inverted terminal repeats (ITRs), wherein the expression cassette comprises a transgene encoding a heavy and a light chain of a substantially full-length or full-length BB5.1 or crovalimab, or an antigen-binding fragment thereof, operably linked to one or more regulatory sequences that promote expression of the transgene in human ocular tissue cells; wherein the transgene encodes a signal sequence at the N-terminus of the heavy chain and the light chain and wherein the signal sequence directs secretion and post translational modification of said substantially full-length or full-length BB5.1 or crovalimab, or an antigen-binding fragment thereof, in human ocular tissue cells.

Embodiment 30. The composition of embodiment 29, wherein the ocular tissue cells are retinal cells, RPE-choroid tissue cells, BrM epithelial cells, choriocapillaris epithelial cells, or photoreceptor cells (rods, cones and/or retinal ganglion cells).

Embodiment 31. The composition of embodiment 29 or embodiment 30, wherein the AAV capsid is AAV8, AAV9, AAV3B, or AAVrh73, or variant thereof.

Embodiment 32. The composition of any one of embodiments 29 to 31, wherein the full-length mAb or the antigen-binding fragment comprises a heavy chain with an amino acid sequence of SEQ ID NO: 1 and optionally an Fc polypeptide with an amino acid sequence of SEQ ID NO: 64 and a light chain with an amino acid sequence of SEQ ID NO: 2; or SEQ ID NO: 15 and a light chain with an amino acid sequence of SEQ ID NO: 16.

Embodiment 33. The composition of any one of embodiments 29 to 32, wherein the transgene comprises a nucleotide sequence of SEQ ID NO: 26 encoding the heavy chain and a nucleotide sequence of SEQ ID NO: 27 encoding the light chain or a nucleotide sequence of SEQ ID NO: 35 encoding the heavy chain and a nucleotide sequence of SEQ ID NO: 36 encoding the light chain.

Embodiment 34. The composition of any one of embodiments 29 to 33, wherein the transgene comprises a Furin/2A linker between the nucleotide sequences coding for the heavy and light chains of said substantially full-length or full-length BB5.1 or crovalimab.

Embodiment 35. The composition of any one of embodiments 29 to 34, wherein the nucleic acid encoding a Furin 2A linker is incorporated into the expression cassette in between the nucleotide sequences encoding the heavy and light chain sequences, resulting in a construct with the structure: Signal sequence-Heavy chain-Furin site-2A site-Signal sequence-Light chain-PolyA.

Embodiment 36. The composition of any one of embodiments 29 to 35, wherein said Furin 2A linker is a Furin/T2A linker having the amino acid sequence RKRR(GSG)APVKQTLNFDLLKLAGDVESNPGP (SEQ ID NOS: 143 or 144).

Embodiment 37. The composition of any one of embodiments 29 to 36, wherein said signal sequence is MYRMQLLLLIALSLALVINS (SEQ ID NO: 85) or a signal sequence from Table 2.

Embodiment 38. The composition of any one of embodiments 29 to 37, wherein the antigen-binding fragment is a Fab, a F(ab′) 2, or an scFv

Embodiment 39. The composition of any one of embodiments 29 to 38, wherein the artificial genome comprises CAG.Crovalimab.full (SEQ ID NO: 44), CAG.Crovalimab.fab (SEQ ID NO: 43), CAG.BB5.1 (SEQ ID NO: 48) or Crovalimab.scFv (SEQ ID NO: 267 or SEQ ID NO: 268 or SEQ ID NO: 269 or SEQ ID NO: 277 or SEQ ID NO: 278 or SEQ ID NO: 279 or SEQ ID NO: 280 or SEQ ID NO: 281 or SEQ ID NO: 282 or SEQ ID NO: 283 or SEQ ID NO: 284 or SEQ ID NO: 285 or SEQ ID NO: 286 or SEQ ID NO: 287 or SEQ ID NO: 288 or SEQ ID NO: 289 or SEQ ID NO: 290 or SEQ ID NO: 291 or SEQ ID NO: 292).

Embodiment 40. The composition of any one of embodiments 29-31, wherein the transgene encodes a scFv with the structure: signal sequence-VH-linker-VL-polyA.

Embodiment 41. The composition of any one of embodiments 29-31, wherein the transgene encodes a scFv with the structure: signal sequence-VL-linker-VH-poly A.

Embodiment 42. The composition of any embodiment 40 or embodiment 41, wherein the linker is GGGGS (SEQ ID NO: 51), GGGGSGGGGS (SEQ ID NO: 52), GGGGSGGGGSGGGGS (SEQ ID NO: 53), GGGGGGGGSGGGGGGGGS (SEQ ID NO: 54) or GGGGSGGGGSGGGGSGGGGSGGGGS (SEQ ID NO: 55).

Embodiment 43. The composition of embodiment 42, wherein the linker is GGGGSGGGGSGGGGS (SEQ ID NO: 53).

Embodiment 44. The composition of any one of embodiments 40-43, wherein the signal sequence is MYRMQLLLLIALSLALVTNS (SEQ ID NO: 85) or a signal sequence from Table 2.

Embodiment 45. The composition of embodiment 44, wherein the signal sequence is MYRMQLLLLIALSLALVTNS (SEQ ID NO: 85).

Embodiment 46. The composition of any one of embodiments 40-45, wherein VH is SEQ ID NO: 251 and VL is SEQ ID NO: 252, wherein VH is SEQ ID NO: 253 and VL is SEQ ID NO: 254, wherein VH is SEQ ID NO: 255 and the VL is SEQ ID NO: 256, wherein VH is SEQ ID NO: 257 and VL is SEQ ID NO: 258, or wherein VH is SEQ ID NO: 259 and VL is SEQ ID NO: 260.

Embodiment 47. The composition of any one of embodiments 40-46, wherein the transgene encodes a polypeptide having an amino acid sequence of SEQ ID NO: 261, SEQ ID NO: 262, SEQ ID NO: 263, SEQ ID NO: 264, SEQ ID NO: 265, SEQ ID NO: 266, SEQ ID NO: 270, SEQ ID NO: 271, SEQ ID NO: 272, SEQ ID NO: 273, SEQ ID NO: 274, SEQ ID NO: 275, or SEQ ID NO: 276 or wherein the artificial genome comprises Crovalimab.scFv (SEQ ID NO: 267 or SEQ ID NO: 268 or SEQ ID NO: 269 or SEQ ID NO: 277 or SEQ ID NO: 278 or SEQ ID NO: 279 or SEQ ID NO: 280 or SEQ ID NO: 281 or SEQ ID NO: 282 or SEQ ID NO: 283 or SEQ ID NO: 284 or SEQ ID NO: 285 or SEQ ID NO: 286 or SEQ ID NO: 287 or SEQ ID NO: 288 or SEQ ID NO: 289 or SEQ ID NO: 290 or SEQ ID NO: 291 or SEQ ID NO: 292).

Embodiment 48: The composition of any one of embodiments 29 to 47 wherein the artificial genome is self-complementary.

Embodiment 49. A method of assaying the efficacy of a gene therapy treatment comprising administering the compositions of any one of embodiments 29 to 48 to a non-human primate or rodent model of AMD, wherein the expression cassette comprises a transgene encoding a heavy and a light chain of a substantially full-length or full-length BB5.1.

Embodiment 50. A composition for use in the treatment of or a method of treating Age Related Macular Degeneration (AMD) in a human subject in need thereof, comprising subretinally, intravitreally, intranasally, intracamerally, suprachoroidally, or systemically administering to the subject a therapeutically effective amount of a composition comprising a recombinant AAV comprising a transgene encoding an anti-C3 mAb or anti-C5 mAb, or antigen-binding fragment thereof, operably linked to one or more regulatory sequences that control expression of the transgene in ocular tissue cells.

Embodiment 51. The composition or method of claim 50 wherein the anti-C5□mAb is crovalimab, eculizumab, ravulizumab or tesidolumab.

Embodiment 52. The composition or method of embodiment 50 or 51, wherein the full-length anti-C5 mAb or the antigen-binding fragment comprises a heavy chain with an amino acid sequence of SEQ ID NO: 1 and optionally an Fc polypeptide with an amino acid sequence of SEQ ID NO: 64 and a light chain with an amino acid sequence of SEQ ID NO: 2; a heavy chain with an amino acid sequence of SEQ ID NO: 3 and optionally an Fc polypeptide with an amino acid sequence of SEQ ID NO: 65 and a light chain with an amino acid sequence of SEQ ID NO: 5; a heavy chain with an amino acid sequence of SEQ ID NO: 4 and optionally an Fc polypeptide with an amino acid sequence of SEQ ID NO: 65 and a light chain with an amino acid sequence of SEQ ID NO: 5; a heavy chain with an amino acid sequence of SEQ ID NO: 6 and optionally an Fc polypeptide with an amino acid sequence of SEQ ID NO: 66 and a light chain with an amino acid sequence of SEQ ID NO: 7 or a heavy chain with an amino acid sequence of SEQ ID NO: 8 and optionally an Fc polypeptide with an amino acid sequence of SEQ ID NO: 67 and a light chain with an amino acid sequence of SEQ ID NO: 9.

Embodiment 53. The composition or method of any of embodiments 50 to 52, wherein the transgene comprises a nucleotide sequence of SEQ ID NO: 26 encoding the heavy chain and a nucleotide sequence of SEQ ID NO: 27 encoding the light chain; a nucleotide sequence of SEQ ID NO: 28 encoding the heavy chain and a nucleotide sequence of SEQ ID NO: 30 encoding the light chain; a nucleotide sequence of SEQ ID NO: 29 encoding the heavy chain and a nucleotide sequence of SEQ ID NO: 30 encoding the light chain; a nucleotide sequence of SEQ ID NO: 31 encoding the heavy chain and a nucleotide sequence of SEQ ID NO: 32 encoding the light chain or a nucleotide sequence of SEQ ID NO: 33 encoding the heavy chain and a nucleotide sequence of SEQ ID NO: 34 encoding the light chain.

Embodiment 54. The composition or method of embodiment 50, wherein the anti-CD3 antibody is NGM621, or an antigen binding fragment thereof.

Embodiment 55. The composition or method of embodiment 50 or embodiment 54, wherein the full-length mAb or the antigen-binding fragment comprises a heavy chain with an amino acid sequence of SEQ ID NO: 10 and optionally an Fc polypeptide with an amino acid sequence of SEQ ID NO: 68 and a light chain with an amino acid sequence of SEQ ID NO: 13 or a heavy chain with an amino acid sequence of SEQ ID NO: 11 and optionally an Fc polypeptide with an amino acid sequence of SEQ ID NO: 68 and a light chain with an amino acid sequence of SEQ ID NO: 13.

Embodiment 56. The composition or method of any one of embodiments 50 to 55, wherein the ocular tissue cell is a retinal cell, an RPE-choroid tissue cell, BrM epithelial cells, choriocapillaris epithelial cells, or photreceptor cells (rods, cones and/or retinal ganglion cells).

Embodiment 57. The composition or method of any one of embodiments 50 to 56 wherein the viral capsid is at least 95% identical to the amino acid sequence of AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).

Embodiment 58. The composition or method of any one of embodiments 50 to 57, wherein the AAV capsid is AAV9, AAV8, AAV3B, or AAVrh73, or a variant thereof.

Embodiment 59. The composition or method of any one of embodiments 50 to 58, wherein the regulatory sequence includes a regulatory sequence from Table 1 or Table 1a.

Embodiment 60. The composition or method of embodiment 59, wherein the regulator sequence is a CAG promoter (SEQ ID NO: 74), a mutated CAG promoter (SEQ ID NO: 304, SEQ ID NO: 305 or SEQ ID NO: 306), a CB promoter (SEQ ID NO: 222 or 223), human rhodopsin kinase (GRK1) promoter (SEQ ID NOS: 77 or 217), a mouse cone arresting (CAR) promoter (SEQ ID NOS: 214-216), a human red opsin (RedO) promoter (SEQ ID NO: 212) or a Best1/GRK1 tandem promoter (SEQ ID NO: 224).

Embodiment 61. The composition or method of any one of embodiments 50 to 60, wherein the transgene comprises a Furin/2A linker between the nucleotide sequences coding for the heavy and light chains of said mAb.

Embodiment 62. The composition or method of embodiment 61, wherein said Furin/2A linker is a Furin/T2A linker having the amino acid sequence RKRR(GSG)APVKQTLNFDLLKLAGDVESNPGP (SEQ ID NOS: 143 or 144).

Embodiment 63. The composition or method of any one of embodiments 50 to 62, wherein the transgene encodes a signal sequence at the N-terminus of the heavy chain and the light chain of said antigen-binding fragment that directs secretion and post translational modification in said human ocular tissue cells.

Embodiment 64. The composition or method of embodiment 63, wherein said signal sequence is MYRMQLLLLIALSLALVTNS (SEQ ID NO: 85) or a signal sequence from Table 2.

Embodiment 65. The composition or method of any of embodiments 50 to 64, wherein transgene has the structure: Signal sequence-Heavy chain-Furin site-2A site-Signal sequence-Light chain-PolyA or Signal sequence-light chain-Furin site-2A site-Signal sequence-heavy chain-PolyA.

Embodiment 66. The composition or method of any one of embodiments 50 to 60, wherein the transgene encodes an scFv with the structure: Signal sequence-VH-linker sequence-VL-poly A.

Embodiment 67. The composition or method of any one of embodiments 50 to 60, wherein the transgene encodes an scFv with the structure: Signal sequence-VL-linker sequence-VH-poly A.

Embodiment 68. The composition or method of embodiment 66 or embodiment 67, wherein the linker is GGGGS (SEQ ID NO: 51), GGGGSGGGGS (SEQ ID NO: 52), GGGGSGGGGSGGGGS (SEQ ID NO: 53), GGGGSGGGGSGGGGSGGGGS (SEQ ID NO: 54) or GGGGSGGGGSGGGGSGGGGSGGGGS (SEQ ID NO: 55).

Embodiment 69. The composition or method of embodiment 68, wherein the linker is GGGGSGGGGSGGGGS (SEQ ID NO: 53).

Embodiment 70. The composition or method of any one of embodiments 66-69, wherein the signal sequence is MYRMQLLLLIALSLALVTNS (SEQ ID NO: 85) or a signal sequence from Table 2.

Embodiment 71. The composition or method of embodiment 70, wherein the signal sequence is MYRMQLLLLIALSLALVTNS (SEQ ID NO: 85).

Embodiment 72. The composition or method of any one of embodiments 66-71, wherein VH is SEQ ID NO: 251 and VL is SEQ ID NO: 252, wherein VH is SEQ ID NO: 253 and VL is SEQ ID NO: 254, wherein VH is SEQ ID NO: 255 and the VL is SEQ ID NO: 256, wherein VH is SEQ ID NO: 257 and VL is SEQ ID NO: 258, or wherein VH is SEQ ID NO: 259 and VL is SEQ ID NO: 260.

Embodiment 73. The composition or method of any one of embodiments 66 to 72, wherein the transgene encodes a polypeptide having an amino acid sequence of SEQ ID NO: 261, SEQ ID NO: 262, SEQ ID NO: 263, SEQ ID NO: 264, SEQ ID NO: 265, SEQ ID NO: 266, SEQ ID NO: 270, or SEQ ID NO: 271 or wherein the artificial genome comprises Crovalimab.scFv (SEQ ID NO: 267 or SEQ ID NO: 268 or SEQ ID NO: 269 or SEQ ID NO: 277 or SEQ ID NO: 278 or SEQ ID NO: 279 or SEQ ID NO: 280 or SEQ ID NO: 281 or SEQ ID NO: 282 or SEQ ID NO: 283 or SEQ ID NO: 284 or SEQ ID NO: 285 or SEQ ID NO: 286 or SEQ ID NO: 287 or SEQ ID NO: 288 or SEQ ID NO: 289 or SEQ ID NO: 290 or SEQ ID NO: 291 or SEQ ID NO: 292).

Embodiment 74. The composition or method of any one of embodiments 50 to 73 in which production of said HuPTM form of said mAb or antigen-binding fragment thereof is confirmed by transducing human ocular tissue cells in culture with said recombinant nucleotide expression vector and expressing said mAb or antigen-binding fragment thereof.

Embodiment 75. The composition or method of any one of embodiments 50 to 74, wherein the therapeutically effective amount is determined to be sufficient to maintain a concentration of at least 10 ng/ml in RPE, retina, aqueous humor or vitreous humor.

Embodiment 76. The composition or method of any one of embodiments 50 to 75, wherein the therapeutically effective amount is determined to be sufficient to improve best corrected visual acuity (BCVA) by >=2 ETDRS lines; to decrease the mean rate of change in geographic atrophy as measured by fundus autofluorescence (FAF); to improve visual function as measured by dark adaptation methodology; to improve contrast sensitivity by the Pelli-Robson test or reduce the drusen area within 10 weeks, 20 weeks, 6 months or 1 year of administration.

Embodiment 77. The composition or method of any one of embodiments 50 to 76, wherein the artificial genome comprises CAG.Eculizumab.full (SEQ ID NO: 47), CAG.Crovalimab.full (SEQ ID NO: 44), CAG.Eculizumab.fab.IgG1 (SEQ ID NO: 45), CAG.Eculizumab.fab.IgG2 (SEQ ID NO: 46), CAG.Crovalimab.fab (SEQ ID NO: 43), or Crovalimab.scFv (SEQ ID NO: 267 or SEQ ID NO: 268 or SEQ ID NO: 269 or SEQ ID NO: 277 or SEQ ID NO: 278 or SEQ ID NO: 279 or SEQ ID NO: 280 or SEQ ID NO: 281 or SEQ ID NO: 282 or SEQ ID NO: 283 or SEQ ID NO: 284 or SEQ ID NO: 285 or SEQ ID NO: 286 or SEQ ID NO: 287 or SEQ ID NO: 288 or SEQ ID NO: 289 or SEQ ID NO: 290 or SEQ ID NO: 291 or SEQ ID NO: 292).

Embodiment 78. The composition or method of any one of embodiments 50 to 77 wherein the artificial genome is self-complementary.

Embodiment 79. The composition or method of any one of embodiments 50 to 78, wherein the anti-C3 mAb or anti-C5 mAb, or antigen-binding fragment thereof inhibits both classical and alternative complement pathways.

Embodiment 80. The method of any one of embodiments 50 to 79, wherein the anti-C3 mAb or anti-C5 mAb, or antigen-binding fragment thereof inhibits membrane attack complex (MAC) formation.

Embodiment 81. A method of producing recombinant AAVs comprising: culturing a host cell containing: (a) an artificial genome comprising a cis expression cassette flanked by AAV ITRs, wherein the cis expression cassette comprises comprising a transgene encoding a substantially full-length or full-length anti-C3 or anti-C5 or antigen-binding fragment thereof, operably linked to one or more regulatory sequences that promote expression of the transgene in human ocular tissue cells; (b) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has ocular tissue cell tropism; (c) sufficient adenovirus helper functions to permit replication and packaging of the artificial genome by the AAV capsid protein; and (d) recovering recombinant AAV encapsidating the artificial genome from the cell culture.

Embodiment 82. The method of embodiment 81, wherein the transgene encodes a substantially full-length or full-length mAb or antigen binding fragment that comprises the heavy and light chain variable domains of BB5.1, crovalimab, eculizumab, ravulizumab, tesidolumab or NGM621, wherein the AAV capsid protein is an AAV8, AAV9, AAV3B, or AAVrh73, or variant thereof capsid protein.

Embodiment 83. The method of embodiment 81 or embodiment 82, wherein the ocular tissue cell is a retinal cell, an RPE-choroid tissue cell, BrM epithelial cells, choriocapillaris epithelial cells, or photreceptor cells (rods, cones and/or retinal ganglion cells).

Embodiment 84. The method of any one of embodiments 81 to 83, wherein the artificial genome comprises CAG.Eculizumab.full (SEQ ID NO: 47), CAG.Crovalimab.full (SEQ ID NO: 44), CAG.Eculizumab.fab.IgG1 (SEQ ID NO: 45), CAG.Eculizumab.fab.IgG2 (SEQ ID NO: 46), CAG.Crovalimab.fab (SEQ ID NO: 43), CAG.BB5.1 (SEQ ID NO: 48), Crovalimab.scFv (SEQ ID NO: 267, SEQ ID NO: 268 or SEQ ID NO: 269 or SEQ ID NO: 277 or SEQ ID NO: 278 or SEQ ID NO: 279 or SEQ ID NO: 280 or SEQ ID NO: 281 or SEQ ID NO: 282 or SEQ ID NO: 283 or SEQ ID NO: 284 or SEQ ID NO: 285 or SEQ ID NO: 286 or SEQ ID NO: 287 or SEQ ID NO: 288 or SEQ ID NO: 289 or SEQ ID NO: 290 or SEQ ID NO: 291 or SEQ ID NO: 292).

Embodiment 85. A host cell comprising: a plasmid comprising a cis expression cassette flanked by AAV ITRs, wherein the cis expression cassette comprises a transgene encoding a substantially full-length or full-length anti-C3 mAb or anti-C5 mAb, or antigen-binding fragment thereof, operably linked to one or more regulatory sequences that promote expression of the transgene in human ocular tissue cells.

Embodiment 86. The host cell of embodiment 85, wherein the transgene encodes a substantially full-length or full-length mAb or antigen binding fragment that comprises the heavy and light chain variable domains of BB5.1, crovalimab, eculizumab, ravulizumab, tesidolumab or NGM621.

Embodiment 87. The host cell of embodiment 85 or embodiment 86, wherein the ocular tissue cell is a retinal cell, an RPE-choroid tissue cell, BrM epithelial cells, choriocapillaris epithelial cells, or photreceptor cells (rods, cones and/or retinal ganglion cells).

Embodiment 88. The host cell of any one of embodiments 85 to 87, wherein the transgene within the construct comprises CAG.Eculizumab.full (SEQ ID NO: 47), CAG.Crovalimab.full (SEQ ID NO: 44), CAG.Eculizumab.fab.IgG1 (SEQ ID NO: 45), CAG.Eculizumab.fab.IgG2 (SEQ ID NO: 46) CAG.Crovalimab.fab (SEQ ID NO: 43), CAG.BB5.1 (SEQ ID NO: 48), Crovalimab.scFv (SEQ ID NO: 267 or SEQ ID NO: 268 or SEQ ID NO: 269 or SEQ ID NO: 277 or SEQ ID NO: 278 or SEQ ID NO: 279 or SEQ ID NO: 280 or SEQ ID NO: 281 or SEQ ID NO. 282 or SEQ ID NO: 283 or SEQ ID NO: 284 or SEQ ID NO: 285 or SEQ ID NO: 286 or SEQ ID NO: 287 or SEQ ID NO: 288 or SEQ ID NO: 289 or SEQ ID NO: 290 or SEQ ID NO: 291 or SEQ ID NO: 292).

Embodiment 89. A pharmaceutical composition for treating AMD in a human subject in need thereof, comprising an adeno-associated virus (AAV) vector having: (a) a viral capsid that has a tropism for ocular tissue cells; and (b) an artificial genome comprising an expression cassette flanked by AAV internal tandem repeats (ITRs), wherein the expression cassette comprises a transgene encoding hCFHL1, operably linked to one or more regulatory sequences that control expression of the transgene in human ocular tissue cells; wherein said AAV vector is formulated for subretinal, intravitreal, intranasal, intracameral, suprachoroidal, or systemic administration to said human subject.

Embodiment 90. The pharmaceutical composition of embodiment 89, wherein the viral capsid is at least 95% identical to the amino acid sequence of AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).

Embodiment 91. The pharmaceutical composition of embodiment 89 or embodiment 87, wherein the AAV capsid is AAV8, AAV9, AAV3B, or AAVrh73, or variant thereof.

Embodiment 92. The pharmaceutical composition of any one of embodiments 89 to 91, wherein the ocular tissue cells are retinal cells, RPE-choroid tissue cells, BrM epithelial cells, choriocapillaris epithelial cells, or photreceptor cells (rods, cones and/or retinal ganglion cells).

Embodiment 93. The pharmaceutical composition of any one of embodiments 89 to 92, wherein the regulatory sequence comprises a regulatory sequence from Table 1.

Embodiment 94. The pharmaceutical composition of embodiment 93, wherein the regulatory sequence is a CAG promoter (SEQ ID NO: 74), a mutated CAG promoter (SEQ ID NO: 304, SEQ ID NO: 305 or SEQ ID NO: 306), a CB promoter (SEQ ID NO: 222 or 223), a human rhodopsin kinase (GRK1) promoter (SEQ ID NOS: 77 or 217), a mouse cone arresting (CAR) promoter (SEQ ID NOS: 214-216), a human red opsin (RedO) promoter (SEQ ID NO: 212) or a Best1/GRK1 tandem promoter (SEQ ID NO: 224).

Embodiment 95. The pharmaceutical composition of any one of embodiments 89 to 94, wherein said transgene comprises the nucleotide sequence of SEQ ID NO: 49.

Embodiment 96. The pharmaceutical composition of any one of embodiments 89 to 95, wherein the hCFHL1 protein has an amino acid sequence of SEQ ID NO: 23.

Embodiment 97. The pharmaceutical composition of any one of embodiments 89 to 96 wherein the artificial genome comprises construct CAG.hCFHL.1 (SEQ ID NO: 50).

Embodiment 98. A composition comprising an adeno-associated virus (AAV) vector having: a. a viral AAV capsid, that is optionally at least 95% identical to the amino acid sequence of AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26); b. an artificial genome comprising an expression cassette flanked by AAV inverted terminal repeats (ITRs), wherein the expression cassette comprises a transgene encoding an hCFHL protein, operably linked to one or more regulatory sequences that promote expression of the transgene in human ocular tissue cells; wherein the transgene encodes a signal sequence at the N-terminus of said hCFHL1 protein that directs secretion and post translational modification of said hCFHL1 protein in ocular tissue cells.

Embodiment 99. The composition of embodiment 98, wherein the hCFHL1 protein has an amino acid sequence of SEQ ID NO: 23.

Embodiment 100. The composition of embodiment 98 or embodiment 99, wherein the ocular tissue cells are retinal cells, RPE-choroid tissue cells, BrM epithelial cells, choriocapillaris epithelial cells, or photoreceptor cells (rods, cones and/or retinal ganglion cells).

Embodiment 101. The composition of any one of embodiments 98 to 100, wherein said transgene comprises the nucleotide sequence of SEQ ID NO: 49.

Embodiment 102. The composition of any one of embodiments 98 to 101, wherein said signal sequence is MYRMQLLLLIALSLALVINS (SEQ ID NO: 85) or a signal sequence from Table 2.

Embodiment 103. The composition of any one of embodiments 98 to 102, wherein the artificial genome is self-complementary.

Embodiment 104. The composition of any one of embodiments 98 to 103 wherein the artificial genome comprises the construct CAG.hCFHL.1 (SEQ ID NO: 50).

Embodiment 105. A method of treating AMD in a human subject in need thereof, comprising subretinally, intravitreally, intranasally, intracamerally, suprachoroidally, or systemically administering to the subject a therapeutically effective amount of a composition comprising a recombinant AAV comprising a transgene encoding an hCFHL1 protein operably linked to one or more regulatory sequences that control expression of the transgene in ocular tissue cells.

Embodiment 106. A method of treating AMD in a human subject in need thereof, comprising: subretinally, intravitreally, intranasally, intracamerally, suprachoroidally, or systemically administering to said subject a therapeutically effective amount of a recombinant nucleotide expression vector comprising a transgene encoding an hCFHL1 protein, operably linked to one or more regulatory sequences that control expression of the transgene in human ocular tissue cells, so that a depot is formed that releases a human post-translationally modified (HuPTM) form of hCFHL1 protein.

Embodiment 107. The method of embodiment 105 or 106, wherein the ocular tissue cell is a retinal cell, an RPE-choroid tissue cell, BrM epithelial cells, choriocapillaris epithelial cells, or photoreceptor cells (rods, cones and/or retinal ganglion cells)

Embodiment 108. The method of any one of embodiments 105 to 107, wherein said transgene has the nucleotide sequence of SEQ ID NO: 49.

Embodiment 109. The method of any one of embodiments 105 to 108, wherein the viral capsid is at least 95% identical to the amino acid sequence of AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).

Embodiment 110. The method of any one of embodiments 105 to 109, wherein the AAV capsid is AAV8, AAV9, AAV3B, or AAVrh73 or variant thereof.

Embodiment 111. The method of any one of embodiments 105 to 110, wherein the regulatory sequence includes a regulatory sequence from Table 1 or Table 1a.

Embodiment 112. The method of embodiment 111, wherein the regulatory sequence is a CAG promoter (SEQ ID NO: 74), a mutated CAG promoter (SEQ ID NO: 304, SEQ ID NO: 305 or SEQ ID NO: 306), a CB promoter (SEQ ID NO: 222 or 223), a human rhodopsin kinase (GRK1) promoter (SEQ ID NOS: 77 or 217), a mouse cone arresting (CAR) promoter (SEQ ID NOS: 214-216), a human red opsin (RedO) promoter (SEQ ID NO: 212) or a Best1/GRK1 tandem promoter (SEQ ID NO: 224).

Embodiment 113. The method of any one of embodiments 105 to 112, wherein the transgene encodes a signal sequence at the N-terminus of the hCFHL1 protein that directs secretion and post translational modification in said human ocular tissue cells.

Embodiment 114. The method of embodiment 113, wherein said signal sequence is MYRMQLLLLIALSLALVTNS (SEQ ID NO: 85) or a signal sequence from Table 2.

Embodiment 115. The method of any one of embodiments 105 to 114 in which production of said HuPTM form of the hCFHL1 protein is confirmed by transducing human ocular cells in culture with said recombinant nucleotide expression vector and expressing said hCFHL1 protein.

Embodiment 116. The method of embodiment 105 or embodiment 106, wherein the therapeutically effective amount is determined to be sufficient to improve best corrected visual acuity (BCVA) by >=2 ETDRS lines or increase in log MAR; to decrease the mean rate of change in geographic atrophy as measured by fundus autofluorescence (FAF); to improve visual function as measured by dark adaptation methodology; to improve contrast sensitivity by the Pelli-Robson test, or reduce the drusen area within 10 weeks, 20 weeks, 6 months or 1 year of administration.

Embodiment 117. The method of any of any one of embodiments 105 to 116 wherein the transgene is within the construct CAG.hCFHL.1 (SEQ ID NO: 50).

Embodiment 118. A method of producing recombinant AAVs comprising: culturing a host cell containing: (a) an artificial genome comprising a cis expression cassette flanked by AAV ITRs, wherein the cis expression cassette comprises a transgene encoding an hCFHL1 protein, operably linked to one or more regulatory sequences that promote expression of the transgene in human ocular tissue cells; (b) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has ocular tissue tropism; (c) sufficient adenovirus helper functions to permit replication and packaging of the artificial genome by the AAV capsid protein; and (d) recovering recombinant AAV encapsidating the artificial genome from the cell culture.

Embodiment 119. The method of embodiment 118, wherein the ocular tissue cell is a retinal cell, an RPE-choroid tissue cell, BrM epithelial cells, choriocapillaris epithelial cells, or photoreceptor cells (rods, cones and/or retinal ganglion cells).

Embodiment 120. The method of embodiment 118 or embodiment 119, wherein the AAV capsid protein is AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).

Embodiment 121. A host cell comprising: a plasmid comprising a cis expression cassette flanked by AAV ITRs, wherein the cis expression cassette comprises a transgene encoding hCFHL1, operably linked to one or more regulatory sequences that promote expression of the transgene in human ocular tissue cells.

Embodiment 122. The host cell of embodiment 121, wherein the transgene encodes hCFHL1 (SEQ ID NO: 23).

### 4. BRIEF DESCRIPTION OF THE DRAWINGS

FIGS. 1A-1C. Schematics of rAAV vector genome constructs containing an expression cassette encoding the heavy and light chains of a therapeutic mAb separated by a Furin-2A linker, operably linked to a promoter, flanked by the AAV ITRs. The transgene can comprise nucleotide sequences encoding the full-length heavy and light chains with Fc regions (A), the heavy and light chains of the Fab portion (B), or a single chain variable fragment (scFv) connecting the heavy and light chains of the antibody with a linker (C).

FIGS. 2A-2G. The amino acid sequence of a transgene construct for the Fab region of crovalimab (A), eculizumab IgG1 (B), eculizumab IgG2 (C) ravulizumab (D), and tesidolumab (E) therapeutic antibodies to C5 and NGM621 (F) and (G) therapeutic antibody to C3. Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIG. 3. Clustal Multiple Sequence Alignment of various capsids with ocular tissue tropism. Amino acid substitutions (shown in bold in the bottom rows) can be made to AAV8 capsids by “recruiting” amino acid residues from the corresponding position of other aligned AAV capsids. Sequence shown in gray=hypervariable regions. The amino acid sequences of the AAV capsids are assigned sequence ID numbers as indicated in FIG. 3.

FIG. 4 Glycans that can be attached to HuGlyFab regions of full length mAbs or the antigen-binding domains. (Adapted from Bondt et al., 2014, Mol & Cell Proteomics 13.1:3029-3039).

FIG. 5. Clustal Multiple Sequence Alignment of constant heavy chain regions (CH2 and CH3) of IgG1 (SEQ ID NO: 183), IgG2 (SEQ ID NO: 184), and IgG4 (SEQ ID NO: 185). The hinge region, from residue 219 to residue 230 of the heavy chain, is shown in italics. The numbering of the amino acids is in EU-format.

FIGS. 6A-6C. (A and B) Expression of full length crovalimab and eculizumab levels after transfection of HEK293T cells with pITR-CAG-Eculizumab and pITR-CAG-crovalimab on non-reducing (A) and reducing (B) gels (C) Expression of full length hCFHL1 levels after transfection of HEK293T cells with pITR-CAG-hCFHL.1

FIGS. 7A and 7B show the alignment of different antibody sequences. A) Heavy chain sequences of antibodies. Top to bottom: amino acids 1-230 of SEQ ID NO: 1, amino acids 1-230 of SEQ ID NO: 3, amino acids 1-220 of SEQ ID NO: 4, amino acids 1-238 of SEQ ID NO: 6, amino acids 1-228 of SEQ ID NO: 8, amino acids 1-238 of SEQ ID NO: 10 and amino acids 1-245 of SEQ ID NO: 11. B) Light chain sequences of antibodies. Top to bottom: amino acids 1-164 of SEQ ID NO: 2, amino acids 1-161 of SEQ ID NO: 5, amino acids 1-161 of SEQ ID NO: 7, amino acids 1-162 of SEQ ID NO: 9 and amino acids 1-161 of SEQ ID NO: 14.

FIGS. 8A and 8B show the results of the ability of cis plasmid-expressed vectorized antibodies in HEK293 cells to suppress complement in a hemolysis inhibition assay with (A) 1.5% normal human serum or (B) 20% normal mouse serum. A) human C5 inhibitors, IgG=Crovalimab full-length mAb, Fab=Crovalimab Fab, scFv=Crovalimab scFv mAb, compared to a recombinant C5 inhibitor (C51) protein and isotype and vehicle controls. B) mouse C5 inhibitors, IgG=BB5.1 full-length mAb, Fab=BB5.1 Fab, scFv=BB5.1 scFv mAb, compared to a recombinant C5 inhibitor (C51) protein and isotype and vehicle controls.

FIGS. 9A-9F show that the recombinant purified forms of each C5 inhibitor suppressed classical and alternative complement pathways in hemolysis inhibition assays against A) 50% human C5, classical complement pathway conditions, testing anti-hC5 (Crovalimab formats) and C5 inhibitor, B) 50% human C5, alternative complement pathway conditions, testing anti-hC5 (Crovalimab formats) and C5 inhibitor C) 50% mouse C5, classical complement pathway conditions, testing anti-hC5 (Crovalimab formats) and anti-mC5 (BB5.1 mAb formats), D) 50% mouse C5, classic complement pathway conditions, comparing anti-hC5 (Crovalimab full-length mAb) and anti-mC5 (BB5.1 full-length mAb), E) 50% mouse C5, classic complement pathway conditions, comparing anti-hC5 (Crovalimab Fab mAb) and anti-mC5 (BB5.1 Fab mAb), and F) 50% mouse C5, classic complement pathway conditions, comparing anti-hC5 (Crovalimab scFv mAb) and anti-mC5 (BB5.1 scFv mAb).

FIGS. 10A-H measure membrane attack complex (MAC) formation in ARPE-19 cells (FIGS. 10A-C) or iPSC-derived RPE cells (FIGS. 10D-H).

FIGS. 11A and 11B show the results of AAV8-encoding C5 inhibitors injected into wild-type mouse eyes via subretinal (SR) administration at 1E8 and 3E8 vg/eye AAV8.CAG.anti-hC5 (Crovalimab) vectors were formatted as IgG (full-length), Fab or scFV vectorized antibodies and administered subretinally (SR) at each dose, and AAV8.CAG.anti-mC5 (BB5.1) vectors were administered SR at each dose, while purified recombinant anti-mC5 IgG (BB5.1) or isotype controls were delivered intraperitoneal (ip). A) represents measurement of transgene product (TP) as ng/eye (RNA transcript); B) represents measurement of transgene product (TP) as pmol/eye (protein).

### 5. DETAILED DESCRIPTION OF THE INVENTION

Compositions and methods are described for the systemic delivery of a fully human post-translationally modified (HuPTM) therapeutic monoclonal antibody (mAb) or a HuPTM antigen-binding fragment of a therapeutic anti-C3 or anti-C5 mAb (for example, a fully human-glycosylated Fab (HuGlyFab) or scFv of a therapeutic mAb) or a HuPTM version of CFHL-1 to a patient (human subject) diagnosed with AMD (including dry AMD) or other indication indicated for treatment with the therapeutic mAb. Delivery may be advantageously accomplished via gene therapy—e.g., by administering a viral vector or other DNA expression construct encoding a therapeutic mAb or its antigen-binding fragment (or a hyperglycosylated derivative of either) or hCFHL-1 to a patient (human subject) diagnosed with a condition indicated for treatment with the therapeutic mAb-to create a permanent depot in a tissue or organ of the patient, particularly the eye, but, in embodiments, liver or muscle, that continuously supplies the HuPTM mAb or antigen-binding fragment of the therapeutic mAb or hCFHL-1, e.g., a human-glycosylated transgene product, into ocular tissues of the subject to where the mAb or antigen-binding fragment there of exerts its therapeutic effect.

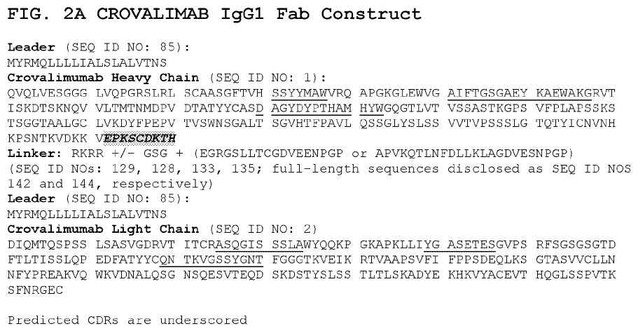

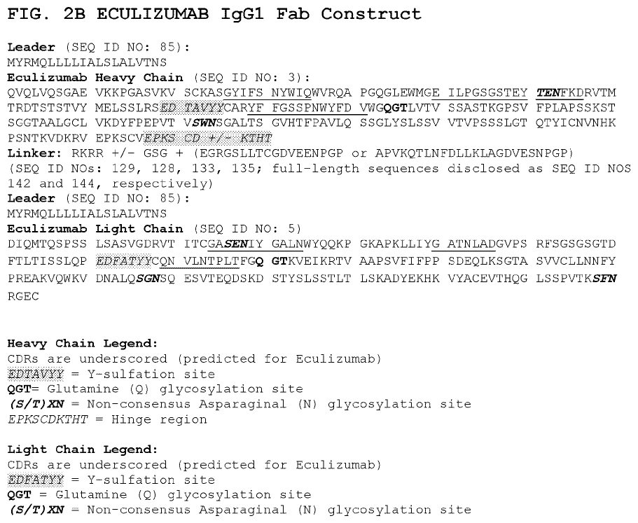

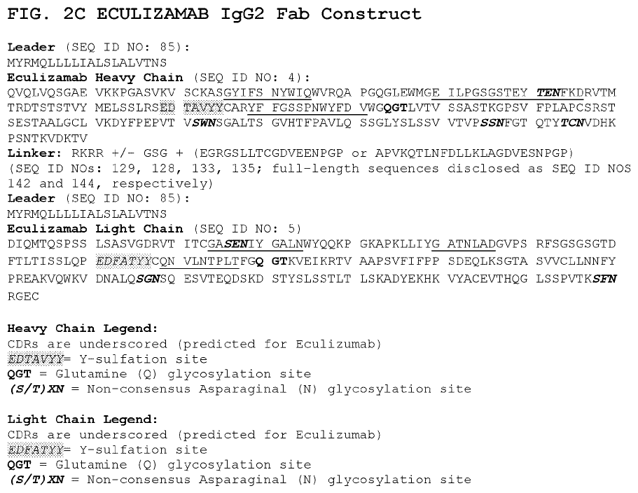

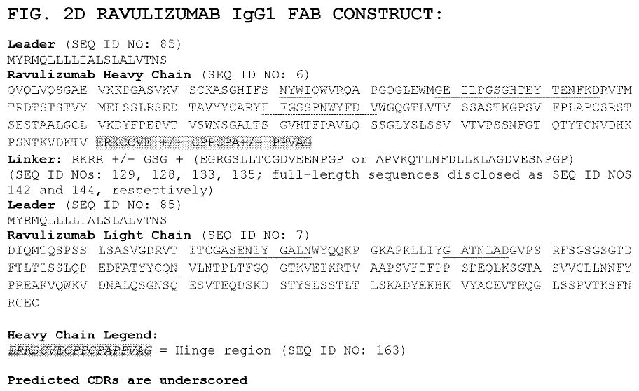

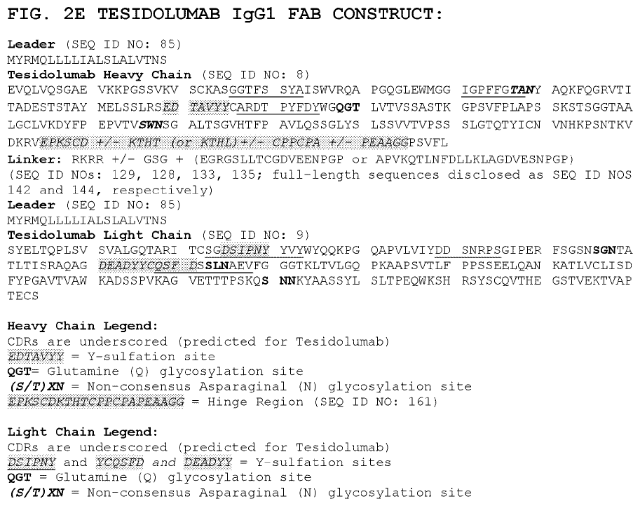

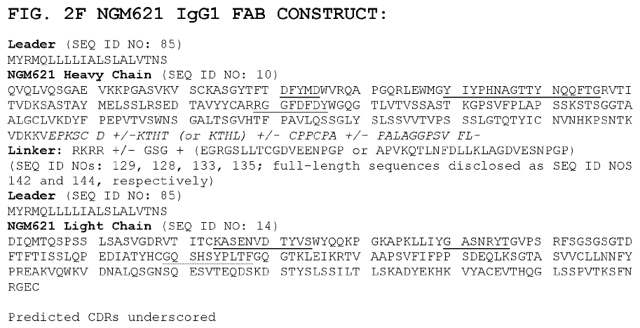

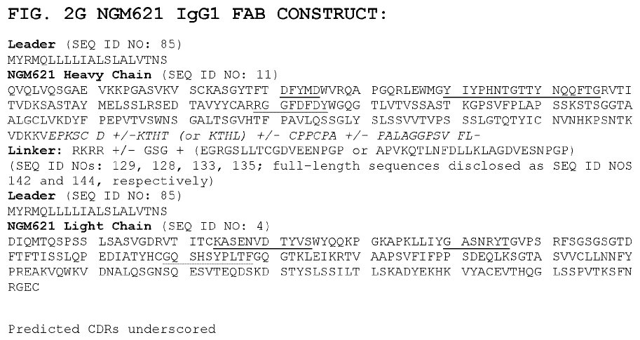

In certain embodiments, the HuPTM mAb or HuPTM antigen-binding fragment encoded by the transgene is, but is not limited to, a full-length or an antigen-binding fragment of a HuPTM mAb or HuPTM Fab or HuPTM scFv that binds C5, particularly crovalimab, eculizumab, ravulizumab or tesidolumab (see FIGS. 2A-2D, respectively, for the heavy and light chain amino acid sequences of the Fab portion of these antibodies (see also Table 7 for the Fab heavy and light chain amino acid sequences and certain full length and scFv amino acid sequences)) and C3, particularly NGM621 (see FIG. 2E for the heavy and light chain amino acid sequences of the Fab portion of NGM621 (and Table 7)). See also Table 7 for the amino acid sequence of hCHFL-1 (SEQ ID NO: 23).

The compositions and methods provided herein ocularly or systemically deliver anti-C5 antibodies or antigen binding fragments thereof, particularly crovalimab, eculizumab, ravulizumab or tesidolumab antibodies or anti-C3 antibodies or antigen binding fragments thereof, particularly, NGM621, or hCFHL-1, from a depot of viral genomes, for example, in the subject's eye (including retinal tissue), or liver/muscle, at a level either in the ocular tissue (e.g., in the vitreous or aqueous humor or retinal tissue, RPE, BrM and/or choroid), or in the serum that is therapeutically or prophylactically effective to treat or ameliorate the symptoms of AMD or other indication that may be treated with an anti-C3 or anti-C5 antibody or antigen binding fragment thereof or hCFHL-1 protein. Identified herein are viral vectors for delivery of transgenes encoding the therapeutic anti-C3 or anti-C5 antibodies or antigen-binding fragments thereof, or hCFHL-1 protein, to cells in the human subject, including, in embodiments, one or more ocular tissue cells, and regulatory elements operably linked to the nucleotide sequence encoding the heavy and light chains of the anti-C3 or anti-C5 antibody or antigen binding fragment that promote the expression of the antibody or antigen binding fragment in the cells, or to the nucleotide sequence encoding hCFHL-1, in embodiments, in the ocular tissue cells. Such regulatory elements, including constitutive promoters, such as CAG, and ocular tissue-specific regulatory elements, are provided in Table 1 and Table 1a and in Example 13 (including certain modified CAG promoters) herein. Accordingly, such viral vectors may be delivered to the human subject at appropriate dosages, such that at least 20, 30, 40, 50 or 60 days after administration, the anti-C3 or anti-C5 antibody or antigen binding fragment thereof or CFHL-1 protein is present at therapeutically effective levels in the serum or in ocular tissues of said human subject. In embodiments, the therapeutically effective level of the anti-C3 or anti-C5 antibody or antigen binding fragment thereof, or CFHL-1 is determined (in human trials, animal models, etc.) to improve best corrected visual acuity (BCVA) by >=2 ETDRS lines, reduction in geographic atrophy (or slow the progression of geographic atrophy relative to untreated individual either based upon controls or natural history of the disease), reduction in drusen deposits or other metric of dry AMD.

The HuPTM mAb or HuPTM antigen-binding fragment encoded by the transgene can include, but is not limited to, a full-length or an antigen-binding fragment of a therapeutic antibody that binds to C3, including but not limited to, NGM621, or to C5, including but not limited to crovalimab, eculizumab, ravulizumab or tesidolumab. The amino acid sequences of the heavy and light chain of antigen binding fragments of the foregoing are provided in Table 7, infra. Heavy chain variable domain being within SEQ ID NO: 1, 3, 4, 6, 8, 10, 11, or 12 (CH1 domain is underlined and VH domain is not) and wherein the Fab fragment is encoded by nucleotide sequence SEQ ID NO: 26, 28, 29, 31, or 33 ((NGM621 coding sequence not provided), respectively) and light chain Fab (with CL 1 domain underlined and VL domain not underlined) having an amino acid sequence of SEQ ID NO: 2, 5, 7, 9, 13, or 14 (encoded by nucleotide sequence SEQ ID NO: 27, 30, 32, or 34, respectively (NGM621 sequence not provided)). Also provided is the amino acid sequence for hCFHL-1 (SEQ ID NO; 23), encoded by nucleotide sequence of SEQ ID NO: 49. Also provided are the heavy and light chains of BB5.1 full length antibody (SEQ ID NO: 15 and 16), encoded by nucleotide sequences SEQ ID NO: 35 and 36, respectively. The HuPTM mAb or HuPTM antigen-binding fragment encoded by the transgene can include, but is not limited to, a full-length or an antigen-binding fragment of a therapeutic antibody or antigen-binding fragments engineered to contain additional glycosylation sites on the Fab domain (e.g., see Courtois et al., 2016, mAbs 8:99-112 which is incorporated by reference herein in its entirety for its description of derivatives of antibodies that are hyperglycosylated on the Fab domain of the full-length antibody).

The recombinant vector used for delivering the transgene includes non-replicating recombinant adeno-associated virus vectors (“rAAV”), rAAVs are particularly attractive vectors for a number of reasons—they can be modified to preferentially target a specific organ of choice; and there are hundreds of capsid serotypes to choose from to obtain the desired tissue specificity, and/or to avoid neutralization by pre-existing patient antibodies to some AAVs. The AAV types for use here in preferentially target the eye, i.e., have a tropism for retinal cells. Such rAAVs include but are not limited to AAV based vectors comprising capsid components from one or more of AAV1, AAV2, AAV3, AAV3B, AAV4, AAV5, AAV6, AAV7, AAV8, AAVrh8, AAV9, AAV9e, AAVrh10, AAVrh20, AAVrh39, AAVhu.37, AAVrh73, AAVrh74, AAV.hu51, AAV.hu21, AAV.hu12, or AAV.hu26. In certain embodiments, AAV based vectors provided herein comprise capsids from one or more of AAV3B, AAV8, AAV9, AAVrh10, AAV10, or AAVrh73 serotypes.

However, other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements.

Gene therapy constructs are designed such that both the heavy and light chains are expressed either as independent polypeptide chains when processed or as a single chain in an scFv. In certain embodiments, the full length heavy and light chains of the antibody are expressed. In certain embodiments, the coding sequences encode for a Fab or F(ab′)₂ or an scFv. The heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. In specific embodiments, the linker separating the heavy and light chains is a Furin-2A linker, for example a Furin-F2A linker RKRR(GSG)APVKQTLNFDLLKLAGDVESNPGP (SEQ ID NOS: 143 or 144) or a Furin-T2A linker RKRR(GSG)EGRGSLLTCGDVEENPGP (SEQ ID NOS: 141 or 142). In certain embodiments, the construct expresses, from the N-terminus to C-terminus, NH2-light chain-IRES or Furin 2A linker-Heavy chain-COOH or NH2-heavy chain-IRES or Furin 2A linker-light chain-COOH. In other embodiments, the construct expresses, from the N-terminus to C-terminus, NH2-signal or localization sequence-light chain-IRES or Furin 2A linker-Heavy chain or NH2-signal or localization sequence-heavy chain-IRES or Furin 2A linker-light chain-COOH. In other embodiments, the constructs express a scFv in which the heavy and light chain variable domains are connected via a flexible, non-cleavable linker, such as GGGGGGGGSGGGGS (SEQ ID NO: 53). Exemplary cleavable and non-cleavable linkers are found in Table 4. In certain embodiments, the construct expresses, from the N-terminus to C-terminus, NH2-VL-linker-VH-COOH or NH2-VH-linker-VL-COOH. In other embodiments, the construct expresses, from the N-terminus to C-terminus, NH2-signal or localization sequence-VL-linker-VH-COOH or NH2-signal or localization sequence-VH-linker-VL-COOH.

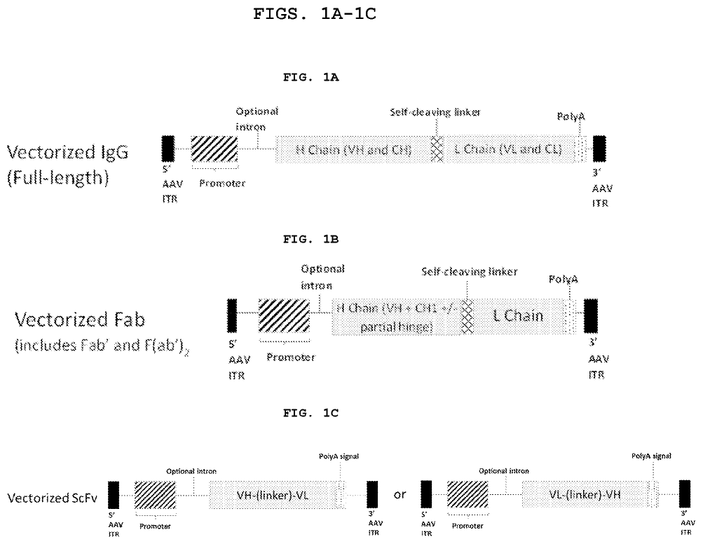

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161) and may also be optimized to reduce CpG dimers. Codon optimized sequences of the crovalimab and eculizumab heavy and light chains and CFHL-1 are provided in Table 8 (SEQ ID NOs: 26 to 30 and 49). Each heavy and light chain requires a signal sequence to ensure proper post-translation processing and secretion (unless expressed as a scFv, in which only the N-terminal chain requires a signal sequence sequence). Useful signal sequences for the expression of the heavy and light chains of the therapeutic antibodies in human cells are disclosed herein, for example in Tables 2 and 3. Exemplary recombinant expression constructs are shown in FIGS. 1A, 1B and 1C.

The production of HuPTM mAb or HuPTM Fab (including an HuPTM scFv) should result in a “biobetter” molecule for the treatment of disease accomplished via gene therapy—e.g., by administering a viral vector or other DNA expression construct encoding a full-length HuPTM mAb or HuPTM Fab or other antigen binding fragment, such as an scFv, of a therapeutic mAb, or a CFHL-1 protein to a patient (human subject) diagnosed with a disease indication for that mAb or protein, to create a permanent depot in the subject that continuously supplies the human-glycosylated, sulfated transgene product produced by the subject's transduced cells. The cDNA construct for the HuPTM mAb or HuPTM Fab or HuPTM scFv should include a signal peptide that ensures proper co- and post-translational processing (glycosylation and protein sulfation) by the transduced human cells.

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil.

As an alternative, or an additional treatment to gene therapy, the full-length HuPTM mAb or HuPTM Fab or HuPTM scFv or other antigen binding fragment thereof, or CFHL-1 protein can be produced in human cell lines by recombinant DNA technology, and the glycoprotein can be administered to patients. Human cell lines that can be used for such recombinant glycoprotein production include but are not limited to human embryonic kidney 293 cells (HEK293), fibro sarcoma HT-1080, HKB-11, CAP, HuH-7, and retinal cell lines, PER.C6, or RPE to name a few (e.g., see Dumont et al., 2015, Crit. Rev. Biotechnol. 36 (6): 1110-1122, which is incorporated by reference in its entirety for a review of the human cell lines that could be used for the recombinant production of the HuPTM mAb, HuPTM Fab or HuPTM scFv product, e.g., HuPTM Fab glycoprotein) or HuPTM CFHL-1 protein. To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in human cells.

It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment of the invention is to slow or arrest the progression of disease.

Combination therapies involving delivery of the full-length HuPTM mAb or HuPTM Fab or HuPTM scFv or antigen binding fragment thereof to the patient accompanied by administration of other available treatments are encompassed by the methods of the invention. The additional treatments may be administered before, concurrently or subsequent to the gene therapy treatment. Such additional treatments can include but are not limited to co-therapy with the therapeutic mAb.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

### 5.1 CONSTRUCTS

Viral vectors or other DNA expression constructs encoding an anti-C5 or anti-C3 mAb or antigen-binding fragment thereof, particularly a HuGlyFab or HuGlyscFv, or a hyperglycosylated derivative of a HuPTM mAb antigen-binding fragment or hCFHL-1 protein are provided herein. The viral vectors and other DNA expression constructs provided herein include any suitable method for delivery of a transgene to a target cell. The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted ocular tissue cells or a vector that has a tropism for ocular tissue cells.

In some aspects, the disclosure provides for a nucleic acid for use, wherein the nucleic acid comprises a nucleotide sequence that encodes a HuPTM mAb or HuPTM Fab or HuPTM scFv or HuGlyFab or other antigen-binding fragment thereof, or hCFHL-1, as a transgene described herein, operatively linked to an ubiquitous promoter, an ocular tissue-specific promoter, or an inducible promoter, wherein the promoter is selected for expression in tissue targeted for expression of the transgene. Promoters may, for example, be a CB7/CAG promoter (SEQ ID NO: 73) and associated upstream regulatory sequences, CAG promoter (CMS early enhancer, Chicken Beta-actin promoter-chicken beta actin intron-rabbit beta-globin splice acceptor) (SEQ ID NO: 74), Chicken Beta-actin promoter-chicken beta actin intron-rabbit beta-globin splice acceptor) with mutations (SEQ ID NO: 304, SEQ ID NO: 305 or SEQ ID NO: 306), cytomegalovirus (CMV) promoter, EF-1 alpha promoter (SEQ ID NO: 76), mU1a (SEQ ID NO: 75), UB6 promoter, chicken beta-actin (CBA) promoter, and ocular-tissue specific promoters, such as human rhodopsin kinase (GRK1) promoter (SEQ ID NOS: 77 or 217), a mouse cone arresting (CAR) promoter (SEQ ID NOS: 214-216), or a human red opsin (RedO) promoter (SEQ ID NO: 212). See Tables 1 and 1a for a list of useful promoters. See also Example 13 for promoters.

In certain embodiments, provided herein are recombinant vectors that comprise one or more nucleic acids (e.g., polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., the nucleotide sequences encoding the heavy and light chains of the HuPTMmAb or HuPTM Fab or HuPTM scFv or HuGlyFab or other antigen-binding fragment or CFHL-1 protein), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV2 inverted terminal repeats that flank the expression cassette; (2) one or more control elements, b) optionally, a chicken β-actin or other intron and c) a rabbit β-globin poly A signal; and (3) nucleic acid sequences coding for the heavy and light chains of a mAb or Fab, separated by a self-cleaving furin (F)/(F/T) 2A linker (SEQ ID NOS: 141-144), ensuring expression of equal amounts of the heavy and the light chain polypeptides or encoding an scFv or CFHL protein. Exemplary constructs are shown in FIGS. 1A, 1B and 1C.

In a specific embodiment, the constructs described herein comprise the following components: (1) AAV2 inverted terminal repeats that flank the expression cassette; (2) GRK1 promoter (SEQ ID NO:77), b) optionally, a VH4 intron (SEQ ID NO:80) or other intron and c) a rabbit β-globin polyA signal (SEQ ID NO:78); and (3) nucleic acid sequences coding for a full-length antibody comprising the heavy and light chain sequences using sequences that encode the Fab portion of the heavy chain, including the hinge region sequence, plus the Fc polypeptide of the heavy chain for the appropriate isotype and the light chain, wherein heavy and light chain nucleotide sequences are separated by a self-cleaving furin (F)/(F/T) 2A linker (SEQ ID NOS: 141-144), ensuring expression of equal amounts of the heavy and the light chain polypeptides or an scFv or CFHL protein.

5.1.1 mRNA Vectors

In certain embodiments, as an alternative to DNA vectors, the vectors provided herein are modified mRNA encoding for the gene of interest (e.g., the transgene, for example, HuPTMmAb or HuGlyFab or other antigen binding fragment thereof or CFHL-1 protein). The synthesis of modified and unmodified mRNA for delivery of a transgene to retinal pigment epithelial cells is taught, for example, in Hansson et al., J. Biol. Chem., 2015, 290 (9): 5661-5672, which is incorporated by reference herein in its entirety. In certain embodiments, provided herein is a modified mRNA encoding for a HuPTMmAb, HuPTM Fab, or HuPTM scFv.

### 5.1.2 Viral Vectors

Viral vectors include adenovirus, adeno-associated virus (AAV, e.g., AAV8, AAV9, AAVrh10, AAV10), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV) and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitis virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

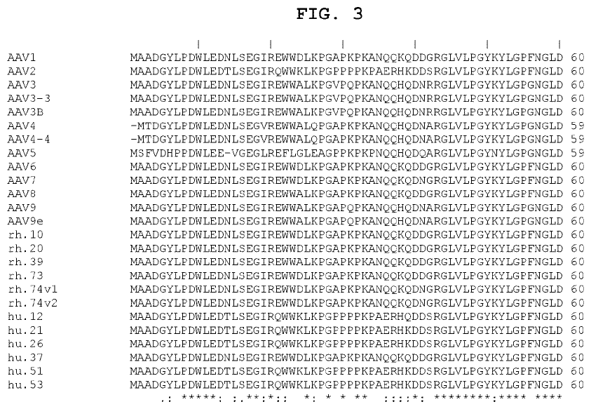

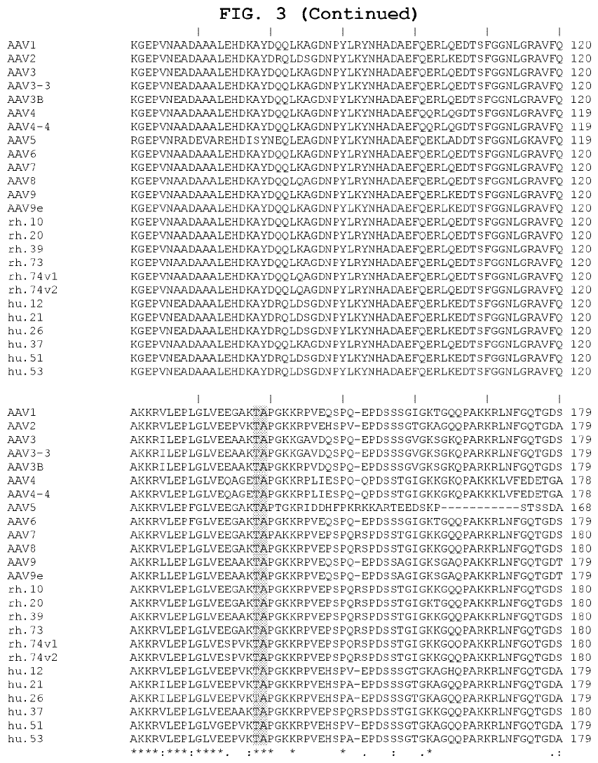

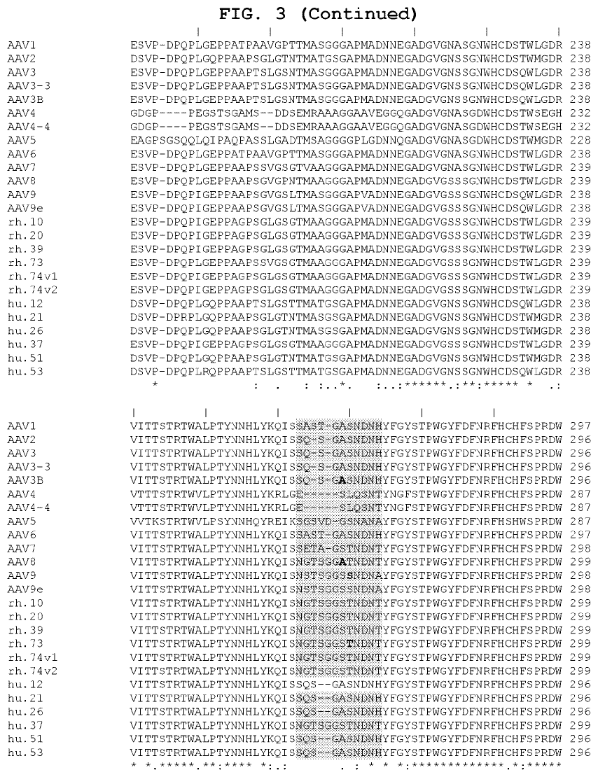

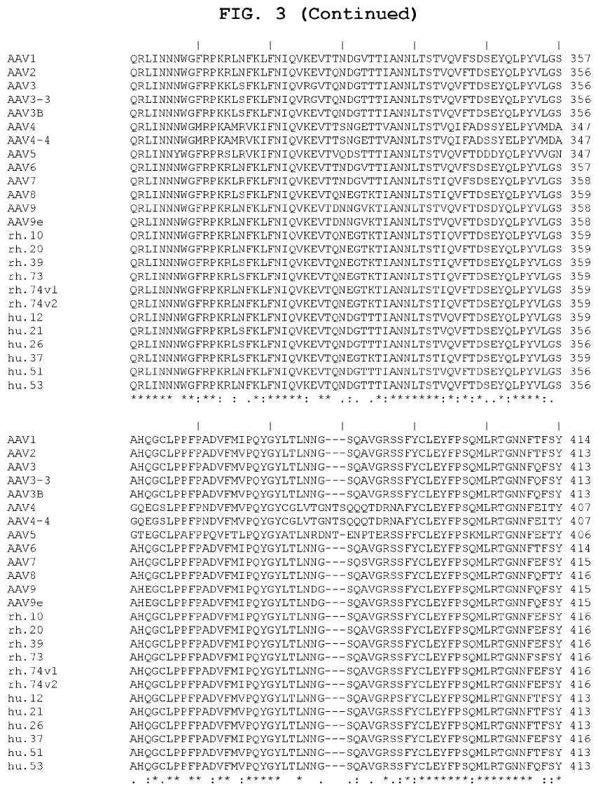

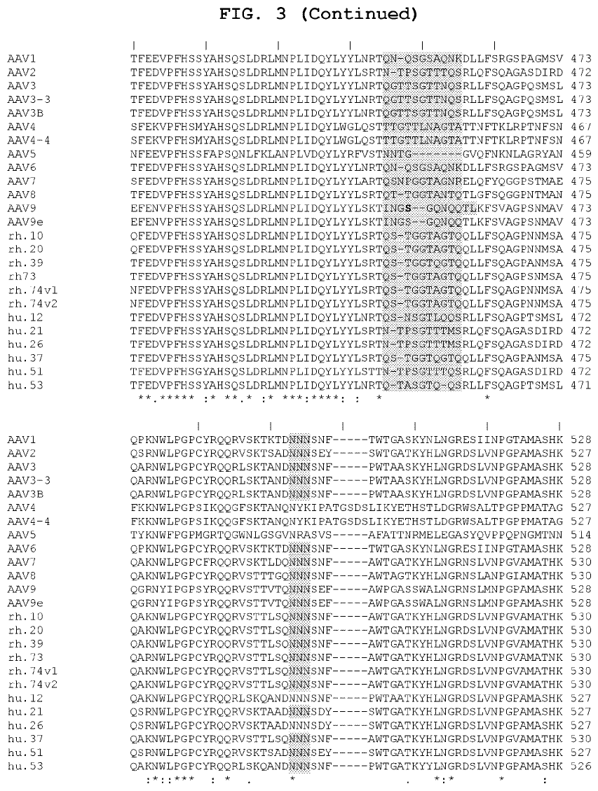

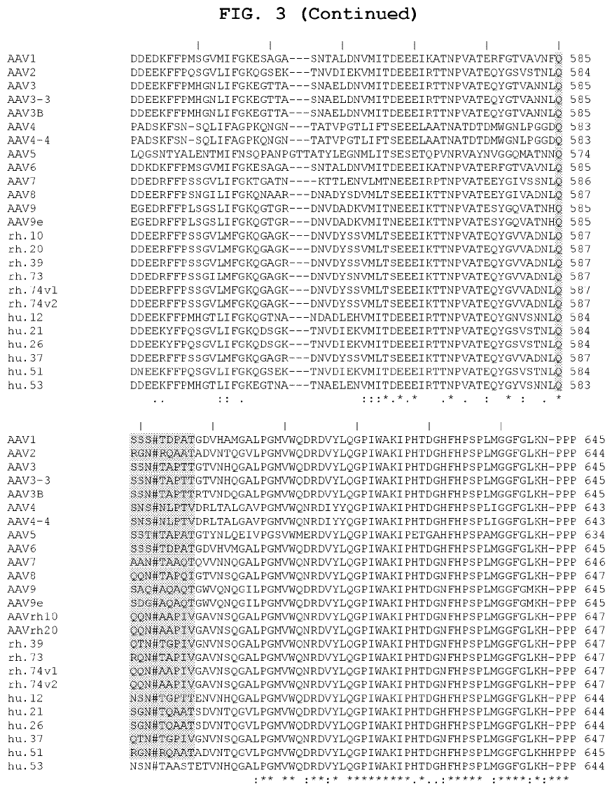

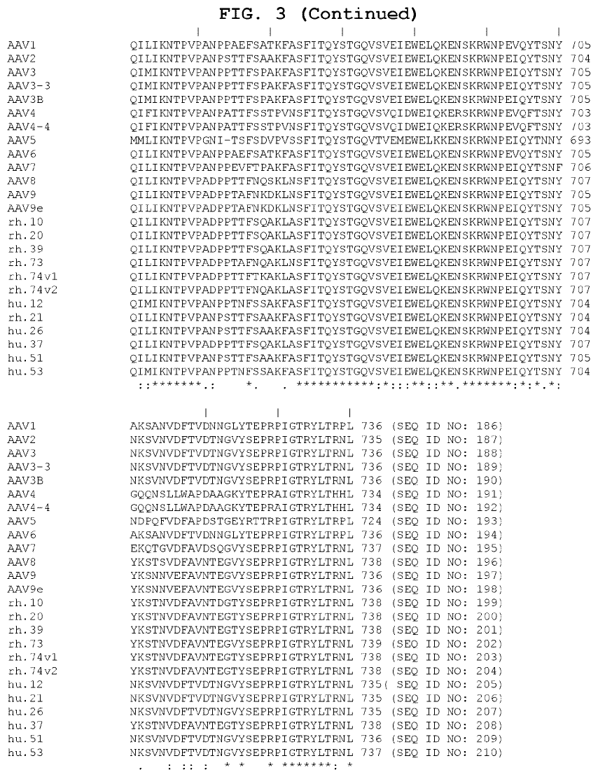

In certain embodiments, the viral vectors provided herein are AAV based viral vectors. In certain embodiments, the AAV-based vectors provided herein do not encode the AAV rep gene (required for replication) and/or the AAV cap gene (required for synthesis of the capsid proteins) (the rep and cap proteins may be provided by the packaging cells in trans). Multiple AAV serotypes have been identified. In certain embodiments, AAV-based vectors provided herein comprise components from one or more serotypes of AAV. In preferred embodiments, AAV-based vectors provided herein comprise components from one or more serotypes of AAV with tropism to ocular tissues, liver and/or muscle. In certain embodiments, AAV based vectors provided herein comprise capsid components from one or more of AAV1, AAV2, AAV3, AAV3B, AAV4, AAV5, AAV6, AAV7, AAV8, AAVrh8, AAV9, AAV9e, AAVrh10, AAVrh20, AAVrh39, AAVhu.37, AAVrh73, AAVrh74, AAV.hu51, AAV.hu21, AAV.hu12, or AAV.hu26. In certain embodiments, AAV based vectors provided herein are or comprise components from one or more of AAV8, AAV3B, AAV9, AAV10, AAVrh73, or AAVrh10 serotypes. Provided are viral vectors in which the capsid protein is a variant of the AAV8 capsid protein (SEQ ID NO: 196), AAV3B capsid protein (SEQ ID NO: 190), or AAVrh73 capsid protein (SEQ ID NO: 202), and the capsid protein is e.g., at least 95%, 96%, 97%, 98%, 99% or 99.9% identical to the amino acid sequence of the AAV8 capsid protein (SEQ ID NO: 196), AAV9 (SEQ ID NO: 197), AAV3B capsid protein (SEQ ID NO:190), or AAVrh73 capsid protein (SEQ ID NO:202), while retaining the biological function of the native capsid. In certain embodiments, the encoded AAV capsid has the sequence of SEQ ID NO: 196 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8, AAV3B, or AAVrh73 capsid. FIG. 3 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV vector comprises an AAV8, AAV3B, or AAVrh73, capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions that are not present at that position in the native AAV capsid sequence as identified in the SUBS row of FIG. 3. Amino acid sequence for AAV8, AAV9, AAV3B, or AAVrh73 capsids are provided in FIG. 3.

The amino acid sequence of hu37 capsid can be found in international application PCT WO 2005/033321 (SEQ ID NO: 88 thereof) and the amino acid sequence for the rh8 capsid can be found in international application PCT WO 03/042397 (SEQ ID NO:97). The amino acid sequence for the rh64R1 sequence is found in WO2006/110689 (a R697W substitution of the Rh.64 sequence, which is SEQ ID NO: 43 of WO 2006/110689).

In some embodiments, AAV-based vectors comprise components from one or more serotypes of AAV. In some embodiments, AAV based vectors provided herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof. In some embodiments, AAV based vectors provided herein comprise components from one or more of AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, AAV.Anc80L65, AAV.7m8, AAV.PHP.B, AAV.PHP.cB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof serotypes. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof.

In particular embodiments, the recombinant AAV for us in compositions and methods herein is AAVS3 (including variants thereof) (see e.g., U.S. patent application No. 20200079821, which is incorporated herein by reference in its entirety). In particular embodiments, rAAV particles comprise the capsids of AAV-LK03 or AAV3B, as described in Puzzo et al., 2017, Sci. Transl. Med. 29 (9): 418, which is incorporated by reference in its entirety. In particular embodiments, the AAV for use in compositions and methods herein is any AAV disclosed in U.S. Pat. No. 10,301,648, such as AAV.rh46 or AAV.rh73. In some embodiments, the recombinant AAV for use in compositions and methods herein is Anc80 or Anc80L65 (see, e.g., Zinn et al., 2015, Cell Rep. 12 (6): 1056-1068, which is incorporated by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLOS One 8 (4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23:857-862 and Georgiadis et al., 2018, Gene Therapy 25:450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001). AAV8-based, AAV3B-based, and AAVrh73-based viral vectors are used in certain of the methods described herein. Nucleotide sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8, AAV3B, AAVrh73, or AAVrh10)-based viral vectors encoding a transgene (e.g., an HuPTM Fab or HuPTM scFv or protein). The amino acid sequences of AAV capsids, including AAV8, AAV3B, AAVrh73 and AAVrh10 are provided in FIG. 3.

In certain embodiments, a single-stranded AAV (ssAAV) may be used supra. In certain embodiments, a self-complementary vector, e.g., scAAV, may be used (see, e.g., Wu, 2007, Human Gene Therapy, 18 (2): 171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the transgene encoding the HuPTMmAb or HuPTM scFv or protein or HuGlyFab or antigen-binding fragment. The recombinant adenovirus can be a first-generation vector, with an E1 deletion, with or without an F3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12: S18-S27, which is incorporated by reference herein in its entirety.

In certain embodiments, the viral vectors used in the methods described herein are lentivirus based viral vectors. A recombinant lentivirus vector may be used to transfer in the transgene encoding the HuPTM mAb antigen binding fragment. Four plasmids are used to make the construct:

Gag/pol sequence containing plasmid, Rev sequence containing plasmids, Envelope protein containing plasmid (e.g., VSV-G), and Cis plasmid with the packaging elements and the anti-C3 or C5 antigen-binding fragment gene or CFHL-1.

For lentiviral vector production, the four plasmids are co-transfected into cells (e.g., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10 (2): 32-43, both of which are incorporated by reference herein in their entireties.

In a specific embodiment, a vector for use in the methods described herein is one that encodes an HuPTM mAb, such that, upon introduction of the vector into a relevant cell, a glycosylated and/or tyrosine sulfated variant of the HuPTM mAb is expressed by the cell.

### 5.1.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters that control expression of the transgene. These promoters (and other regulatory elements that control transcription, such as enhancers) may be constitutive (promote ubiquitous expression) or may specifically or selectively express in the eye. In certain embodiments, the promoter is a constitutive promoter.

In certain embodiments, the promoter is a CAG promoter (SEQ ID NO: 74) (see Dinculescu et al., 2005, Hum Gene Ther 16:649-663, incorporated by reference herein in its entirety). In some embodiments, the CAG (SEQ ID NO: 74) or CB7 promoter (SEQ ID NO: 73) or a mutated CAG promoter (SEQ ID NO: 304, SEQ ID NO: 305 or SEQ ID NO: 306), includes other expression control elements that enhance expression of the transgene driven by the vector. In certain embodiments, the other expression control elements include chicken β-actin intron and/or rabbit β-globin polyA signal (SEQ ID NO: 78). In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs.

In certain embodiments, the vectors provided herein comprise one or more tissue specific promoters (e.g., a retinal-specific promoter). In particular embodiments, the viral vectors provided herein comprises a ocular tissue cell specific promoter, such as, human rhodopsin kinase (GRK1) promoter (SEQ ID NOS: 77 or 217), a mouse cone arresting (CAR) promoter (SEQ ID NOS: 214-216), or a human red opsin (RedO) promoter (SEQ ID NO: 212).

Provided are nucleic acid regulatory elements that are chimeric with respect to arrangements of elements in tandem in the expression cassette. Regulatory elements, in general, have multiple functions as recognition sites for transcription initiation or regulation, coordination with cell-specific machinery to drive expression upon signaling, and to enhance expression of the downstream gene.

In certain embodiments, the promoter is an inducible promoter. In certain embodiments the promoter is a hypoxia-inducible promoter. In certain embodiments, the promoter comprises a hypoxia-inducible factor (HIF) binding site. In certain embodiments, the promoter comprises a HIF-1a binding site. In certain embodiments, the promoter comprises a HIF-2a binding site. In certain embodiments, the HIF binding site comprises an RCGTG (SEQ ID NO: 227) motif. For details regarding the location and sequence of HIF binding sites, see, e.g., Schödel, et al., Blood, 2011, 117 (23): e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety. In specific embodiments, the hypoxia-inducible promoter is the human N-WASP promoter, see, e.g., Salvi, 2017, Biochemistry and Biophysics Reports 9:13-21 (incorporated by reference for the teaching of the N-WASP promoter) or is the hypoxia-induced promoter of human Epo, see, e.g., Tsuchiya et al., 1993, J. Biochem. 113:395-400 (incorporated by reference for the disclosure of the Epo hypoxia-inducible promoter). In other embodiments, the promoter is a drug inducible promoter, for example, a promoter that is induced by administration of rapamycin or analogs thereof. See, e.g., the disclosure of rapamycin inducible promoters in PCT publications WO94/18317, WO 96/20951, WO 96/41865, WO 99/10508, WO 99/10510, WO 99/36553, and WO 99/41258, and U.S. Pat. No. 7,067,526, which are hereby incorporated by reference in their entireties for the disclosure of drug inducible promoters.

Provided herein are constructs containing certain ubiquitous and tissue-specific promoters. Such promoters include synthetic and tandem promoters. Examples and nucleotide sequences of promoters are provided in Tables 1 and 1a below. Table 1 also includes the nucleotide sequences of other regulatory elements useful for the expression cassettes provided herein.

TABLE 1
Promoter and Other Regulatory Element Sequences

| Name/ | |
|---|---|
| SEQ ID NO. | Sequence |
| CAG/CB7 | gacattgattattgactagttattaatagtaatcaattacggggtcattagttcatagcccat |
| SEQ ID NO: 73 | atatggagttccgcgttacataacttacggtaaatggcccgcctggctgaccgcccaacgacc |
| | cccgcccattgacgtcaataatgacgtatgttcccatagtaacgccaatagggactttccatt |
| | gacgtcaatgggtggagtatttacggtaaactgcccacttggcagtacatcaagtgtatcata |
| | tgccaagtacgccccctattgacgtcaatgacggtaaatggcccgcctggcattatgcccagt |
| | acatgaccttatgggactttcctacttggcagtacatctacgtattagtcatcgctattacca |
| | tggtcgaggtgagccccacgttctgcttcactctccccatctcccccccctccccacccccaa |
| | ttttgtatttatttattttttaattattttgtgcagcgatgggggcggggggggggggggggc |
| | gcgcgccaggcggggcggggcggggcgaggggcggggcggggcgaggcggagaggtgcggcgg |
| | cagccaatcagagcggcgcgctccgaaagtttccttttatggcgaggcggcggcggcggcggc |
| | cctataaaaagcgaagcgcgcggcgggcgggagtcgctgcgcgctgccttcgccccgtgcccc |
| | gctccgccgccgcctcgcgccgcccgccccggctctgactgaccgcgttactcccacaggtga |
| | gcgggcgggacggcccttctcctccgggctgtaattagcgcttggtttaatgacggcttgttt |
| | cttttctgtggctgcgtgaaagccttgaggggctccgggagggccctttgtgcggggggagcg |
| | gctcggggggtgcgtgcgtgtgtgtgtgcgtggggagcgccgcgtgcggctccgcgctgcccg |
| | gcggctgtgagcgctgcgggcgcggcgcggggctttgtgcgctccgcagtgtgcgcgagggga |
| | gcgcggccgggggcggtgccccgcggtgcggggggggctgcgaggggaacaaaggctgcgtgc |
| | ggggtgtgtgcgtgggggggtgagcagggggtgtgggcgcgtcggtcgggctgcaaccccccc |
| | tgcacccccctccccgagttgctgagcacggcccggcttcgggtgcggggctccgtacggggc |
| | gtggcgcggggctcgccgtgccgggcggggggtggcggcaggtgggggtgccgggcggggcgg |
| | ggccgcctcgggccggggagggctcgggggaggggcgcggcggcccccggagcgccggcggct |
| | gtcgaggcgcggcgagccgcagccattgccttttatggtaatcgtgcgagagggcgcagggac |
| | ttcctttgtcccaaatctgtgcggagccgaaatctgggaggcgccgccgcaccccctctagcg |
| | ggcgcggggcgaagcggtgcggcgccggcaggaaggaaatgggcggggagggccttcgtgcgt |
| | cgccgcgccgccgtccccttctccctctccagcctcggggctgtccgcggggggacggctgcc |
| | ttcgggggggacggggcagggcggggttcggcttctggcgtgtgaccggcggctctagagcct |
| | ctgctaaccatgttcatgccttcttctttttcctacagctcctgggcaacgtgctggttattg |
| | tgctgtctcatcattttggcaaag |
| CAG (CMV- | gacattgattattgactagttattaatagtaatcaattacggggtcattagttcatagcccat |
| Chicken B-Actin | atatggagttccgcgttacataacttacggtaaatggcccgcctggctgaccgcccaacgacc |
| Promoter- | cccgcccattgacgtcaataatgacgtatgttcccatagtaacgccaatagggactttccatt |
| chimeric intron) | gacgtcaatgggtggagtatttacggtaaactgcccacttggcagtacatcaagtgtatcata |
| SEQ ID NO: 74 | tgccaagtacgccccctattgacgtcaatgacggtaaatggcccgcctggcattatgcccagt |
| | acatgaccttatgggactttcctacttggcagtacatctacgtattagtcatcgctattacca |
| | tggtcgaggtgagccccacgttctgcttcactctccccatctcccccccctccccacccccaa |
| | ttttgtatttatttattttttaattattttgtgcagcgatgggggcggggggggggggggggc |
| | gcgcgccaggcggggcggggcggggcgaggggcggggcggggcgaggcggagaggtgcggcgg |
| | cagccaatcagagcggcgcgctccgaaagtttccttttatggcgaggcggcggcggcggcggc |
| | cctataaaaagcgaagcgcgcggcgggcgggagtcgctgcgcgctgccttcgccccgtgcccc |
| | gctccgccgccgcctcgcgccgcccgccccggctctgactgaccgcgttactcccacaggtga |
| | gcgggcgggacggcccttctcctccgggctgtaattagcgcttggtttaatgacggcttgttt |
| | cttttctgtggctgcgtgaaagccttgaggggctccgggagggccctttgtgcggggggagcg |
| | gctcggggggtgcgtgcgtgtgtgtgtgcgtggggagcgccgcgtgcggctccgcgctgcccg |
| | gcggctgtgagcgctgcgggcgcggcgcggggctttgtgcgctccgcagtgtgcgcgagggga |
| | gcgcggccgggggcggtgccccgcggtgcggggggggctgcgaggggaacaaaggctgcgtgc |
| | ggggtgtgtgcgtgggggggtgagcagggggtgtgggcgcgtcggtcgggctgcaaccccccc |
| | tgcacccccctccccgagttgctgagcacggcccggcttcgggtgcggggctccgtacggggc |
| | gtggcgcggggctcgccgtgccgggcggggggtggcggcaggtgggggtgccgggcggggcgg |
| | ggccgcctcgggccggggagggctcgggggaggggcgcggcggcccccggagcgccggcggct |
| | gtcgaggcgcggcgagccgcagccattgccttttatggtaatcgtgcgagagggcgcagggac |
| | ttcctttgtcccaaatctgtgcggagccgaaatctgggaggcgccgccgcaccccctctagcg |
| | ggcgcggggcgaagcggtgcggcgccggcaggaaggaaatgggcggggagggccttcgtgcgt |
| | cgccgcgccgccgtccccttctccctctccagcctcggggctgtccgcggggggacggctgcc |
| | ttcgggggggacggggcagggcggggttcggcttctggcgtgtgaccggcggctctagagcct |
| | ctgctaaccatgttcatgccttcttctttttcctacag |
| mU1a | atggaggcggtactatgtagatgagaattcaggagcaaactgggaaaagcaactgcttccaaa |
| SEQ ID NO: 75 | tatttgtgatttttacagtgtagttttggaaaaactcttagcctaccaattcttctaagtgtt |
| | ttaaaatgtgggagccagtacacatgaagttatagagtgttttaatgaggcttaaatatttac |
| | cgtaactatgaaatgctacgcatatcatgctgttcaggctccgtggccacgcaactcatact |
| EF-1α (core) | gggcagagcgcacatcgcccacagtccccgagaagttggggggaggggtcggcaattgaacgg |
| SEQ ID NO: 76 | gtgcctagagaaggtggcgcggggtaaactgggaaagtgatgtcgtgtactggctccgccttt |
| | ttcccgaggggggggagaaccgtatataagtgcagtagtcgccgtgaacgttctttttcgca |
| | acgggtttgccgccagaacacag |
| GRK1 promoter | GGGCCCCAGAAGCCTGGTGGTTGTTTGTCCTTCTCAGGGGAAAAGTGAGGCGGCCCCTTGGAG |
| SEQ ID NO: 77 | GAAGGGGCCGGGCAGAATGATCTAATCGGATTCCAAGCAGCTCAGGGGATTGTCTTTTTCTAG |
| | CACCTTCTTGCCACTCCTAAGCGTCCTCCGTGACCCCGGCTGGGATTTAGCCTGGTGCTGTGT |
| | CAGCCCCGGGCTCCCAGGGGCTTCCCAGTGGTCCCCAGGAACCCTCGACAGGGCCAGGGCGTC |
| | TCTCTCGTCCAGCAAGGGCAGGGACGGGCCACAGGCCAAGGGC |
| Rabbit beta- | gatctttttccctctgccaaaaattatggggacatcatgaagccccttgagcatctgacttct |
| globin polyA | ggctaataaaggaaatttattttcattgcaatagtgtgttggaattttttgtgtctctcactc |
| SEQ ID NO: 78 | g |
| Chimeric Intron | gtaagtatcaaggttacaagacaggtttaaggagaccaatagaaactgggcttgtcgagacag |
| SEQ ID NO: 79 | agaagactcttgcgtttctgataggcacctattggtcttactgacatccactttgcctttctc |
| | tccacag |
| VH4 Intron | gtgagtatctcagggatccagacatggggatatgggaggtgcctctgatcccagggctcactg |
| SEQ ID NO: 80 | tgggtctctctgttcacag |
| 5′ITR | ctgcgcgctcgctcgctcactgaggccgcccgggcaaagcccgggcgtcgggcgacctttggt |
| SEQ ID NO: 81 | cgcccggcctcagtgagcgagcgagcgcgcagagagggagtggccaactccatcactaggggt |
| | tcct |
| 5′-ITR | ctgcgcgctcgctcgctcactgaggccgcccgggcaaagcccgggcgtcgggcgacctttggt |
| (Deleted D- | cgcccggcctcagtgagcgagcgagcgcgcagagagggagtgg |
| sequence for self- | |
| complimentary | |
| AAV) | |
| SEQ ID NO: 82 | |
| 3′-ITRAAV | gaacccctagtgatggagttggccactccctctctgcgcgctcgctcgctcactgaggccgcc |
| SEQ ID NO: 83 | cgggcaaagcccgggcgtcgggcgacctttggtcgcccggcctcagtgagcgagcgagcgcgc |
| | agagagggagtggccaa |
| 3′-ITR | ttggccactccctctctgcgcgctcgctcgctcactgaggccgggcgaccaaaggtcgcccga |
| (Deleted D- | cgcccgggctttgcccgggcggcctcagtgagcgagcgagcgcgcag |
| sequence for self- | |
| complimentary | |
| AAV) | |
| SEQ ID NO: 84 | |
| Human UbC | Gtctaacaaaaaagccaaaaacggccagaatttagcggacaatttactagtctaacactgaa |
| SEQ ID NO: 211 | Aattacatattgacccaaatgattacatttcaaaaggtgcctaaaaaacttcacaaaacacac |
| | tCgccaaccccgagcgcatagttcaaaaccggagcttcagctacttaagaagataggtacata |
| | aAaccgaccaaagaaactgacgcctcacttatccctcccctcaccagaggtccggcgcctgtc |
| | gaTtcaggagagcctaccctaggcccgaaccctgcgtcctgcgacggagaaaagcctaccgca |
| | caCctaccggcaggtggccccaccctgcattataagccaacagaacgggtgacgtcacgacac |
| | gaCgagggcgcgcgctcccaaaggtacgggtgcactgcccaacggcaccgccataactgccgc |
| | ccCcgcaacagacgacaaaccgagttctccagtcagtgacaaacttcacgtcagggtccccag |
| | atgGtgccccagcccatctcacccgaataagagctttcccgcattagcgaaggcctcaagacc |
| | ttgggTtcttgccgcccaccatgccccccaccttgtttcaacgacctcacagcccgcctcaca |
| | agcgtcttcCattcaagactcgggaacagccgccattttgctgcgctccccccaacccccagt |
| | tcagggcaaccTtgctcgcggacccagactacagcccttggcggtctctccacacgcttccgt |
| | cccaccgagcggccCggcggccacgaaagccccggccagcccagcagcccgctactcaccaag |
| | tgacgatcacagcgaTccacaaacaagaaccgcgacccaaatcccggctgcgacggaactagc |
| | tgtgccacacccggcgCgtccttatataatcatcggcgttcaccgccccacggagatccctcc |
| | gcagaatcgccgagaagggActacttttcctcgcctgttccgctctctggaaagaaaaccagt |
| | gccctagagtcacccaagtcccgtCctaaaatgtccttctgctgatactggggttctaaggcc |
| | gagtcttatgagcagcgggccgctgtcctGagcgtccgggcggaaggatcaggacgctcgctg |
| | cgcccttcgtctgacgtggcagcgctcgccgtgaggaggggggcgcccgcgggaggcgccaaa |
| | acccggcgcggaggcc |
| Human red opsin | ggaggctgaggggtggggaaagggcatgggtgtttcatgaggacagagcttccgtttcat |
| (RedO) | gcaatgaaaagagtttggagacggatggtggtgactggactatacacttacacacggtag |
| (photoreceptor- | cgatggtacactttgtattatgtatattttaccacgatctttttaaagtgtcaaaggcaa |
| specific) | atggccaaatggttccttgtcctatagctgtagcagccatcggctgttagtgacaaagcc |
| SEQ ID NO: 212 | cctgagtcaagatgacagcagcccccataactcctaatcggctctcccgcgtggagtcat |
| | ttaggagtagtcgcattagagacaagtccaacatctaatcttccaccctggccagggccc |
| | cagctggcagcgagggtgggagactccgggcagagcagagggcgctgacattggggcccg |
| | gcctggcttgggtccctctggcctttccccaggggccctctttccttggggctttcttgg |
| | gccgccactgctcccgctcctctccccccatcccaccccctcaccccctcgttcttcata |
| | tccttctctagtgctccctccactttcatccacccttctgcaagagtgtgggaccacaaa |
| | tgagttttcacctggcctggggacacacgtgcccccacaggtgctgagtgactttctagg |
| | acagtaatctgctttaggctaaaatgggacttgatcttctgttagccctaatcatcaatt |
| | agcagagccggtgaaggtgcagaacctaccgcctttccaggcctcctcccacctctgcca |
| | cctccactctccttcctgggatgtgggggctggcacacgtgtggcccagggcattggtgg |
| | gattgcactgagctgggtcattagcgtaatcctggacaagggcagacagggcgagcggag |
| | ggccagctccggggctcaggcaaggctgggggcttcccccagacaccccactectcctct |
| | gctggacccccacttcatagggcacttcgtgttctcaaagggcttccaaatagcatggtg |
| | gccttggatgcccagggaagcctcagagttgcttatctccctctagacagaaggggaatc |
| | tcggtcaagagggagaggtcgccctgttcaaggccacccagccagctcatggcggtaatg |
| | ggacaaggctggccagccatcccaccctcagaagggacccggtggggcaggtgatctcag |
| | aggaggctcacttctgggtctcacattcttggatccggttccaggcctcggccctaaata |
| | gtctccctgggctttcaagagaaccacatgagaaaggaggattcgggctctgagcagttt |
| | caccacccaccccccagtctgcaaatcctgacccgtgggtccacctgccccaaaggcgga |
| | cgcaggacagtagaagggaacagagaacacataaacacagagagggccacagcggctccc |
| | acagtcaccgccaccttcctggcggggatgggtggggcgtctgagtttggttcccagcaa |
| | atccctctgagccgcccttgcgggctcgcctcaggagcaggggagcaagaggtgggagga |
| | ggaggtctaagtcccaggcccaattaagagatcaggtagtgtagggtttgggagctttta |
| | aggtgaagaggcccgggctgatcccacaggccagtataaagcgccgtgaccctcaggtga |
| | tgcgccagggccggctgccgtcggggacagggctttccatagcc |
| Human | Agatcttcccc a cctagccacc tggcaaactg ctccttctct caaaggccca |
| rhodopsin (Rho) | aacatggcct cccagactgc aacccccagg cagtcaggcc ctgtctccac |
| (photoreceptor- | aacctcacag ccaccctgga cggaatctgc ttcttcccac atttgagtcc |
| specific) | tcctcagccc ctgagctcct ctgggcaggg ctgtttcttt ccatctttgt |
| SEQ ID NO: 213 | attcccaggg gcctgcaaat aaatgtttaa tgaacgaaca agagagtgaa |
| | ttccaattcc atgcaacaag gattgggctc ctgggcccta ggctatgtgt |
| | ctggcaccag aaacggaagc tgcaggttgc agcccctgcc ctcatggagc |
| | tcctcctgtc agaggagtgt ggggactgga tgactccaga ggtaacttgt |
| | gggggaacga acaggtaagg ggctgtgtga cgagatgaga gactgggaga |
| | ataaaccaga aagtctctag ctgtccagag gacatagcac agaggcccat |
| | ggtccctatt tcaaacccag gccaccagac tgagctggga ccttgggaca |
| | gacaagtcat gcagaagtta ggggaccttc tectcccttt tcctggatgg |
| | atcctgagta ccttctcctc cctgacctca ggcttcctcc tagtgtcacc |
| | ttggcccctc ttagaagcca attaggccct cagtttctgc agcggggatt |
| | aatatgatta tgaacacccc caatctccca gatgctgatt cagccaggag |
| | cttaggaggg ggaggtcact ttataagggt ctgggggggt cagaacccag |
| | agtc |
| | (start of exon 1 at 5339; See also Bennett, J., Sun, D. and |
| | Kariko, K. Sequence analysis of the 5.34-kb 5′ flanking region |
| | of the human rhodopsin-encoding gene. Gene 167 (1-2), 317-320 |
| | (1995) ) |
| Mouse cone | TGGCCCTGGCATTCCCCTATACTGGGACATAGAACCTTCACAGGACCAAGGGCCTCTCCTCCC |
| arrestin promoter | ATTGATGACTGACTAGGCCATCCTCTAGCTACATAGGTGGTGGAGCCTAGAGTCCCTCCTTGT |
| (CAR) | GTACTCTTTGGTGGTGGTTACTCTGGGGGTACTAGGTTAGTTCGTATTGTTGTTCCTCCTAGG |
| (photoreceptor- | GGACTGCAAACCCCTTCAGCTCCTTGGGTCCTTTCTCTAGTTCCTTCTTTGGGGACCCTGTGC |
| specific) | TCAGTTCAATGGATGGCCAATTTCCTTCTTAAATGCCCCTAGCAGTAACTGTTAGGTCTCAAT |
| SEQ ID NO: 214 | CCCAAGACAAATGTCTGAGGTGCCTATTTAACAGATCAAAGCGGACCTGTCCTCAGGTTAACC |
| | CAGTCACTCCCTGTACCTCAGTCCCTACCCATCACAATTCTCCAGCCCATGAGCTTCGGGCTG |
| | TACTTCCCCAACGGGTTCTCCCATTTTGGGTACATGGCCTTTTTTTTTTACCTTTTTGGTTCC |
| | TTTGGCCTTTTGGCTTTTGGCTTCCAGGGCTTCTGGATCCCCCCCAACCCCTCCCATACACA |
| | TACACATGTGCACTCGTGCACTCAACCCAGCACAGGATAATGTTCATTCTTGACCTTTCCACA |
| | TACATCTGGCTATGTTCTCTCTCTTATCTACAATAAATCTCCTCCACTATACTTAGGAGCAGT |
| | TATGTTCTTCTTCTTTCTTTCTTTTTTTTTTTTTTTTCATTCAGTAACATCATCAGAATCCCC |
| | TAGCTCTGGCCTACCTCCTCAGTAACAATCAGCTGATCCCTGGCCACTAATCTGTACTCACTA |
| | ATCTGTTTTCCAAACTCTTGGCCCCTGAGCTAATTATAGCAGTGCTTCATGCCACCCACCCCA |
| | ACCCTATCCTTGTTCTCTGACTCCCACTAATCTACACATTCAGAGGATTGTGGATATAAGAGG |
| | CTGGGAGGCCAGCTTAGCAACCAGAGCTCGAGGCTGATGCGAGCTTCATCTCTTCCCTC |
| Mouse cone | TTTTTTTTTTACCTTTTTGGTTCCTTTGGCCTTTTGGCTTTTGGCTTCCAGGGCTTCTGGATC |
| arrestin promoter | CCCCCCAACCCCTCCCATACACATACACATGTGCACTCGTGCACTCAACCCAGCACAGGATA |
| (CAR) | ATGTTCATTCTTGACCTTTCCACATACATCTGGCTATGTTCTCTCTCTTATCTACAATAAATC |
| (photoreceptor- | TCCTCCACTATACTTAGGAGCAGTTATGTTCTTCTTCTTTCTTTCTTTTTTTTTTTTTTTTCA |
| specific) | TTCAGTAACATCATCAGAATCCCCTAGCTCTGGCCTACCTCCTCAGTAACAATCAGCTGATCC |
| SEQ ID NO: 215 | CTGGCCACTAATCTGTACTCACTAATCTGTTTTCCAAACTCTTGGCCCCTGAGCTAATTATAG |
| | CAGTGCTTCATGCCACCCACCCCAACCCTATCCTTGTTCTCTGACTCCCACTAATCTACACAT |
| | TCAGAGGATTGTGGATATAAGAGGCTGGGAGGCCAGCTTAGCAACCAGAGCTCGAGGCTGATG |
| | CGAGCTTCATCTCTTCCCTC |
| Mouse cone | TGATCCCTGGCCACTAATCTGTACTCACTAATCTGTTTTCCAAACTCTTGGCCCCTGAGCTAA |
| arrestin promoter | TTATAGCAGTGCTTCATGCCACCCACCCCAACCCTATCCTTGTTCTCTGACTCCCACTAATCT |
| (CAR) | ACACATTCAGAGGATTGTGGATATAAGAGGCTGGGAGGCCAGCTTAGCAACCAGAGCTCGAGG |
| (photoreceptor- | CTGATGCGAGCTTCATCTCTTCCCTC |
| specific) | |
| SEQ ID NO: 216 | |
| Human | gggcccca gaagcctggt ggttgtttgt ccttctcagg ggaaaagtga ggcggcccct |
| rhodopsin kinase | tggaggaagg ggccgggcag aatgatctaa tcggattcca agcagctcag |
| (RK) or GRK1 | gggattgtct ttttctagca ccttcttgcc actcctaagc gtcctccgtg |
| (photoreceptor- | accccggctg ggatttagcc tggtgctgtg tcagccccgg |
| specific) | |
| SEQ ID NO: 217 | |
| Human | AAGG ACTCCTTTGT GGAGGTCCTG GCTTAGGGAG TCAAGTGACG |
| Bestrophin 1 | GCGGCTCAGC ACTCACGTGG GCAGTGCCAG CCTCTAAGAG TGGGCAGGGG |
| (BEST1) | CACTGGCCAC AGAGTCCCAG GGAGTCCCAC CAGCCTAGTC GCCAGACC +38 |
| promoter | |
| (a.k.a. hVMD2 | |
| promoter) | |
| SEQ ID NO: 218 | |
| Human | GTCAAGTGACG GCGGCTCAGC ACTCACGTGG GCAGTGCCAG CCTCTAAGAG |
| Bestrophin 1 | TGGGCAGGGG CACTGGCCAC AGAGTCCCAG GGAGTCCCAC CAGCCTAGTC |
| (BEST1) | GCCAGACC |
| promoter | (transcriptional start site residue underlined at +1) |
| (a.k.a. hVMD2 | |
| promoter) | |
| SEQ ID NO: 219 | |
| Human | CTCAGC ACTCACGTGG GCAGTGCCAG CCTCTAAGAG TGGGCAGGGG CACTGGCCAC |
| Bestrophin 1 | AGAGTCCCAG GGAGTCCCAC CAGCCTAGTC GCCAGAC |
| (BEST1) | (transcriptional start site residue underlined at +1) |
| promoter | |
| (a.k.a. hVMD2 | |
| promoter) | |
| SEQ ID NO: 220 | |
| Human | TTAATAAACA TTTGGGCGAT TCTTACGGCC TCTAAAGACC AAGAACCACT |
| Bestrophin 1 | GCTGCCTAGA GCTCTGCTCT CTTCATTGAA CAATACAAGA GGAGTGTGTA |
| (BEST1) | GGTAGACACC CACCACTTCC AACAGCTTAG GAGAGCCCTT GAGTATGGAT |
| promoter | TGATGTATTA AAATTTATTG AATCACATGC TGAGATTTTC ACCAGCTGCC |
| (a.k.a. hVMD2 | CGTGGGGATC TGGGCATTTA TTCCCATATT GCACTGGCTG GCTGGAAGCC |
| promoter) | AGCAGCATAA ACTCCAGGGC TGTTCTGTCA ACCCCCACCA GACTCACCCC |
| SEQ ID NO: 221 | GCTCCACCAG CCCCGGCAGG CTTCTCCTTC CATCTCTCTG AAGCAACTTA |
| | CTGATGGGCC CTGCCAGCCA ATCACAGCCA GAATAACGTA TGATGTCACC |
| | AGCAGCCAAT CAGAGCTCCT CGTCAGCATA TGCAGAATTC TGTCATTTTA |
| | CTAGGGTGAT GAAATTCCCA AGCAACACCA TCCTTTTCAGATAAGGGCAC |
| | TGAGGCTGAG AGAGGAGCTG AAACCTACCC GGCGTCACCA CACACAGGTG |
| | GCAAGGCTGG GACCAGAAAC CAGGACTGTT GACTGCAGCC CGGTATTCAT |
| | TCTTTCCATA GCCCACAGGG CTGTCAAAGA CCCCAGGGCC TAGTCAGAGG |
| | CTCCTCCTTC CTGGAGAGTT CCTGGCACAG AAGTTGAAGC TCAGCACAGC |
| | CCCCTAACCC CCAACTCTCT CTGCAAGGCC TCAGGGGTCA GAACACTGGT |
| | GGAGCAGATC CTTTAGCCTC TGGATTTTAG GGCCATGGTA GAGGGGGTGT |
| | TGCCCTAAAT TCCAGCCCTG GTCTCAGCCC AACACCCTCC AAGAAGAAAT |
| | TAGAGGGGCC ATGGCCAGGC TGTGCTAGCC GTTGCTTCTG AGCAGATTAC |
| | AAGAAGGGAC CAAGACAAGG ACTCCTTTGT GGAGGTCCTG GCTTAGGGAG |
| | TCAAGTGACG GCGGCTCAGC ACTCACGTGG GCAGTGCCAG CCTCTAAGAG |
| | TGGGCAGGGG CACTGGCCAC AGAGTCCCAG GGAGTCCCAC CAGCCTAGTC |
| | GCCAGACCTT CTGTGGGATC ATCGGACCCA |
| | (transcriptional start site residue underlined at +1; minimal |
| | active promoter region in bold) |
| CB Promoter | gacgacattgattattgactagttattaatagtaatcaattacggggtcattagttcatagcc |
| SEQ ID NO: 222 | catatatggagttccgcgttacataacttacggtaaatggcccgcctggctgaccgcccaacg |
| | acccccgcccattgacgtcaataatgacgtatgttcccatagtaacgccaatagggactttcc |
| | attgacgtcaatgggtggagtatttacggtaaactgcccacttggcagtacatcaagtgtatc |
| | atatgccaagtacgccccctattgacgtcaatgacggtaaatggcccgcctggcattatgccc |
| | agtacatgaccttatgggactttcctacttggcagtacatctacgtattagtcatcgctatta |
| | ccatggtcgaggtgagccccacgttctgcttcactctccccatctcccccccctccccacccc |
| | caattttgtatttatttattttttaattattttgtgcagcgatgggggcgggggggggggggg |
| | ggcgcgcgccaggcggggcggggcggggcgaggggcggggcggggcgaggcggagaggtgcgg |
| | cggcagccaatcagagcggcgcgctccgaaagtttccttttatggcgaggcggcggcggcggc |
| | ggccctataaaaagcgaagcgcgcggcgggcgg |
| CB Long | gacgacattgattattgactagttattaatagtaatcaattacggggtcattagttcatagcc |
| Promoter SEQ | catatatggagttccgcgttacataacttacggtaaatggcccgcctggctgaccgcccaacg |
| ID NO: 223 | acccccgcccattgacgtcaataatgacgtatgttcccatagtaacgccaatagggactttcc |
| | attgacgtcaatgggtggagtatttacggtaaactgcccacttggcagtacatcaagtgtatc |
| | atatgccaagtacgccccctattgacgtcaatgacggtaaatggcccgcctggcattatgccc |
| | agtacatgaccttatgggactttcctacttggcagtacatctacgtattagtcatcgctatta |
| | ccatggtcgaggtgagccccacgttctgcttcactctccccatctcccccccctccccacccc |
| | caattttgtatttatttattttttaattattttgtgcagcgatgggggcgggggggggggggg |
| | ggcgcgcgccaggcggggcggggcggggcgaggggcggggcggggcgaggcggagaggtgcgg |
| | cggcagccaatcagagcggcgcgctccgaaagtttccttttatggcgaggcggcggcggcggc |
| | ggccctataaaaagcgaagcgcgcggcgggcgggagtcgctgcgcgctgccttcgccccgtgc |
| | cccgctccgccgccgcctcgcgccgcccgccccggctctgactgaccgcgttactcccacagg |
| | tgagcg |
| Best/GRK1 | GTCGACAAATTCTGTCATTTTACTAGGGTGATGAAATTCCCAAGCAACACCATCCTTTTCAGA |
| Tandem | TAAGGGCACTGAGGCTGAGAGAGGAGCTGAAACCTACCCGGGGTCACCACACACAGGTGGCAA |
| Promoter | GGCTGGGACCAGAAACCAGGACTGTTGACTGCAGCCCGGTATTCATTCTTTCCATAGCCCACA |
| SEQ ID NO: 224 | GGGCTGTCAAAGACCCCAGGGCCTAGTCAGAGGCTCCTCCTTCCTGGAGAGTTCCTGGCACAG |
| | AAGTTGAAGCTCAGCACAGCCCCCTAACCCCCAACTCTCTCTGCAAGGCCTCAGGGGTCAGAA |
| | CACTGGTGGAGCAGATCCTTTAGCCTCTGGATTTTAGGGCCATGGTAGAGGGGGTGTTGCCCT |
| | AAATTCCAGCCCTGGTCTCAGCCCAACACCCTCCAAGAAGAAATTAGAGGGGCCATGGCCAGG |
| | CTGTGCTAGCCGTTGCTTCTGAGCAGATTACAAGAAGGGACTAAGACAAGGACTCCTTTGTGG |
| | AGGTCCTGGCTTAGGGAGTCAAGTGACGGCGGCTCAGCACTCACGTGGGCAGTGCCAGCCTCT |
| | AAGAGTGGGCAGGGGCACTGGCCACAGAGTCCCAGGGAGTCCCACCAGCCTAGTCGCCAGAtC |
| | TAGAGGGCCCCAGAAGCCTGGTGGTTGTTTGTCCTTCTCAGGGGAAAAGTGAGGCGGCCCCTT |
| | GGAGGAAGGGGCCGGGCAGAAGATCTAATCGGATTCCAAGCAGCTCAGGGGATTGTCTTTTTC |
| | TAGCACCTTCTTGCCACTCCTAAGCGTCCTCCGTGACCCCGGCTGGGATTTAGCCTGGTGCTG |
| | TGTCAGCCCCGGGCTCCCAGGGGCTTCCCAGTGGTCCCCAGGAACCCTCGACAGGGCCAGGGC |
| | GTCTCTCTCGTCCAGCAAGGGCAGGGACGGGCCACAGGCCAAGGGCCTTAAGC |
| SV40 Intron | gtaagtttagtctttttgtcttttatttcaggtcccggatccggtggtggtgcaaatcaaaga |
| SEQ ID NO: 225 | actgctcctcagtggatgttgcctttacttctag |
| Upstream Kozak | GCCACC |
| sequence | |
| SEQ ID NO: 25 | |
| CAG(DEL5) | gactagttattaatagtaatcaattacggggtcattagttcatagcccatatatggagttccg |
| SEQ ID NO: 304 | cgttacataacttacggtaaatggcccgcctggctgaccgcccaacgacccccgcccattgac |
| | gtcaataatgacgtatgttcccatagtaacgccaatagggactttccattgacgtcaatgggt |
| | ggagtatttacggtaaactgcccacttggcagtacatcaagtgtatcatatgccaagtacgcc |
| | ccctattgacgtcaatgacggtaaatggcccgcctggcattatgcccagtacatgaccttatg |
| | ggactttcctacttggcagtacatctacgtattagtcatcgctattaccatggtcgaggtgag |
| | ccccacgttctgcttcactctccccatctcccccccctccccacccccaattttgtatttatt |
| | tattttttaattattttgtgcagcgatgggggcggggggggggggggggcgcgcgccaggcgg |
| | ggcggggcggggcgaggggcggggcggggcgaggcggagaggtgcggcggcagccaatcagag |
| | cggcgcgctccgaaagtttccttttatggcgaggcggcggcggcggcggccctataaaaagcg |
| | aagcgcgcggcgggcgggagtcgctgcgcgctgccttcgccccgtgccccgctccgccgccgc |
| | ctcgcgccgcccgccccggctctgactgaccgcgttactcccacaggtgagcgggcgggacgg |
| | cccttctcctccgggctgtgcgagccgcagccattgccttttatggtaatcgtgcgagagggc |
| | gcagggacttcctttgtcccaaatctgtgcggagccgaaatctgggaggcgccgccgcacccc |
| | ctctagcgggcgcggggcgaagcggtgcggcgccggcaggaaggaaatgggcggggagggcct |
| | tcgtgcgtcgccgcgccgccgtccccttctccctctccagcctcggggctgtccgcgggggga |
| | cggctgccttcgggggggacggggcagggggggttcggcttctggcgtgtgaccggcggctc |
| | tagagcctctgctaaccatgttcatgccttcttctttttcctacagctcctgggcaacgtgct |
| | ggttattgtgctgtctcatcattttggcaaag |
| CAG(Delm) | gactagttattaatagtaatcaattacggggtcattagttcatagcccatatatggagttccg |
| SEQ ID NO: 305 | cgttacataacttacggtaaatggcccgcctggctgaccgcccaacgacccccgcccattgac |
| | gtcaataatgacgtatgttcccatagtaacgccaatagggactttccattgacgtcaatgggt |
| | ggagtatttacggtaaactgcccacttggcagtacatcaagtgtatcatatgccaagtacgcc |
| | ccctattgacgtcaatgacggtaaatggcccgcctggcattatgcccagtacatgaccttatg |
| | ggactttcctacttggcagtacatctacgtattagtcatcgctattaccatggtcgaggtgag |
| | ccccacgttctgcttcactctccccatctcccccccctccccacccccaattttgtatttatt |
| | tattttttaattattttgtgcagcgatgggggcggggggggggggggggcgcgcgccaggcgg |
| | ggcggggcggggcgaggggcggggcggggcgaggcggagaggtgcggcggcagccaatcagag |
| | cggcgcgctccgaaagtttccttttatggcgaggcggcggcggcggcggccctataaaaagcg |
| | aagcgcgcggcgggcgggagtcgctgcgcgctgccttcgccccgtgccccgctccgccgccgc |
| | ctcgcgccgcccgccccggctctgactgaccgcgttactcccacaggtgagcgggcgggacgg |
| | cccttctcctccgggctgtaattagcgcttggtttaatgacggcttgtttcttttctgtggct |
| | gcgtgaaagccttgaggggctccgggagggccctttgtgcggggggagcggctcggggggtgc |
| | gtgcgtgtgtgtgtgcgtggggagcgccgcgtgcggctccgaatgggcggggagggccttcgt |
| | gcgtcgccgcgccgccgtccccttctccctctccagcctcggggctgtccgcggggggacggc |
| | tgccttcgggggggacggggcagggcggggttcggcttctggcgtgtgaccggcggctctaga |
| | gcctctgctaaccatgttcatgccttcttctttttcctacagctcctgggcaacgtgctggtt |
| | attgtgctgtctcatcattttggcaaag |
| CAG(Del3) | gactagttattaatagtaatcaattacggggtcattagttcatagcccatatatggagttccg |
| SEQ ID NO: 306 | cgttacataacttacggtaaatggcccgcctggctgaccgcccaacgacccccgcccattgac |
| | gtcaataatgacgtatgttcccatagtaacgccaatagggactttccattgacgtcaatgggt |
| | ggagtatttacggtaaactgcccacttggcagtacatcaagtgtatcatatgccaagtacgcc |
| | ccctattgacgtcaatgacggtaaatggcccgcctggcattatgcccagtacatgaccttatg |
| | ggactttcctacttggcagtacatctacgtattagtcatcgctattaccatggtcgaggtgag |
| | ccccacgttctgcttcactctccccatctcccccccctccccacccccaattttgtatttatt |
| | tattttttaattattttgtgcagcgatgggggcggggggggggggggggcgcgcgccaggcgg |
| | ggcggggcggggcgaggggcggggcggggcgaggcggagaggtgcggcggcagccaatcagag |
| | cggcgcgctccgaaagtttccttttatggcgaggcggcggcggcggcggccctataaaaagcg |
| | aagcgcgcggcgggcgggagtcgctgcgcgctgccttcgccccgtgccccgctccgccgccgc |
| | ctcgcgccgcccgccccggctctgactgaccgcgttactcccacaggtgagcgggcgggacgg |
| | cccttctcctccgggctgtaattagcgcttggtttaatgacggcttgtttcttttctgtggct |
| | gcgtgaaagccttgaggggctccgggagggccctttgtgcggggggagcggctcggggggtgc |
| | gtgcgtgtgtgtgtgcgtggggagcgccgcgtgcggctccgcgctgcccggcggctgtgagcg |
| | ctgcgggcgcggcgcggggctttgtgcgctccgcagtgtgcgcgaggggagcgcggccggggg |
| | cggtgccccgcggtgcggggggggctgcgaggggaacaaaggctgcgtgcgggctctagagcc |
| | tctgctaaccatgttcatgccttcttctttttcctacagctcctgggcaacgtgctggttatt |
| | gtgctgtctcatcattttggcaaag |

TABLE 1a
Other regulatory sequences

| | |
|---|---|
| LSPX1 | aggttaatttttaaaaagcagtcaaaagtccaagtggcccttggcagcatttactctctctgt |
| SEQ ID NO: 228 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgtctagaaggctc |
| | agaggcacacaggagtttctgggctcaccctgcccccttccaacccctcagttcccatcctcc |
| | agcagctgtttgtgtgctgcctctgaagtccacactgaacaaacttcagcctactcatgtccc |
| | taaaatgggcaaacattgcaagcagcaaacagcaaacacacagccctccctgcctgctgacct |
| | tggagctggggcagaggtcagagacctctctgggcccatgccacctccaacatccactcgacc |
| | ccttggaatttcggtggagaggagcagaggttgtcctggcgtggtttaggtagtgtgagaggg |
| | gtacccggggatottgctaccagtggaacagccactaaggattctgcagtgagagcagagggc |
| | cagctaagtggtactctcccagagactgtctgactcacgccaccccctccaccttggacacag |
| | gacgctgtggtttctgagccaggtacaatgactcctttcggtaagtgcagtggaagctgtaca |
| | ctgcccaggcaaagcgtccgggcagcgtaggcgggcgactcagatcccagccagtggacttag |
| | cccctgtttgctcctccgataactggggtgaccttggttaatattcaccagcagcctcccccg |
| | ttgcccctctggatccactgcttaaatacggacgaggacagggccctgtctcctcagcttcag |
| | gcaccaccactgacctgggacagt |
| LSXP2 | aggctcagaggcacacaggagtttctgggctcaccctgcccccttccaacccctcagttccca |
| SEQ ID NO: 229 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagaaggctcagaggcacacaggagtttctgggctcaccctgcccccttccaacc |
| | cctcagttcccatcctccagcagctgtttgtgtgctgcctctgaagtccacactgaacaaact |
| | tcagcctactcatgtccctaaaatgggcaaacattgcaagcagcaaacagcaaacacacagcc |
| | ctccctgcctgctgaccttggagctggggcagaggtcagagacctctctgggcccatgccacc |
| | tccaacatccactcgaccccttggaatttcggtggagaggagcagaggttgtcctggcgtggt |
| | ttaggtagtgtgagaggggtacccggggatottgctaccagtggaacagccactaaggattct |
| | gcagtgagagcagagggccagctaagtggtactctcccagagactgtctgactcacgccaccc |
| | cctccaccttggacacaggacgctgtggtttctgagccaggtacaatgactcctttcggtaag |
| | tgcagtggaagctgtacactgcccaggcaaagcgtccgggcagcgtaggcgggcgactcagat |
| | cccagccagtggacttagcccctgtttgctcctccgataactggggtgaccttggttaatatt |
| | caccagcagcctcccccgttgcccctctggatccactgcttaaatacggacgaggacagggcc |
| | ctgtctcctcagcttcaggcaccaccactgacctgggacagt |
| LTP1 | aggttaatttttaaaaagcagtcaaaagtccaagtggcccttggcagcatttactctctctgt |
| SEQ ID NO: 230 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgacatcatttcct |
| | ctgcgaatgcatgtataatttctacagaacctattagaaaggatcacccagcctctgcttttg |
| | tacaactttcccttaaaaaactgccaattccactgctgtttggcccaatagtgagaacttttt |
| | cctgctgcctcttggtgcttttgcctatggcccctattctgcctgctgaagacactcttgcca |
| | gcatggacttaaacccctccagctctgacaatcctctttctcttttgttttacatgaagggtc |
| | tggcagccaaagcaatcactcaaagttcaaaccttatcattttttgctttgttcctcttggcc |
| | ttggttttgtacatcagctttgaaaataccatcccagggttaatgctggggttaatttataac |
| | taagagtgctctagttttgcaatacaggacatgctataaaaatggaaagatgttgctttctga |
| | gaggatcttgctaccagtggaacagccactaaggattctgcagtgagagcagagggccagcta |
| | agtggtactctcccagagactgtctgactcacgccaccccctccaccttggacacaggacgct |
| | gtggtttctgagccaggtacagtgactcctttcggtaagtgcagtggaagctgtacactgccc |
| | aggcaaagcgtccgggcagcgtaggcgggcgactcagatcccagccagtggacttagcccctg |
| | tttgctcctccgataactggggtgaccttggttaatattcaccagcagcctcccccgttgccc |
| | ctctggatccactgcttaaatacggacgaggacagggccctgtctcctcagcttcaggcacca |
| | ccactgacctgggacagt |
| LTP2 | aggctcagaggcacacaggagtttctgggctcaccctgcccccttccaacccctcagttccca |
| SEQ ID NO: 231 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagagcccttaagctagcaggttaatttttaaaaagcagtcaaaagtccaagtgg |
| | cccttggcagcatttactctctctgtttgctctggttaataatctcaggagcacaaacattcc |
| | agatccaggttaatttttaaaaagcagtcaaaagtccaagtggcccttggcagcatttactct |
| | ctctgtttgctctggttaataatctcaggagcacaaacattccagatccggcgcgccagggct |
| | ggaagctacctttgacatcatttcctctgcgaatgcatgtataatttctacagaacctattag |
| | aaaggatcacccagcctctgcttttgtacaactttcccttaaaaaactgccaattccactgct |
| | gtttggcccaatagtgagaactttttcctgctgcctcttggtgcttttgcctatggcccctat |
| | tctgcctgctgaagacactcttgccagcatggacttaaacccctccagctctgacaatcctct |
| | ttctcttttgttttacatgaagggtctggcagccaaagcaatcactcaaagttcaaaccttat |
| | cattttttgctttgttcctcttggccttggttttgtacatcagctttgaaaataccatcccag |
| | ggttaatgctggggttaatttataactaagagtgctctagttttgcaatacaggacatgctat |
| | aaaaatggaaagatgttgctttctgagaggatcttgctaccagtggaacagccactaaggatt |
| | ctgcagtgagagcagagggccagctaagtggtactctcccagagactgtctgactcacgccac |
| | cccctccaccttggacacaggacgctgtggtttctgagccaggtacagtgactcctttcggta |
| | agtgcagtggaagctgtacactgcccaggcaaagcgtccgggcagcgtaggcgggcgactcag |
| | atcccagccagtggacttagcccctgtttgctcctccgataactggggtgaccttggttaata |
| | ttcaccagcagcctcccccgttgcccctctggatccactgcttaaatacggacgaggacaggg |
| | ccctgtctcctcagcttcaggcaccaccactgacctgggacagt |
| LTP3 | aggttaatttttaaaaagcagtcaaaagtccaagtggcccttggcagcatttactctctctgt |
| SEQ ID NO: 232 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgacatcatttcct |
| | ctgcgaatgcatgtataatttctacagaacctattagaaaggatcacccagcctctgcttttg |
| | tacaactttcccttaaaaaactgccaattccactgctgtttggcccaatagtgagaacttttt |
| | cctgctgcctcttggtgcttttgcctatggcccctattctgcctgctgaagacactcttgcca |
| | gcatggacttaaacccctccagctctgacaatcctctttctcttttgttttacatgaagggtc |
| | tggcagccaaagcaatcactcaaagttcaaaccttatcattttttgctttgttcctcttggcc |
| | ttggttttgtacatcagctttgaaaataccatcccagggttaatgctggggttaatttataac |
| | taagagtgctctagttttgcaatacaggacatgctataaaaatggaaagatgttgctttctga |
| | gaggatcttgctaccagtggaacagccactaaggattctgcagtgagagcagagggccagcta |
| | agtggtactctcccagagactgtctgactcacgccaccccctccaccttggacacaggacgct |
| | gtggtttctgagccaggtacagtgactcctttcggtaagtgcagtggaagctgtacactgccc |
| | aggcaaagcgtccgggcagcgtaggcgggcgactcagatcccagccagtggacttagcccctg |
| | tttgctcctccgataactggggtgaccttggttaatattcaccagcagcctcccccgttgccc |
| | ctctggatccactgcttaaatacggacgaggacagggccctgtctcctcagcttcaggcacca |
| | ccactgacctgggacagtaaaacaggtaagtccgctgtttgtgtgctgcctctgaagtccaca |
| | ctgaacaaacttcagcctactcatgtccctaaaatgggcaaacattgcaagcagcaaacagca |
| | aacacacagccctccctgcctgctgaccttggagctggggcagaggtcagagacctctctggc |
| | ctctactaaccatgttcatgttttctttttttttctacaggtcctgggtgacgaacag |
| LMTP6 | aggctcagaggcacacaggagtttctgggctcaccctgcccccttccaacccctcagttccca |
| SEQ ID NO: 233 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccactacgggtttaggctgcccatgtaaggaggcaaggcctggggacacccgagatg |
| | cctggttataattaacccagacatgtggctgccccccccccccccaacacctgctgcctctaa |
| | aaataaccctgtccctggtggatcccactacgggtttaggctgcccatgtaaggaggcaaggc |
| | ctggggacacccgagatgcctggttataattaacccagacatgtggctgcccccccccccccc |
| | aacacctgctgcctctaaaaataaccctgtccctggtggatcccactacgggtttaggctgcc |
| | catgtaaggaggcaaggcctggggacacccgagatgcctggttataattaacccagacatgtg |
| | gctgccccccccccccccaacacctgctgcctctaaaaataaccctgtccctggtggatcccc |
| | tgcatgcgaagatcttcgaacaaggctgtgggggactgagggcaggctgtaacaggcttgggg |
| | gccagggcttatacgtgcctgggactcccaaagtattactgttccatgttcccggcgaagggc |
| | cagctgtcccccgccagctagactcagcacttagtttaggaaccagtgagcaagtcagccctt |
| | ggggcagcccatacaaggccatggggctgggcaagctgcacgcctgggtccggggtgggcacg |
| | gtgcccgggcaacgagctgaaagctcatctgctctcaggggcccctccctggggacagcccct |
| | cctggctagtcacaccctgtaggctcctctatataacccaggggcacaggggctgccctcatt |
| | ctaccaccacctccacagcacagacagacactcaggagccagccagcgtcgagatcttgctac |
| | cagtggaacagccactaaggattctgcagtgagagcagagggccagctaagtggtactctccc |
| | agagactgtctgactcacgccaccccctccaccttggacacaggacgctgtggtttctgagcc |
| | aggtacagtgactcctttcggtaagtgcagtggaagctgtacactgcccaggcaaagcgtccg |
| | ggcagcgtaggcgggcgactcagatcccagccagtggacttagcccctgtttgctcctccgat |
| | aactggggtgaccttggttaatattcaccagcagcctcccccgttgcccctctggatccactg |
| | cttaaatacggacgaggacagggccctgtctcctcagcttcaggcaccaccactgacctggga |
| | cagt |
| LMTP13 | aggctcagaggcacacaggagtttctgggctcaccctgcccccttccaacccctcagttccca |
| SEQ ID NO: 234 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agaggggtacccggggatcttgctaccagtctagaggccgtccgccctcggcaccatcctcac |
| | gacacccaaatatggcgacgggtgaggaatggtggggagttatttttagagcggtgaggaagg |
| | tgggcaggcagcaggtgttggcgctctaaaaataactcccgggagttatttttagagcggagg |
| | aatggtggacacccaaatatggcgacggttcctcacccgtcgccatatttgggtgtccgccct |
| | cggccggggccgcattcctgggggccgggcggtgctcccgcccgcctcgataaaaggctccgg |
| | ggccggcggcggcccacgagctacccggaggagcgggaggcgccaagcgtgagtatcgatctt |
| | gctaccagtggaacagccactaaggattctgcagtgagagcagagggccagctaagtggtact |
| | ctcccagagactgtctgactcacgccaccccctccaccttggacacaggacgctgtggtttct |
| | gagccaggtacagtgactcctttcggtaagtgcagtggaagctgtacactgcccaggcaaagc |
| | gtccgggcagcgtaggcgggcgactcagatcccagccagtggacttagcccctgtttgctcct |
| | ccgataactggggtgaccttggttaatattcaccagcagcctcccccgttgcccctctggatc |
| | cactgcttaaatacggacgaggacagggccctgtctcctcagcttcaggcaccaccactgacc |
| | tgggacagt |
| LMTP14 | gaatggtggacacccaaatatggcgacggttcctcacccgtcgccatatttgggtgtccgccc |
| SEQ ID NO: 235 | tcggccggggccgcattcctgggggccgggcggtgctcccgcccgcctcgataaaaggctccg |
| | gggccggcggcggcccacgagctacccggaggagcgggaggcgccaagcgatcttgctaccag |
| | tggaacagccactaaggattctgcagtgagagcagagggccagctaagtggtactctcccaga |
| | gactgtctgactcacgccaccccctccaccttggacacaggacgctgtggtttctgagccagg |
| | tacagtgactcctttcggtaagtgcagtggaagctgtacactgcccaggcaaagcgtccgggc |
| | agcgtaggcgggcgactcagatcccagccagtggacttagcccctgtttgctcctccgataac |
| | tggggtgaccttggttaatattcaccagcagcctcccccgttgcccctctggatccactgctt |
| | aaatacggacgaggacagggccctgtctcctcagcttcaggcaccaccactgacctgggacag |
| | t |
| LMTP15 | aggctcagaggcacacaggagtttctgggctcaccctgcccccttccaacccctcagttccca |
| SEQ ID NO: 236 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagagaatggtggacacccaaatatggcgacggttcctcacccgtcgccatattt |
| | gggtgtccgccctcggccggggccgcattcctgggggccgggcggtgctcccgcccgcctcga |
| | taaaaggctccggggccggcggcggcccacgagctacccggaggagcgggaggcgccaagcga |
| | tcttgctaccagtggaacagccactaaggattctgcagtgagagcagagggccagctaagtgg |
| | tactctcccagagactgtctgactcacgccaccccctccaccttggacacaggacgctgtggt |
| | ttctgagccaggtacagtgactcctttcggtaagtgcagtggaagctgtacactgcccaggca |
| | aagcgtccgggcagcgtaggcgggcgactcagatcccagccagtggacttagcccctgtttgc |
| | tcctccgataactggggtgaccttggttaatattcaccagcagcctcccccgttgcccctctg |
| | gatccactgcttaaatacggacgaggacagggccctgtctcctcagcttcaggcaccaccact |
| | gacctgggacagt |
| LMTP18 | aggctcagaggcacacaggagtttctgggctcaccctgcccccttccaacccctcagttccca |
| SEQ ID NO: 237 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccactacgggtttaggctgcccatgtaaggaggcaaggcctggggacacccgagatg |
| | cctggttataattaacccagacatgtggctgccccccccccccccaacacctgctgcctctaa |
| | aaataaccctgtccctggtggatcccctgcatgcgaagatcttcgaacaaggctgtgggggac |
| | tgagggcaggctgtaacaggcttgggggccagggcttatacgtgcctgggactcccaaagtat |
| | tactgttccatgttcccggcgaagggccagctgtcccccgccagctagactcagcacttagtt |
| | taggaaccagtgagcaagtcagcccttggggcagcccatacaaggccatggggctgggcaagc |
| | tgcacgcctgggtccggggtgggcacggtgcccgggcaacgagctgaaagctcatctgctctc |
| | aggggcccctccctggggacagcccctcctggctagtcacaccctgtaggctcctctatataa |
| | cccaggggcacaggggctgccctcattctaccaccacctccacagcacagacagacactcagg |
| | agccagccagcgtcgagatcttgctaccagtggaacagccactaaggattctgcagtgagagc |
| | agagggccagctaagtggtactctcccagagactgtctgactcacgccaccccctccaccttg |
| | gacacaggacgctgtggtttctgagccaggtacagtgactcctttcggtaagtgcagtggaag |
| | ctgtacactgcccaggcaaagcgtccgggcagcgtaggcgggcgactcagatcccagccagtg |
| | gacttagcccctgtttgctcctccgataactggggtgaccttggttaatattcaccagcagcc |
| | tcccccgttgcccctctggatccactgcttaaatacggacgaggacagggccctgtctcctca |
| | gcttcaggcaccaccactgacctgggacagt |
| LMTP19 | aggctcagaggcacacaggagtttctgggctcaccctgcccccttccaacccctcagttccca |
| SEQ ID NO: 238 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccctgcatgcgaagatcttcgaacaaggctgtgggggactgagggcaggctgtaaca |
| | ggcttgggggccagggcttatacgtgcctgggactcccaaagtattactgttccatgttcccg |
| | gcgaagggccagctgtcccccgccagctagactcagcacttagtttaggaaccagtgagcaag |
| | tcagcccttggggcagcccatacaaggccatggggctgggcaagctgcacgcctgggtccggg |
| | gtgggcacggtgcccgggcaacgagctgaaagctcatctgctctcaggggcccctccctgggg |
| | acagcccctcctggctagtcacaccctgtaggctcctctatataacccaggggcacaggggct |
| | gccctcattctaccaccacctccacagcacagacagacactcaggagccagccagcgtcgaga |
| | tcttgctaccagtggaacagccactaaggattctgcagtgagagcagagggccagctaagtgg |
| | tactctcccagagactgtctgactcacgccaccccctccaccttggacacaggacgctgtggt |
| | ttctgagccaggtacagtgactcctttcggtaagtgcagtggaagctgtacactgcccaggca |
| | aagcgtccgggcagcgtaggcgggcgactcagatcccagccagtggacttagcccctgtttgc |
| | tcctccgataactggggtgaccttggttaatattcaccagcagcctcccccgttgcccctctg |
| | gatccactgcttaaatacggacgaggacagggccctgtctcctcagcttcaggcaccaccact |
| | gacctgggacagt |
| LMTP20 | aggctcagaggcacacaggagtttctgggctcaccctgcccccttccaacccctcagttccca |
| SEQ ID NO: 239 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggcccttcagattaaaaataactgaggtaagggcctgggtaggggaggtggtgtgagac |
| | gctcctgtctctcctctatctgcccatcggccctttggggaggaggaatgtgcccaaggacta |
| | aaaaaaggccatggagccagaggggcgagggcaacagacctttcatgggcaaaccttggggcc |
| | ctgctgaagctttggcccactacgggtttaggctgcccatgtaaggaggcaaggcctggggac |
| | acccgagatgcctggttataattaacccagacatgtggctgccccccccccccccaacacctg |
| | ctgcctctaaaaataaccctgtccctggtggatcccctgcatgcgaagatcttcgaacaaggc |
| | tgtgggggactgagggcaggctgtaacaggcttgggggccagggcttatacgtgcctgggact |
| | cccaaagtattactgttccatgttcccggcgaagggccagctgtcccccgccagctagactca |
| | gcacttagtttaggaaccagtgagcaagtcagcccttggggcagcccatacaaggccatgggg |
| | ctgggcaagctgcacgcctgggtccggggtgggcacggtgcccgggcaacgagctgaaagctc |
| | atctgctctcaggggcccctccctggggacagcccctcctggctagtcacaccctgtaggctc |
| | ctctatataacccaggggcacaggggctgccctcattctaccaccacctccacagcacagaca |
| | gacactcaggagccagccagcgtcgagatcttgctaccagtggaacagccactaaggattctg |
| | cagtgagagcagagggccagctaagtggtactctcccagagactgtctgactcacgccacccc |
| | ctccaccttggacacaggacgctgtggtttctgagccaggtacagtgactcctttcggtaagt |
| | gcagtggaagctgtacactgcccaggcaaagcgtccgggcagcgtaggcgggcgactcagatc |
| | ccagccagtggacttagcccctgtttgctcctccgataactggggtgaccttggttaatattc |
| | accagcagcctcccccgttgcccctctggatccactgcttaaatacggacgaggacagggccc |
| | tgtctcctcagcttcaggcaccaccactgacctgggacagt |
| ApoE.hAAT | aggctcagaggcacacaggagtttctgggctcaccctgcccccttccaacccctcagttccca |
| SEQ ID NO: 240 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agaggggtacccggggatottgctaccagtggaacagccactaaggattctgcagtgagagca |
| | gagggccagctaagtggtactctcccagagactgtctgactcacgccaccccctccaccttgg |
| | acacaggacgctgtggtttctgagccaggtacaatgactcctttcggtaagtgcagtggaagc |
| | tgtacactgcccaggcaaagcgtccgggcagcgtaggcgggcgactcagatcccagccagtgg |
| | acttagcccctgtttgctcctccgataactggggtgaccttggttaatattcaccagcagcct |
| | cccccgttgcccctctggatccactgcttaaatacggacgaggacagggccctgtctcctcag |
| | cttcaggcaccaccactgacctgggacagt |
| Alpha-Mic/Bik | aggttaatttttaaaaagcagtcaaaagtccaagtggcccttggcagcatttactctctctgt |
| Enhancer | ttgctctggttaataatctcaggagcacaaacattcc |
| (Mic/BikE) | |
| SEQ ID NO: 241 | |
| Tandem (2) | aggttaatttttaaaaagcagtcaaaagtccaagtggcccttggcagcatttactctctctgt |
| alpha-Mic/Bik | ttgctctggttaataatctcaggagcacaaacattccaggttaatttttaaaaagcagtcaaa |
| Enhancers | agtccaagtggcccttggcagcatttactctctctgtttgctctggttaataatctcaggagc |
| (2 Mic/BikE) | acaaacattcc |
| SEQ ID NO: 242 | |
| ApoE Hepatic | aggctcagaggcacacaggagtttctgggctcaccctgcccccttccaacccctcagttccca |
| Control Region | tcctccagcagctgtttgtgtgctgcctctgaagtccacactgaacaaacttcagcctactca |
| containing ApoE | tgtccctaaaatgggcaaacattgcaagcagcaaacagcaaacacacagccctccctgcctgc |
| Enhancer | tgaccttggagctggggcagaggtcagagacctctctgggcccatgccacctccaacatccac |
| SEQ ID NO: 243 | tcgaccccttggaatttcggtggagaggagcagaggttgtcctggcgtggtttaggtagtgtg |
| | agaggg |
| Tandem (2) | aggctcagaggcacacaggagtttctgggctcaccctgcccccttccaacccctcagttccca |
| ApoE Enhancers | tcctccagcagctgtttgtgtgctgcctctgaagtccacactgaacaaacttcagcctactca |
| SEQ ID NO: 244 | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagaaggctcagaggcacacaggagtttctgggctcaccctgcccccttccaacc |
| | cctcagttcccatcctccagcagctgtttgtgtgctgcctctgaagtccacactgaacaaact |
| | tcagcctactcatgtccctaaaatgggcaaacattgcaagcagcaaacagcaaacacacagcc |
| | ctccctgcctgctgaccttggagctggggcagaggtcagagacctctctg |
| hAAT Promoter | gatcttgctaccagtggaacagccactaaggattctgcagtgagagcagagggccagctaagt |
| SEQ ID NO: 245 | ggtactctcccagagactgtctgactcacgccaccccctccaccttggacacaggacgctgtg |
| | gtttctgagccaggtacaatgactcctttcggtaagtgcagtggaagctgtacactgcccagg |
| | caaagcgtccgggcagcgtaggcgggcgactcagatcccagccagtggacttagcccctgttt |
| | gctcctccgataactggggtgaccttggttaatattcaccagcagcctcccccgttgcccctc |
| | tggatccactgcttaaatacggacgaggacagggccctgtctcctcagcttcaggcaccacca |
| | ctgacctgggacagt |
| hAAT(ΔATG) | gatcttgctaccagtggaacagccactaaggattctgcagtgagagcagagggccagctaagt |
| Promoter | ggtactctcccagagactgtctgactcacgccaccccctccaccttggacacaggacgctgtg |
| SEQ ID NO: 246 | gtttctgagccaggtacagtgactcctttcggtaagtgcagtggaagctgtacactgcccagg |
| | caaagcgtccgggcagcgtaggcgggcgactcagatcccagccagtggacttagcccctgttt |
| | gctcctccgataactggggtgaccttggttaatattcaccagcagcctcccccgttgcccctc |
| | tggatccactgcttaaatacggacgaggacagggccctgtctcctcagcttcaggcaccacca |
| | ctgacctgggacagt |
| Mck Enhancer | ccactacgggtttaggctgcccatgtaaggaggcaaggcctggggacacccgagatgcctggt |
| (MckE) | tataattaacccagacatgtggctgccccccccccccccaacacctgctgcctctaaaaataa |
| SEQ ID NO: 247 | ccctgtccctggtggatc |
| Tandem (2) Mck | ccactacgggtttaggctgcccatgtaaggaggcaaggcctggggacacccgagatgcctggt |
| Enhancers | tataattaacccagacatgtggctgccccccccccccccaacacctgctgcctctaaaaataa |
| (2 MckE) | ccctgtccctggtggatcccactacgggtttaggctgcccatgtaaggaggcaaggcctgggg |
| SEQ ID NO: 248 | acacccgagatgcctggttataattaacccagacatgtggctgccccccccccccccaacacc |
| | tgctgcctctaaaaataaccctgtccctggtggatc |
| Tandem Mck (3) | ccactacgggtttaggctgcccatgtaaggaggcaaggcctggggacacccgagatgcctggt |
| Enhancers | tataattaacccagacatgtggctgccccccccccccccaacacctgctgcctctaaaaataa |
| (3 MckE) | ccctgtccctggtggatcccactacgggtttaggctgcccatgtaaggaggcaaggcctgggg |
| SEQ ID NO: 249 | acacccgagatgcctggttataattaacccagacatgtggctgccccccccccccccaacacc |
| | tgctgcctctaaaaataaccctgtccctggtggatcccactacgggtttaggctgcccatgta |
| | aggaggcaaggcctggggacacccgagatgcctggttataattaacccagacatgtggctgcc |
| | ccccccccccccaacacctgctgcctctaaaaataaccctgtccctggtggatc |
| Myosin heavy | cccttcagattaaaaataactgaggtaagggcctgggtaggggaggtggtgtgagacgctcct |
| chain enhancer | gtctctcctctatctgcccatcggccctttggggaggaggaatgtgcccaaggactaaaaaaa |
| (MhcE) | ggccatggagccagaggggcgagggcaacagacctttcatgggcaaaccttggggccctgctg |
| SEQ ID NO: 250 | aagctttggc |

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron (e.g. VH4 intron (SEQ ID NO: 80), SV40 intron (SEQ ID NO: 225), or a chimeric intron (β-globin/Ig Intron) (SEQ ID NO: 79). The viral vectors may also include a Kozak sequence to promote translation of the transgene product, for example GCCACC (SEQ ID NO: 25).

In certain embodiments, the viral vectors provided herein comprise a polyadenylation sequence downstream of the coding region of the transgene. Any poly A site that signals termination of transcription and directs the synthesis of a poly A tail is suitable for use in AAV vectors of the present disclosure. Exemplary poly A signals are derived from, but not limited to, the following: the SV40 late gene, the rabbit β-globin gene (SEQ ID NO: 78), the bovine growth hormone (BPH) gene, the human growth hormone (hGH) gene, the synthetic polyA (SPA) site, and the bovine growth hormone (bGH) gene. See, e.g., Powell and Rivera-Soto, 2015, Discov. Med., 19 (102): 49-57.

### 5.1.4 Signal Peptides

In certain embodiments, the vectors provided herein comprise components that modulate protein delivery. In certain embodiments, the viral vectors provided herein comprise one or more signal peptides. Signal peptides (also referred to as “signal sequences”) may also be referred to herein as “leader sequences” or “leader peptides”. In certain embodiments, the signal peptides allow for the transgene product to achieve the proper packaging (e.g., glycosylation) in the cell. In certain embodiments, the signal peptides allow for the transgene product to achieve the proper localization in the cell. In certain embodiments, the signal peptides allow for the transgene product to achieve secretion from the cell.

There are two general approaches to select a signal sequence for protein production in a gene therapy context or in cell culture. One approach is to use a signal peptide from proteins homologous to the protein being expressed. For example, a human antibody signal peptide may be used to express IgGs in CHO or other cells. Another approach is to identify signal peptides optimized for the particular host cells used for expression. Signal peptides may be interchanged between different proteins or even between proteins of different organisms, but usually the signal sequences of the most abundant secreted proteins of that cell type are used for protein expression. For example, the signal peptide of human albumin, the most abundant protein in plasma, was found to substantially increase protein production yield in CHO cells. However, certain signal peptides may retain function and exert activity after being cleaved from the expressed protein as “post-targeting functions”. Thus, in specific embodiments, the signal peptide is selected from signal peptides of the most abundant proteins secreted by the cells used for expression to avoid the post-targeting functions. In a certain embodiment, the signal sequence is fused to both the heavy and light chain sequences. In another embodiment, one signal sequence is present in the transgene and is fused to whichever sequence (heavy or light chain) is at the N-terminus of the transgene. An exemplary sequence is MYRMQLLLLIALSLALVINS (SEQ ID NO: 85) which can be encoded by a nucleotide sequence of SEQ ID NO: 90 (see Table 2, FIGS. 2A-2G). Alternatively, signal sequences that are appropriate for expression, and may cause selective expression or directed expression of the HuPTM mAb or Fab or scFv in the eye/CNS, muscle, or liver are provided in Tables 2, 3, and 4, respectively, below.

TABLE 2Signal peptides for expression in eye/CNS

| | SEQ | |
|---|---|---|
| | ID | |
| Signal Peptide Origin | NO: | Sequence |
| Mutant interleukin 2 | 85 | MYRMQLLLLIALSLALVINS |
| signal peptide | | |
| Mutant interleukin 2 | 86 | atgtataggatgcaactgc |
| signal peptide coding | | tcctcctgattgctctgag |
| sequence | | cctggctcttgtgaccaac |
| | | tct |
| VEGF-A signal | 87 | MNFLLSWVHWSLALLLYL |
| peptide | | HHAKWSQA |
| Fibulin-1 signal | 88 | MERAAPSRRVPLPLLLLG |
| peptide | | GLALLAAGVDA |
| Vitronectin signal | 89 | MAPLRPLLILALLAWVALA |
| peptide | | |
| Complement Factor H | 90 | MRLLAKIICLMLWAICVA |
| signal peptide | | |
| Opticin signal peptide | 91 | MRLLAFLSLLALVLQETGT |
| Albumin signal | 92 | MKWVTFISLLFLFSSAYS |
| peptide | | |
| Chymotrypsinogen | 93 | MAFLWLLSCWALLGTTFG |
| signal peptide | | |
| Interleukin-2 signal | 94 | MYRMQLLSCIALILALVTNS |
| peptide | | |
| Trypsinogen-2 signal | 95 | MNLLLILTFVAAAVA |
| peptide | | |

TABLE 3
Signal peptides for expression in liver (SEQ ID Nos 96-116) and muscle
(SEQ ID NOs: 117-127, 226, and 178-182) cells.

| | SEQ ID | |
|---|---|---|
| Signal Peptide Origin | NO: | Sequence |
| Human Serum albumin | 96 | MKWVTFISLLFLFSSAYS |
| Human α-1 Antitrypsin | 97 | MPSSVSWGILLLAGLCCLVPVSLA |
| (SERPINA1) | | |
| Human Apolipoprotein A- | 98 | MKAAVLTLAVLFLTGSQA |
| 1 | | |
| Human Apolipoprotein A- | 99 | MKLLAATVLLLTICSLEG |
| 2 | | |
| Human Apolipoprotein B- | 100 | MDPPRPALLALLALPALLLLLLAGARA |
| 100 | | |
| Human Coagulation Factor | 101 | MQRVNMIMAESPGLITICLLGYLLSAEC |
| IX | | |
| Human Complement C2 | 102 | MGPLMVLFCLLFLYPGLADS |
| Human Complement | 103 | MWLLVSVILISRISSVGG |
| Factor H-related Protein 2 | | |
| (CFHR2) | | |
| Human Complement | 104 | MLLLFSVILISWVSTVGG |
| Factor H-related Protein 5 | | |
| (CFHR5) | | |
| Human Fibrinogen α-chain | 105 | MFSMRIVCLVLSVVGTAWT |
| (FGA) | | |
| Human Fibrinogen β-chain | 106 | MKRMVSWSFHKLKTMKHLLLLLLCVFLVKS |
| (FGB) | | |
| Human Fibrinogen γ-chain | 107 | MSWSLHPRNLILYFYALLFLSSTCVA |
| (FGG) | | |
| Human α-2-HS- | 108 | MKSLVLLLCLAQLWGCHS |
| Glycoprotein (AHSG) | | |
| Human Hemopexin (HPX) | 109 | MARVLGAPVALGLWSLCWSLAIA |
| Human Kininogen-1 | 110 | MKLITILFLCSRLLLSLT |
| Human Mannose-binding | 111 | MSLFPSLPLLLLSMVAASYS |
| protein C (MBL2) | | |
| Human Plasminogen | 112 | MEHKEVVLLLLLFLKSGQG |
| (PLMN) | | |
| Human Prothrombin | 113 | MAHVRGLQLPGCLALAALCSLVHS |
| (Coagulation Factor II) | | |
| Human Secreted | 114 | MISRMEKMTMMMKILIMFALGMNYWSCSG |
| Phosphoprotein 24 | | |
| Human Anti-thrombin-III | 115 | MYSNVIGTVTSGKRKVYLLSLLLIGFWDCVTC |
| (SERPINC1) | | |
| Human Serotransferrin | 116 | MRLAVGALLVCAVLGLCLA |
| (TF) | | |
| Human SPARC | 117 | MRAWIFFLLCLAGRALA |
| Human Collagen alpha-1(I) | 118 | MFSFVDLRLLLLLAATALLTHG |
| chain | | |
| Human Lactotransferrin | 119 | MKLVFLVLLFLGALGLCLA |
| Human Complement C3 | 120 | MGPTSGPSLLLLLLTHLPLALG |
| Human Lumican | 121 | MSLSAFTLFLALIGGTSG |
| Human Gelsolin isoform 1 | 122 | MAPHRPAPALLCALSLALCALSLPVRA |
| Human Pro-cathepsin H | 123 | MWATLPLLCAGAWLLGVPVCGA |
| Human SERPINF1 | 124 | MQALVLLLCIGALLGHSSC |
| Human SERPINE1 | 125 | MQMSPALTCLVLGLALVFGEGSA |
| Human Cathepsin D | 126 | MQPSSLLPLALCLLAAPASA |
| Human TIMP1 | 127 | MAPFEPLASGILLLLWLIAPSRA |
| Human Fibronectin | 226 | MLRGPGPGLLLLAVQCLGTAVPSTGASKSKR |
| Human Complement C1s | 178 | MWCIVLFSLLAWVYA |
| subcomponent | | |
| Human Cathepsin L1 | 179 | MNPTLILAAFCLGIASA |
| Human Cathepsin B | 180 | MWQLWASLCCLLVLANA |
| Human Salivary acidic | 181 | MLLILLSVALLAFSSA |
| proline-rich | | |
| phosphoprotein ½ | | |
| Human Follistatin-related | 182 | MWKRWLALALALVAVAWVRA |
| protein 1 | | |

5.1.5 Polycistronic Messages-IRES and 2A Linkers and scFv Constructs

Internal ribosome entry sites. A single construct can be engineered to encode both the heavy and light chains separated by a cleavable linker or IRES so that separate heavy and light chain polypeptides are expressed by the transduced cells. In certain embodiments, the viral vectors provided herein provide polycistronic (e.g., bicistronic) messages. For example, the viral construct can encode the heavy and light chains separated by an internal ribosome entry site (IRES) elements (for examples of the use of IRES elements to create bicistronic vectors see, e.g., Gurtu et al., 1996, Biochem. Biophys. Res. Comm. 229 (1): 295-8, which is herein incorporated by reference in its entirety). IRES elements bypass the ribosome scanning model and begin translation at internal sites. The use of IRES in AAV is described, for example, in Furling et al., 2001, Gene Ther 8 (11): 854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based, AAV3B-based or AAVrh73-based vector).

Furin-2A linkers. In other embodiments, the viral vectors provided herein encode the heavy and light chains separated by a cleavable linker such as the self-cleaving 2A and 2A-like peptides, with or without upstream furin cleavage sites, e.g. Furin/2A linkers, such as furin/F2A (F/F2A) or furin/T2A (F/T2A) linkers (Fang et al., 2005, Nature Biotechnology 23:584-590, Fang, 2007, Mol Ther 15:1153-9, and Chang, J. et al, MAbs 2015, 7 (2): 403-412, each of which is incorporated by reference herein in its entirety). For example, a furin/2A linker may be incorporated into an expression cassette to separate the heavy and light chain coding sequences, resulting in a construct with the structure:

Signal sequence-Heavy chain-Furin site-2A site-Signal Sequence-Light chain-PolyA. A 2A site or 2A-like site, such as an F2A site comprising the amino acid sequence RKRR(GSG)APVKQTLNFDLLKLAGDVESNPGP (SEQ ID NOS: 143 or 144) or a T2A site comprising the amino acid sequence RKRR(GSG)EGRGSLLTCGDVEENPGP (SEQ ID NOS: 141 or 142), is self-processing, resulting in “cleavage” between the final G and P amino acid residues. Several linkers, with or without an upstream flexible Gly-Ser-Gly (GSG) linker sequence (SEQ ID NO:128), that could be used include but are not limited to:

T2A:
(SEQ ID NOS: 133 or 134)
(GSG)EGRGSLLTCGDVEENPGP;
P2A:
(SEQ ID NOS: 135 or 136)
(GSG)ATNFSLLKQAGDVEENPGP;
E2A:
(SEQ ID NOS: 137 or 138)
(GSG)QCTNYALLKLAGDVESNPGP;
F2A:
(SEQ ID NOS: 139 or 140)
(GSG)APVKQTLNFDLLKLAGDVESNPGP

(see also, e.g., Szymczak, et al., 2004, Nature Biotechnol 22 (5): 589-594, and Donnelly, et al., 2001, J Gen Virol, 82:1013-1025, each of which is incorporated herein by reference). Exemplary amino acid sequences (SEQ ID Nos: 128-144 and 51-55) and nucleotide sequences (SEQ ID NOs 145-149) encoding different parts of the flexible linker are described in Table 4.

TABLE 4
Linker Sequences

| | SEQ | |
|---|---|---|
| | ID | |
| ID | NO: | Sequence |
| GSG linker | 128 | GSG |
| Furin linker | 129 | RKRR |
| Furin linker | 130 | RRRR |
| Furin linker | 131 | RRKR |
| Furin linker | 132 | RKKR |
| T2A | 133 | EGRGSLLTCGDVEENPGP |
| T2A | 134 | GSGEGRGSLLTCGDVEENPGP |
| P2A | 135 | ATNFSLLKQAGDVEENPGP |
| P2A | 136 | GSGATNFSLLKQAGDVEENPGP |
| E2A | 137 | QCTNYALLKLAGDVESNPGP |
| E2A | 138 | GSGQCTNYALLKLAGDVESNPGP |
| F2A | 139 | APVKQTLNFDLLKLAGDVESNPGP |
| F2A | 140 | GSGAPVKQTLNFDLLKLAGDVESNPGP |
| Furin-T2A | 141 | RKRREGRGSLLTCGDVEENPGP |
| Furin-GSG-T2A | 142 | RKRRGSGEGRGSLLTCGDVEENPGP |
| Furin-F2A | 143 | RKRRAPVKQTLNFDLLKLAGDVESN |
| | | PGP |
| Furin-GSG-F2A | 144 | RKRRGSGAPVKQTLNFDLLKLAGDVE |
| | | SNPGP |
| Furin- | 145 | agaaagagaagaggctctggagaaggc |
| GSG-T2A | | agaggctccctgctgacatgtggggat |
| | | gttgaagagaatcctgggcct |
| Furin | 146 | agaaagagaaga |
| Furin-GSG | 147 | agaaagagaagaggctctgga |
| linker | | |
| GSG linker | 148 | ggctctgga |
| T2A | 149 | gaaggcagaggctccctgctgacatgt |
| | | ggggatgttgaagagaatcctgggcct |
| G4S | 51 | GGGGS |
| 4GS2 | 52 | GGGGSGGGGS |
| 4GS3 | 53 | GGGGSGGGGSGGGGS |
| 4GS4 | 54 | GGGGSGGGGSGGGGSGGGGS |
| 4GS5 | 55 | GGGGSGGGGSGGGGSGGGGSGGGGS |

In certain embodiments an additional proteolytic cleavage site, e.g. a furin cleavage site, is incorporated into the expression construct adjacent to the self-processing cleavage site (e.g. 2A or 2A like sequence), thereby providing a means to remove additional amino acids that remain following cleavage by the self processing cleavage sequence. Without being bound to any one theory, a peptide bond is skipped when the ribosome encounters the 2A sequence in the open reading frame, resulting in the termination of translation, or continued translation of the downstream sequence (the light chain). This self-processing sequence results in a string of additional amino acids at the end of the C-terminus of the heavy chain. However, such additional amino acids can then be cleaved by host cell Furin at the furin cleavage site(s), e.g. located immediately prior to the 2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15 (6): 1153-1159; Luke, 2012, Innovations in Biotechnology, Ch. 8, 161-186). Furin linkers that may be used comprise a series of four basic amino acids, for example, RKRR (SEQ ID NO: 129), RRRR (SEQ ID NO: 130), RRKR (SEQ ID NO:131), or RKKR (SEQ ID NO:132). Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR (SEQ ID NO: 129), RRRR (SEQ ID NO:130), RRKR (SEQ ID NO:131), or RKKR (SEQ ID NO:132). In certain embodiments, once the linker is cleaved by a carboxypeptidase, no additional amino acids remain. In certain embodiments, 0.5% to 1%, 1% to 2%, 5%, 10%, 15%, or 20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R-X-K/R-R, such that the additional amino acids on the C-terminus of the heavy chain are R, RX, RXK, RXR, RXKR, or RXRR, where X is any amino acid, for example, alanine (A). In certain embodiments, no additional amino acids may remain on the C-terminus of the heavy chain.

Flexible peptide linker. In some embodiments, a single construct can be engineered to encode both the heavy and light chains (e.g. the heavy and light chain variable domains) separated by a flexible peptide linker such as those encoding a scFv. A flexible peptide linker can be composed of flexible residues like glycine and serine so that the adjacent heavy chain and light chain domains are free to move relative to one another. The construct may be arranged such that the heavy chain variable domain is at the N-terminus of the scFv, followed by the linker and then the light chain variable domain. Alternatively, the construct may be arranged such that the light chain variable domain is at the N-terminus of the scFv, followed by the linker and then the heavy chain variable domain. That is, the components may be arranged as NH₂-V_{L}-linker-V_{H}-COOH or NH₂-V_{H}-linker-V_{L}-COOH.

In certain embodiments, an expression cassette described herein is contained within a viral vector with a restraint on the size of the polynucleotide(s) therein. In certain embodiments, the expression cassette is contained within an AAV virus-based vector. Due to the size restraints of certain vectors, the vector may or may not accommodate the coding sequences for the full heavy and light chains of the therapeutic antibody but may accommodate the coding sequences of the heavy and light chains of antigen binding fragments, such as the heavy and light chains of a Fab or F(ab′)₂ fragment or an scFv. In particular, the AAV vectors described herein may accommodate a transgene of approximately 4.7 kilobases. Substitution of smaller expression elements would permit the expression of larger protein products, such as full-length therapeutic antibodies.

Commonly used flexible linkers have sequences consisting primarily of stretches of four Gly and one Ser residue (“GS” linker), an example of the most widely used flexible linker having the sequence of (Gly-Gly-Gly-Gly-Ser)n (GGGGS or G4S; SEQ ID NO:51). By adjusting the copy number “n”, the length of this GS linker can be optimized to achieve appropriate separation of the functional domains, or to maintain necessary inter-domain interactions. Examples include, but are not limited to (Gly-Gly-Gly-Gly-Ser) 2 (SEQ ID NO: 52), (Gly-Gly-Gly-Gly-Ser) 3 (SEQ ID NO: 53), (Gly-Gly-Gly-Gly-Ser) 4 (SEQ ID NO: 54), and (Gly-Gly-Gly-Gly-Ser) 5 (SEQ ID NO: 55). Besides the GS linkers, many other flexible linkers have been designed for recombinant fusion proteins (Chen, X. et al, Adv Drug Deliv Rev. 2013 Oct. 15; 65 (10): 1357-1369). See, e.g., Table 4.

### 5.1.6 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half-life of the transgene. In certain embodiments, the UTRs are optimized for the stability of the mRNA of the transgene. In certain embodiments, the UTRs are optimized for the secondary structure of the mRNA of the transgene.

### 5.1.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79 (1): 364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety). In preferred embodiments, nucleotide sequences encoding the ITRs may, for example, comprise the nucleotide sequences of SEQ ID NOS: 81 (5′-ITR) or 82 (3′-ITR). In certain embodiments, the modified ITRs used to produce self-complementary vector, e.g., scAAV, may be used (see, e.g., Wu, 2007, Human Gene Therapy, 18 (2): 171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety). In preferred embodiments, nucleotide sequences encoding the modified ITRs may, for example, comprise the nucleotide sequences of SEQ ID NOS: 81 (5′-ITR) or 83 (3′-ITR) or modified for scAAV, SEQ ID NO:82 (m 5′ITR) or SEQ ID NO:84 (m 3′ ITR).

### 5.1.8 Transgenes

The transgenes encode a HuPTM mAb, either as a full-length antibody or an antigen binding fragment thereof, e.g. a Fab fragment (an HuGlyFab) or a F(ab′)₂, nanobody, or an scFv based upon a therapeutic antibody disclosed herein. In specific embodiments, the HuPTM mAb or antigen binding fragment, particularly the HuGlyFab, are engineered to contain additional glycosylation sites on the Fab domain (e.g., see Courtois et al., 2016, mAbs 8:99-112 which is incorporated by reference herein in its entirety for it description of sites of hyperglycosylation on a Fab domain). In addition, for the HuPTM mAb comprising an Fc domain, the Fc domain may be engineered to alter the glycosylation site at N297 to prevent glycosylation at that site (for example, a substitution at N297 for another amino acid and/or a substitution at T297 for a residue that is not a T or S to knock out the glycosylation site). Such Fc domains are “aglycosylated”.

### 5.1.8.1 Constructs for Expression of Full Length HuPTM mAb

In certain embodiments, the transgenes encode a full length heavy chain (including the heavy chain variable domain, the heavy chain constant domain 1 (C_{H}1), the hinge and Fc domain) and a full length light chain (light chain variable domain and light chain constant domain) that upon expression associate to form antigen-binding antibodies with Fc domains. The recombinant AAV constructs express the intact (i.e., full length) or substantially intact HuPTM mAb in a cell, cell culture, or in a subject. (“Substantially intact” refers to mAb having a sequence that is at least 95% identical to the full-length mAb sequence.) The nucleotide sequences encoding the heavy and light chains may be codon optimized for expression in human cells and have reduced incidence of CpG dimers in the sequence to promote expression in human cells. See for example, the codon optimized sequences of crovalimab (SEQ ID NOs: 26, 27, 37, or 38) or eculizumab (SEQ ID NO: 28-30, or 39-41) of Table 8. In embodiments, the transgenes encode a full-length form of any of the therapeutic antibodies disclosed herein, for example, the Fab fragment of which depicted in FIGS. 2A-2G herein and including, in certain embodiments, the associated Fc domain provided in Table 6.

The full length mAb encoded by the transgene described herein preferably have the Fc domain of the full-length therapeutic antibody or is an Fc domain of the same type of immunoglobulin as the therapeutic antibody to be expressed. In certain embodiments, the Fc region is an IgG Fc region, but in other embodiments, the Fc region may be an IgA, IgD, IgE, or IgM. The Fc domain is preferably of the same isotype as the therapeutic antibody to be expressed, for example, if the therapeutic antibody is an IgG1 isotype, then the antibody expressed by the transgene comprises an IgG1 Fc domain. The antibody expressed from the transgene may have an IgG1, IgG2, IgG3 or IgG4 Fc domain.

The Fc region of the intact mAb has one or more effector functions that vary with the antibody isotype. The effector functions can be the same as that of the wild-type or the therapeutic antibody or can be modified therefrom to add, enhance, modify, or inhibit one or more effector functions using the Fc modifications disclosed in Section 5.1.9, infra. In certain embodiments, the HuPTM mAb transgene encodes a mAb comprising an Fc polypeptide comprising an amino acid sequence that is at least 85%, 86%, 87%, 88%, 89%, 90%, 91%, 92%, 93%, 94%, 95%, 96%, 97%, 98% or 99% identical to the sequence set forth in the Fc domain polypeptides of the therapeutic antibodies described herein as set forth in Table 6 for crovalimab, eculizumab, ravulizumab, tesidolumab or NGM621, or an exemplary Fc domain of an IgG1, IgG2 or IgG4 isotype as set forth in Table 6. In some embodiments, the HuPTM mAb comprises a Fc polypeptide of a sequence that is a variant of the Fc polypeptide sequence in Table 6 in that the sequence has been modified with one or more of the techniques described in Section 5.1.9, infra, to alter the Fc polypeptide's effector function. In other embodiments, the transgene encodes a surrogate antibody for use in NHP or other animal models, for example the antibody BB5.1 as a C5 binding surrogate for at least eculizumab or ravulizumab.

In some embodiments, provided are exemplary recombinant AAV constructs such as the constructs shown in FIG. 1A, for gene therapy administration to a human subject in order to express an intact or substantially intact HuPTM mAb in the subject. Gene therapy constructs are designed such that both the heavy and light chains are expressed in tandem from the vector including the Fc domain polypeptide of the heavy chain. In certain embodiments, the transgene encodes a transgene with heavy and light chain Fab fragment polypeptides as shown in Table 7, yet have a heavy chain that further comprises an Fc domain polypeptide C terminal to the hinge region of the heavy chain (including an IgG1, IgG2 or IgG4 Fc domain or the crovalimab, eculizumab, ravulizumab, tesidolumab or NGM621 Fc as in Table 6). In specific embodiments, the transgene is a nucleotide sequence that encodes the following: Signal sequence-heavy chain Fab portion (including hinge region)-heavy chain Fc polypeptide-Furin-2A linker-signal sequence-light chain Fab portion.

In specific embodiments for expressing an intact or substantially intact mAb in ocular tissue cell types, the constructs described herein comprise the following components: (1) AAV2 inverted terminal repeats that flank the expression cassette: (2) Control elements, which include a) an ocular-tissue specific promoter or constitutive promoter, b) optionally an intron, such as a chicken β-actin intron or VH4 intron and c) a rabbit β-globin poly A signal; and (3) nucleic acid sequences coding for the heavy chain Fab of an anti-C5 or anti-C3 mAb (e.g. the crovalimab, eculizumab, ravulizumab, tesidolumab or NGM621); an Fc polypeptide associated with the therapeutic antibody (Table 6) or of the same isotype as the native form of the therapeutic antibody, such as an IgG isotype amino acid sequence from Table 6; and the light chain of an anti-C5 or anti-C3 mAb (e.g. the crovalimab, eculizumab, ravulizumab, tesidolumab or NGM621), wherein the heavy chain (Fab and Fc region) and the light chain are separated by a self-cleaving furin (F)/F2A or T2A or flexible linker, ensuring expression of equal amounts of the heavy and the light chain polypeptides. Exemplary constructs are provided in FIGS. 1A and 1B.

In specific embodiments for expressing an scFv form of a mAb in ocular tissue cell types, the constructs described herein comprise the following components: (1) AAV2 inverted terminal repeats that flank the expression cassette; (2) Control elements, which include a) an ocular-tissue specific promoter or constitutive promoter, b) optionally an intron, such as a chicken β-actin intron or VH4 intron and c) a rabbit β-globin poly A signal; and (3) nucleic acid sequences coding an scFv in which the heavy and light chain variable domains are connected via a flexible, non-cleavable linker, such as GGGGSGGGGSGGGGS (SEQ ID NO: 53). In certain embodiments, the construct expresses, from the N-terminus, NH₂-signal sequence-V_{L}-linker-V_{H}-COOH or NH₂-signal sequence-V_{H}-linker-V_{L}-COOH. In certain embodiments, the construct encodes, from the N-terminus, NH₂-signal sequence-V_{L}-GGGGSGGGGSGGGGS-V_{H}-COOH or NH₂-signal sequence-V_{H}-GGGGGGGGSGGGGS-V_{L}-COOH. In certain embodiments, the linker is GGGGS (SEQ ID NO: 51), GGGGSGGGGS (SEQ ID NO: 52), GGGGSGGGGSGGGGS (SEQ ID NO: 53), GGGGSGGGGSGGGGSGGGGS (SEQ ID NO: 54) or GGGGSGGGGSGGGGSGGGGSGGGGS (SEQ ID NO: 55). In certain embodiments, the signal sequence is MYRMQLLLLIALSLALVTNS (SEQ ID NO: 85) or a signal sequence from Table 2. In certain embodiments, the VH is SEQ ID NO: 251 and VL is SEQ ID NO: 252, wherein VH is SEQ ID NO: 253 and VL is SEQ ID NO: 254, wherein VH is SEQ ID NO: 255 and the VL is SEQ ID NO: 256, wherein VH is SEQ ID NO: 257 and VL is SEQ ID NO: 258, or wherein VH is SEQ ID NO: 259 and VL is SEQ ID NO: 260. Exemplary constructs are provided in FIG. 1C and Tables 7 (amino acid sequences) and 8 (nucleotide sequences).

In specific embodiments, provided are A AV vectors comprising a viral capsid that is at least 95% identical to the amino acid sequence of an AAV8 capsid (SEQ ID NO: 196), or, alternatively, an AAV9, AAV3B, or AAVrh73 capsid (or a variant thereof); and an artificial genome comprising an expression cassette flanked by AAV inverted terminal repeats (ITRs), wherein the expression cassette comprises a transgene encoding an intact or substantially intact anti-C5 or anti-C3 mAb; operably linked to one or more regulatory sequences that control expression of the transgene in ocular tissue type cells, such as RPE cells, BrM cells, choriocapillaris cells, photoreceptor cells (rods and/or cones), retinal ganglion cells.

The rAAV vectors that encode and express the full-length therapeutic antibodies may be administered to treat or prevent or ameliorate symptoms of a disease or condition amenable to treatment, prevention or amelioration of symptoms with the therapeutic antibodies, such as dry AMD. Also provided are methods of expressing HuPTM mAbs in human cells using the rAAV vectors and constructs encoding them.

### 5.1.8.2 Constructs for Expression of Antigen Binding Fragments

In some embodiments, the transgenes express antigen binding fragments, e.g. a Fab fragment (an HuGlyFab) or a F(ab′)₂, nanobody, or an scFv based upon a therapeutic antibody disclosed herein. FIGS. 2A-2G and section 5.4. provide the amino acid sequence of the heavy and light chains of the Fab fragments of the therapeutic antibodies (see also Table 7, which provides the amino acid sequences of the Fab heavy and light chains of the therapeutic antibodies).

Certain of these nucleotide sequences are codon optimized for expression in human cells. See for example, the codon optimized sequences encoding crovalimab (SEQ ID NOs: 26, 27, 37, 38) or eculizumab (SEQ ID Nos: 28-30, and 39-41) in Table 8. The transgene may encode a Fab fragment using nucleotide sequences encoding the amino acid sequences provided in Table 7, but not including the portion of the hinge region on the heavy chain that forms interchain di-sulfide bonds (e.g., the portion containing the sequence CPPCPA (SEQ ID NO: 151)). Heavy chain Fab domain sequences that do not contain a CPPCP (SEQ ID NO: 151) sequence of the hinge region at the C-terminus will not form intrachain disulfide bonds and, thus, will form Fab fragments with the corresponding light chain Fab domain sequences, whereas those heavy chain Fab domain sequences with a portion of the hinge region at the C-terminus containing the sequence CPPCP (SEQ ID NO: 151) will form intrachain disulfide bonds and, thus, will form Fab₂ fragments. For example, in some embodiments, the transgene may encode a scFv comprising a light chain variable domain and a heavy chain variable domain connected by a flexible linker in between (where the heavy chain variable domain may be either at the N-terminal end or the C-terminal end of the scFv), and optionally, may further comprise a Fc polypeptide (e.g., IgG1, IgG2, IgG3, or IgG4) on the C-terminal end of the heavy chain. Alternatively, in other embodiments, the transgene may encode F(ab′)₂ fragments comprising a nucleotide sequence that encodes the light chain and the heavy chain sequence that includes at least the sequence CPPCA (SEQ ID NO: 152) of the hinge region, as depicted in FIGS. 2A-2G which depict various regions of the hinge region that may be included at the C-terminus of the heavy chain sequence. In embodiments, the hinge region is the sequence EPKSCDKTH (SEQ ID NO: 307). Pre-existing anti-hinge antibodies (AHA) may cause immunogenicity and reduce efficacy. Thus, in certain embodiments, for the IgG1 isotype, C-terminal ends with D221 or ends with a mutation T225L or with L242 can reduce binding to AHA. (See, e.g., Brezski, 2008, J Immunol 181:3183-92 and Kim, 2016, 8:1536-1547). For IgG2, the risk of AHA is lower since the hinge region of IgG2 is not as susceptible to enzymatic cleavage required to generate endogenous AHA. (See, e.g., Brezski, 2011, MAbs 3:558-567).

TABLE 5
Hinge Regions

| SEQ ID NO: | Sequence |
|---|---|
| 150 | CPPCP |
| 151 | CPPCPA |
| 152 | CPPCA |
| 153 | EPKSCDKTHTCPPCPAPELLGG |
| 154 | EPKSCDKTHLCPPCPAPELLGG |
| 155 | EPKSCDKTHL |
| 156 | EPKSCDKTHT |
| 157 | EPKSCDKTHTCPPCPA |
| 158 | EPKSCDKTHLCPPCPA |
| 159 | EPKSCDKTHTCPPCPAPELLGGPSVFL |
| 160 | EPKSCDKTHLCPPCPAPELLGGPSVFL |
| 161 | EPKSCDKTHTCPPCPAPEAAGG |
| 162 | EPKSCDKTHTCPPCPAPEAAGGPSVFL |
| 163 | EPKSCDKTHLCPPCPAPEAAGGPSVFL |
| 164 | ERKSCVECPPCPAPPVAG |
| 165 | ERKSCVECPPCPA |
| 166 | ESKYGPPCPPCPAPEFLGG |
| 167 | ESKYGPPCPPCPA |
| 168 | ESKYGPPCPSCPA |
| 169 | ESKYGPPCPPCPAPEFLGGPSVFL |
| 170 | ERKCCVECPPCPAPPVAG |
| 171 | ERKCCVECPPCPA |
| 172 | EPKSCDKTHTCPPCPAPELAGA |
| 173 | EPKSCDKTHTCPPCPAPELAGAPSVFL |
| 174 | EPKSCDKTHLCPPCPAPELAGAPSVFL |
| 175 | EPKSCDKTHTCPPCPAPEFEGG |
| 176 | EPKSCDKTHTCPPCPAPEFEGGPSVFL |
| 177 | EPKSCDKTHLCPPCPAPEFEGGPSVFL |
| 307 | EPKSCDKTH |
| 308 | ESKYGPPCPSCPAPEFLGGPSVFL |

In certain embodiments, the viral vectors provided herein comprise the following elements in the following order: a) a constitutive or inducible (e.g., hypoxia-inducible or rifamycin-inducible) promoter sequence or a tissue specific promoter/regulatory region, for example, one of the regulatory regions provided in Table 1 or 1a, and b) a sequence encoding the transgene (e.g., a HuGlyFab or scFv). In certain embodiments, the sequence encoding the transgene comprises multiple ORFs separated by IRES elements. In certain embodiments, the ORFs encode the heavy and light chain domains of the HuGlyFab. In certain embodiments, the sequence encoding the transgene comprises multiple subunits in one ORF separated by F/F2A sequences or F/T2A sequences. In certain embodiments, the sequence comprising the transgene encodes the heavy and light chain domains of the HuGlyFab separated by an F/F2A sequence or a F/T2A sequence. In certain embodiments, the sequence comprising the transgene encodes the heavy and light chain variable domains of the HuGlyFab separated by a flexible peptide linker (as an scFv). In certain embodiments, the viral vectors provided herein comprise the following elements in the following order: a) a constitutive or an inducible promoter sequence or a tissue specific promoter, such as one of the promoters or regulatory regions in Table 1 or 1a, and b) a sequence encoding the transgene (e.g., a HuGlyFab), wherein the transgene comprises a nucleotide sequence encoding a signal peptide, a light chain and a heavy chain Fab portion separated by an IRES element. In certain embodiments, the viral vectors provided herein comprise the following elements in the following order: a) a constitutive or a hypoxia-inducible promoter sequence or regulatory element listed in Table 1 or 1a, and b) a sequence encoding the transgene comprising a signal peptide, a light chain and a heavy chain sequence separated by a cleavable F/F2A sequence (SEQ ID NOS: 143 or 144) or a F/T2A sequence (SEQ ID NOS: 141 or 142) or a flexible peptide linker.

In certain embodiments, the viral vectors provided herein comprise the following elements in the following order: a) a first ITR sequence, b) a first linker sequence, c) a constitutive or an inducible promoter sequence or a tissue specific promoter or regulatory region, d) a second linker sequence, e) an intron sequence, f) a third linker sequence, g) a first UTR sequence, h) a sequence encoding the transgene (e.g., a HuGlyFab), i) a second UTR sequence, j) a fourth linker sequence, k) a poly A sequence, 1) a fifth linker sequence, and m) a second ITR sequence.

In certain embodiments, the viral vectors provided herein comprise the following elements in the following order: a) a first ITR sequence, b) a first linker sequence, c) a constitutive or an inducible promoter sequence or a tissue specific regulatory region, d) a second linker sequence, e) an intron sequence, f) a third linker sequence, g) a first UTR sequence, h) a sequence encoding the transgene (e.g., HuGlyFab), i) a second UTR sequence, j) a fourth linker sequence, k) a poly A sequence, 1) a fifth linker sequence, and m) a second ITR sequence, wherein the transgene comprises a signal, and wherein the transgene encodes a light chain and a heavy chain sequence separated by a cleavable F/2A sequence.

In certain embodiments, the viral vectors provided herein comprise the following elements in the following order: a) a first ITR sequence, b) a first linker sequence, c) a constitutive or an inducible promoter sequence or a tissue specific regulatory region, d) a second linker sequence, e) an intron sequence, f) a third linker sequence, g) a first UTR sequence, h) a sequence encoding the transgene (e.g., VH-(linker)-VL or VL-(linker)-VH), i) a second UTR sequence, j) a fourth linker sequence, k) a poly A sequence, 1) a fifth linker sequence, and m) a second ITR sequence.

### 5.1.9. Fc Region Modifications

In certain embodiments, the transgenes encode full length or substantially full length heavy and light chains that associate to form a full length or intact antibody. (“Substantially intact” or “substantially full length” refers to a mAb having a heavy chain sequence that is at least 95% identical to the full-length heavy chain mAb amino acid sequence and a light chain sequence that is at least 95% identical to the full-length light chain mAb amino acid sequence). Accordingly, the transgenes comprise nucleotide sequences that encode, for example, the light and heavy chains of the Fab fragments including the hinge region of the heavy chain and C-terminal of the heavy chain of the Fab fragment, an Fc domain peptide. Table 6 provides the amino acid sequence of the Fc polypeptides for crovalimab, eculizumab, ravulizumab, tesidolumab and NGM621. Alternatively, an IgG1, IgG2, or IgG4 Fc domain, the sequences of which are provided in Table 6 may be utilized.

The term “Fc region” refers to a dimer of two “Fc polypeptides” (or “Fc domains”), each “Fc polypeptide” comprising the heavy chain constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to at least the last two constant region immunoglobulin domains of IgA, IgD, and IgG, or the last three constant region immunoglobulin domains of IgE and IgM and may also include part or all of the flexible hinge N-terminal to these domains. For IgG, e.g., “Fc polypeptide” comprises immunoglobulin domains Cgamma2 (Cγ2, often referred to as CH2 domain) and Cgamma3 (Cγ3, also referred to as CH3 domain) and may include the lower part of the hinge domain between Cgamma1 (Cγ1, also referred to as CH1 domain) and CH2 domain. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, Va.). For IgA, e.g., Fc polypeptide comprises immunoglobulin domains Calpha2 (Cα2) and Calpha3 (Cα3) and may include the lower part of the hinge between Calpha1 (Cα1) and Cα2.

In certain embodiments, the Fc polypeptide is that of the therapeutic antibody or is the Fc polypeptide corresponding to the isotype of the therapeutic antibody). In still other embodiments, the Fc polypeptide is an IgG Fc polypeptide. The Fc polypeptide may be from the IgG1, IgG2, or IgG4 isotype (see Table 6) or may be an IgG3 Fc domain, depending, for example, upon the desired effector activity of the therapeutic antibody. In some embodiments, the engineered heavy chain constant region (CH), which includes the Fc domain, is chimeric. As such, a chimeric CH region combines CH domains derived from more than one immunoglobulin isotype and/or subtype. For example, the chimeric (or hybrid) CH region comprises part or all of an Fc region from IgG, IgA and/or IgM. In other examples, the chimeric CH region comprises part or all a CH2 domain derived from a human IgG1, human IgG2, or human IgG4 molecule, combined with part or all of a CH3 domain derived from a human IgG1, human IgG2, or human IgG4 molecule. In other embodiments, the chimeric CH region contains a chimeric hinge region.

TABLE 6
Table of Fc Domain Amino Acid Sequences

| | Chain/ | |
|---|---|---|
| | SEQ ID | |
| Fc | NO. | Sequence |
| IgG1 | Fc | FPPKPKDTLM ISRTPEVTCV VVDVSHEDPE VKENWYVDGV |
| | domain | EVHNAKTKPR EEQYNSTYRV VSVLTVLHQD WINGKEYKCK |
| | /SEQ ID | VSNKALPAPI EKTISKAKGQ PREPQVYTLP PSRDELTKNQ |
| | NO: 61 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| IgG2 | Fc | FPPKPKDTLM ISRTPEVTCV VVDVSHEDPE VQFNWYVDGV |
| | domain | FPPKPKDTLM ISRTPEVTCV VVDVSHEDPE VQFNWYVDGV |
| | /SEQ ID | EVHNAKTKPR EEQFNSTFRV VSVLTVVHQD WLNGKEYKCK |
| | NO: 62 | VSNKGLPAPI EKTISKTKGQ PREPQVYTLP PSREEMTKNQ |
| | | VSLTCLVKGF YPSDISVEWE SNGQPENNYK TTPPMLDSDG |
| | | SFFLYSKLTV DKSRWQQGNV FSCSVMHEAL HNHYTQKSLS LSPGK |
| IgG4 | Fc | FPPKPKDTLM ISRTPEVTCV VVDVSQEDPE VQFNWYVDGV |
| | domain | EVHNAKTKPR EEQFNSTYRV VSVLTVLHQD WLNGKEYKCK |
| | /SEQ ID | VSNKGLPSSI EKTISKAKGQ PREPQVYTLP PSQEEMTKNQ |
| | NO: 63 | VSLTCLVKGF YPSDIAVEWE SNGQPENNYK TTPPVLDSDG |
| | | SFFLYSRLTV DKSRWQEGNV FSCSVMHEAL HNHYTQKSLS LSLGK |
| Crovalimab | Fc | FPPKPKDTLM ISRTPEVTCV VVDVSHEDPE VKENWYVDGV |
| | domain | EVHNAKTKPR EEQYNSTYRV VSVLTVLHQD WINGKEYKCK |
| | /SEQ ID | VSNKGLPSSI EKTISKAKGQ PREPQVYTLP PSREEMTKNQ |
| | NO: 64 | VSLTCLVKGF YPSDIAVEWE SNGQPENNYK TTPPVLDSDG |
| | | SFFLYSKLTV DKSRWQQGNV FSCSVLHEAL HAHYTRKELS LSP |
| Eculizumab | Fc | FPPKPKDTLM ISRTPEVTCV VVDVSQEDPE VQFNWYVDGV |
| | domain | EVHNAKTKPR EEQFNSTYRV VSVLTVLHQD WINGKEYKCK |
| | /SEQ ID | VSNKGLPSSI EKTISKAKGQ PREPQVYTLP PSQEEMTKNQ |
| | NO: 65 | VSLTCLVKGF YPSDIAVEWE SNGQPENNYK TTPPVLDSDG |
| | | SFFLYSRLTV DKSRWQEGNV FSCSVMHEAL HNHYTQKSLS LSLGK |
| Ravulizumab | Fc | FPPKPKDTLM ISRTPEVTCV VVDVSQEDPE VQFNWYVDGV |
| | domain/ | EVHNAKTKPR EEQENSTYRV VSVLTVLHQD WINGKEYKCK |
| | SEQ ID | VSNKGLPSSI EKTISKAKGQ PREPQVYTLP PSQEEMTKNQ |
| | NO: 66 | VSLTCLVKGF YPSDIAVEWE SNGQPENNYK TTPPVLDSDG |
| | | SFFLYSRLTV DKSRWQEGNV FSCSVLHEAL HSHYTQKSIS LSLGK |
| Tesidolumab | Fc | FPPKPKDTLM ISRTPEVTCV VVDVSHEDPE VKENWYVDGV |
| | domain | EVHNAKTKPR EEQYNSTYRV VSVLTVLHQD WLNGKEYKCK |
| | /SEQ ID | VSNKALPAPI EKTISKAKGQ PREPQVYTLP PSREEMTKNQ |
| | NO: 67 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |
| NGM621 | Fc | FPPKPKDTLM ISRIPEVTCV VVDVSHEDPE VKENWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSREEMTKNQ |
| | NO: 68 | VSLTCLVKGF YPSDIAVEWE SNGQPENNYK TTPPVLDSDG |
| | | SFFLYSKLTV DKSRWQQGNV FSCSVMHEAL HNHYTQKSLS LSPGK |

In some embodiments, the recombinant vectors encode therapeutic antibodies comprising an engineered (mutant) Fc regions, e.g. engineered Fc regions of an IgG constant region. Modifications to an antibody constant region, Fc region or Fc fragment of an IgG antibody may alter one or more effector functions such as Fc receptor binding or neonatal Fc receptor (FcRn) binding and thus half-life, CDC activity, ADCC activity, and/or ADPC activity, compared to a corresponding antibody having a wild-type IgG constant region, or an IgG heavy chain constant region without the recited modification(s). Accordingly, in some embodiments, the antibody may be engineered to provide an antibody constant region, Fc region or Fc fragment of an IgG antibody that exhibits altered binding (as compared to a reference or wild-type constant region without the recited modification(s)) to one or more Fc receptors (e.g., FcγRI, FcγRIIA, FcγRIIB, FcγRIIIA, FcγRIIIB, FcγRIV, or FcRn receptor). In some embodiments, the antibody an antibody constant region, Fc region or Fc fragment of an IgG antibody that exhibits a one or more altered effector functions such as CDC, ADCC, or ADCP activity, compared to a corresponding antibody having a wild-type IgG constant region, or an IgG constant without the recited modification(s).

“Effector function” refers to a biochemical event that results from the interaction of an antibody Fc region with an Fc receptor or ligand. Effector functions include FcγR-mediated effector functions such as ADCC and ADCP and complement-mediated effector functions such as CDC.

An “effector cell” refers to a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and T cells, and may be from any organism including but not limited to humans, mice, rats, rabbits, and monkeys.

“ADCC” or “antibody dependent cell-mediated cytotoxicity” refers to the cell-mediated reaction wherein nonspecific cytotoxic effector (immune) cells that express FcγRs recognize bound antibody on a target cell and subsequently cause lysis of the target cell.

“ADCP” or “antibody dependent cell-mediated phagocytosis” refers to the cell-mediated reaction wherein nonspecific cytotoxic effector (immune) cells that express FcγRs recognize bound antibody on a target cell and subsequently cause phagocytosis of the target cell.

“CDC” or “complement-dependent cytotoxicity” refers to the reaction wherein one or more complement protein components recognize bound antibody on a target cell and subsequently cause lysis of the target cell.

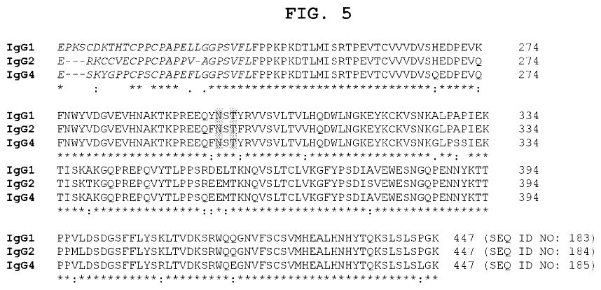

In some embodiments, the modifications of the Fc domain include, but are not limited to, the following modifications and combinations thereof, with reference to EU numbering of an IgG constant region (see FIG. 5): 233, 234, 235, 236, 237, 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

In certain embodiments, the Fc region comprises an amino acid addition, deletion, or substitution of one or more of amino acid residues 251-256, 285-290, 308-314, 385-389, and 428-436 of the IgG. In some embodiments, 251-256, 285-290, 308-314, 385-389, and 428-436 (EU numbering of Kabat; see FIG. 5) is substituted with histidine, arginine, lysine, aspartic acid, glutamic acid, serine, threonine, asparagine, or glutamine. In some embodiments, a non-histidine residue is substituted with a histidine residue. In some embodiments, a histidine residue is substituted with a non-histidine residue.

Enhancement of FcRn binding by an antibody having an engineered Fc leads to preferential binding of the affinity-enhanced antibody to FcRn as compared to antibody having wild-type Fc, and thus leads to a net enhanced recycling of the FcRn-affinity-enhanced antibody, which results in further increased antibody half-life. An enhanced recycling approach allows highly effective targeting and clearance of antigens, including e.g. “high titer” circulating antigens, such as C5, cytokines, or bacterial or viral antigens.

Provided in certain embodiments are modified constant region, Fc region or Fc fragment of an IgG antibody with enhanced binding to FcRn in serum as compared to a wild-type Fc region (without engineered modifications). In some instances, antibodies, e.g. IgG antibodies, are engineered to bind to FcRn at a neutral pH, e.g., at or above pH 7.4, to enhance pH-dependence of binding to FcRn as compared to a wild-type Fc region (without engineered modifications). In some instances, antibodies, e.g. IgG antibodies, are engineered to exhibit enhanced binding (e.g. increased affinity or K_{D}) to FcRn in endosomes (e.g., at an acidic pH, e.g., at or below pH 6.0) relative to a wild-type IgG and/or reference antibody binding to FcRn at an acidic pH, as well as in comparison to binding to FcRn in serum (e.g., at a neutral pH, e.g., at or above pH 7.4). Provided are antibodies with an engineered antibody constant region, Fc region or Fc fragment of an IgG antibody that exhibits an improved serum or resident tissue half-life, compared to a corresponding antibody having a wild-type IgG constant region, or an IgG constant without the recited modification(s);

Non-limiting examples of such Fc modifications include, e.g., a modification at position 250 (e.g., E or Q); 250 and 428 (e.g., L or F); 252 (e.g., LN/Y/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y or A), including 428L and 434A; or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P) (EU numbering; see FIG. 5).

In some embodiments, the Fc region can be a mutant form such as hIgG1 Fc including M252 mutations, e.g. M252Y and S254T and T256E (“YTE mutation”) exhibit enhanced affinity for human FcRn (Dall'Acqua, et al., 2002, J Immunol 169:5171-5180) and subsequent crystal structure of this mutant antibody bound to hFcRn resulting in the creation of two salt bridges (Oganesyan, et al. 2014, JBC 289 (11): 7812-7824). Antibodies having the YTE mutation have been administered to monkeys and humans, and have significantly improved pharmacokinetic properties (Haraya, et al., 2019, Drug Metabolism and Pharmacokinetics, 34 (1): 25-41).

In some embodiments, modifications to one or more amino acid residues in the Fc region may reduce half-life in systemic circulation (serum), however result in improved retainment in tissues (e.g. in the eye) by disabling FcRn binding (e.g. H435A, EU numbering of Kabat) (Ding et al., 2017, MAbs 9:269-284; and Kim, 1999, Eur J Immunol 29:2819).

In some embodiments, the Fc domain may be engineered to activate all, some, or none of the normal Fc effector functions, without affecting the Fc polypeptide's (e.g. antibody's) desired pharmacokinetic properties. Fc polypeptides having altered effector function may be desirable as they may reduce unwanted side effects, such as activation of effector cells, by the therapeutic protein.

Methods to alter or even ablate effector function may include mutation(s) or modification(s) to the hinge region amino acid residues of an antibody. For example, IgG Fc domain mutants comprising 234A, 237A, and 238S substitutions, according to the EU numbering system, exhibit decreased complement dependent lysis and/or cell mediated destruction. Deletions and/or substitutions in the lower hinge, e.g. where positions 233-236 within a hinge domain (EU numbering) are deleted or modified to glycine, have been shown in the art to significantly reduce ADCC and CDC activity.

In specific embodiments, the Fc domain is an aglycosylated Fc domain that has a substitution at residue 297 or 299 to alter the glycosylation site at 297 such that the Fc domain is not glycosylated. Such aglycosylated Fc domains may have reduced ADCC or other effector activity.

Non-limiting examples of proteins comprising mutant and/or chimeric CH regions having altered effector functions, and methods of engineering and testing mutant antibodies, are described in the art, e.g. K. L. Amour, et al., Eur. J. Immunol. 1999, 29:2613-2624; Lazar et al., Proc. Natl. Acad. Sci. USA 2006, 103:4005; US Patent Application Publication No. 20070135620A1 published Jun. 14, 2007; US Patent Application Publication No. 20080154025 A1, published Jun. 26, 2008; US Patent Application Publication No. 20100234572 A1, published Sep. 16, 2010; US Patent Application Publication No. 20120225058 A1, published Sep. 6, 2012; US Patent Application Publication No. 20150337053 A1, published Nov. 26, 2015; International Publication No. WO20/16161010A2 published Oct. 6, 2016; U.S. Pat. No. 9,359,437, issued Jun. 7, 2016; and U.S. Pat. No. 10,053,517, issued Aug. 21, 2018, all of which are herein incorporated by reference.

The C-terminal lysines (−K) conserved in the heavy chain genes of all human IgG subclasses are generally absent from antibodies circulating in serum- the C-terminal lysines are cleaved off in circulation, resulting in a heterogeneous population of circulating IgGs. (van den Bremer et al., 2015, mAbs 7:672-680). In the vectored constructs for full length mAbs, the DNA encoding the C-terminal lysine (−K) or glycine-lysine (−GK) of the Fc terminus can be deleted to produce a more homogeneous antibody product in situ. (See, Hu et al., 2017 Biotechnol. Prog. 33:786-794 which is incorporated by reference herein in its entirety).

### 5.1.10 Manufacture and Testing of Vectors

The viral vectors provided herein may be manufactured using host cells. The viral vectors provided herein may be manufactured using mammalian host cells, for example, A549, WEHI, 10T1/2, BHK, MDCK, COSI, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (e.g., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

Alternatively, baculovirus expression systems in insect cells may be used to produce AAV vectors. For a review, see Aponte-Ubillus et al., 2018, Appl. Microbiol. Biotechnol. 102:1045-1054 which is incorporated by reference herein in its entirety for manufacturing techniques.

In vitro assays, e.g., cell culture assays, can be used to measure transgene expression from a vector described herein, thus indicating, e.g., potency of the vector. In addition, in vitro neutralization assays can be used to measure the activity of the transgene expressed from a vector described herein. For example, Vero-E6 cells, a cell line derived from the kidney of an African green monkey, or HeLa cells engineered to stably express the ACE2 receptor (HeLa-ACE2), can be used to assess neutralization activity of transgenes expressed from a vector described herein. In addition, other characteristics of the expressed product can be determined, for example determination of the glycosylation and tyrosine sulfation patterns associated with the HuGlyFab. Glycosylation patterns and methods of determining the same are discussed in Section 5.3, while tyrosine sulfation patterns and methods of determining the same are discussed in Section 5.3. In addition, benefits resulting from glycosylation/sulfation of the cell-expressed HuGlyFab can be determined using assays known in the art, e.g., the methods described in Section 5.3.

Vector genome concentration (GC) or vector genome copies can be evaluated using digital PCR (dPCR) or ddPCR™ (BioRad Technologies, Hercules, CA, USA). In one example, ocular tissue samples, such as aqueous and/or vitreous humor samples, are obtained at several timepoints. In another example, several mice are sacrificed at various timepoints post injection. Ocular tissue samples are subjected to total DNA extraction and dPCR assay for vector copy numbers. Copies of vector genome (transgene) per gram of tissue may be measured in a single biopsy sample, or measured in various tissue sections at sequential timepoints will reveal spread of AAV throughout the eye. Total DNA from collected ocular fluid or tissue is extracted with the DNeasy Blood & Tissue Kit and the DNA concentration measured using a Nanodrop spectrophotometer. To determine the vector copy numbers in each tissue sample, digital PCR is performed with Naica Crystal Digital PCR system (Stilla technologies). Two color multiplexing system is applied to simultaneously measure the transgene AAV and an endogenous control gene. In brief, the transgene probe can be labelled with FAM (6-carboxyfluorescein) dye while the endogenous control probe can be labelled with VIC fluorescent dye. The copy number of delivered vector in a specific tissue section per diploid cell is calculated as: (vector copy number)/(endogenous control)×2. Vector copy in specific cell types or tissues, such as cornea, iris, ciliary body, schlemm's canal cells, trabecular meshwork, retinal cells, RPE cells, RPE-choroid tissue, or optic nerve cells, over time may indicate sustained expression of the transgene by the tissue.

### 5.1.11 Compositions

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides: proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

### 5.2 Methods of Treating Dry AMD

In another aspect, methods for treating dry AMD (age-related AMD) or other indication that can be treated with an anti-C5 or anti-C3 antibody, or CHFL-1 protein, in a subject in need thereof (a composition for use in the treatment of dry AMD or other indication that can be treated with an anti-C5, anti-C3 or CHFL-1 protein) comprising the administration of recombinant AAV particles comprising an expression cassette encoding an anti-C5 or C3 antibody or antibody-binding fragments and variants thereof, or CFHL-1 proteins, are provided. A subject in need thereof includes a subject suffering from dry AMD, or a subject pre-disposed thereto, e.g., a subject at risk of developing dry AMD, or other indication that may be treated with an anti-C5 or C3 antibody or CFHL-1 protein. Subjects to whom such gene therapy is administered can be those responsive to anti-C5 or C3 antibody or CFHL-1 protein e.g., crovalimab, eculizumab, ravulizumab, tesidolumab, or NGM621 antibody. In particular embodiments, the methods encompass treating patients who have been diagnosed with dry AMD, and, in certain embodiments, identified as responsive to treatment with an anti-C5 or anti-C3 antibody or CFHL-1 protein, or considered a good candidate for therapy with an anti-C5 or anti-C3 antibody or CFHL-1 protein. In specific embodiments, the patients have previously been treated with an anti-C5 or anti-C3 antibody or CFHL-1 protein. To determine responsiveness, the anti- an anti-C5 or anti-C3 antibody or antigen-binding fragment transgene product, or CFHL-1 protein (e.g., produced in human cell culture, bioreactors, etc.) may be administered directly to the subject.

In specific embodiments, provided are methods of treating dry AMD or other indication amenable to treatment with an anti-C3 or C5 antibody of hCFHL-1 protein in a human subject in need thereof comprising: administering to the eye, for example, intravitreal, subretinal, suprachoroidal, intracameral, or intranasal, or liver and/or muscle by systemic administration (including intravenous or intramuscular) of said subject a therapeutically effective amount of a recombinant nucleotide expression vector, such as an AAV vector, comprising a transgene encoding a substantially full-length or full-length anti-C5 or C3 antibody having an Fc region, or an antigen-binding fragment thereof, including an scFv, or hCFHL-1, operably linked to one or more regulatory sequences that control expression of the transgene in human ocular tissue cells (such as retinal cells, BrM cells, choriocapillaris cells, RPE cells and/or choroid cells), so that a depot is formed that releases a HuPTM form of mAb or antigen-binding fragment thereof or CFHL-1 protein. Subretinal, intravitreal, intracameral, or suprachoroidal administration should result in expression of the transgene product in one or more of the following retinal cell types: Bruch's membrane (BrM), including epithelial cells thereof, choriocapillaris, human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells or other ocular tissue cell: cornea cells, iris cells, ciliary body cells, a schlemm's canal cells, a trabecular meshwork cells, RPE-choroid tissue cells, or optic nerve cells.

Recombinant vectors and pharmaceutical compositions for treating diseases or disorders in a subject in need thereof are described in Section 5.1. Such vectors should have a tropism for human ocular tissue, or liver and/or muscle cells and can include non-replicating rAAV, particularly those bearing an AAV3B, AAV8, AAAV9, AAV10, AAVrh10, or AAVrh73 capsid. The recombinant vectors can be administered in any manner such that the recombinant vector enters ocular tissue cells, e.g., by introducing the recombinant vector into the eye. Such vectors should further comprise one or more regulatory sequences that control expression of the transgene in human ocular tissue cells and/or human liver and muscle cells include, but are not limited to, human rhodopsin kinase (GRK1) promoter (SEQ ID NOS: 77 or 217), a mouse cone arresting (CAR) promoter (SEQ ID NOS: 214-216), a human red opsin (RedO) promoter (SEQ ID NO: 212), a CAG promoter (SEQ ID NO: 74), a mutated CAG promoter (SEQ ID NO: 304, SEQ ID NO: 305 or SEQ ID NO: 306), a CB promoter or CBlong promoter (SEQ ID NO: 222 or 223) or a Best1/GRK1 tandem promoter (SEQ ID NO: 224) (see also Tables 1 and 1a).

The methods described herein treat, slow the progression of, reduce the severity of or prevent dry (age related) AMD in a human subject in need of the treatment. The treatment, slowing progression of, reduction of severity or prevention may be assessed relative to the subject prior to treatment, a comparable untreated subject or according to the natural history of the disease. In particular, method of the invention may reduce the progression of geographic atrophy, including within the fovea, slow retinal cell loss, slow the loss of central vision, increase or slow the loss of visual acuity, etc.

The subject may be at risk or have a predisposition to develop dry AMD based upon age, and/or risk factors such as history of smoking, obesity, cardiovascular disease or diabetes.

### 5.3.N-Glycosylation, Tyrosine Sulfation, and O-Glycosylation

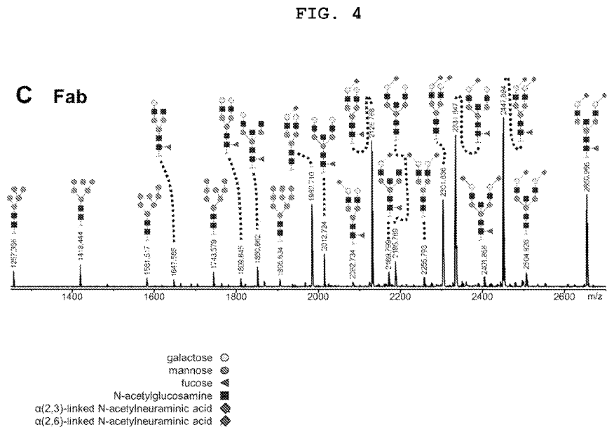

The amino acid sequence (primary sequence) of HuGlyFabs or HuPTM Fabs, HuPTMmAbs, and HuPTM scFvs disclosed herein each comprises at least one site at which N-glycosylation or tyrosine sulfation takes place (see exemplary FIG. 4) for glycosylation and/or sulfation positions within the amino acid sequences of the Fab fragments of the therapeutic antibodies). Post-translational modification also occurs in the Fc domain of full length antibodies, particularly at residue N297 (by EU numbering, see Table 6).

Alternatively, mutations may be introduced into the Fc domain to alter the glycosylation site at residue N297 (EU numbering, see Table 6), in particular substituting another amino acid for the asparagine at 297 or the threonine at 299 to remove the glycosylation site resulting in an aglycosylated Fc domain.

### 5.3.1. N-Glycosylation

### Reverse Glycosylation Sites

The canonical N-glycosylation sequence is known in the art to be Asn-X-Ser (or Thr), wherein X can be any amino acid except Pro. However, it recently has been demonstrated that asparagine (Asn) residues of human antibodies can be glycosylated in the context of a reverse consensus motif, Ser (or Thr)-X-Asn, wherein X can be any amino acid except Pro. See Valliere-Douglass et al., 2009, J. Biol. Chem. 284:32493-32506; and Valliere-Douglass et al., 2010, J. Biol. Chem. 285:16012-16022. As disclosed herein, certain HuGlyFabs and HuPTM scFvs disclosed herein comprise such reverse consensus sequences.

### Non-Consensus Glycosylation Sites

In addition to reverse N-glycosylation sites, it recently has been demonstrated that glutamine (Gln) residues of human antibodies can be glycosylated in the context of a non-consensus motif, Gln-Gly-Thr. See Valliere-Douglass et al., 2010, J. Biol. Chem. 285:16012-16022. Surprisingly, certain of the HuGlyFab fragments disclosed herein comprise such non-consensus sequences. In addition, O-glycosylation comprises the addition of N-acetyl-galactosamine to serine or threonine residues by the enzyme. It has been demonstrated that amino acid residues present in the hinge region of antibodies can be O-glycosylated. The possibility of O-glycosylation confers another advantage to the therapeutic antibodies provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098.)

### Engineered N-Glycosylation Sites

In certain embodiments, a nucleic acid encoding a HuPTM mAb, HuGlyFab or HuPTM scFv is modified to include 1, 2, 3, 4, 5, 6, 7, 8, 9, 10, or more N-glycosylation sites (including the canonical N-glycosylation consensus sequence, reverse N-glycosylation site, and non-consensus N-glycosylation sites) than would normally be associated with the HuPTM mAb, HuGlyFab or HuPTM scFv (e.g., relative to the number of N-glycosylation sites associated with the HuPTM mAb, HuGlyFab or HuPTM scFv in its unmodified state). In specific embodiments, introduction of glycosylation sites is accomplished by insertion of N-glycosylation sites (including the canonical N-glycosylation consensus sequence, reverse N-glycosylation site, and non-consensus N-glycosylation sites) anywhere in the primary structure of the antigen-binding fragment, so long as said introduction does not impact binding of the antibody or antigen-binding fragment to its antigen. Introduction of glycosylation sites can be accomplished by, e.g., adding new amino acids to the primary structure of the antigen-binding fragment, or the antibody from which the antigen-binding fragment is derived (e.g., the glycosylation sites are added, in full or in part), or by mutating existing amino acids in the antigen-binding fragment, or the antibody from which the antigen-binding fragment is derived, in order to generate the N-glycosylation sites (e.g., amino acids are not added to the antigen-binding fragment/antibody, but selected amino acids of the antigen-binding fragment/antibody are mutated so as to form N-glycosylation sites). Those of skill in the art will recognize that the amino acid sequence of a protein can be readily modified using approaches known in the art, e.g., recombinant approaches that include modification of the nucleic acid sequence encoding the protein.

In a specific embodiment, a HuGlyMab or antigen-binding fragment is modified such that, when expressed in mammalian cells, such as retina, CNS, liver or muscle cells, it can be hyperglycosylated. See Courtois et al., 2016, mAbs 8:99-112 which is incorporated by reference herein in its entirety.

N-Glycosylation of HuPTM mAbs and HuPTM Antigen-Binding Fragments

Unlike small molecule drugs, biologics usually comprise a mixture of many variants with different modifications or forms that could have a different potency, pharmacokinetics, and/or safety profile. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment provided herein can be, for example, to slow or arrest the progression of a disease or abnormal condition or to reduce the severity of one or more symptoms associated with the disease or abnormal condition.

When a HuPTM mAb, HuGlyFab or HuPTM scFv is expressed in a human cell, the N-glycosylation sites of the antigen-binding fragment can be glycosylated with various different glycans. N-glycans of antigen-binding fragments and the Fc domain have been characterized in the art. For example, Bondt et al., 2014, Mol. & Cell. Proteomics 13.11:3029-3039 (incorporated by reference herein in its entirety for its disclosure of Fab-associated N-glycans) characterizes glycans associated with Fabs, and demonstrates that Fab and Fc portions of antibodies comprise distinct glycosylation patterns, with Fab glycans being high in galactosylation, sialylation, and bisection (e.g., with bisecting GlcNAc) but low in fucosylation with respect to Fc glycans. Like Bondt, Huang et al., 2006, Anal. Biochem. 349:197-207 (incorporated by reference herein in its entirety for it disclosure of Fab-associated N-glycans) found that most glycans of Fabs are sialylated. However, in the Fab of the antibody examined by Huang (which was produced in a murine cell background), the identified sialic residues were N-Glycolylneuraminic acid (“Neu5Gc” or “NeuGc”) (which is not natural to humans) instead of N-acetylneuraminic acid (“Neu5Ac,” the predominant human sialic acid). In addition, Song et al., 2014, Anal. Chem. 86:5661-5666 (incorporated by reference herein in its entirety for its disclosure of Fab-associated N-glycans) describes a library of N-glycans associated with commercially available antibodies.

Glycosylation of the Fc domain has been characterized and is a single N-linked glycan at asparagine 297 (EU numbering; see Table 6). The glycan plays an integral structural and functional role, impacting antibody effector function, such as binding to Fc receptor (see, for example, Jennewein and Alter, 2017, Trends In Immunology 38:358 for a discussion of the role of Fc glycosylation in antibody function). Removal of the Fc region glycan almost completely ablates effector function (Jennewein and Alter at 362). The composition of the Fc glycan has been shown to impact effector function, for example hypergalactosylation and reduction in fucosylation have been shown to increase ADCC activity while sialylation correlates with anti-inflammatory effects (Id. at 364). Disease states, genetics and even diet can impact the composition of the Fc glycan in vivo. For recombinantly expressed antibodies, the glycan composition can differ significantly by the type of host cell used for recombinant expression and strategies are available to control and modify the composition of the glycan in therapeutic antibodies recombinantly expressed in cell culture, such as CHO to alter effector function (see, for example, US 2014/0193404 by Hansen et al.). Accordingly, the HuPTM mAbs provided herein may advantageously have a glycan at N297 that is more like the native, human glycan composition than antibodies expressed in non-human host cells.

Importantly, when the HuPTM mAb, HuGlyFab or HuPTM scFv are expressed in human cells, the need for in vitro production in prokaryotic host cells (e.g., E. coli) or eukaryotic host cells (e.g., CHO cells or NS0 cells) is circumvented. Instead, as a result of the methods described herein, N-glycosylation sites of the HuPTM mAb, HuGlyFab or HuPTM scFv are advantageously decorated with glycans relevant to and beneficial to treatment of humans. Such an advantage is unattainable when CHO cells, NS0 cells, or E. coli are utilized in antibody/antigen-binding fragment production, because e.g., CHO cells (1) do not express 2,6 sialyltransferase and thus cannot add 2,6 sialic acid during N-glycosylation; (2) can add Neu5Gc as sialic acid instead of Neu5Ac; and (3) can also produce an immunogenic glycan, the α-Gal antigen, which reacts with anti-α-Gal antibodies present in most individuals, which at high concentrations can trigger anaphylaxis; and because (4) E. coli does not naturally contain components needed for N-glycosylation.

Assays for determining the glycosylation pattern of antibodies, including antigen-binding fragments are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. Glycans may also be released using enzymes such as glycosidases or endoglycosidases, such as PNGase F and Endo H, which cleave cleanly and with fewer side reactions than hydrazines. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304 (1): 70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low or high molecular weight can be analyzed by MALDI-MS/MS and the result used to confirm the glycan sequence. Each peak in the chromatogram corresponds to a polymer, e.g., glycan, consisting of a certain number of repeat units and fragments, e.g., sugar residues, thereof. The chromatogram thus allows measurement of the polymer, e.g., glycan, length distribution. The elution time is an indication for polymer length, while fluorescence intensity correlates with molar abundance for the respective polymer, e.g., glycan. Other methods for assessing glycans associated with antigen-binding fragments include those described by Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666.

Homogeneity or heterogeneity of the glycan patterns associated with antibodies (including antigen-binding fragments), as it relates to both glycan length or size and numbers glycans present across glycosylation sites, can be assessed using methods known in the art, e.g., methods that measure glycan length or size and hydrodynamic radius. HPLC, such as size exclusion, normal phase, reversed phase, and anion exchange HPLC, as well as capillary electrophoresis, allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can be measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

In certain embodiments, the HuPTM mAbs, or antigen binding fragments thereof, also do not contain detectable NeuGc and/or α-Gal. By “detectable NeuGc” or “detectable α-Gal” or “does not contain or does not have NeuGc or α-Gal” means herein that the HuPTM mAb or antigen-binding fragment, does not contain NeuGc or α-Gal moieties detectable by standard assay methods known in the art. For example, NeuGc may be detected by HPLC according to Hara et al., 1989, “Highly Sensitive Determination of N-Acetyl- and N-Glycolylneuraminic Acids in Human Serum and Urine and Rat Serum by Reversed-Phase Liquid Chromatography with Fluorescence Detection.” J. Chromatogr., B: Biomed. 377, 111 119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring α-Gal epitope expression on cells by a monoclonal anti-Gal antibody.” Transplantation. 65 (8): 1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5 (5): 699-710. See also the references cited in Platts-Mills et al., 2015, “Anaphylaxis to the Carbohydrate Side-Chain Alpha-gal” Immunol Allergy Clin North Am. 35 (2): 247-260.

### Benefits of N-Glycosylation

N-glycosylation confers numerous benefits on the HuPTM mAb, HuGlyFab or HuPTM scFv described herein. Such benefits are unattainable by production of antigen-binding fragments in E. coli, because E. coli does not naturally possess components needed for N-glycosylation. Further, some benefits are unattainable through antibody production in, e.g., CHO cells (or murine cells such as NS0 cells), because CHO cells lack components needed for addition of certain glycans (e.g., 2,6 sialic acid and bisecting GlcNAc) and because either CHO or murine cell lines add N—N-Glycolylneuraminic acid (“Neu5Gc” or “NeuGc”) which is not natural to humans (and potentially immunogenic), instead of N-Acetylneuraminic acid (“Neu5Ac”) the predominant human sialic acid. See, e.g., Dumont et al., 2015, Crit. Rev. Biotechnol. 36 (6): 1110-1122; Huang et al., 2006, Anal. Biochem. 349:197-207 (NeuGc is the predominant sialic acid in murine cell lines such as SP2/0 and NS0); and Song et al., 2014, Anal. Chem. 86:5661-5666, each of which is incorporated by reference herein in its entirety). Moreover, CHO cells can also produce an immunogenic glycan, the α-Gal antigen, which reacts with anti-α-Gal antibodies present in most individuals, which at high concentrations can trigger anaphylaxis. See, e.g., Bosques, 2010, Nat. Biotech. 28:1153-1156. The human glycosylation pattern of the HuGlyFab of HuPTM scFv described herein should reduce immunogenicity of the transgene product and improve efficacy.

While non-canonical glycosylation sites usually result in low level glycosylation (e.g., 1-5%) of the antibody population, the functional benefits may be significant (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement.

The presence of sialic acid on HuPTM mAb, HuGlyFab or HuPTM scFv used in the methods described herein can impact clearance rate of the HuPTM mAb, HuGlyFab or HuPTM scFv. Accordingly, sialic acid patterns of a HuPTM mAb, HuGlyFab or HuPTM scFv can be used to generate a therapeutic having an optimized clearance rate. Methods of assessing antigen-binding fragment clearance rate are known in the art. See, e.g., Huang et al., 2006, Anal. Biochem. 349:197-207.

In another specific embodiment, a benefit conferred by N-glycosylation is reduced aggregation. Occupied N-glycosylation sites can mask aggregation prone amino acid residues, resulting in decreased aggregation. Such N-glycosylation sites can be native to an antigen-binding fragment used herein or engineered into an antigen-binding fragment used herein, resulting in HuGlyFab or HuPTM scFv that is less prone to aggregation when expressed, e.g., expressed in human cells. Methods of assessing aggregation of antibodies are known in the art. See, e.g., Courtois et al., 2016, mAbs 8:99-112 which is incorporated by reference herein in its entirety.

In another specific embodiment, a benefit conferred by N-glycosylation is reduced immunogenicity. Such N-glycosylation sites can be native to an antigen-binding fragment used herein or engineered into an antigen-binding fragment used herein, resulting in HuPTM mAb, HuGlyFab or HuPTM scFv that is less prone to immunogenicity when expressed, e.g., expressed in human ocular tissue cells, human CNS cells, human liver cells or human muscle cells.

In another specific embodiment, a benefit conferred by N-glycosylation is protein stability. N-glycosylation of proteins is well-known to confer stability on them, and methods of assessing protein stability resulting from N-glycosylation are known in the art. See, e.g., Sola and Griebenow, 2009, J Pharm Sci., 98 (4): 1223-1245.

In another specific embodiment, a benefit conferred by N-glycosylation is altered binding affinity. It is known in the art that the presence of N-glycosylation sites in the variable domains of an antibody can increase the affinity of the antibody for its antigen. See, e.g., Bovenkamp et al., 2016, J. Immunol. 196:1435-1441. Assays for measuring antibody binding affinity are known in the art. See, e.g., Wright et al., 1991, EMBO J. 10:2717-2723; and Leibiger et al., 1999, Biochem. J. 338:529-538.

### 5.3.2 Tyrosine Sulfation

Tyrosine sulfation occurs at tyrosine (Y) residues with glutamate (E) or aspartate (D) within +5 to −5 position of Y, and where position −1 of Y is a neutral or acidic charged amino acid, but not a basic amino acid, e.g., arginine (R), lysine (K), or histidine (H) that abolishes sulfation. The HuGlyFabs and HuPTM scFvs described herein comprise tyrosine sulfation sites (see exemplary FIG. 2).

Importantly, tyrosine-sulfated antigen-binding fragments cannot be produced in E. coli, which naturally does not possess the enzymes required for tyrosine-sulfation. Further, CHO cells are deficient for tyrosine sulfation—they are not secretory cells and have a limited capacity for post-translational tyrosine-sulfation. See, e.g., Mikkelsen & Ezban, 1991, Biochemistry 30:1533-1537. Advantageously, the methods provided herein call for expression of HuPTM Fab in human cells that are secretory and have capacity for tyrosine sulfation.

Tyrosine sulfation is advantageous for several reasons. For example, tyrosine-sulfation of the antigen-binding fragment of therapeutic antibodies against targets has been shown to dramatically increase avidity for antigen and activity. See, e.g., Loos et al., 2015, PNAS 112:12675-12680, and Choe et al., 2003, Cell 114:161-170. Assays for detection tyrosine sulfation are known in the art. See, e.g., Yang et al., 2015, Molecules 20:2138-2164.

### 5.3.3 O-Glycosylation

O-glycosylation comprises the addition of N-acetyl-galactosamine to serine or threonine residues by the enzyme. It has been demonstrated that amino acid residues present in the hinge region of antibodies can be O-glycosylated. In certain embodiments, the HuGlyFab comprise all or a portion of their hinge region, and thus are capable of being O-glycosylated when expressed in human cells. The possibility of O-glycosylation confers another advantage to the HuGlyFab provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098.) O-glycosylated HuGlyFab, by virtue of possessing glycans, shares advantageous characteristics with N-glycosylated HuGlyFab (as discussed above).

### 5.4 Anti-C5, Anti-C3 or CFHL Constructs and Formulations for Dry AMD

Compositions and methods are described for the delivery of HuPTM mAb or the antigen-binding fragment thereof, such as HuPTM Fab, that bind to C3 or C5 (and inhibit complement activation) derived from an anti-C5 or C3 antibody and indicated for treating dry AMD. In certain embodiments, the HuPTM mAb has the amino acid sequence of crovalimab, eculizumab, ravulizumab, tesidolumab or NGM621 or an antigen binding fragment thereof. The amino acid sequence of Fab fragment of these antibodies is provided in FIGS. 2A-2G (see also Table 7, which also provides amino acid sequences of certain full length and scFv constructs). In other embodiments, provided are compositions and methods for delivery of HuPTM CFHL-1 (amino acid sequence in Table 7). Delivery may be accomplished via gene therapy—e.g., by administering a viral vector or other DNA expression construct encoding an anti-C5 or anti-C3 HuPTM mAb (or an antigen binding fragment and/or a hyperglycosylated derivative or other derivative, thereof) or CFHL-1 to patients (human subjects) diagnosed with dry AMD to create a permanent depot that continuously supplies the human PTM, e.g., human-glycosylated, transgene product.

### Transgenes

Provided are recombinant vectors containing a transgene encoding a HuPTM mAb or HuPTM Fab (or other antigen binding fragment of the HuPTM mAb) that binds to C5 (and inhibits C5 cleavage and complement activation) or C3 (and inhibits C3 cleavage and complement activation) of CFHL-1, that can be administered to deliver the HuPTM mAb or antigen binding fragment or CFHL-1 in a patient. The transgene is a nucleic acid comprising the nucleotide sequences encoding an antigen binding fragment of an antibody that binds to C5, such as crovalimab, eculizumab, ravulizumab, or tesidolumab, or that binds to C3, such as NGM621, or the CFHL-1 protein, or variants thereof, as detailed herein. The transgene may also encode an C3 or C5 antigen binding fragment that contains additional glycosylation sites (e.g., see Courtois et al.).

In certain embodiments, the anti-C5 antigen-binding fragment transgene comprises the nucleotide sequences encoding the heavy and light chains of the Fab portion of crovalimab (having amino acid sequences of SEQ ID NOs. 1 and 2, respectively, see Table 7 and FIG. 2A). The nucleotide sequences may be codon optimized for expression in human cells. Nucleotide sequences may, for example, comprise the nucleotide sequences of SEQ ID NO: 26 (encoding the crovalimab heavy chain Fab portion) and SEQ ID NO: 27 (encoding the crovalimab light chain portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO: 85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Table 3 infra.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-C5-antigen binding domain has a heavy chain Fab domain of SEQ ID NO: 1 with additional hinge region sequence starting after the C-terminal valine (V), contains all or a portion of the amino acid sequence EPKSCDKTHTCPPCPAPELLRR (SEQ ID NO: 153), and specifically, EPKSCDKTH (SEQ ID NO: 307), EPKSCDKTHL (SEQ ID NO: 155), EPKSCDKTHT (SEQ ID NO: 156), EPKSCDKTHTCPPCPA (SEQ ID NO: 157), EPKSCDKTHLCPPCPA (SEQ ID NO:158), EPKSCDKTHTCPPCPAPELLGGPSVFL (SEQ ID NO: 159) or EPKSCDKTHLCPPCPAPELLGGPSVFL (SEQ ID NO:160) as set forth in FIG. 2A. In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 64 (Table 6) or an IgG1 Fc domain, such as SEQ ID No. 61 or as depicted in FIG. 5, or a mutant or variant thereof. The Fc domain may be engineered for altered binding to one or more Fc receptors and/or effector function as disclosed in Section 5.1.9, infra. In embodiments, the Fc domain has enhanced FcRn binding and recued FcγRI and C1q binding, for example has substitutions M428L and N434A.

In specific embodiments, provided are constructs encoding a full length crovalimab, including the Fc domain, particularly nucleotide sequence of full length crovalimab (SEQ ID NO: 38 encoding the expressed transgene polypeptide (including the leader and linker sequences)), or an antigen-binding fragment of crovalimab, particularly a Fab fragment (SEQ ID NO: 37 encoding the expressed transgene polypeptide (including the leader and linker sequences) as set forth in Table 8, herein, in certain cases depleted for CpG dimers. The transgene may also comprises a nucleotide sequence that encodes a signal peptide MYRMQLLLLIALSLALVTNS (SEQ ID NO: 85); for example at the N-terminal of the heavy and/or the light chain) which may be encoded by the nucleotide sequence of SEQ ID NO: 86. The nucleotide sequences encoding the light chain and heavy chain may be separated by a Furin-2A linker (SEQ ID NOs: 146-149, see also amino acid sequences of SEQ ID NOs: 142 and 144) to create a bicistronic vector. Alternatively, the nucleotide sequences of the light chain and heavy chain are separated by a Furin-T2A linker, such as SEQ ID NO: 145. Expression of crovalimab may be directed by a constitutive or a tissue specific promoter. In certain embodiments, the transgene contains a CAG promoter (SEQ ID NO: 74), a mutated (SEQ ID NO: 304, SEQ ID NO: 305 or SEQ ID NO: 306), a CB promoter or CB long promoter (SEQ ID NO: 222 or 223), a GRK1 (SEQ ID NO:77) promoter. Alternatively, the promoter may be a tissue specific promoter (or regulatory sequence including promoter and enhancer elements) such as the GRK1 promoter (SEQ ID NO: 77 or 217), (a mouse cone arresting (CAR) promoter (SEQ ID NOS: 214-216), a human red opsin (RedO) promoter (SEQ ID NO: 212) or a Best1/GRK1 tandem promoter (SEQ ID NO: 224). In embodiments, a intron sequence is positioned between the promoter and the coding sequence, for example a VH4 intron sequence (SEQ ID NO: 70). The transgenes may contain elements provided in Table 1 or 1a. Exemplary transgenes encoding full length and Fab fragments and scFv forms of crovalimab and artificial genomes (or constructs encoding artificial genomes) in which ITR sequences are added to the 3′ and 5′ ends, which are provided in Table 8, and include CAG.crovalimab.full (SEQ ID NO: 44) or CAG.crovalimab.Fab (SEQ ID NO: 43) or Crovalimab.scFv (SEQ ID NO: 267 or SEQ ID NO: 268 or SEQ ID NO: 269 or SEQ ID NO: 277 or SEQ ID NO: 278 or SEQ ID NO: 279 or SEQ ID NO: 280 or SEQ ID NO: 281 or SEQ ID NO: 282 or SEQ ID NO: 283 or SEQ ID NO: 284 or SEQ ID NO: 285 or SEQ ID NO: 286 or SEQ ID NO: 287 or SEQ ID NO: 288 or SEQ ID NO: 289 or SEQ ID NO: 290 or SEQ ID NO: 291 or SEQ ID NO: 292). The transgenes may be packaged into AAV, particularly AAV8. In embodiments, the artificial genome is self-complementary.

In certain embodiments, the anti-C5 antigen-binding fragment transgene encodes an C5 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-C5 antigen-binding fragment transgene encodes a C5 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-C5 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In specific embodiments, the C5 antigen binding fragment comprises a heavy chain comprising an amino acid sequence of SEQ ID NO: 1 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2A). In specific embodiments, the C5 antigen binding fragment comprises a light chain comprising an amino acid sequence of SEQ ID NO: 2 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2A).

In certain embodiments, the anti-C5 antigen-binding fragment transgene encodes an antigen-binding fragment and comprises the nucleotide sequences encoding the six crovalimab CDRs which are underlined in the heavy and light chain variable domain sequences of FIG. 2A which are spaced between framework regions, generally human framework regions, and associated with constant domains depending upon the form of the antigen-binding molecule, as is known in the art to form the heavy and/or light chain variable domain of an anti-C5 antibody or antigen-binding fragment thereof.

In certain embodiments, the anti-C5 antigen-binding fragment transgene comprises the nucleotide sequences encoding the heavy chain of the Fab portion of eculizumab (including either an IgG1 CH1 domain or an IgG2 CH1 domain (having amino acid sequences of SEQ ID NOs. 3 and 4 (heavy chain Fab with IgG1 and IgG2 CH1 domains, respectively) and light chain of eculizumab (SEQ ID NO: 5 see Table 7 and FIGS. 2B and 2C). The nucleotide sequences may be codon optimized for expression in human cells. Nucleotide sequences may, for example, comprise the nucleotide sequences of SEQ ID NO: 28 or 29 (encoding the eculizumab heavy chain IgG1 or IgG2 Fab portions, respectively) and SEQ ID NO: 30 (encoding the eculizumab light chain) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVINS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Table 3 infra. The nucleotide sequences encoding the light chain and heavy chain may be separated by a Furin-2A linker (SEQ ID NOs: 146-149, see also amino acid sequences of SEQ ID NOs: 142 and 144) to create a bicistronic vector. Alternatively, the nucleotide sequences of the light chain and heavy chain are separated by a Furin-T2A linker, such as SEQ ID NO: 145. Expression of eculizumab may be directed by a constitutive or a tissue specific promoter. In certain embodiments, the transgene contains a CAG promoter (SEQ ID NO: 74), a mutated CAG promoter (SEQ ID NO: 304, SEQ ID NO: 305 or SEQ ID NO: 306), a CB promoter or CB long promoter (SEQ ID NO: 222 or 223), a GRK1 (SEQ ID NO: 77) promoter. Alternatively, the promoter may be a tissue specific promoter (or regulatory sequence including promoter and enhancer elements) such as the GRK1 promoter (SEQ ID NO: 77 or 217), (a mouse cone arresting (CAR) promoter (SEQ ID NOS: 214-216), a human red opsin (RedO) promoter (SEQ ID NO: 212) or a Best1/GRK1 tandem promoter (SEQ ID NO: 224). In embodiments, an intron sequence is positioned between the promoter and the coding sequence, for example a VH4 intron sequence (SEQ ID NO: 70). The transgenes may contain elements provided in Table 1 or 1a. Exemplary transgenes encoding full length eculizumab and Fab fragments (both IgG1 and IgG2) of eculizumab and artificial genomes (or constructs encoding artificial genomes) in which ITR sequences are added to the 3′ and 5′ ends are provided in Table 8 and include CAG.eculizumab.full (SEQ ID NO: 47) or CAG.eculizumab.Fab.IgG1 (SEQ ID NO: 45) or CAG.eculizumab.Fab.IgG2 (SEQ ID NO: 46). The transgenes may be packaged into AAV, particularly AAV8.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-C5-antigen binding domain has a heavy chain Fab domain of SEQ ID NO: 3 or 4 with additional hinge region sequence starting after the C-terminal valine (V), contains all or a portion of the amino acid sequence EPKSCDKTHTCPPCPAPELLGG (SEQ ID NO:153), and specifically. EPKSCDKTHL (SEQ ID NO: 155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO: 159) or EPKSCDKTHLCPPCPAPELLGGPSVFL (SEQ ID NO: 160) as set forth in FIGS. 2B and 2C. In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 65 (Table 6) or an IgG1 Fc domain, such as SEQ ID No. 61 or as depicted in FIG. 5, or a mutant or variant thereof. The Fc domain may be engineered for altered binding to one or more Fc receptors and/or effector function as disclosed in Section 5.1.9, infra. In embodiments, the transgene comprises the nucleotide sequence of SEQ ID NO: 37 (encoding the crovalimab Fab fragment) or SEQ ID NO: 38 (encoding the crovalimab full length antibody).

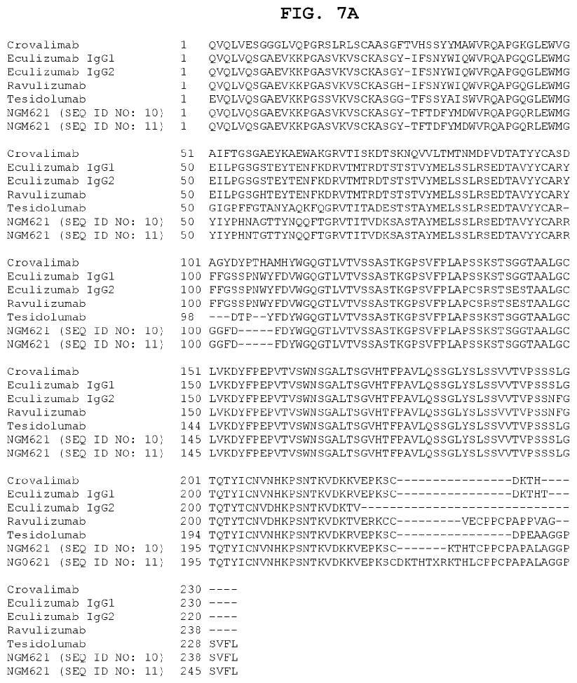

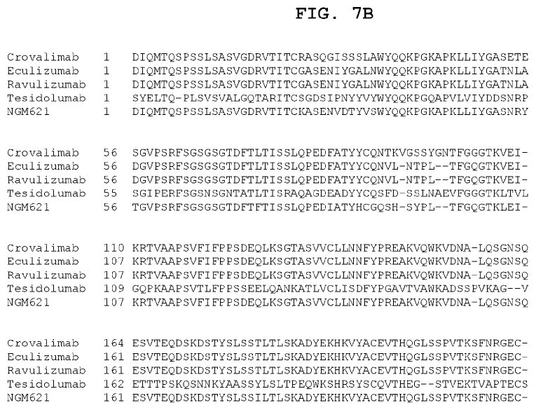

In certain embodiments, the anti-C5 antigen-binding fragment transgene encodes an C5 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 5. In certain embodiments, the anti-C5 antigen-binding fragment transgene encodes a C5 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3 or 4. In certain embodiments, the anti-C5 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 5 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3 or 4. In specific embodiments, the C5 antigen binding fragment comprises a heavy chain comprising an amino acid sequence of SEQ ID NO: 3 or 4 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIGS. 2B and 2C) or are substitutions with an amino acid present at that position in the heavy chain of one or more of the other therapeutic antibodies, for example, as identified by the alignment in FIG. 7A. In specific embodiments, the C5 antigen binding fragment comprises a light chain comprising an amino acid sequence of SEQ ID NO: 45 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIG. 2B or 2C) or are substitutions with an amino acid present at that position in the light chain of one or more of the other therapeutic antibodies, for example, as identified by the alignment in FIG. 7B.

In certain embodiments, the anti-C5 antigen-binding fragment transgene encodes an antigen-binding fragment and comprises the nucleotide sequences encoding the six eculizumab CDRs which are underlined in the heavy and light chain variable domain sequences of FIGS. 2B and 2C which are spaced between framework regions, generally human framework regions, and associated with constant domains depending upon the form of the antigen-binding molecule, as is known in the art to form the heavy and/or light chain variable domain of an anti-C5 antibody or antigen-binding fragment thereof.

In certain embodiments, the anti-C5 antigen-binding fragment transgene comprises the nucleotide sequences encoding the heavy and light chains of the Fab portion of ravulizumab (having amino acid sequences of SEQ ID NOs. 6 and 7, respectively, see Table 7 and FIG. 2D). The nucleotide sequences may be codon optimized for expression in human cells. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Table 3 infra. The nucleotide sequences encoding the light chain and heavy chain may be separated by a Furin-2A linker (SEQ ID NOs: 146-149, see also amino acid sequences of SEQ ID NOs: 142 and 144) to create a bicistronic vector. Alternatively, the nucleotide sequences of the light chain and heavy chain are separated by a Furin-T2A linker, such as SEQ ID NO:145. Expression of ravulizumab may be directed by a constitutive or a tissue specific promoter. In certain embodiments, the transgene contains a CAG promoter (SEQ ID NO: 74), a mutated CAG promoter (SEQ ID NO: 304, SEQ ID NO: 305 or SEQ ID NO: 306), a CB promoter or CB long promoter (SEQ ID NO: 222 or 223), a GRK1 (SEQ ID NO: 77) promoter. Alternatively, the promoter may be a tissue specific promoter (or regulatory sequence including promoter and enhancer elements) such as the GRK1 promoter (SEQ ID NO:77 or 217), (a mouse cone arresting (CAR) promoter (SEQ ID NOS: 214-216), a human red opsin (RedO) promoter (SEQ ID NO: 212) or a Best1/GRK1 tandem promoter (SEQ ID NO: 224). In embodiments, an intron sequence is positioned between the promoter and the coding sequence, for example a VH4 intron sequence (SEQ ID NO: 70). The transgenes may contain elements provided in Table 1 or 1a. The transgenes may be packaged into AAV, particularly AAV8.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-C5-antigen binding domain has a heavy chain variable domain of SEQ ID NO: 6 with additional hinge region sequence starting after the C-terminal valine (V), contains all or a portion of the amino acid sequence EPKSCDKTHTCPPCPAPELLGG (SEQ ID NO:153), and specifically, EPKSCDKTHL, (SEQ ID NO: 155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO: 159) or EPKSCDKTHLCPPCPAPELLGGPSVFL (SEQ ID NO:160) as set forth in FIG. 2D. In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 66 (Table 6) or an IgG1 Fc domain, such as SEQ ID No. 61 or as depicted in FIG. 5, or a mutant or variant thereof. The Fc domain may be engineered for altered binding to one or more Fc receptors and/or effector function as disclosed in Section 5.1.9, infra.

In certain embodiments, the anti-C5 antigen-binding fragment transgene encodes an C5 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 7. In certain embodiments, the anti-C5 antigen-binding fragment transgene encodes an C5 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 6. In certain embodiments, the anti-C5 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 7 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 6. In specific embodiments, the C5 antigen binding fragment comprises a heavy chain comprising an amino acid sequence of SEQ ID NO: 6 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIG. 2D) or are substitutions with an amino acid present at that position in the heavy chain of one or more of the other therapeutic antibodies, for example, as identified by the alignment in FIG. 7A. In specific embodiments, the C5 antigen binding fragment comprises a light chain comprising an amino acid sequence of SEQ ID NO: 7 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2D) or are substitutions with an amino acid present at that position in the light chain of one or more of the other therapeutic antibodies, for example, as identified by the alignment in FIG. 7B.

In certain embodiments, the anti-C5 antigen-binding fragment transgene encodes an antigen-binding fragment and comprises the nucleotide sequences encoding the six ravulizumab CDRs which are underlined in the heavy and light chain variable domain sequences of FIG. 2D which are spaced between framework regions, generally human framework regions, and associated with constant domains depending upon the form of the antigen-binding molecule, as is known in the art to form the heavy and/or light chain variable domain of an anti-C5 antibody or antigen-binding fragment thereof.

In certain embodiments, the anti-C5 antigen-binding fragment transgene comprises the nucleotide sequences encoding the heavy and light chains of the Fab portion of tesidolumab (having amino acid sequences of SEQ ID NOs. 8 and 9, respectively, see Table 7 and FIG. 2E). The nucleotide sequences may be codon optimized for expression in human cells. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO: 85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Table 3 infra. The nucleotide sequences encoding the light chain and heavy chain may be separated by a Furin-2A linker (SEQ ID NOs: 146-149, see also amino acid sequences of SEQ ID NOs: 142 and 144) to create a bicistronic vector. Alternatively, the nucleotide sequences of the light chain and heavy chain are separated by a Furin-T2A linker, such as SEQ ID NO:145. Expression of tesidolumab may be directed by a constitutive or a tissue specific promoter. In certain embodiments, the transgene contains a CAG promoter (SEQ ID NO: 74), a mutated CAG promoter (SEQ ID NO: 304, SEQ ID NO: 305 or SEQ ID NO: 306), a CB promoter or CB long promoter (SEQ ID NO: 222 or 223), a GRK1 (SEQ ID NO: 77) promoter. Alternatively, the promoter may be a tissue specific promoter (or regulatory sequence including promoter and enhancer elements) such as the GRK1 promoter (SEQ ID NO:77 or 217), (a mouse cone arresting (CAR) promoter (SEQ ID NOS: 214-216), a human red opsin (RedO) promoter (SEQ ID NO: 212) or a Best1/GRK1 tandem promoter (SEQ ID NO: 224). In embodiments, an intron sequence is positioned between the promoter and the coding sequence, for example a VH4 intron sequence (SEQ ID NO: 70). The transgenes may contain elements provided in Table 1 or 1a. The transgenes may be packaged into AAV, particularly AAV8.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-C5-antigen binding domain has a heavy chain variable domain of SEQ ID NO: 8 with additional hinge region sequence starting after the C-terminal valine (V), contains all or a portion of the amino acid sequence EPKSCDKTHTCPPCPAPELLGG (SEQ ID NO:153), and specifically. EPKSCDKTHL (SEQ ID NO: 155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO: 159) or EPKSCDKTHLCPPCPAPELLGGPSVFL (SEQ ID NO:160) as set forth in FIG. 2E. In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 67 (Table 6) or an IgG1 Fc domain, such as SEQ ID No. 61 or as depicted in FIG. 5, or a mutant or variant thereof. The Fc domain may be engineered for altered binding to one or more Fc receptors and/or effector function as disclosed in Section 5.1.9, infra.

In certain embodiments, the anti-C5 antigen-binding fragment transgene encodes an C5 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO. 9. In certain embodiments, the anti-C5 antigen-binding fragment transgene encodes an C5 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 8. In certain embodiments, the anti-C5 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 9 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 68. In specific embodiments, the C5 antigen binding fragment comprises a heavy chain comprising an amino acid sequence of SEQ ID NO: 6 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIG. 2E) or are substitutions with an amino acid present at that position in the heavy chain of one or more of the other therapeutic antibodies, for example, as identified by the alignment in FIG. 7A. In specific embodiments, the C5 antigen binding fragment comprises a light chain comprising an amino acid sequence of SEQ ID NO: 9 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2E) or are substitutions with an amino acid present at that position in the light chain of one or more of the other therapeutic antibodies, for example, as identified by the alignment in FIG. 7B.

In certain embodiments, the anti-C5 antigen-binding fragment transgene encodes an antigen-binding fragment and comprises the nucleotide sequences encoding the six tesidolumab CDRs which are underlined in the heavy and light chain variable domain sequences of FIG. 2E which are spaced between framework regions, generally human framework regions, and associated with constant domains depending upon the form of the antigen-binding molecule, as is known in the art to form the heavy and/or light chain variable domain of an anti-C5 antibody or antigen-binding fragment thereof.

In certain embodiments, the anti-C3 antigen-binding fragment transgene comprises the nucleotide sequences encoding the heavy and light chains of the Fab portion of NGM621 (the heavy chain having amino acid sequences of SEQ ID NOs. 10 or 11 and light chain having an amino acid sequence of SEQ ID NO: 14), respectively, see Table 7 and FIGS. 2F and 2G). The nucleotide sequences may be codon optimized for expression in human cells. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Table 3 infra. The nucleotide sequences encoding the light chain and heavy chain may be separated by a Furin-2A linker (SEQ ID NOs: 146-149, see also amino acid sequences of SEQ ID NOs: 142 and 144) to create a bicistronic vector. Alternatively, the nucleotide sequences of the light chain and heavy chain are separated by a Furin-T2A linker, such as SEQ ID NO:145. Expression of NGM621 may be directed by a constitutive or a tissue specific promoter. In certain embodiments, the transgene contains a CAG promoter (SEQ ID NO: 74), a mutated CAG promoter (SEQ ID NO: 304, SEQ ID NO: 305 or SEQ ID NO: 306), a CB promoter or CB long promoter (SEQ ID NO: 222 or 223), a GRK1 (SEQ ID NO:77) promoter. Alternatively, the promoter may be a tissue specific promoter (or regulatory sequence including promoter and enhancer elements) such as the GRK1 promoter (SEQ ID NO:77 or 217). (a mouse cone arresting (CAR) promoter (SEQ ID NOS: 214-216), a human red opsin (RedO) promoter (SEQ ID NO: 212) or a Best1/GRK1 tandem promoter (SEQ ID NO: 224). In embodiments, an intron sequence is positioned between the promoter and the coding sequence, for example a VH4 intron sequence (SEQ ID NO: 70). The transgenes may contain elements provided in Table 1 or 1a. The transgenes may be packaged into AAV, particularly AAV8.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-C53-antigen binding domain has a heavy chain variable domain of SEQ ID NO: 6 with additional hinge region sequence starting after the C-terminal valine (V), contains all or a portion of the amino acid sequence EPKSCDKTHTCPPCPAPELLGG (SEQ ID NO:153), and specifically, EPKSCDKTHL (SEQ ID NO: 155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO: 159) or EPKSCDKTHLCPPCPAPELLGGPSVFL (SEQ ID NO: 160) as set forth in FIGS. 2F and 2G. In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 68 (Table 6) or an IgG1 Fc domain, such as SEQ ID No. 61 or as depicted in FIG. 5, or a mutant or variant thereof. The Fc domain may be engineered for altered binding to one or more Fc receptors and/or effector function as disclosed in Section 5.1.9, infra.

In certain embodiments, the anti-C3 antigen-binding fragment transgene encodes an C3 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 11. In certain embodiments, the anti-C3 antigen-binding fragment transgene encodes an C3 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 10. In certain embodiments, the anti-C3 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 11 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 10. In specific embodiments, the C3 antigen binding fragment comprises a heavy chain comprising an amino acid sequence of SEQ ID NO: 10 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIGS. 2F and 2G) or are substitutions with an amino acid present at that position in the heavy chain of one or more of the other therapeutic antibodies, for example, as identified by the alignment in FIG. 7A. In specific embodiments, the C3 antigen binding fragment comprises a light chain comprising an amino acid sequence of SEQ ID NO: 11 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2F or 2G) or are substitutions with an amino acid present at that position in the light chain of one or more of the other therapeutic antibodies, for example, as identified by the alignment in FIG. 7B.

In certain embodiments, the anti-C3 antigen-binding fragment transgene encodes an antigen-binding fragment and comprises the nucleotide sequences encoding the six ravulizumabNGM621 CDRs which are underlined in the heavy and light chain variable domain sequences of FIGS. 2F and 2G which are spaced between framework regions, generally human framework regions, and associated with constant domains depending upon the form of the antigen-binding molecule, as is known in the art to form the heavy and/or light chain variable domain of an anti-C3 antibody or antigen-binding fragment thereof.

Also provided are transgenes, and recombinant AAV vectors comprising the transgenes, encoding BB5.1, an anti-C5 antibody that binds C5 in non-human primates and can be used as a surrogate antibody for at least eculizumab and ravulizumab in pre-clinical studies of the recombinant vectors described herein that encode those anti-C5 antibodies and antigen-binding fragments thereof. Accordingly, the transgenes encode the heavy and light chains of BB5.1 (SEQ ID Nos: 15 and 16 are the full length heavy chain and the light chain of BB5.1) (see Table 7). Nucleotide sequences encoding the BB5.1 heavy and light chains include SEQ ID NO: 35 and 36, respectively. The BB5.1 heavy and light chains may be expressed from a transgene, heavy and light chains linked by a linker which has an amino acid sequence of SEQ ID NO:22 and may be encoded by a nucleotide sequence of SEQ ID NO: 42. In embodiments, the transgene encoding the BB5.1 heavy and light chains may be operably linked to regulatory sequences for expression in ocular tissue cells and may be flanked by ITR sequences. For example, the construct encoding or the artificial genome has a nucleotide sequence of SEQ ID NO: 48.

In certain embodiments, provided are vectors, including AAV vectors comprising a transgene encoding the human complement factor H-like-1 protein (CFHL-1) having an amino acid sequence of SEQ ID NO: 23 (Table 7). The CFHL-1 protein may be encoded by a nucleotide sequence comprising SEQ ID NO. 49 (see Table 8). Alternatively, provided are vectors, including AAV vectors comprising a transgene encoding the human complement factor H protein, having an amino acid sequence of SEQ ID NO: 24 (see UniProtKB-P08603). The first eighteen amino acids of SEQ ID NO: 23 and SEQ ID NO: 24 are the CFHL-1 or CFH signal sequence (also SEQ ID NO: 90, Table 2). The nucleotide sequences may be codon optimized for expression in human cells. The CFHL-1 protein may have a signal or leader sequence at the N-terminus appropriate for expression and secretion in human cells, in particular, human ocular tissue cells (e.g., retinal cells) or liver and/or muscle cells. The signal sequence may have the amino acid sequence of the endogenous CFHL-1 (or CFH) sequence, which is MRLLAKIICLMLWAICVA (SEQ ID NO: 90) (underlined in Table 7) or may be MYRMQLLLLIALSLALVINS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Table 3 infra. Expression of CHLH-1 may be directed by a constitutive or a tissue specific promoter. In certain embodiments, the transgene contains a CAG promoter (SEQ ID NO: 74), a mutated CAG promoter (SEQ ID NO: 304, SEQ ID NO: 305 or SEQ ID NO: 306), a CB promoter or CB long promoter (SEQ ID NO: 222 or 223), a GRK1 (SEQ ID NO:77) promoter. Alternatively, the promoter may be a tissue specific promoter (or regulatory sequence including promoter and enhancer elements) such as the GRK1 promoter (SEQ ID NO:77 or 217), (a mouse cone arresting (CAR) promoter (SEQ ID NOS: 214-216), a human red opsin (RedO) promoter (SEQ ID NO: 212) or a Best1/GRK1 tandem promoter (SEQ ID NO: 224). In embodiments, an intron sequence is positioned between the promoter and the coding sequence, for example a VH4 intron sequence (SEQ ID NO: 70). The transgenes may contain elements provided in Table 1 or 1a. The transgenes may be packaged into AAV, particularly AAV8.

In certain embodiments, provided is a construct encoding or an artificial genome in which the transgene operably linked to regulatory sequences is flanked by ITR sequences. In some embodiments, the artificial genome is self-complementary. The construct or artificial genome may comprise or consist of the nucleotide sequence of SEQ ID NO: 48. The artificial genome may comprise of the nucleotide sequence of any one of SEQ ID NO: 26 to 50 or any one of SEQ ID Nos: 267 to 269 or any one of SEQ ID Nos: 277 to 303. The artificial genome may comprise of the nucleotide sequence at least about 80%, at least about 85%, at least about 90%, at least about 91%, at least about 92%, at least about 93%, at least about 94%, at least about 95%, at least about 96%, at least 97%, at least about 98%, or at least about 99% identical to any one of SEQ ID NOs: 26 to 50 or any one of SEQ ID Nos: 267 to 269 or any one of SEQ ID Nos: 277 to 303 which encodes and expresses a C5 or C3 antibody or antigen binding fragment thereof as described herein.

Table 7 provides the amino acid sequences of Fab, scFv and full length heavy and light chains of the anti-C3 and anti-C5 antibodies, and the expression products of the transgenes, including the signal sequences and linkers, such as Furin/T2a linkers, and the amino acid sequence of CFHL-1. Table 8 provides a nucleotide sequence encoding the Fab and full length heavy and light chains of the antibodies and of the CFHL-1 protein, transgene coding sequences, and artificial genomes disclosed herein.

TABLE 7
Amino Acid Sequences of Heavy and Light Chains and Protein

| | Chain/ | |
|---|---|---|
| | SEQ ID | |
| mAb or Protein | NO: | Sequences |
| Crovalimab | Heavy/ | QVQLVESGGG LVQPGRSLRL SCAASGFTVH SSYYMAWVRQ |
| Fab | SEQ ID | APGKGLEWVG AIFTGSGAEY KAEWAKGRVT ISKDTSKNQV |
| | NO: 1 | VLTMTNMDPV DTATYYCASD AGYDYPTHAM HYWGQGTLVT |
| | | VSSASTKGPS VFPLAPSSKS TSGGTAALGC LVKDYFPEPV |
| | | TVSWNSGALT SGVHTFPAVL QSSGLYSLSS VVTVPSSSLG |
| | | TQTYICNVNH KPSNTKVDKK VEPKSCDKTH |
| Crovalimab | Light/ | DIQMTQSPSS LSASVGDRVT ITCRASQGIS SSLAWYQQKP |
| | SEQ ID | GKAPKLLIYG ASETESGVPS RFSGSGSGTD FTLTISSLQP |
| | NO: 2 | EDFATYYCQN TKVGSSYGNT FGGGTKVEIK RTVAAPSVFI |
| | | FPPSDEQLKS GTASVVCLLN NFYPREAKVQ WKVDNALQSG |
| | | NSQESVTEQD SKDSTYSLSS TLTLSKADYE KHKVYACEVT |
| | | HQGLSSPVTK SENRGEC |
| Eculizumab | Heavy/ | QVQLVQSGAE VKKPGASVKV SCKASGYIFS NYWIQWVRQA |
| Fab IgG1 | SEQ ID | PGQGLEWMGE ILPGSGSTEY TENFKDRVTM TRDTSTSTVY |
| | NO: 3 | MELSSLRSED TAVYYCARYF FGSSPNWYFD VWGQGTLVTV |
| | | SSASTKGPSV FPLAPSSKST SGGTAALGCL VKDYFPEPVT |
| | | VSWNSGALTS GVHTFPAVLQ SSGLYSLSSV VTVPSSSLGT |
| | | QTYICNVNHK PSNTKVDKRV EPKSCDKTHT |
| Eculizumab | Heavy/ | QVQLVQSGAE VKKPGASVKV SCKASGYIFS NYWIQWVRQA |
| Fab IgG2 | SEQ ID | PGQGLEWMGE ILPGSGSTEY TENFKDRVTM TRDTSTSTVY |
| | NO: 4 | MELSSLRSED TAVYYCARYF FGSSPNWYFD VWGQGTLVTV |
| | | SSASTKGPSV FPLAPCSRST SESTAALGCL VKDYFPEPVT |
| | | VSWNSGALTS GVHTFPAVLQ SSGLYSLSSV VTVPSSNFGT |
| | | QTYTCNVDHK PSNTKVDKTV |
| Eculizumab | Light/ | DIQMTQSPSS LSASVGDRVT ITCGASENIY GALNWYQQKP |
| | SEQ ID | GKAPKLLIYG ATNLADGVPS RESGSGSGTD FTLTISSLQP |
| | NO: 5 | EDFATYYCQN VLNTPLTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSEN RGEC |
| Ravulizumab | Heavy/ | QVQLVQSGAE VKKPGASVKV SCKASGHIFS NYWIQWVRQA |
| Fab | SEQ ID | PGQGLEWMGE ILPGSGHTEY TENFKDRVTM TRDTSTSTVY |
| | NO: 6 | MELSSLRSED TAVYYCARYF FGSSPNWYFD VWGQGTLVTV |
| | | SSASTKGPSV FPLAPCSRST SESTAALGCL VKDYFPEPVT |
| | | VSWNSGALTS GVHTFPAVLQ SSGLYSLSSV VTVPSSNEGT |
| | | QTYTCNVDHK PSNTKVDKTV ERKCCVE +/−CPPCPA+/−PPVAG |
| Ravulizumab | Light/ | DIQMTQSPSS LSASVGDRVT ITCGASENIY GALNWYQQKP |
| | SEQ ID | GKAPKLLIYG ATNLADGVPS RESGSGSGTD FTLTISSLQP |
| | NO: 7 | EDFATYYCQN VINTPLTEGQ GTKVEIKRTV AAPSVEIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSEN RGEC |
| Tesidolumab | Heavy/ | EVQLVQSGAE VKKPGSSVKV SCKASGGTFS SYAI SWVRQA |
| Fab | SEQ ID | PGQGLEWMGG IGPFFGTANY AQKFQGRVTI TADESTSTAY |
| | NO: 8 | MELSSLRSED TAVYYCARDT PYFDYWGQGT LVTVSSASTK |
| | | GPSVFPLAPS SKSTSGGTAA LGCLVKDYFP EPVTVSWNSG |
| | | ALTSGVHTFP AVLQSSGLYS LSSVVTVPSS SLGTQTYICN |
| | | VNHKPSNTKV DKRVEPKSCD +/−KTHT (or KTHL) |
| | | +/−CPPCPA +/−PEAAGGPSVFL |
| Tesidolumab | Light/ | SYELTQPLSV SVALGQTARI TCSGDSIPNY YVYWYQQKPG |
| | SEQ ID | QAPVLVIYDD SNRPSGIPER FSGSNSGNTA TLTISRAQAG |
| | NO: 9 | DEADYYCQSF DSSLNAEVFG GGTKLTVLGQ PKAAPSVTLF |
| | | PPSSEELQAN KATLVCLISD FYPGAVTVAW KADSSPVKAG |
| | | VETTTPSKQS NNKYAASSYL SLTPEQWKSH RSYSCQVTHE |
| | | GSTVEKTVAP TECS |
| NGM621 | Heavy/ | QVQLVQSGAE VKKPGASVKV SCKASGYTFT DFYMDWVRQA |
| Fab | SEQ ID | PGQRLEWMGY IYPHNAGTTY NQQFTGRVTI TVDKSASTAY |
| | NO: 10 | MELSSLRSED TAVYYCARRG GFDFDYWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC D =/−KTHT (or KTHL) CPPCP |
| | | APALAGGPSV FL |
| NGM621 | Heavy/ | QVQLVQSGAE VKKPGASVKV SCKASGYTFT DFYMDWVRQA |
| Fab | SEQ ID | PGQRLEWMGY IYPHNTGTTY NQQFTGRVTI TVDKSASTAY |
| | NO: 11 | MELSSLRSED TAVYYCARRG GFDFDYWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC D =/−KTHT (or KTHL) CPPCP |
| | | APALAGGPSV FL |
| NGM621 | Heavy/ | QVQLVQSGAE VKKPGASVKV SCKASGYTFT DFYMDWVRQA |
| Full length | SEQ ID | PGQRLEWMGY IYPHNAGTTY NQQFTGRVTI TVDKSASTAY |
| heavy chain | NO: 12 | MELSSLRSED TAVYYCARRG GFDFDYWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC DKTHTCPPCP APALAGGPSV |
| | | FLFPPKPKDT LMISRIPEVT CVVVDVSHED PEVKENWYVD |
| | | GVEVHNAKTK PREEQYNSTY RVVSVLTVLH QDWLNGKEYK |
| | | CKVSNKALPA PIEKTISKAK GQPREPQVYT LPPSREEMTK |
| | | NQVSLTCLVK GFYPSDIAVE WESNGQPENN YKTTPPVLDS |
| | | DGSFFLYSKL TVDKSRWQQG NVFSCSVMHE ALHNHYTQKS LSLSPGK |
| NGM621 | Light/ | DIQMTQSPSS LSASVGDRVT ITCKASENVD TYVSWYQQKP |
| | SEQ ID | GKAPKLLIYG ASNRYTGVPS RFSGSGSGTD FTFTISSLQP |
| | NO: 13 | EDIATYHCGQ SHSYPLTFGQ GTKLEIK |
| NGM621 | Light/ | DIQMTQSPSS LSASVGDRVT ITCKASENVD TYVSWYQQKP |
| | SEQ ID | GKAPKLLIYG ASNRYTGVPS RFSGSGSGTD FTFTISSLQP |
| | NO: 14 | EDIATYHCGQ SHSYPLTFGQ GTKLEIKRTV AAPSVFIFPP |
| | | SDEQLKSGTA SVVCLLNNFY PREAKVQWKV DNALQSGNSQ |
| | | ESVTEQDSKD STYSLSSILT LSKADYEKHK VYACEVTHQG |
| | | LSSPVTKSEN RGEC |
| BB5.1 | Heavy/ | QVQLQQPGAE LVRPGTSVKL SCKASGYTFT SSWMHWVKQR |
| Full length | SEQ ID | PGQGLEWIGV IDPSDSYTNY NQKFKGKATL TVDTSSSTAY |
| heavy chain | NO: 15 | MQLSSLTSED SAVYYCARGG GSSYNRYFDV WGTGTTVTVS |
| | | SAKTTPPSVY PLAPGSAAQT NSMVTLGCLV KGYFPEPVTV |
| | | TWNSGSLSSG VHTFPAVLQS DLYTLSSSVT VPSSPRPSET |
| | | VTCNVAHPAS STKVDKKIVP RDCGCKPCIC TVPEVSSVFI |
| | | FPPKPKDVLT ITLTPKVTCV VVDISKDDPE VQFSWFVDDV |
| | | EVHTAQTQPR EEQFNSTERS VSELPIMHQD WLNGKEFKCR |
| | | VNSAAFPAPI EKTISKTKGR PKAPQVYTIP PPKEQMAKDK |
| | | VSLTCMITDF FPEDITVEWQ WNGQPAENYK NTQPIMNING |
| | | SYFVYSKLNV QKSNWEAGNT FTCSVLHEGL HNHHTEKSLS HSPGK |
| BB5.1 | Light/ | NIMMTQSPSS LAVSAGEKVT MSCKSSQSVL YSSNQKNYLA |
| | SEQ ID | WYQQKPGQSP KLLIYWASTR ESGVPDRFTG SGSGTDFTLT |
| | NO: 16 | ISSVQAEDLA VYYCHQYLSS RTFGGGTKLE IKRADAAPTV |
| | | SIFPPSSEQL TSGGASVVCF LNNFYPKDIN VKWKIDGSER |
| | | QNGVLNSWTD QDSKDSTYSM SSTLTLTKDE YERHNSYTCE |
| | | ATHKTSTSPI VKSFNRGEC |
| Crovalimab | SEQ ID | MYRMQLLLLI ALSLALVINS QVQLVESGGG LVQPGRSLRL |
| Vectorized Fab- | NO: 17 | SCAASGFTVH SSYYMAWVRQ APGKGLEWVG AIFTGSGAEY |
| fully encoded | | KAEWAKGRVT ISKDTSKNQV VLTMTNMDPV DTATYYCASD |
| protein | | AGYDYPTHAM HYWGQGTLVT VSSASTKGPS VFPLAPSSKS |
| | | TSGGTAALGC LVKDYFPEPV TVSWNSGALT SGVHTFPAVL |
| | | QSSGLYSLSS VVTVPSSSLG TQTYICNVNH KPSNTKVDKK |
| | | VEPKSCDKTH RKRRGSGEGR GSLLTCGDVE ENPGPMYRMQ |
| | | LLLLIALSLA LVTNSDIQMT QSPSSLSASV GDRVTITCRA |
| | | SQGISSSLAW YQQKPGKAPK LLIYGASETE SGVPSRESGS |
| | | GSGTDFTLTI SSLQPEDFAT YYCQNTKVGS SYGNTFGGGT |
| | | KVEIKRTVAA PSVFIFPPSD EQLKSGTASV VCLLNNFYPR |
| | | EAKVQWKVDN ALQSGNSQES VTEQDSKDST YSLSSTLTLS |
| | | KADYEKHKVY ACEVTHQGLS SPVTKSENRG EC |
| | | Leaders are shown in bold; Furin-2A linker is |
| | | underlined; hinge region is shown in italic |
| Crovalimab | SEQ ID | MYRMQLLLLI ALSLALVINS QVQLVESGGG LVQPGRSLRL |
| Vectorized fully | NO: 18 | SCAASGFTVH SSYYMAWVRQ APGKGLEWVG AIFTGSGAEY |
| encoded protein | | KAEWAKGRVT ISKDTSKNQV VLTMTNMDPV DTATYYCASD |
| | | AGYDYPTHAM HYWGQGTLVT VSSASTKGPS VFPLAPSSKS |
| | | TSGGTAALGC LVKDYFPEPV TVSWNSGALT SGVHTFPAVL |
| | | QSSGLYSLSS VVTVPSSSLG TQTYICNVNH KPSNTKVDKK |
| | | VEPKSCDKTH TCPPCPAPEL RRGPKVFLFP PKPKDTLMIS |
| | | RTPEVTCVVV DVSHEDPEVK FNWYVDGVEV HNAKTKPREE |
| | | QYNSTYRVVS VLTVLHQDWL NGKEYKCKVS NKGLPSSIEK |
| | | TISKAKGQPR EPQVYTLPPS REEMTKNQVS LTCLVKGFYP |
| | | SDIAVEWESN GQPENNYKTT PPVLDSDGSF FLYSKLTVDK |
| | | SRWQQGNVFS CSVLHEALHA HYTRKELSLS PRKRRGSGEG |
| | | RGSLLTCGDV EENPGPMYRM QLLLLIALSL ALVTNSDIQM |
| | | TQSPSSLSAS VGDRVTITCR ASQGISSSLA WYQQKPGKAP |
| | | KLLIYGASET ESGVPSRFSG SGSGTDFTLT ISSLQPEDFA |
| | | TYYCQNTKVG SSYGNTFGGG TKVEIKRTVA APSVFIFPPS |
| | | DEQLKSGTAS VVCLINNFYP REAKVQWKVD NALQSGNSQE |
| | | SVTEQDSKDS TYSLSSTLTL SKADYEKHKV YACEVTHQGL |
| | | SSPVTKSENR GEC |
| | | Leaders are shown in bold; Furin-2A linker is |
| | | underlined; hinge region is shown in italic |
| Eculizumab | SEQ ID | MYRMQLLLLI ALSLALVTNS QVQLVQSGAE VKKPGASVKV |
| Vectorized Fab- | NO: 19 | SCKASGYIFS NYWIQWVRQA PGQGLEWMGE ILPGSGSTEY |
| IgG1 fully | | TENFKDRVTM TRDTSTSTVY MELSSLRSED TAVYYCARYF |
| encoded protein | | FGSSPNWYFD VWGQGTLVTV SSASTKGPSV FPLAPSSKST |
| | | SGGTAALGCL VKDYFPEPVT VSWNSGALTS GVHTFPAVLQ |
| | | SSGLYSLSSV VTVPSSSLGT QTYICNVNHK PSNTKVDKRV |
| | | EPKSCDKTHT RKRRGSGEGR GSLLTCGDVE ENPGPMYRMQ |
| | | LLLLIALSLA LVTNSDIQMT QSPSSLSASV GDRVTITCGA |
| | | SENIYGALNW YQQKPGKAPK LLIYGATNLA DGVPSRESGS |
| | | GSGTDFTLTI SSLQPEDFAT YYCQNVLNTP LTFGQGTKVE |
| | | IKRTVAAPSV FIFPPSDEQL KSGTASVVCL LNNFYPREAK |
| | | VQWKVDNALQ SGNSQESVTE QDSKDSTYSL SSTLTLSKAD |
| | | YEKHKVYACE VTHQGLSSPV TKSENRGEC |
| | | Leaders are shown in bold; Furin-2A linker is |
| | | underlined; hinge region is shown in italic |
| Eculizumab | SEQ ID | MYRMQLLLLI ALSLALVTNS QVQLVQSGAE VKKPGASVKV |
| Vectorized Fab- | NO: 20 | SCKASGYIFS NYWIQWVRQA PGQGLEWMGE ILPGSGSTEY |
| IgG2 fully | | TENFKDRVTM TRDTSTSTVY MELSSLRSED TAVYYCARYF |
| encoded protein | | FGSSPNWYFD VWGQGTLVTV SSASTKGPSV FPLAPCSRST |
| | | SESTAALGCL VKDYFPEPVT VSWNSGALTS GVHTFPAVLQ |
| | | SSGLYSLSSV VTVPSSNFGT QTYTCNVDHK PSNTKVDKTV |
| | | RKRRGSGEGR GSLLTCGDVE ENPGPMYRMQ LLLLIALSLA |
| | | LVTNSDIQMT QSPSSLSASV GDRVTITCGA SENIYGALNW |
| | | YQQKPGKAPK LLIYGATNLA DGVPSRESGS GSGTDFTLTI |
| | | SSLQPEDFAT YYCQNVLNTP LTFGQGTKVE IKRTVAAPSV |
| | | FIFPPSDEQL KSGTASVVCL LNNFYPREAK VQWKVDNALQ |
| | | SGNSQESVTE QDSKDSTYSL SSTLTLSKAD YEKHKVYACE |
| | | VTHQGLSSPV TKSFNRGEC |
| | | Leaders are shown in bold; Furin-2A linker is |
| | | underlined; hinge region is shown in italic |
| Eculizumab | SEQ ID | MYRMQLLLLI ALSLALVTNS QVQLVQSGAE VKKPGASVKV |
| Vectorized fully | NO: 21 | SCKASGYIFS NYWIQWVRQA PGQGLEWMGE ILPGSGSTEY |
| encoded protein | | TENFKDRVTM TRDTSTSTVY MELSSLRSED TAVYYCARYF |
| | | FGSSPNWYFD VWGQGTLVTV SSASTKGPSV FPLAPCSRST |
| | | SESTAALGCL VKDYFPEPVT VSWNSGALTS GVHTFPAVLQ |
| | | SSGLYSLSSV VTVPSSNFGT QTYTCNVDHK PSNTKVDKTV |
| | | ERKCCVECPP CPAPPVAGPS VFLFPPKPKD TLMISRTPEV |
| | | TCVVVDVSQE DPEVQFNWYV DGVEVHNAKT KPREEQENST |
| | | YRVVSVLTVL HQDWINGKEY KCKVSNKGLP SSIEKTISKA |
| | | KGQPREPQVY TLPPSQEEMT KNQVSLTCLV KGFYPSDIAV |
| | | EWESNGQPEN NYKTTPPVLD SDGSFFLYSR LTVDKSRWQE |
| | | GNVFSCSVMH EALHNHYTQK SLSLSLGKRK RRGSGEGRGS |
| | | LLTCGDVEEN PGPMYRMQLL LLIALSLALV TNSDIQMTQS |
| | | PSSLSASVGD RVTITCGASE NIYGALNWYQ QKPGKAPKLL |
| | | IYGATNLADG VPSRESGSGS GTDFTLTISS LQPEDFATYY |
| | | CQNVLNTPLT FGQGTKVEIK RTVAAPSVFI FPPSDEQLKS |
| | | GTASVVCLLN NFYPREAKVQ WKVDNALQSG NSQESVTEQD |
| | | SKDSTYSLSS TLTLSKADYE KHKVYACEVT HQGLSSPVTK SFNRGEC |
| | | Leaders are shown in bold; Furin-2A linker is |
| | | underlined; hinge region is shown in italic |
| BB5.1 | SEQ ID | MYRMQLLLLIALSLALVTNSQVQLQQPGAELVRPGTSVKLSCKASGYTFTSS |
| Vectorized full | NO: 22 | WMHWVKQRPGQGLEWIGVIDPSDSYTNYNQKFKGKATLTVDTSSSTAYMQLS |
| length mAb- | | SLTSEDSAVYYCARGGGSSYNRYFDVWGTGTTVTVSSAKTTPPSVYPLAPGS |
| fully encoded | | AAQTNSMVTLGCLVKGYFPEPVTVTWNSGSLSSGVHTFPAVLQSDLYTLSSS |
| protein | | VTVPSSPRPSETVTCNVAHPASSTKVDKKIVPRDCGCKPCICTVPEVSSVFI |
| | | FPPKPKDVLTITLTPKVTCVVVDISKDDPEVQFSWFVDDVEVHTAQTQPREE |
| | | QFNSTFRSVSELPIMHQDWLNGKEFKCRVNSAAFPAPIEKTISKTKGRPKAP |
| | | QVYTIPPPKEQMAKDKVSLTCMITDFFPEDITVEWQWNGQPAENYKNTQPIM |
| | | NTNGSYFVYSKLNVQKSNWEAGNTFTCSVLHEGLHNHHTEKSLSHSPGKRKR |
| | | RGSGEGRGSLLTCGDVEENPGPMYRMQLLLLIALSLALVTNSNIMMTQSPSS |
| | | LAVSAGEKVTMSCKSSQSVLYSSNQKNYLAWYQQKPGQSPKLLIYWASTRES |
| | | GVPDRFTGSGSGTDFTLTISSVQAEDLAVYYCHQYLSSRTFGGGTKLEIKRA |
| | | DAAPTVSIFPPSSEQLTSGGASVVCFLNNFYPKDINVKWKIDGSERQNGVLN |
| | | SWTDQDSKDSTYSMSSTLTLTKDEYERHNSYTCEATHKTSTSPIVKSENRGE |
| | | C |
| | | Leaders are shown in bold; Furin-2A linker is |
| | | underlined; hinge region is shown in italic |
| CFHL1 | SEQ ID | MRLLAKIICLMLWAICVAEDCNELPPRRNTEILTGSWSDQTYPEGTQAIYKC |
| | NO: 23 | RPGYRSLGNVIMVCRKGEWVALNPLRKCQKRPCGHPGDTPFGTFTLTGGNVF |
| | | EYGVKAVYTCNEGYQLLGEINYRECDTDGWTNDIPICEVVKCLPVTAPENGK |
| | | IVSSAMEPDREYHFGQAVRFVCNSGYKIEGDEEMHCSDDGFWSKEKPKCVEI |
| | | SCKSPDVINGSPISQKIIYKENERFQYKCNMGYEYSERGDAVCTESGWRPLP |
| | | SCEEKSCDNPYIPNGDYSPLRIKHRTGDEITYQCRNGFYPATRGNTAKCTST |
| | | GWIPAPRCTLKPCDYPDIKHGGLYHENMRRPYFPVAVGKYYSYYCDEHFETP |
| | | SGSYWDHIHCTQDGWSPAVPCLRKCYFPYLENGYNQNHGRKFVQGKSIDVAC |
| | | HPGYALPKAQTTVTCMENGWSPTPRCIRVSFTL* |
| | | Leader sequence in bold. |
| Human | SEQ ID | MRLLAKIICLMLWAICVAEDCNELPPRRNTEILTGSWSDQTYPEGTQAIYKC |
| Complement | NO: 24 | RPGYRSLGNVIMVCRKGEWVALNPLRKCQKRPCGHPGDTPFGTFTLTGGNVF |
| Factor H | | EYGVKAVYTCNEGYQLLGEINYRECDTDGWTNDIPICEVVKCLPVTAPENGK |
| (UniProtKB- | | IVSSAMEPDREYHFGQAVRFVCNSGYKIEGDEEMHCSDDGFWSKEKPKCVEI |
| P08603) | | SCKSPDVINGSPISQKIIYKENERFQYKCNMGYEYSERGDAVCTESGWRPLP |
| | | SCEEKSCDNPYIPNGDYSPLRIKHRTGDEITYQCRNGFYPATRGNTAKCTST |
| | | GWIPAPRCTLKPCDYPDIKHGGLYHENMRRPYFPVAVGKYYSYYCDEHFETP |
| | | SGSYWDHIHCTQDGWSPAVPCLRKCYFPYLENGYNQNYGRKFVQGKSIDVAC |
| | | HPGYALPKAQTTVTCMENGWSPTPRCIRVKTCSKSSIDIENGFISESQYTYA |
| | | LKEKAKYQCKLGYVTADGETSGSITCGKDGWSAQPTCIKSCDIPVEMNARTK |
| | | NDFTWFKLNDTLDYECHDGYESNTGSTTGSIVCGYNGWSDLPICYERECELP |
| | | KIDVHLVPDRKKDQYKVGEVLKFSCKPGFTIVGPNSVQCYHFGLSPDLPICK |
| | | EQVQSCGPPPELLNGNVKEKTKEEYGHSEVVEYYCNPRFLMKGPNKIQCVDG |
| | | EWTTLPVCIVEESTCGDIPELEHGWAQLSSPPYYYGDSVEFNCSESFTMIGH |
| | | RSITCIHGVWTQLPQCVAIDKLKKCKSSNLIILEEHLKNKKEFDHNSNIRYR |
| | | CRGKEGWIHTVCINGRWDPEVNCSMAQIQLCPPPPQIPNSHNMTTTLNYRDG |
| | | EKVSVLCQENYLIQEGEEITCKDGRWQSIPLCVEKIPCSQPPQIEHGTINSS |
| | | RSSQESYAHGTKLSYTCEGGFRISEENETTCYMGKWSSPPQCEGLPCKSPPE |
| | | ISHGVVAHMSDSYQYGEEVTYKCFEGFGIDGPAIAKCLGEKWSHPPSCIKTD |
| | | CLSLPSFENAIPMGEKKDVYKAGEQVTYTCATYYKMDGASNVTCINSRWTGR |
| | | PTCRDTSCVNPPTVQNAYIVSRQMSKYPSGERVRYQCRSPYEMFGDEEVMCL |
| | | NGNWTEPPQCKDSTGKCGPPPPIDNGDITSFPLSVYAPASSVEYQCQNLYQL |
| | | EGNKRITCRNGQWSEPPKCLHPCVISREIMENYNIALRWTAKQKLYSRTGES |
| | | VEFVCKRGYRLSSRSHTLRTTCWDGKLEYPTCAKR |
| | | Leader sequence is in bold. |
| Crovalimab | SEQ ID | QVQLVESGGG LVQPGRSLRL SCAASGFTVH SSYYMAWVRQ |
| VH | NO: 251 | APGKGLEWVG AIFTGSGAEY KAEWAKGRVT ISKDTSKNQV |
| | | VLTMTNMDPV DTATYYCASD AGYDYPTHAM HYWGQGTLVT VSSA |
| Crovalimab | SEQ ID | DIQMTQSPSS LSASVGDRVT ITCRASQGIS SSLAWYQQKP |
| VL | NO: 252 | GKAPKLLIYG ASETESGVPS RFSGSGSGTD FTLTISSLQP |
| | | EDFATYYCQN TKVGSSYGNT FGGGTKVEIK R |
| Eculizumab | SEQ ID | QVQLVQSGAE VKKPGASVKV SCKASGYIFS NYWIQWVRQA |
| VH | NO: 253 | PGQGLEWMGE ILPGSGSTEY TENFKDRVTM TRDTSTSTVY |
| | | MELSSLRSED TAVYYCARYF FGSSPNWYFD VWGQGTLVTV |
| | | SSASTKGPSV FPLAPSSKST SGGTAALGCL VKDYFPEPVT |
| | | VSWNSGALTS GVHTFPAVLQ SSGLYSLSSV VTVPSSSLGT |
| | | QTYICNVNHK PSNTKVDKRV EPKSCDKTHT |
| Eculizumab | SEQ ID | DIQMTQSPSS LSASVGDRVT ITCGASENIY GALNWYQQKP |
| VL | NO: 254 | GKAPKLLIYG ATNLADGVPS RFSGSGSGTD FTLTISSLQP |
| | | EDFATYYCQN VLNTPLTFGQ GTKVEIKR |
| Ravulizumab | SEQ ID | QVQLVQSGAE VKKPGASVKV SCKASGHIFS NYWIQWVRQA |
| VH | NO: 255 | PGQGLEWMGE ILPGSGHTEY TENEKDRVTM TRDTSTSTVY |
| | | MELSSLRSED TAVYYCARYF FGSSPNWYFD VWGQGTLVTV SS |
| Ravulizumab | SEQ ID | DIQMTQSPSS LSASVGDRVT ITCGASENIY GALNWYQQKP |
| VL | NO: 256 | GKAPKLLIYG ATNLADGVPS RESGSGSGTD FTLTISSLQP |
| | | EDFATYYCQN VINTPLTEGQ GTKVEIKR |
| Tesidolumab | SEQ ID | EVQLVQSGAE VKKPGSSVKV SCKASGGTES SYAISWVRQA |
| VH | NO: 257 | PGQGLEWMGG IGPFFGTANY AQKFQGRVTI TADESTSTAY |
| | | MELSSLRSED TAVYYCARDT PYFDYWGQGT LVTVSS |
| Tesidolumab | SEQ ID | SYELTQPLSV SVALGQTARI TCSGDSIPNY YVYWYQQKPG |
| VL | NO: 258 | QAPVLVIYDD SNRPSGIPER FSGSNSGNTA TLTISRAQAG |
| | | DEADYYCQSF DSSLNAEVFG GGTKLTVL |
| NGM621 | SEQ ID | QVQLVQSGAE VKKPGASVKV SCKASGYTFT DFYMDWVRQA |
| VH | NO: 259 | PGQRLEWMGY IYPHNAGTTY NQQFTGRVTI TVDKSASTAY |
| | | MELSSLRSED TAVYYCARRG GFDEDYWGQG TLVTVSS |
| NGM621 | SEQ ID | DIQMTQSPSS LSASVGDRVT ITCKASENVD TYVSWYQQKP |
| VL | NO: 260 | GKAPKLLIYG ASNRYTGVPS RESGSGSGTD FTFTISSLQP |
| | | EDIATYHCGQ SHSYPLTFGQ GTKLEIK |
| Crovalimab | SEQ ID | QVQLVESGGGLVQPGRSLRLSCAASGFTVHSSYYMAWVRQAPG |
| Vectorized scFv | NO: 261 | KGLEWVGAIFTGSGAEYKAEWAKGRVTISKDTSKNQVVLTMIN |
| mAb- fully | | MDPVDTATYYCASDAGYDYPTHAMHYWGQGTLVTVSS |
| encoded | | GGGGSGGGGSGGGGS |
| protein | | DIQMTQSPSSLSASVGDRVTITCRASQGISSSLAWYQQKPGKA |
| (H-L) | | PKLLIYGASETESGVPSRFSGSGSGTDFTLTISSLQPEDFATY |
| | | YCQNTKVGSSYGNTFGGGTKVEIKR |
| | | Heavy chain + linker + Light chain |
| Crovalimab | SEQ ID | DIQMTQSPSSLSASVGDRVTITCRASQGISSSLAWYQQKPGKA |
| Vectorized scFv | NO: 262 | PKLLIYGASETESGVPSRFSGSGSGTDFTLTISSLQPEDFATY |
| mAb- fully | | YCQNTKVGSSYGNTFGGGTKVEIKR |
| encoded protein | | GGGGSGGGGSGGGGS |
| (L-H) | | QVQLVESGGGLVQPGRSLRLSCAASGFTVHSSYYMAWVRQAPG |
| | | KGLEWVGAI FTGSGAEYKAEWAKGRVTISKDTSKNQVVLTMTN |
| | | MDPVDTATYYCASDAGYDYPTHAMHYWGQGTLVTVSS |
| | | Light chain + linker + Heavy chain |
| Eculizumab | SEQ ID | QVQLVQSGAE VKKPGASVKV SCKASGYIFS NYWIQWVRQA |
| Vectorized scFv | NO: 263 | PGQGLEWMGE ILPGSGSTEY TENFKDRVTM TRDTSTSTVY |
| mAb- fully | | MELSSLRSED TAVYYCARYF FGSSPNWYFD VWGQGTLVTV |
| encoded protein | | SSA |
| (H-L) | | GGGGGGGGSGGGGS |
| | | DIQMTQSPSS LSASVGDRVT ITCGASENIY GALNWYQQKP |
| | | GKAPKLLIYG ATNLADGVPS RFSGSGSGTD FTLTISSLQP |
| | | EDFATYYCQN VLNTPLTFGQ GTKVEIKR |
| | | Heavy chain + linker + Light chain |
| Eculizumab | SEQ ID | DIQMTQSPSS LSASVGDRVT ITCGASENIY GALNWYQQKP |
| Vectorized scFv | NO: 264 | GKAPKLLIYG ATNLADGVPS RFSGSGSGTD FTLTISSLQP |
| mAb- fully | | EDFATYYCQN VLNTPLTFGQ GTKVEIKR |
| encoded protein | | GGGGSGGGGSGGGGS |
| (L-H) | | QVQLVQSGAE VKKPGASVKV SCKASGYIFS NYWIQWVRQA |
| | | PGQGLEWMGE ILPGSGSTEY TENFKDRVTM TRDTSTSTVY |
| | | MELSSLRSED TAVYYCARYF FGSSPNWYFD VWGQGTLVTV |
| | | SSA |
| | | Light chain + linker + Heavy chain |
| Ravulizumab | SEQ ID | QVQLVQSGAE VKKPGASVKV SCKASGHIFS NYWIQWVRQA |
| Vectorized scFv | NO: 265 | PGQGLEWMGE ILPGSGHTEY TENFKDRVTM TRDTSTSTVY |
| mAb- fully | | MELSSIRSED TAVYYCARYF FGSSPNWYFD VWGQGTLVTV SS |
| encoded protein | | GGGGSGGGGSGGGGS |
| (H-L) | | DIQMTQSPSS LSASVGDRVT ITCGASENIY GALNWYQQKP |
| | | GKAPKLLIYG ATNLADGVPS RESGSGSGTD FILTISSLQP |
| | | EDFATYYCQN VENTPLTEGQ GTKVEIKR |
| | | Heavy chain + linker + Light chain |
| Ravulizumab | SEQ ID | DIQMTQSPSS LSASVGDRVT ITCGASENIY GALNWYQQKP |
| Vectorized scFv | NO: 266 | GKAPKLLIYG ATNLADGVPS RESGSGSGTD FILTISSLQP |
| mAb- fully | | EDFATYYCQN VINTPLTFGQ GTKVEIKR |
| encoded protein | | GGGGSGGGGSGGGGS |
| (L-H) | | QVQLVQSGAE VKKPGASVKV SCKASGHIFS NYWIQWVRQA |
| | | PGQGLEWMGE ILPGSGHTEY TENEKDRVTM TRDTSTSTVY |
| | | MELSSLRSED TAVYYCARYF FGSSPNWYFD VWGQGTLVTV SS |
| | | Light chain + linker + Heavy chain |
| Tesidolumab | SEQ ID | EVQLVQSGAE VKKPGSSVKV SCKASGGTFS SYAISWVRQA |
| Vectorized scFv | NO: 270 | PGQGLEWMGG IGPFFGTANY AQKFQGRVTI TADESTSTAY |
| mAb- fully | | MELSSLRSED TAVYYCARDT PYFDYWGQGT LVTVSS |
| encoded protein | | GGGGSGGGGSGGGGS |
| (H-L) | | SYELTQPLSV SVALGQTARI TCSGDSIPNY YVYWYQQKPG |
| | | QAPVLVIYDD SNRPSGIPER FSGSNSGNTA TLTISRAQAG |
| | | DEADYYCQSF DSSLNAEVFG GGTKLTVL |
| | | Heavy chain + linker + Light chain |
| Tesidolumab | SEQ ID | SYELTQPLSV SVALGQTARI TCSGDSIPNY YVYWYQQKPG |
| Vectorized scFv | NO: 271 | QAPVLVIYDD SNRPSGIPER FSGSNSGNTA TLTISRAQAG |
| mAb- fully | | DEADYYCQSF DSSLNAEVFG GGTKLTVL |
| encoded protein | | GGGGSGGGGSGGGGS |
| (L-H) | | EVQLVQSGAE VKKPGSSVKV SCKASGGTFS SYAISWVRQA |
| | | PGQGLEWMGG IGPFFGTANY AQKFQGRVTI TADESTSTAY |
| | | MELSSLRSED TAVYYCARDT PYFDYWGQGT LVTVSS |
| | | Light chain + linker + Heavy chain |
| Crovalimab.ScFv | SEQ ID | MYRMQLLLLIALSLALVTNSQVQLVESGGGLVQPGRSLRLSCAASGFT |
| .HL (signal | NO: 272 | VHSSYYMAWVRQAPGKGLEWVGAIFTGSGAEYKAEWAKGRVTISKDTS |
| sequence | | TKNQVVLTMNMDPVDTATYYCASDAGYDYPTHAMHYWGQGTLVTVSSG |
| underlined) | | GGGSGGGGSGGGGSDIQMTQSPSSLSASVGDRVTITCRASQGISSSLA |
| | | WYQQKPGKAPKLLIYGASETESGVPSRFSGSGSGTDFTLTISSLQPED |
| | | FATYYCQNTKVGSSYGNTFGGGTKVEIKR |
| Crovalimab.scFv. | SEQ ID | MYRMQLLLLIALSLALVTNSDIQMTQSPSSLSASVGDRVTITCRASQ |
| LH (signal | NO: 273 | GISSSLAWYQQKPGKAPKLLIYGASETESGVPSRFSGSGSGTDFTLT |
| sequence | | ISSLQPEDFATYYCQNTKVGSSYGNTFGGGTKVEIKRGGGGSGGGGS |
| underlined) | | GGGGSQVQLVESGGGLVQPGRSLRLSCAASGFTVHSSYYMAWVRQAP |
| | | GKGLEWVGAIFTGSGAEYKAEWAKGRVTISKDTSKNQVVLTMTNMDP |
| | | VDTATYYCASDAGYDYPTHAMHYWGQGTLVTVSS |
| BB5.1.scFv.HL | SEQ ID | MYRMQLLLLIALSLALVTNS |
| (signal sequence | NO: 274 | QVQLQQPGAELVRPGTSVKLSCKASGYTFTSSWMHWVKQRPGQGLE |
| underlined) | | WIGVIDPSDSYTNYNQKFKGKATLTVDTSSSTAYMQLSSLTSEDSA |
| | | VYYCARGGGSSYNRYFDVWGTGTTVTVSSGGGGSGGGGSGGGGSNI |
| | | MMTQSPSSLAVSAGEKVTMSCKSSQSVLYSSNQKNYLAWYQQKPGQ |
| | | SPKLLIYWASTRESGVPDRFTGSGSGTDFTLTISSVQAEDLAVYYC |
| | | HQYLSSRTFGGGTKLEIKR |
| BB5.1.scFv.LH | SEQ ID | MYRMQLLLLIALSLALVTNS |
| (signal sequence | NO: 275 | NIMMTQSPSSLAVSAGEKVTMSCKSSQSVLYSSNQKNYLAWYQQKP |
| underlined) | | GQSPKLLIYWASTRESGVPDRFTGSGSGTDFTLTISSVQAEDLAVY |
| | | YCHQYLSSRTFGGGTKLEIKRGGGGSGGGGSGGGGSQVQLQQPGAE |
| | | LVRPGTSVKLSCKASGYTFTSSWMHWVKQRPGQGLEWIGVIDPSDS |
| | | YTNYNQKFKGKATLTVDTSSSTAYMQLSSLTSEDSAVYYCARGGGS |
| | | SYNRYFDVWGTGTTVTVSS |
| BB5.1.scFv.LH- | SEQ ID | NIMMTQSPSSLAVSAGEKVTMSCKSSQSVLYSSNQKNYLAWYQQKP |
| FLAG-Tag | NO: 276 | GQSPKLLIYWASTRESGVPDRFTGSGSGTDFTLTISSVQAEDLAVY |
| (protein) | | YCHQYLSSRTFGGGTKLEIKRGGGGSGGGGSGGGGSQVQLQQPGAE |
| | | LVRPGTSVKLSCKASGYTFTSSWMHWVKQRPGQGLEWIGVIDPSDS |
| | | YTNYNQKFKGKATLTVDTSSSTAYMQLSSLTSEDSAVYYCARGGGS |
| | | SYNRYFDVWGTGTTVTVSS |

TABLE 8
Nucleotide Sequences of Heavy and Light Chains, Expressed Polypeptides, Proteins,
and Genomes

| | Chain/ | |
|---|---|---|
| mAb, Protein or | SEQ ID | |
| construct name | NO: | Sequences |
| Crovalimab | Heavy/ | CAGGTGCAGCTGGTTGAATCTGGTGGTGGACTGGTGCAGCCTGGCAGAAGCC |
| Heavy Chain | SEQ ID | TGAGACTGTCTTGTGCTGCCTCTGGCTTCACAGTGCACAGCAGCTACTACAT |
| Fab | NO: 26 | GGCCTGGGTTAGACAGGCCCCTGGCAAAGGACTTGAGTGGGTTGGAGCCATC |
| | | TTCACAGGCTCTGGGGCTGAGTATAAGGCTGAGTGGGCCAAGGGCAGAGTGA |
| | | CCATCAGCAAGGACACCAGCAAGAACCAGGTGGTGCTGACCATGACCAACAT |
| | | GGACCCTGTGGACACAGCCACCTACTACTGTGCCTCTGATGCTGGCTATGAC |
| | | TACCCCACACATGCCATGCACTATTGGGGCCAGGGCACCCTGGTTACAGTGT |
| | | CCTCTGCCTCTACAAAGGGCCCCTCTGTGTTTCCTCTGGCTCCTAGCAGCAA |
| | | GAGCACCAGTGGTGGAACAGCTGCCCTGGGCTGTCTGGTCAAGGATTACTTC |
| | | CCTGAGCCAGTGACTGTGTCCTGGAACTCTGGTGCACTGACCTCTGGGGTGC |
| | | ACACCTTTCCAGCTGTGCTGCAGTCCTCTGGCCTGTACTCTCTGTCCTCTGT |
| | | GGTCACAGTGCCTAGCTCTAGCCTGGGCACCCAGACCTACATCTGCAATGTG |
| | | AACCACAAGCCTAGCAACACCAAGGTGGACAAGAAGGTGGAACCCAAGAGCT |
| | | GTGACAAGACCCAC |
| Crovalimab | Light/ | GACATCCAGATGACACAGAGCCCTAGCAGCCTGTCTGCCTCTGTGGGAGACA |
| light chain | SEQ ID | GAGTGACCATCACCTGTAGAGCCAGCCAGGGCATCTCTAGCAGTCTGGCCTG |
| | NO: 27 | GTATCAGCAGAAGCCTGGAAAGGCCCCTAAGCTGCTGATCTATGGGGCCTCT |
| | | GAGACAGAATCTGGGGTGCCCAGCAGATTTTCAGGCTCTGGCTCTGGCACAG |
| | | ACTTCACCCTGACCATTTCTAGCCTGCAGCCTGAGGACTTTGCCACCTACTA |
| | | CTGCCAGAACACCAAAGTGGGCAGCAGCTATGGCAACACCTTTGGTGGTGGC |
| | | ACCAAGGTGGAAATCAAGAGAACAGTGGCTGCCCCTTCTGTGTTCATCTTCC |
| | | CACCATCTGATGAGCAGCTGAAGAGTGGCACAGCCTCTGTTGTGTGCCTGCT |
| | | GAACAACTTCTACCCCAGAGAAGCCAAGGTGCAGTGGAAGGTGGACAATGCC |
| | | CTGCAGTCAGGCAACAGCCAAGAGTCTGTGACTGAGCAGGACAGCAAGGACT |
| | | CCACCTACAGCCTGAGCAGCACCCTGACACTGAGCAAGGCTGACTATGAGAA |
| | | GCACAAAGTGTATGCCTGTGAAGTGACCCACCAGGGGCTGAGCAGCCCTGTG |
| | | ACCAAGAGCTTCAATAGAGGGGAGTGC |
| Eculizumab Fab | Heavy/ | CAGGTGCAGCTGGTTCAGTCTGGGGCTGAAGTGAAGAAACCTGGGGCCTCTG |
| IgG1 | SEQ ID | TGAAGGTGTCCTGCAAGGCCTCTGGCTACATCTTCAGCAACTATTGGATCCA |
| | NO: 28 | GTGGGTCAGACAGGCCCCTGGCCAAGGACTTGAGTGGATGGGAGAGATCCTG |
| | | CCTGGCTCTGGCAGCACAGAGTACACAGAGAACTTCAAGGACAGAGTGACCA |
| | | TGACCAGAGACACCAGCACCTCCACAGTGTACATGGAACTGAGCAGCCTGAG |
| | | ATCTGAGGACACAGCAGTGTACTATTGTGCCAGATACTTCTTTGGCAGCAGC |
| | | CCCAACTGGTACTTTGATGTGTGGGGCCAGGGCACCCTGGTTACAGTGTCCT |
| | | CTGCCTCTACAAAGGGCCCCTCTGTGTTTCCTCTGGCTCCTAGCAGCAAGAG |
| | | CACCAGTGGTGGAACAGCTGCCCTGGGCTGTCTGGTCAAGGATTACTTCCCT |
| | | GAGCCTGTGACTGTGTCCTGGAACTCTGGTGCACTGACCTCTGGGGTGCACA |
| | | CCTTTCCAGCTGTGCTGCAGTCCTCTGGCCTGTACTCTCTGTCCTCTGTGGT |
| | | CACAGTGCCTAGCTCTAGCCTGGGCACCCAGACCTACATCTGCAATGTGAAC |
| | | CACAAGCCTAGCAACACCAAGGTGGACAAGAGAGTGGAACCCAAGAGCTGTG |
| | | ACAAGACCCACACC |
| Eculizumab Fab | Heavy/ | CAGGTGCAGCTGGTTCAGTCTGGGGCTGAAGTGAAGAAACCTGGGGCCTCTG |
| IgG2 | SEQ ID | TGAAGGTGTCCTGCAAGGCCTCTGGCTACATCTTCAGCAACTACTGGATTCA |
| | NO: 29 | GTGGGTCAGACAGGCCCCTGGCCAAGGACTTGAGTGGATGGGAGAGATCCTG |
| | | CCTGGCTCTGGCAGCACAGAGTACACAGAGAACTTCAAGGACAGAGTGACCA |
| | | TGACCAGAGACACCAGCACCTCCACAGTGTACATGGAACTGAGCAGCCTGAG |
| | | ATCTGAGGACACAGCAGTGTACTATTGTGCCAGATACTTCTTTGGCAGCAGC |
| | | CCCAACTGGTACTTTGATGTGTGGGGCCAGGGCACCCTGGTTACAGTGTCCT |
| | | CTGCCTCTACAAAGGGCCCCTCTGTGTTCCCTCTGGCTCCTTGTAGCAGAAG |
| | | CACCTCTGAGTCTACAGCTGCCCTGGGCTGCCTGGTCAAGGATTACTTTCCT |
| | | GAGCCTGTGACTGTGTCCTGGAACTCTGGTGCTCTGACCTCTGGGGTGCACA |
| | | CCTTTCCAGCTGTGCTGCAGTCCTCTGGCCTGTACTCTCTGTCCTCTGTGGT |
| | | CACAGTGCCCAGCAGCAATTTTGGCACCCAGACCTACACCTGTAATGTGGAC |
| | | CACAAGCCTAGCAACACCAAGGTGGACAAGACAGTG |
| Eculizumab | Light/ | GACATCCAGATGACACAGAGCCCTAGCAGCCTGTCTGCCTCTGTGGGAGACA |
| light chain | SEQ ID | GAGTGACAATCACCTGTGGGGCCTCTGAGAACATCTATGGGGCCCTGAACTG |
| | NO: 30 | GTATCAGCAGAAGCCTGGAAAGGCCCCTAAGCTGCTGATATATGGGGCCACC |
| | | AATCTGGCTGATGGGGTGCCCTCTAGATTTTCTGGCAGTGGCTCTGGCACAG |
| | | ACTTCACCCTGACCATCAGTAGCCTGCAGCCTGAGGACTTTGCCACCTACTA |
| | | CTGTCAGAATGTGCTGAACACCCCTCTGACCTTTGGCCAGGGCACCAAGGTG |
| | | GAAATCAAGAGAACAGTGGCTGCCCCTTCTGTaTTCATCTTCCCACCATCTG |
| | | ATGAGCAGCTGAAGAGTGGCACAGCCTCTGTTGTGTGCCTGCTGAACAACTT |
| | | CTACCCCAGAGAAGCCAAGGTGCAGTGGAAGGTGGACAATGCCCTGCAGTCT |
| | | GGCAACAGCCAAGAGTCTGTGACTGAGCAGGACAGCAAGGACTCCACCTACA |
| | | GCCTGAGCAGCACCCTGACACTGAGCAAGGCTGACTATGAGAAGCACAAAGT |
| | | GTATGCCTGTGAAGTGACCCACCAGGGGCTGAGCAGCCCTGTGACCAAGAGC |
| | | TTCAATAGAGGGGAGTGC |
| Ravulizumab | Heavy/ | caggtgcagctggtgcagagcggcgcggaagtgaaaaaaccgggcgcgagcg |
| Heavy Chain | SEQ ID | tgaaagtgagctgcaaagcgagcggccatatttttagcaactattggattca |
| Fab | NO: 31 | gtgggtgcgccaggcgccgggccagggcctggaatggatgggcgaaattctg |
| | | ccgggcagcggccataccgaatataccgaaaactttaaagatcgcgtgacca |
| | | tgacccgcgataccagcaccagcaccgtgtatatggaactgagcagcctgcg |
| | | cagcgaagataccgcggtgtattattgcgcgcgctatttttttggcagcagc |
| | | ccgaactggtattttgatgtgtggggccagggcaccctggtgaccgtgagca |
| | | gcgcgagcaccaaaggcccgagcgtgtttccgctggcgccgtgcagccgcag |
| | | caccagcgaaagcaccgcggcgctgggctgcctggtgaaagattattttccg |
| | | gaaccggtgaccgtgagctggaacagcggcgcgctgaccagcggcgtgcata |
| | | cctttccggcggtgctgcagagcagcggcctgtatagcctgagcagcgtggt |
| | | gaccgtgccgagcagcaactttggcacccagacctatacctgcaacgtggat |
| | | cataaaccgagcaacaccaaagtggataaaaccgtggaacgcaaatgctgcg |
| | | tggaa +/− TGCCCCCCCTGCCCCGCC +/ − CCCCCCGTGGCCGGC |
| Ravulizumab | Light/ | gatattcagatgacccagagcccgagcagcctgagcgcgagcgtgggcgatc |
| light chain | SEQ ID | gcgtgaccattacctgcggcgcgagcgaaaacatttatggcgcgctgaactg |
| | NO: 32 | gtatcagcagaaaccgggcaaagcgccgaaactgctgatttatggcgcgacc |
| | | aacctggcggatggcgtgccgagccgctttagcggcagcggcagcggcaccg |
| | | attttaccctgaccattagcagcctgcagccggaagattttgcgacctatta |
| | | ttgccagaacgtgctgaacaccccgctgacctttggccagggcaccaaagtg |
| | | gaaattaaacgcaccgtggcggcgccgagcgtgtttatttttccgccgagcg |
| | | atgaacagctgaaaagcggcaccgcgagcgtggtgtgcctgctgaacaactt |
| | | ttatccgcgcgaagcgaaagtgcagtggaaagtggataacgcgctgcagagc |
| | | ggcaacagccaggaaagcgtgaccgaacaggatagcaaagatagcacctata |
| | | gcctgagcagcaccctgaccctgagcaaagcggattatgaaaaacataaagt |
| | | gtatgcgtgcgaagtgacccatcagggcctgagcagcccggtgaccaaaagc |
| | | tttaaccgcggcgaatgc |
| Tesidolumab | Heavy/ | GAGGTGCAGCTGGTGCAGAGCGGCGCCGAGGTGAAGAAGCCCGGCAGCAGCG |
| Heavy Chain | SEQ ID | TGAAGGTGAGCTGCAAGGCCAGCGGCGGCACCTTCAGCAGCTACGCCATCAG |
| Fab | NO: 33 | CTGGGTGAGGCAGGCCCCCGGCCAGGGCCTGGAGTGGATGGGCGGCATCGGC |
| | | CCCTTCTTCGGCACCGCCAACTACGCCCAGAAGTTCCAGGGCAGGGTGACCA |
| | | TCACCGCCGACGAGAGCACCAGCACCGCCTACATGGAGCTGAGCAGCCTGAG |
| | | GAGCGAGGACACCGCCGTGTACTACTGCGCCAGGGACACCCCCTACTTCGAC |
| | | TACTGGGGCCAGGGCACCCTGGTGACCGTGAGCAGCGCCAGCACCAAGGGCC |
| | | CCAGCGTGTTCCCCCTGGCCCCCAGCAGCAAGAGCACCAGCGGCGGCACCGC |
| | | CGCCCTGGGCTGCCTGGTGAAGGACTACTTCCCCGAGCCCGTGACCGTGAGC |
| | | TGGAACAGCGGCGCCCTGACCAGCGGCGTGCACACCTTCCCCGCCGTGCTGC |
| | | AGAGCAGCGGCCTGTACAGCCTGAGCAGCGTGGTGACCGTGCCCAGCAGCAG |
| | | CCTGGGCACCCAGACCTACATCTGCAACGTGAACCACAAGCCCAGCAACACC |
| | | AAGGTGGACAAGAGGGTGGAGCCCAAGAGCTGCGAC+/− AAGACCCACACC |
| | | (or AAGACCCACCTG) +/ − TGCCCCCCCTGCCCCGCC +/ − |
| | | CCCGAGGCCGCCGGCGGCCCCAGCGTGTTCCTG |
| Tesidolumab | Light/ | AGCTACGAGCTGACCCAGCCCCTGAGCGTGAGCGTGGCCCTGGGCCAGACCG |
| light chain | SEQ ID | CCAGGATCACCTGCAGCGGCGACAGCATCCCCAACTACTACGTGTACTGGTA |
| | NO: 34 | CCAGCAGAAGCCCGGCCAGGCCCCCGTGCTGGTGATCTACGACGACAGCAAC |
| | | AGGCCCAGCGGCATCCCCGAGAGGTTCAGCGGCAGCAACAGCGGCAACACCG |
| | | CCACCCTGACCATCAGCAGGGCCCAGGCCGGCGACGAGGCCGACTACTACTG |
| | | CCAGAGCTTCGACAGCAGCCTGAACGCCGAGGTGTTCGGCGGCGGCACCAAG |
| | | CTGACCGTGCTGGGCCAGCCCAAGGCCGCCCCCAGCGTGACCCTGTTCCCCC |
| | | CCAGCAGCGAGGAGCTGCAGGCCAACAAGGCCACCCTGGTGTGCCTGATCAG |
| | | CGACTTCTACCCCGGCGCCGTGACCGTGGCCTGGAAGGCCGACAGCAGCCCC |
| | | GTGAAGGCCGGCGTGGAGACCACCACCCCCAGCAAGCAGAGCAACAACAAGT |
| | | ACGCCGCCAGCAGCTACCTGAGCCTGACCCCCGAGCAGTGGAAGAGCCACAG |
| | | GAGCTACAGCTGCCAGGTGACCCACGAGGGCAGCACCGTGGAGAAGACCGTG |
| | | GCCCCCACCGAGTGCAGC |
| BB5.1 | Heavy/ | CAGGTTCAGCTGCAGCAACCTGGGGCTGAGCTTGTCAGACCTGGCACATCTG |
| (full length | SEQ ID | TGAAGCTGAGCTGCAAGGCCTCTGGCTACACCTTCACAAGCAGCTGGATGCA |
| heavy chain | NO: 35 | CTGGGTCAAGCAGAGGCCTGGACAGGGCCTTGAGTGGATTGGAGTGATTGAC |
| | | CCCTCTGACAGCTACACCAACTACAACCAGAAGTTCAAGGGCAAAGCCACAC |
| | | TGACAGTGGACACCAGCAGCAGCACAGCCTACATGCAGCTGAGCAGCCTGAC |
| | | CTCTGAGGACTCTGCTGTGTACTACTGTGCCAGAGGTGGTGGCAGCAGCTAC |
| | | AACAGATACTTTGATGTGTGGGGCACAGGCACCACAGTGACTGTGTCCTCTG |
| | | CCAAGACAACCCCTCCTTCTGTGTACCCTCTGGCTCCTGGATCTGCTGCCCA |
| | | GACCAACTCTATGGTCACACTGGGCTGTCTGGTCAAGGGCTACTTCCCTGAG |
| | | CCTGTGACAGTGACCTGGAACTCTGGATCTCTGTCCTCTGGGGTGCACACAT |
| | | TCCCTGCAGTGCTGCAGTCTGATCTGTACACCCTGAGCAGCTCTGTGACTGT |
| | | GCCTAGCAGCCCCAGACCTTCTGAGACTGTGACCTGCAATGTGGCTCACCCT |
| | | GCCAGCAGCACCAAGGTGGACAAGAAAATTGTGCCCAGAGACTGTGGCTGCA |
| | | AGCCCTGCATCTGTACAGTGCCTGAGGTTTCCTCTGTGTTCATCTTCCCACC |
| | | TAAGCCTAAGGATGTGCTGACCATCACACTGACCCCTAAAGTGACCTGTGTG |
| | | GTGGTGGACATCAGCAAGGATGACCCTGAGGTGCAGTTCTCTTGGTTTGTGG |
| | | ATGATGTGGAAGTGCACACAGCCCAGACACAGCCTAGAGAGGAACAGTTCAA |
| | | CAGCACCTTCAGATCAGTGTCTGAGCTGCCCATCATGCACCAGGACTGGCTG |
| | | AATGGCAAAGAGTTCAAGTGCAGAGTGAACTCTGCTGCTTTCCCTGCTCCTA |
| | | TTGAAAAGACCATCTCCAAGACCAAGGGCAGACCCAAGGCTCCCCAGGTGTA |
| | | CACAATCCCTCCACCTAAAGAACAGATGGCCAAGGACAAGGTGTCCCTGACC |
| | | TGCATGATCACAGATTTCTTCCCAGAGGATATCACAGTTGAGTGGCAGTGGA |
| | | ATGGCCAGCCTGCTGAGAACTACAAGAACACCCAGCCTATCATGAACACCAA |
| | | TGGCAGCTACTTTGTGTACAGCAAGCTGAATGTGCAGAAGTCCAACTGGGAG |
| | | GCTGGCAACACCTTTACCTGCTCTGTGCTGCATGAGGGCCTGCACAACCACC |
| | | ATACAGAGAAGTCCCTGTCTCACAGCCCTGGCAAG |
| BB5.1 light | Light/ | AATATCATGATGACACAGAGCCCCAGCAGCCTGGCTGTGTCTGCTGGGGAGA |
| chain | SEQ ID | AAGTGACCATGAGCTGCAAGAGCAGCCAGTCTGTGCTGTACTCCAGCAACCA |
| - | NO: 36 | GAAGAACTACCTGGCCTGGTATCAGCAGAAGCCTGGCCAGTCTCCTAAGCTG |
| | | CTGATCTACTGGGCCAGCACCAGAGAATCTGGGGTGCCAGACAGATTCACAG |
| | | GCTCTGGCTCTGGCACAGACTTCACCCTGACAATCAGCTCTGTGCAGGCTGA |
| | | GGACCTGGCAGTGTACTACTGTCACCAGTACCTGAGCAGCAGAACCTTTGGT |
| | | GGTGGCACCAAGCTGGAAATCAAGAGGGCTGATGCTGCCCCTACAGTGTCTA |
| | | TCTTCCCACCTAGCTCTGAGCAGCTGACCAGTGGTGGTGCCTCTGTTGTGTG |
| | | CTTCCTGAACAACTTCTACCCCAAGGACATCAATGTGAAGTGGAAGATTGAT |
| | | GGCTCTGAGAGGCAGAATGGGGTGCTGAACAGCTGGACAGACCAGGACAGCA |
| | | AGGACTCCACCTACAGCATGAGCAGCACACTGACCCTGACCAAGGATGAGTA |
| | | TGAGAGGCACAACAGCTACACCTGTGAAGCCACACACAAGACCAGCACAAGC |
| | | CCCATTGTGAAGTCCTTCAACAGGGGAGAGTGC |
| Vectorized | SEQ ID | ATGTACAGAATGCAGCTGCTGCTGCTCATTGCCCTGTCTCTGGCCCTGGTCA |
| Crovalimab Fab | NO: 37 | CCAATTCTCAGGTGCAGCTGGTTGAATCTGGTGGTGGACTGGTGCAGCCTGG |
| coding sequence | | CAGAAGCCTGAGACTGTCTTGTGCTGCCTCTGGCTTCACAGTGCACAGCAGC |
| | | TACTACATGGCCTGGGTTAGACAGGCCCCTGGCAAAGGACTTGAGTGGGTTG |
| | | GAGCCATCTTCACAGGCTCTGGGGCTGAGTATAAGGCTGAGTGGGCCAAGGG |
| | | CAGAGTGACCATCAGCAAGGACACCAGCAAGAACCAGGTGGTGCTGACCATG |
| | | ACCAACATGGACCCTGTGGACACAGCCACCTACTACTGTGCCTCTGATGCTG |
| | | GCTATGACTACCCCACACATGCCATGCACTATTGGGGCCAGGGCACCCTGGT |
| | | TACAGTGTCCTCTGCCTCTACAAAGGGCCCCTCTGTGTTTCCTCTGGCTCCT |
| | | AGCAGCAAGAGCACCAGTGGTGGAACAGCTGCCCTGGGCTGTCTGGTCAAGG |
| | | ATTACTTCCCTGAGCCAGTGACTGTGTCCTGGAACTCTGGTGCACTGACCTC |
| | | TGGGGTGCACACCTTTCCAGCTGTGCTGCAGTCCTCTGGCCTGTACTCTCTG |
| | | TCCTCTGTGGTCACAGTGCCTAGCTCTAGCCTGGGCACCCAGACCTACATCT |
| | | GCAATGTGAACCACAAGCCTAGCAACACCAAGGTGGACAAGAAGGTGGAACC |
| | | CAAGAGCTGTGACAAGACCCACAGAAAGAGAAGAGGCTCTGGAGAAGGCAGA |
| | | GGCTCCCTGCTGACATGTGGGGATGTTGAAGAGAATCCTGGGCCTATGTATA |
| | | GGATGCAACTGCTCCTCCTGATTGCTCTGAGCCTGGCTCTTGTGACCAACTC |
| | | TGACATCCAGATGACACAGAGCCCTAGCAGCCTGTCTGCCTCTGTGGGAGAC |
| | | AGAGTGACCATCACCTGTAGAGCCAGCCAGGGCATCTCTAGCAGTCTGGCCT |
| | | GGTATCAGCAGAAGCCTGGAAAGGCCCCTAAGCTGCTGATCTATGGGGCCTC |
| | | TGAGACAGAATCTGGGGTGCCCAGCAGATTTTCAGGCTCTGGCTCTGGCACA |
| | | GACTTCACCCTGACCATTTCTAGCCTGCAGCCTGAGGACTTTGCCACCTACT |
| | | ACTGCCAGAACACCAAAGTGGGCAGCAGCTATGGCAACACCTTTGGTGGTGG |
| | | CACCAAGGTGGAAATCAAGAGAACAGTGGCTGCCCCTTCTGTGTTCATCTTC |
| | | CCACCATCTGATGAGCAGCTGAAGAGTGGCACAGCCTCTGTTGTGTGCCTGC |
| | | TGAACAACTTCTACCCCAGAGAAGCCAAGGTGCAGTGGAAGGTGGACAATGC |
| | | CCTGCAGTCAGGCAACAGCCAAGAGTCTGTGACTGAGCAGGACAGCAAGGAC |
| | | TCCACCTACAGCCTGAGCAGCACCCTGACACTGAGCAAGGCTGACTATGAGA |
| | | AGCACAAAGTGTATGCCTGTGAAGTGACCCACCAGGGGCTGAGCAGCCCTGT |
| | | GACCAAGAGCTTCAATAGAGGGGAGTGCTGA |
| Vectorized | SEQ ID | ATGTACAGAATGCAGCTGCTGCTGCTCATTGCCCTGTCTCTGGCCCTGGTCA |
| Crovalimab Full | NO: 38 | CCAATTCTCAGGTGCAGCTGGTTGAATCTGGTGGTGGACTGGTGCAGCCTGG |
| Length coding | | CAGAAGCCTGAGACTGTCTTGTGCTGCCTCTGGCTTCACAGTGCACAGCAGC |
| sequence | | TACTACATGGCCTGGGTTAGACAGGCCCCTGGCAAAGGACTTGAGTGGGTTG |
| | | GAGCCATCTTCACAGGCTCTGGGGCTGAGTATAAGGCTGAGTGGGCCAAGGG |
| | | CAGAGTGACCATCAGCAAGGACACCAGCAAGAACCAGGTGGTGCTGACCATG |
| | | ACCAACATGGACCCTGTGGACACAGCCACCTACTACTGTGCCTCTGATGCTG |
| | | GCTATGACTACCCCACACATGCCATGCACTATTGGGGCCAGGGCACCCTGGT |
| | | TACAGTGTCCTCTGCCTCTACAAAGGGCCCCTCTGTGTTTCCTCTGGCTCCT |
| | | AGCAGCAAGAGCACCAGTGGTGGAACAGCTGCCCTGGGCTGTCTGGTCAAGG |
| | | ATTACTTCCCTGAGCCAGTGACTGTGTCCTGGAACTCTGGTGCACTGACCTC |
| | | TGGGGTGCACACCTTTCCAGCTGTGCTGCAGTCCTCTGGCCTGTACTCTCTG |
| | | TCCTCTGTGGTCACAGTGCCTAGCTCTAGCCTGGGCACCCAGACCTACATCT |
| | | GCAATGTGAACCACAAGCCTAGCAACACCAAGGTGGACAAGAAGGTGGAACC |
| | | CAAGAGCTGTGACAAGACCCACACCTGTCCTCCATGTCCTGCTCCAGAGCTG |
| | | AGAAGAGGCCCCAAGGTGTTCCTGTTTCCTCCAAAGCCTAAGGACACCCTGA |
| | | TGATCAGCAGAACCCCTGAAGTGACCTGTGTGGTGGTTGATGTGTCCCATGA |
| | | GGACCCAGAAGTGAAGTTCAATTGGTATGTGGATGGGGTTGAAGTGCACAAT |
| | | GCCAAGACCAAGCCTAGAGAGGAACAGTACAACAGCACCTACAGAGTGGTGT |
| | | CTGTGCTGACAGTGCTGCATCAGGACTGGCTGAATGGCAAAGAGTACAAGTG |
| | | CAAGGTGTCCAACAAGGGCCTGCCAAGCAGCATTGAGAAAACCATCTCCAAG |
| | | GCCAAGGGGCAGCCCAGAGAACCTCAGGTTTACACCCTGCCTCCAAGCAGGG |
| | | AAGAGATGACCAAGAATCAGGTGTCCCTGACCTGCCTGGTTAAGGGCTTCTA |
| | | CCCCTCTGACATTGCTGTGGAATGGGAGAGCAATGGCCAGCCTGAGAACAAC |
| | | TACAAGACAACCCCTCCTGTGCTGGACTCTGATGGCTCATTCTTCCTGTACA |
| | | GCAAGCTGACTGTGGACAAGTCCAGATGGCAGCAGGGCAATGTGTTCAGCTG |
| | | CAGTGTGCTGCATGAAGCCCTGCATGCCCACTACACCAGAAAAGAGCTGTCT |
| | | CTGAGCCCCAGAAAGAGAAGAGGCTCTGGAGAAGGCAGAGGCTCCCTGCTGA |
| | | CATGTGGGGATGTTGAAGAGAATCCTGGGCCTATGTATAGGATGCAACTGCT |
| | | CCTCCTGATTGCTCTGAGCCTGGCTCTTGTGACCAACTCTGACATCCAGATG |
| | | ACACAGAGCCCTAGCAGCCTGTCTGCCTCTGTGGGAGACAGAGTGACCATCA |
| | | CCTGTAGAGCCAGCCAGGGCATCTCTAGCAGTCTGGCCTGGTATCAGCAGAA |
| | | GCCTGGAAAGGCCCCTAAGCTGCTGATCTATGGGGCCTCTGAGACAGAATCT |
| | | GGGGTGCCCAGCAGATTTTCAGGCTCTGGCTCTGGCACAGACTTCACCCTGA |
| | | CCATTTCTAGCCTGCAGCCTGAGGACTTTGCCACCTACTACTGCCAGAACAC |
| | | CAAAGTGGGCAGCAGCTATGGCAACACCTTTGGTGGTGGCACCAAGGTGGAA |
| | | ATCAAGAGAACAGTGGCTGCCCCTTCTGTGTTCATCTTCCCACCATCTGATG |
| | | AGCAGCTGAAGAGTGGCACAGCCTCTGTTGTGTGCCTGCTGAACAACTTCTA |
| | | CCCCAGAGAAGCCAAGGTGCAGTGGAAGGTGGACAATGCCCTGCAGTCAGGC |
| | | AACAGCCAAGAGTCTGTGACTGAGCAGGACAGCAAGGACTCCACCTACAGCC |
| | | TGAGCAGCACCCTGACACTGAGCAAGGCTGACTATGAGAAGCACAAAGTGTA |
| | | TGCCTGTGAAGTGACCCACCAGGGGCTGAGCAGCCCTGTGACCAAGAGCTTC |
| | | AATAGAGGGGAGTGCTGA |
| Vectorized | SEQ ID | ATGTACAGAATGCAGCTGCTGCTGCTCATTGCCCTGTCTCTGGCCCTGGTCA |
| Eculizumab Fab | NO: 39 | CCAATTCTCAGGTGCAGCTGGTTCAGTCTGGGGCTGAAGTGAAGAAACCTGG |
| IgG1 coding | | GGCCTCTGTGAAGGTGTCCTGCAAGGCCTCTGGCTACATCTTCAGCAACTAT |
| sequence | | TGGATCCAGTGGGTCAGACAGGCCCCTGGCCAAGGACTTGAGTGGATGGGAG |
| | | AGATCCTGCCTGGCTCTGGCAGCACAGAGTACACAGAGAACTTCAAGGACAG |
| | | AGTGACCATGACCAGAGACACCAGCACCTCCACAGTGTACATGGAACTGAGC |
| | | AGCCTGAGATCTGAGGACACAGCAGTGTACTATTGTGCCAGATACTTCTTTG |
| | | GCAGCAGCCCCAACTGGTACTTTGATGTGTGGGGCCAGGGCACCCTGGTTAC |
| | | AGTGTCCTCTGCCTCTACAAAGGGCCCCTCTGTGTTTCCTCTGGCTCCTAGC |
| | | AGCAAGAGCACCAGTGGTGGAACAGCTGCCCTGGGCTGTCTGGTCAAGGATT |
| | | ACTTCCCTGAGCCTGTGACTGTGTCCTGGAACTCTGGTGCACTGACCTCTGG |
| | | GGTGCACACCTTTCCAGCTGTGCTGCAGTCCTCTGGCCTGTACTCTCTGTCC |
| | | TCTGTGGTCACAGTGCCTAGCTCTAGCCTGGGCACCCAGACCTACATCTGCA |
| | | ATGTGAACCACAAGCCTAGCAACACCAAGGTGGACAAGAGAGTGGAACCCAA |
| | | GAGCTGTGACAAGACCCACACCAGAAAGAGAAGAGGCTCTGGAGAAGGCAGA |
| | | GGCTCCCTGCTGACATGTGGGGATGTTGAAGAGAATCCTGGGCCTATGTATA |
| | | GGATGCAACTGCTCCTCCTGATTGCTCTGAGCCTGGCTCTTGTGACCAACTC |
| | | TGACATCCAGATGACACAGAGCCCTAGCAGCCTGTCTGCCTCTGTGGGAGAC |
| | | AGAGTGACAATCACCTGTGGGGCCTCTGAGAACATCTATGGGGCCCTGAACT |
| | | GGTATCAGCAGAAGCCTGGAAAGGCCCCTAAGCTGCTGATATATGGGGCCAC |
| | | CAATCTGGCTGATGGGGTGCCCTCTAGATTTTCTGGCAGTGGCTCTGGCACA |
| | | GACTTCACCCTGACCATCAGTAGCCTGCAGCCTGAGGACTTTGCCACCTACT |
| | | ACTGTCAGAATGTGCTGAACACCCCTCTGACCTTTGGCCAGGGCACCAAGGT |
| | | GGAAATCAAGAGAACAGTGGCTGCCCCTTCTGTaTTCATCTTCCCACCATCT |
| | | GATGAGCAGCTGAAGAGTGGCACAGCCTCTGTTGTGTGCCTGCTGAACAACT |
| | | TCTACCCCAGAGAAGCCAAGGTGCAGTGGAAGGTGGACAATGCCCTGCAGTC |
| | | TGGCAACAGCCAAGAGTCTGTGACTGAGCAGGACAGCAAGGACTCCACCTAC |
| | | AGCCTGAGCAGCACCCTGACACTGAGCAAGGCTGACTATGAGAAGCACAAAG |
| | | TGTATGCCTGTGAAGTGACCCACCAGGGGCTGAGCAGCCCTGTGACCAAGAG |
| | | CTTCAATAGAGGGGAGTGCTGA |
| Vectorized | SEQ ID | ATGTACAGAATGCAGCTGCTGCTGCTCATTGCCCTGTCTCTGGCCCTGGTCA |
| Eculizumab Fab | NO: 40 | CCAATTCTCAGGTGCAGCTGGTTCAGTCTGGGGCTGAAGTGAAGAAACCTGG |
| IgG2 coding | | GGCCTCTGTGAAGGTGTCCTGCAAGGCCTCTGGCTACATCTTCAGCAACTAC |
| sequence | | TGGATTCAGTGGGTCAGACAGGCCCCTGGCCAAGGACTTGAGTGGATGGGAG |
| | | AGATCCTGCCTGGCTCTGGCAGCACAGAGTACACAGAGAACTTCAAGGACAG |
| | | AGTGACCATGACCAGAGACACCAGCACCTCCACAGTGTACATGGAACTGAGC |
| | | AGCCTGAGATCTGAGGACACAGCAGTGTACTATTGTGCCAGATACTTCTTTG |
| | | GCAGCAGCCCCAACTGGTACTTTGATGTGTGGGGCCAGGGCACCCTGGTTAC |
| | | AGTGTCCTCTGCCTCTACAAAGGGCCCCTCTGTGTTCCCTCTGGCTCCTTGT |
| | | AGCAGAAGCACCTCTGAGTCTACAGCTGCCCTGGGCTGCCTGGTCAAGGATT |
| | | ACTTTCCTGAGCCTGTGACTGTGTCCTGGAACTCTGGTGCTCTGACCTCTGG |
| | | GGTGCACACCTTTCCAGCTGTGCTGCAGTCCTCTGGCCTGTACTCTCTGTCC |
| | | TCTGTGGTCACAGTGCCCAGCAGCAATTTTGGCACCCAGACCTACACCTGTA |
| | | ATGTGGACCACAAGCCTAGCAACACCAAGGTGGACAAGACAGTGAGAAAGAG |
| | | AAGAGGCTCTGGAGAAGGCAGAGGCTCCCTGCTGACATGTGGGGATGTTGAA |
| | | GAGAATCCTGGGCCTATGTATAGGATGCAACTGCTCCTCCTGATTGCTCTGA |
| | | GCCTGGCTCTTGTGACCAACTCTGACATCCAGATGACACAGAGCCCTAGCAG |
| | | CCTGTCTGCCTCTGTGGGAGACAGAGTGACAATCACCTGTGGGGCCTCTGAG |
| | | AACATCTATGGGGCCCTGAACTGGTATCAGCAGAAGCCTGGAAAGGCCCCTA |
| | | AGCTGCTGATATATGGGGCCACCAATCTGGCTGATGGGGTGCCCTCTAGATT |
| | | TTCTGGCAGTGGCTCTGGCACAGACTTCACCCTGACCATCAGTAGCCTGCAG |
| | | CCTGAGGACTTTGCCACCTACTACTGTCAGAATGTGCTGAACACCCCTCTGA |
| | | CCTTTGGCCAGGGCACCAAGGTGGAAATCAAGAGAACAGTGGCTGCCCCTTC |
| | | TGTaTTCATCTTCCCACCATCTGATGAGCAGCTGAAGAGTGGCACAGCCTCT |
| | | GTTGTGTGCCTGCTGAACAACTTCTACCCCAGAGAAGCCAAGGTGCAGTGGA |
| | | AGGTGGACAATGCCCTGCAGTCTGGCAACAGCCAAGAGTCTGTGACTGAGCA |
| | | GGACAGCAAGGACTCCACCTACAGCCTGAGCAGCACCCTGACACTGAGCAAG |
| | | GCTGACTATGAGAAGCACAAAGTGTATGCCTGTGAAGTGACCCACCAGGGGC |
| | | TGAGCAGCCCTGTGACCAAGAGCTTCAATAGAGGGGAGTGCTGA |
| Vectorized | SEQ ID | ATGTACAGAATGCAGCTGCTGCTGCTCATTGCCCTGTCTCTGGCCCTGGTCA |
| Eculizumab Full | NO: 41 | CCAATTCTCAGGTGCAGCTGGTTCAGTCTGGGGCTGAAGTGAAGAAACCTGG |
| Length coding | | GGCCTCTGTGAAGGTGTCCTGCAAGGCCTCTGGCTACATCTTCAGCAACTAC |
| sequence | | TGGATTCAGTGGGTCAGACAGGCCCCTGGCCAAGGACTTGAGTGGATGGGAG |
| | | AGATCCTGCCTGGCTCTGGCAGCACAGAGTACACAGAGAACTTCAAGGACAG |
| | | AGTGACCATGACCAGAGACACCAGCACCTCCACAGTGTACATGGAACTGAGC |
| | | AGCCTGAGATCTGAGGACACAGCAGTGTACTATTGTGCCAGATACTTCTTTG |
| | | GCAGCAGCCCCAACTGGTACTTTGATGTGTGGGGCCAGGGCACCCTGGTTAC |
| | | AGTGTCCTCTGCCTCTACAAAGGGCCCCTCTGTGTTCCCTCTGGCTCCTTGT |
| | | AGCAGAAGCACCTCTGAGTCTACAGCTGCCCTGGGCTGCCTGGTCAAGGATT |
| | | ACTTTCCTGAGCCTGTGACTGTGTCCTGGAACTCTGGTGCTCTGACCTCTGG |
| | | GGTGCACACCTTTCCAGCTGTGCTGCAGTCCTCTGGCCTGTACTCTCTGTCC |
| | | TCTGTGGTCACAGTGCCCAGCAGCAATTTTGGCACCCAGACCTACACCTGTA |
| | | ATGTGGACCACAAGCCTAGCAACACCAAGGTGGACAAGACAGTGGAAAGAAA |
| | | GTGCTGTGTGGAATGCCCTCCTTGTCCTGCTCCTCCAGTGGCTGGACCTTCT |
| | | GTGTTTCTGTTCCCTCCAAAGCCTAAGGACACCCTGATGATCAGCAGAACCC |
| | | CTGAAGTGACCTGTGTGGTGGTGGATGTGTCCCAAGAGGACCCTGAGGTGCA |
| | | GTTCAATTGGTATGTGGATGGGGTTGAAGTGCACAATGCCAAGACCAAGCCT |
| | | AGAGAGGAACAGTTCAACAGCACCTACAGAGTGGTGTCTGTGCTGACAGTGC |
| | | TGCACCAGGACTGGCTGAATGGCAAAGAGTACAAGTGCAAGGTGTCCAACAA |
| | | GGGCCTGCCTAGCAGCATTGAGAAAACCATCAGCAAGGCCAAGGGCCAGCCA |
| | | AGAGAACCCCAAGTGTACACCCTGCCTCCAAGCCAAGAGGAAATGACCAAGA |
| | | ACCAGGTGTCCCTGACCTGCCTTGTGAAGGGCTTCTACCCCTCTGACATTGC |
| | | TGTGGAATGGGAGTCCAATGGCCAGCCTGAAAACAACTACAAGACCACACCT |
| | | CCTGTGCTGGACTCTGATGGCAGCTTCTTCCTGTACAGCAGACTGACAGTGG |
| | | ACAAGTCCAGATGGCAAGAGGGCAATGTGTTCAGCTGCTCTGTGATGCATGA |
| | | GGCCCTGCACAACCACTACACCCAGAAGTCTCTGTCTCTGAGCCTGGGCAAG |
| | | AGAAAGAGAAGAGGCTCTGGAGAAGGCAGAGGCTCCCTGCTGACATGTGGGG |
| | | ATGTTGAAGAGAATCCTGGGCCTATGTATAGGATGCAACTGCTCCTCCTGAT |
| | | TGCTCTGAGCCTGGCTCTTGTGACCAACTCTGACATCCAGATGACACAGAGC |
| | | CCTAGCAGCCTGTCTGCCTCTGTGGGAGACAGAGTGACAATCACCTGTGGGG |
| | | CCTCTGAGAACATCTATGGGGCCCTGAACTGGTATCAGCAGAAGCCTGGAAA |
| | | GGCCCCTAAGCTGCTGATATATGGGGCCACCAATCTGGCTGATGGGGTGCCC |
| | | TCTAGATTTTCTGGCAGTGGCTCTGGCACAGACTTCACCCTGACCATCAGTA |
| | | GCCTGCAGCCTGAGGACTTTGCCACCTACTACTGTCAGAATGTGCTGAACAC |
| | | CCCTCTGACCTTTGGCCAGGGCACCAAGGTGGAAATCAAGAGAACAGTGGCT |
| | | GCCCCTTCTGTaTTCATCTTCCCACCATCTGATGAGCAGCTGAAGAGTGGCA |
| | | CAGCCTCTGTTGTGTGCCTGCTGAACAACTTCTACCCCAGAGAAGCCAAGGT |
| | | GCAGTGGAAGGTGGACAATGCCCTGCAGTCTGGCAACAGCCAAGAGTCTGTG |
| | | ACTGAGCAGGACAGCAAGGACTCCACCTACAGCCTGAGCAGCACCCTGACAC |
| | | TGAGCAAGGCTGACTATGAGAAGCACAAAGTGTATGCCTGTGAAGTGACCCA |
| | | CCAGGGGCTGAGCAGCCCTGTGACCAAGAGCTTCAATAGAGGGGAGTGCTGA |
| Vectorized | SEQ ID | ATGTACAGAATGCAGCTGCTGCTGCTCATTGCCCTGTCTCTGGCCCTGGTCA |
| BB5.1 Full | NO: 42 | CCAATTCTCAGGTTCAGCTGCAGCAACCTGGGGCTGAGCTTGTCAGACCTGG |
| Length coding | | CACATCTGTGAAGCTGAGCTGCAAGGCCTCTGGCTACACCTTCACAAGCAGC |
| sequence | | TGGATGCACTGGGTCAAGCAGAGGCCTGGACAGGGCCTTGAGTGGATTGGAG |
| | | TGATTGACCCCTCTGACAGCTACACCAACTACAACCAGAAGTTCAAGGGCAA |
| | | AGCCACACTGACAGTGGACACCAGCAGCAGCACAGCCTACATGCAGCTGAGC |
| | | AGCCTGACCTCTGAGGACTCTGCTGTGTACTACTGTGCCAGAGGTGGTGGCA |
| | | GCAGCTACAACAGATACTTTGATGTGTGGGGCACAGGCACCACAGTGACTGT |
| | | GTCCTCTGCCAAGACAACCCCTCCTTCTGTGTACCCTCTGGCTCCTGGATCT |
| | | GCTGCCCAGACCAACTCTATGGTCACACTGGGCTGTCTGGTCAAGGGCTACT |
| | | TCCCTGAGCCTGTGACAGTGACCTGGAACTCTGGATCTCTGTCCTCTGGGGT |
| | | GCACACATTCCCTGCAGTGCTGCAGTCTGATCTGTACACCCTGAGCAGCTCT |
| | | GTGACTGTGCCTAGCAGCCCCAGACCTTCTGAGACTGTGACCTGCAATGTGG |
| | | CTCACCCTGCCAGCAGCACCAAGGTGGACAAGAAAATTGTGCCCAGAGACTG |
| | | TGGCTGCAAGCCCTGCATCTGTACAGTGCCTGAGGTTTCCTCTGTGTTCATC |
| | | TTCCCACCTAAGCCTAAGGATGTGCTGACCATCACACTGACCCCTAAAGTGA |
| | | CCTGTGTGGTGGTGGACATCAGCAAGGATGACCCTGAGGTGCAGTTCTCTTG |
| | | GTTTGTGGATGATGTGGAAGTGCACACAGCCCAGACACAGCCTAGAGAGGAA |
| | | CAGTTCAACAGCACCTTCAGATCAGTGTCTGAGCTGCCCATCATGCACCAGG |
| | | ACTGGCTGAATGGCAAAGAGTTCAAGTGCAGAGTGAACTCTGCTGCTTTCCC |
| | | TGCTCCTATTGAAAAGACCATCTCCAAGACCAAGGGCAGACCCAAGGCTCCC |
| | | CAGGTGTACACAATCCCTCCACCTAAAGAACAGATGGCCAAGGACAAGGTGT |
| | | CCCTGACCTGCATGATCACAGATTTCTTCCCAGAGGATATCACAGTTGAGTG |
| | | GCAGTGGAATGGCCAGCCTGCTGAGAACTACAAGAACACCCAGCCTATCATG |
| | | AACACCAATGGCAGCTACTTTGTGTACAGCAAGCTGAATGTGCAGAAGTCCA |
| | | ACTGGGAGGCTGGCAACACCTTTACCTGCTCTGTGCTGCATGAGGGCCTGCA |
| | | CAACCACCATACAGAGAAGTCCCTGTCTCACAGCCCTGGCAAGAGAAAGAGA |
| | | AGAGGCTCTGGAGAAGGCAGAGGCTCCCTGCTGACATGTGGGGATGTTGAAG |
| | | AGAATCCTGGGCCTATGTATAGGATGCAACTGCTCCTCCTGATTGCTCTGAG |
| | | CCTGGCTCTTGTGACCAACTCTAATATCATGATGACACAGAGCCCCAGCAGC |
| | | CTGGCTGTGTCTGCTGGGGAGAAAGTGACCATGAGCTGCAAGAGCAGCCAGT |
| | | CTGTGCTGTACTCCAGCAACCAGAAGAACTACCTGGCCTGGTATCAGCAGAA |
| | | GCCTGGCCAGTCTCCTAAGCTGCTGATCTACTGGGCCAGCACCAGAGAATCT |
| | | GGGGTGCCAGACAGATTCACAGGCTCTGGCTCTGGCACAGACTTCACCCTGA |
| | | CAATCAGCTCTGTGCAGGCTGAGGACCTGGCAGTGTACTACTGTCACCAGTA |
| | | CCTGAGCAGCAGAACCTTTGGTGGTGGCACCAAGCTGGAAATCAAGAGGGCT |
| | | GATGCTGCCCCTACAGTGTCTATCTTCCCACCTAGCTCTGAGCAGCTGACCA |
| | | GTGGTGGTGCCTCTGTTGTGTGCTTCCTGAACAACTTCTACCCCAAGGACAT |
| | | CAATGTGAAGTGGAAGATTGATGGCTCTGAGAGGCAGAATGGGGTGCTGAAC |
| | | AGCTGGACAGACCAGGACAGCAAGGACTCCACCTACAGCATGAGCAGCACAC |
| | | TGACCCTGACCAAGGATGAGTATGAGAGGCACAACAGCTACACCTGTGAAGC |
| | | CACACACAAGACCAGCACAAGCCCCATTGTGAAGTCCTTCAACAGGGGAGAG |
| | | TGCTGA |
| CAG.Crovalimab. | SEQ ID | ctgcgcgctcgctcgctcactgaggccgcccgggcaaagcccgggcgtcggg |
| Fab (5′ ITR to | NO: 43 | cgacctttggtcgcccggcctcagtgagcgagcgagcgcgcagagagggagt |
| 3′ ITR) ITR | | ggccaactccatcactaggggttcctCATATGcagggtaatggggatcctct |
| sequences in | | agataggtaccctagtcgacattgattattgactagttattaatagtaatca |
| italics | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTCAGGTGC |
| | | AGCTGGTTGAATCTGGTGGTGGACTGGTGCAGCCTGGCAGAAGCCTGAGACT |
| | | GTCTTGTGCTGCCTCTGGCTTCACAGTGCACAGCAGCTACTACATGGCCTGG |
| | | GTTAGACAGGCCCCTGGCAAAGGACTTGAGTGGGTTGGAGCCATCTTCACAG |
| | | GCTCTGGGGCTGAGTATAAGGCTGAGTGGGCCAAGGGCAGAGTGACCATCAG |
| | | CAAGGACACCAGCAAGAACCAGGTGGTGCTGACCATGACCAACATGGACCCT |
| | | GTGGACACAGCCACCTACTACTGTGCCTCTGATGCTGGCTATGACTACCCCA |
| | | CACATGCCATGCACTATTGGGGCCAGGGCACCCTGGTTACAGTGTCCTCTGC |
| | | CTCTACAAAGGGCCCCTCTGTGTTTCCTCTGGCTCCTAGCAGCAAGAGCACC |
| | | AGTGGTGGAACAGCTGCCCTGGGCTGTCTGGTCAAGGATTACTTCCCTGAGC |
| | | CAGTGACTGTGTCCTGGAACTCTGGTGCACTGACCTCTGGGGTGCACACCTT |
| | | TCCAGCTGTGCTGCAGTCCTCTGGCCTGTACTCTCTGTCCTCTGTGGTCACA |
| | | GTGCCTAGCTCTAGCCTGGGCACCCAGACCTACATCTGCAATGTGAACCACA |
| | | AGCCTAGCAACACCAAGGTGGACAAGAAGGTGGAACCCAAGAGCTGTGACAA |
| | | GACCCACAGAAAGAGAAGAGGCTCTGGAGAAGGCAGAGGCTCCCTGCTGACA |
| | | TGTGGGGATGTTGAAGAGAATCCTGGGCCTATGTATAGGATGCAACTGCTCC |
| | | TCCTGATTGCTCTGAGCCTGGCTCTTGTGACCAACTCTGACATCCAGATGAC |
| | | ACAGAGCCCTAGCAGCCTGTCTGCCTCTGTGGGAGACAGAGTGACCATCACC |
| | | TGTAGAGCCAGCCAGGGCATCTCTAGCAGTCTGGCCTGGTATCAGCAGAAGC |
| | | CTGGAAAGGCCCCTAAGCTGCTGATCTATGGGGCCTCTGAGACAGAATCTGG |
| | | GGTGCCCAGCAGATTTTCAGGCTCTGGCTCTGGCACAGACTTCACCCTGACC |
| | | ATTTCTAGCCTGCAGCCTGAGGACTTTGCCACCTACTACTGCCAGAACACCA |
| | | AAGTGGGCAGCAGCTATGGCAACACCTTTGGTGGTGGCACCAAGGTGGAAAT |
| | | CAAGAGAACAGTGGCTGCCCCTTCTGTGTTCATCTTCCCACCATCTGATGAG |
| | | CAGCTGAAGAGTGGCACAGCCTCTGTTGTGTGCCTGCTGAACAACTTCTACC |
| | | CCAGAGAAGCCAAGGTGCAGTGGAAGGTGGACAATGCCCTGCAGTCAGGCAA |
| | | CAGCCAAGAGTCTGTGACTGAGCAGGACAGCAAGGACTCCACCTACAGCCTG |
| | | AGCAGCACCCTGACACTGAGCAAGGCTGACTATGAGAAGCACAAAGTGTATG |
| | | CCTGTGAAGTGACCCACCAGGGGCTGAGCAGCCCTGTGACCAAGAGCTTCAA |
| | | TAGAGGGGAGTGCTGATAACtcgaggacggggtgaactacgcctgaggatcc |
| | | gatctttttccctctgccaaaaattatggggacatcatgaagccccttgagc |
| | | atctgacttctggctaataaaggaaatttattttcattgcaatagtgtgttg |
| | | gaattttttgtgtctctcactcggaagcaattcgttgatctgaatttcgacc |
| | | acccataatacccattaccctggtagataagtagcatggcgggttaatcatt |
| | | aactacaCCAGGGTAATGGGCTAGCTGCGGCCGCaggaacccctagtgatgg |
| | | agttggccactccctctctgcgcgctcgctcgctcactgaggccgggcgacc |
| | | aaaggtcgcccgacgcccgggctttgcccgggcggcctcagtgagcgagcga |
| | | gcgcgcag |
| CAG.Crovalimab | SEQ ID | ctgcgcgctcgctcgctcactgaggccgcccgggcaaagcccgggcgtcggg |
| Full Length | NO: 44 | cgacctttggtcgcccggcctcagtgagcgagcgagcgcgcagagagggagt |
| (5′ ITR to 3′TR) | | ggccaactccatcactaggggttcctCATATGcagggtaatggggatcctct |
| ITR sequences | | agataggtaccctagtcgacattgattattgactagttattaatagtaatca |
| in italics | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTCAGGTGC |
| | | AGCTGGTTGAATCTGGTGGTGGACTGGTGCAGCCTGGCAGAAGCCTGAGACT |
| | | GTCTTGTGCTGCCTCTGGCTTCACAGTGCACAGCAGCTACTACATGGCCTGG |
| | | GTTAGACAGGCCCCTGGCAAAGGACTTGAGTGGGTTGGAGCCATCTTCACAG |
| | | GCTCTGGGGCTGAGTATAAGGCTGAGTGGGCCAAGGGCAGAGTGACCATCAG |
| | | CAAGGACACCAGCAAGAACCAGGTGGTGCTGACCATGACCAACATGGACCCT |
| | | GTGGACACAGCCACCTACTACTGTGCCTCTGATGCTGGCTATGACTACCCCA |
| | | CACATGCCATGCACTATTGGGGCCAGGGCACCCTGGTTACAGTGTCCTCTGC |
| | | CTCTACAAAGGGCCCCTCTGTGTTTCCTCTGGCTCCTAGCAGCAAGAGCACC |
| | | AGTGGTGGAACAGCTGCCCTGGGCTGTCTGGTCAAGGATTACTTCCCTGAGC |
| | | CAGTGACTGTGTCCTGGAACTCTGGTGCACTGACCTCTGGGGTGCACACCTT |
| | | TCCAGCTGTGCTGCAGTCCTCTGGCCTGTACTCTCTGTCCTCTGTGGTCACA |
| | | GTGCCTAGCTCTAGCCTGGGCACCCAGACCTACATCTGCAATGTGAACCACA |
| | | AGCCTAGCAACACCAAGGTGGACAAGAAGGTGGAACCCAAGAGCTGTGACAA |
| | | GACCCACACCTGTCCTCCATGTCCTGCTCCAGAGCTGAGAAGAGGCCCCAAG |
| | | GTGTTCCTGTTTCCTCCAAAGCCTAAGGACACCCTGATGATCAGCAGAACCC |
| | | CTGAAGTGACCTGTGTGGTGGTTGATGTGTCCCATGAGGACCCAGAAGTGAA |
| | | GTTCAATTGGTATGTGGATGGGGTTGAAGTGCACAATGCCAAGACCAAGCCT |
| | | AGAGAGGAACAGTACAACAGCACCTACAGAGTGGTGTCTGTGCTGACAGTGC |
| | | TGCATCAGGACTGGCTGAATGGCAAAGAGTACAAGTGCAAGGTGTCCAACAA |
| | | GGGCCTGCCAAGCAGCATTGAGAAAACCATCTCCAAGGCCAAGGGGCAGCCC |
| | | AGAGAACCTCAGGTTTACACCCTGCCTCCAAGCAGGGAAGAGATGACCAAGA |
| | | ATCAGGTGTCCCTGACCTGCCTGGTTAAGGGCTTCTACCCCTCTGACATTGC |
| | | TGTGGAATGGGAGAGCAATGGCCAGCCTGAGAACAACTACAAGACAACCCCT |
| | | CCTGTGCTGGACTCTGATGGCTCATTCTTCCTGTACAGCAAGCTGACTGTGG |
| | | ACAAGTCCAGATGGCAGCAGGGCAATGTGTTCAGCTGCAGTGTGCTGCATGA |
| | | AGCCCTGCATGCCCACTACACCAGAAAAGAGCTGTCTCTGAGCCCCAGAAAG |
| | | AGAAGAGGCTCTGGAGAAGGCAGAGGCTCCCTGCTGACATGTGGGGATGTTG |
| | | AAGAGAATCCTGGGCCTATGTATAGGATGCAACTGCTCCTCCTGATTGCTCT |
| | | GAGCCTGGCTCTTGTGACCAACTCTGACATCCAGATGACACAGAGCCCTAGC |
| | | AGCCTGTCTGCCTCTGTGGGAGACAGAGTGACCATCACCTGTAGAGCCAGCC |
| | | AGGGCATCTCTAGCAGTCTGGCCTGGTATCAGCAGAAGCCTGGAAAGGCCCC |
| | | TAAGCTGCTGATCTATGGGGCCTCTGAGACAGAATCTGGGGTGCCCAGCAGA |
| | | TTTTCAGGCTCTGGCTCTGGCACAGACTTCACCCTGACCATTTCTAGCCTGC |
| | | AGCCTGAGGACTTTGCCACCTACTACTGCCAGAACACCAAAGTGGGCAGCAG |
| | | CTATGGCAACACCTTTGGTGGTGGCACCAAGGTGGAAATCAAGAGAACAGTG |
| | | GCTGCCCCTTCTGTGTTCATCTTCCCACCATCTGATGAGCAGCTGAAGAGTG |
| | | GCACAGCCTCTGTTGTGTGCCTGCTGAACAACTTCTACCCCAGAGAAGCCAA |
| | | GGTGCAGTGGAAGGTGGACAATGCCCTGCAGTCAGGCAACAGCCAAGAGTCT |
| | | GTGACTGAGCAGGACAGCAAGGACTCCACCTACAGCCTGAGCAGCACCCTGA |
| | | CACTGAGCAAGGCTGACTATGAGAAGCACAAAGTGTATGCCTGTGAAGTGAC |
| | | CCACCAGGGGCTGAGCAGCCCTGTGACCAAGAGCTTCAATAGAGGGGAGTGC |
| | | TGATAACtcgaggacggggtgaactacgcctgaggatccgatctttttccct |
| | | ctgccaaaaattatggggacatcatgaagccccttgagcatctgacttctgg |
| | | ctaataaaggaaatttattttcattgcaatagtgtgttggaattttttgtgt |
| | | ctctcactcggaagcaattcgttgatctgaatttcgaccacccataataccc |
| | | attaccctggtagataagtagcatggcgggttaatcattaactacaCCAGGG |
| | | TAATGGGCTAGCTGCGGCCGCaggaacccctagtgatggagttggccactcc |
| | | ctctctgcgcgctcgctcgctcactgaggccgggcgaccaaaggtcgcccga |
| | | cgcccgggctttgcccgggcggcctcagtgagcgagcgagcgcgcag |
| CAG.Eculizumab. | SEQ ID | ctgcgcgctcgctcgctcactgaggccgcccgggcaaagcccgggcgtcggg |
| Fab.IgG1 (5′ | NO: 45 | cgacctttggtcgcccggcctcagtgagcgagcgagcgcgcagagagggagt |
| ITR to 3′ ITR) | | ggccaactccatcactaggggttcctCATATGcagggtaatggggatcctct |
| ITR sequences | | agataggtaccctagtcgacattgattattgactagttattaatagtaatca |
| in italics | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTCAGGTGC |
| | | AGCTGGTTCAGTCTGGGGCTGAAGTGAAGAAACCTGGGGCCTCTGTGAAGGT |
| | | GTCCTGCAAGGCCTCTGGCTACATCTTCAGCAACTATTGGATCCAGTGGGTC |
| | | AGACAGGCCCCTGGCCAAGGACTTGAGTGGATGGGAGAGATCCTGCCTGGCT |
| | | CTGGCAGCACAGAGTACACAGAGAACTTCAAGGACAGAGTGACCATGACCAG |
| | | AGACACCAGCACCTCCACAGTGTACATGGAACTGAGCAGCCTGAGATCTGAG |
| | | GACACAGCAGTGTACTATTGTGCCAGATACTTCTTTGGCAGCAGCCCCAACT |
| | | GGTACTTTGATGTGTGGGGCCAGGGCACCCTGGTTACAGTGTCCTCTGCCTC |
| | | TACAAAGGGCCCCTCTGTGTTTCCTCTGGCTCCTAGCAGCAAGAGCACCAGT |
| | | GGTGGAACAGCTGCCCTGGGCTGTCTGGTCAAGGATTACTTCCCTGAGCCTG |
| | | TGACTGTGTCCTGGAACTCTGGTGCACTGACCTCTGGGGTGCACACCTTTCC |
| | | AGCTGTGCTGCAGTCCTCTGGCCTGTACTCTCTGTCCTCTGTGGTCACAGTG |
| | | CCTAGCTCTAGCCTGGGCACCCAGACCTACATCTGCAATGTGAACCACAAGC |
| | | CTAGCAACACCAAGGTGGACAAGAGAGTGGAACCCAAGAGCTGTGACAAGAC |
| | | CCACACCAGAAAGAGAAGAGGCTCTGGAGAAGGCAGAGGCTCCCTGCTGACA |
| | | TGTGGGGATGTTGAAGAGAATCCTGGGCCTATGTATAGGATGCAACTGCTCC |
| | | TCCTGATTGCTCTGAGCCTGGCTCTTGTGACCAACTCTGACATCCAGATGAC |
| | | ACAGAGCCCTAGCAGCCTGTCTGCCTCTGTGGGAGACAGAGTGACAATCACC |
| | | TGTGGGGCCTCTGAGAACATCTATGGGGCCCTGAACTGGTATCAGCAGAAGC |
| | | CTGGAAAGGCCCCTAAGCTGCTGATATATGGGGCCACCAATCTGGCTGATGG |
| | | GGTGCCCTCTAGATTTTCTGGCAGTGGCTCTGGCACAGACTTCACCCTGACC |
| | | ATCAGTAGCCTGCAGCCTGAGGACTTTGCCACCTACTACTGTCAGAATGTGC |
| | | TGAACACCCCTCTGACCTTTGGCCAGGGCACCAAGGTGGAAATCAAGAGAAC |
| | | AGTGGCTGCCCCTTCTGTaTTCATCTTCCCACCATCTGATGAGCAGCTGAAG |
| | | AGTGGCACAGCCTCTGTTGTGTGCCTGCTGAACAACTTCTACCCCAGAGAAG |
| | | CCAAGGTGCAGTGGAAGGTGGACAATGCCCTGCAGTCTGGCAACAGCCAAGA |
| | | GTCTGTGACTGAGCAGGACAGCAAGGACTCCACCTACAGCCTGAGCAGCACC |
| | | CTGACACTGAGCAAGGCTGACTATGAGAAGCACAAAGTGTATGCCTGTGAAG |
| | | TGACCCACCAGGGGCTGAGCAGCCCTGTGACCAAGAGCTTCAATAGAGGGGA |
| | | GTGCTGATAACtcgaggacggggtgaactacgcctgaggatccgatcttttt |
| | | ccctctgccaaaaattatggggacatcatgaagccccttgagcatctgactt |
| | | ctggctaataaaggaaatttattttcattgcaatagtgtgttggaatttttt |
| | | gtgtctctcactcggaagcaattcgttgatctgaatttcgaccacccataat |
| | | acccattaccctggtagataagtagcatggcgggttaatcattaactacaCC |
| | | AGGGTAATGGGCTAGCTGCGGCCGCaggaacccctagtgatggagttggcca |
| | | ctccctctctgcgcgctcgctcgctcactgaggccgggcgaccaaaggtcgc |
| | | ccgacgcccgggctttgcccgggcggcctcagtgagcgagcgagcgcgcag |
| CAG.Eculizumab. | SEQ ID | ctgcgcgctcgctcgctcactgaggccgcccgggcaaagcccgggcgtcggg |
| Fab.IgG2 (5′ | NO:46 | cgacctttggtcgcccggcctcagtgagcgagcgagcgcgcagagagggagt |
| ITR to 3′ ITR) | | ggccaactccatcactaggggttcctCATATGcagggtaatggggatcctct |
| ITR sequences | | agataggtaccctagtcgacattgattattgactagttattaatagtaatca |
| in italics | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTCAGGTGC |
| | | AGCTGGTTCAGTCTGGGGCTGAAGTGAAGAAACCTGGGGCCTCTGTGAAGGT |
| | | GTCCTGCAAGGCCTCTGGCTACATCTTCAGCAACTACTGGATTCAGTGGGTC |
| | | AGACAGGCCCCTGGCCAAGGACTTGAGTGGATGGGAGAGATCCTGCCTGGCT |
| | | CTGGCAGCACAGAGTACACAGAGAACTTCAAGGACAGAGTGACCATGACCAG |
| | | AGACACCAGCACCTCCACAGTGTACATGGAACTGAGCAGCCTGAGATCTGAG |
| | | GACACAGCAGTGTACTATTGTGCCAGATACTTCTTTGGCAGCAGCCCCAACT |
| | | GGTACTTTGATGTGTGGGGCCAGGGCACCCTGGTTACAGTGTCCTCTGCCTC |
| | | TACAAAGGGCCCCTCTGTGTTCCCTCTGGCTCCTTGTAGCAGAAGCACCTCT |
| | | GAGTCTACAGCTGCCCTGGGCTGCCTGGTCAAGGATTACTTTCCTGAGCCTG |
| | | TGACTGTGTCCTGGAACTCTGGTGCTCTGACCTCTGGGGTGCACACCTTTCC |
| | | AGCTGTGCTGCAGTCCTCTGGCCTGTACTCTCTGTCCTCTGTGGTCACAGTG |
| | | CCCAGCAGCAATTTTGGCACCCAGACCTACACCTGTAATGTGGACCACAAGC |
| | | CTAGCAACACCAAGGTGGACAAGACAGTGAGAAAGAGAAGAGGCTCTGGAGA |
| | | AGGCAGAGGCTCCCTGCTGACATGTGGGGATGTTGAAGAGAATCCTGGGCCT |
| | | ATGTATAGGATGCAACTGCTCCTCCTGATTGCTCTGAGCCTGGCTCTTGTGA |
| | | CCAACTCTGACATCCAGATGACACAGAGCCCTAGCAGCCTGTCTGCCTCTGT |
| | | GGGAGACAGAGTGACAATCACCTGTGGGGCCTCTGAGAACATCTATGGGGCC |
| | | CTGAACTGGTATCAGCAGAAGCCTGGAAAGGCCCCTAAGCTGCTGATATATG |
| | | GGGCCACCAATCTGGCTGATGGGGTGCCCTCTAGATTTTCTGGCAGTGGCTC |
| | | TGGCACAGACTTCACCCTGACCATCAGTAGCCTGCAGCCTGAGGACTTTGCC |
| | | ACCTACTACTGTCAGAATGTGCTGAACACCCCTCTGACCTTTGGCCAGGGCA |
| | | CCAAGGTGGAAATCAAGAGAACAGTGGCTGCCCCTTCTGTaTTCATCTTCCC |
| | | ACCATCTGATGAGCAGCTGAAGAGTGGCACAGCCTCTGTTGTGTGCCTGCTG |
| | | AACAACTTCTACCCCAGAGAAGCCAAGGTGCAGTGGAAGGTGGACAATGCCC |
| | | TGCAGTCTGGCAACAGCCAAGAGTCTGTGACTGAGCAGGACAGCAAGGACTC |
| | | CACCTACAGCCTGAGCAGCACCCTGACACTGAGCAAGGCTGACTATGAGAAG |
| | | CACAAAGTGTATGCCTGTGAAGTGACCCACCAGGGGCTGAGCAGCCCTGTGA |
| | | CCAAGAGCTTCAATAGAGGGGAGTGCTGATAACtcgaggacggggtgaacta |
| | | cgcctgaggatccgatctttttccctctgccaaaaattatggggacatcatg |
| | | aagccccttgagcatctgacttctggctaataaaggaaatttattttcattg |
| | | caatagtgtgttggaattttttgtgtctctcactcggaagcaattcgttgat |
| | | ctgaatttcgaccacccataatacccattaccctggtagataagtagcatgg |
| | | cgggttaatcattaactacaCCAGGGTAATGGGCTAGCTGCGGCCGCaggaa |
| | | cccctagtgatggagttggccactccctctctgcgcgctcgctcgctcactg |
| | | aggccgggcgaccaaaggtcgcccgacgcccgggctttgcccgggcggcctc |
| | | agtgagcgagcgagcgcgcag |
| CAG.Eculizumab. | SEQ ID | ctgcgcgctcgctcgctcactgaggccgcccgggcaaagcccgggcgtcggg |
| Full (5′ ITR to | NO: 47 | cgacctttggtcgcccggcctcagtgagcgagcgagcgcgcagagagggagt |
| 3′ ITR) ITR | | ggccaactccatcactaggggttcctCATATGcagggtaatggggatcctct |
| sequences in | | agataggtaccctagtcgacattgattattgactagttattaatagtaatca |
| italics | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTCAGGTGC |
| | | AGCTGGTTCAGTCTGGGGCTGAAGTGAAGAAACCTGGGGCCTCTGTGAAGGT |
| | | GTCCTGCAAGGCCTCTGGCTACATCTTCAGCAACTACTGGATTCAGTGGGTC |
| | | AGACAGGCCCCTGGCCAAGGACTTGAGTGGATGGGAGAGATCCTGCCTGGCT |
| | | CTGGCAGCACAGAGTACACAGAGAACTTCAAGGACAGAGTGACCATGACCAG |
| | | AGACACCAGCACCTCCACAGTGTACATGGAACTGAGCAGCCTGAGATCTGAG |
| | | GACACAGCAGTGTACTATTGTGCCAGATACTTCTTTGGCAGCAGCCCCAACT |
| | | GGTACTTTGATGTGTGGGGCCAGGGCACCCTGGTTACAGTGTCCTCTGCCTC |
| | | TACAAAGGGCCCCTCTGTGTTCCCTCTGGCTCCTTGTAGCAGAAGCACCTCT |
| | | GAGTCTACAGCTGCCCTGGGCTGCCTGGTCAAGGATTACTTTCCTGAGCCTG |
| | | TGACTGTGTCCTGGAACTCTGGTGCTCTGACCTCTGGGGTGCACACCTTTCC |
| | | AGCTGTGCTGCAGTCCTCTGGCCTGTACTCTCTGTCCTCTGTGGTCACAGTG |
| | | CCCAGCAGCAATTTTGGCACCCAGACCTACACCTGTAATGTGGACCACAAGC |
| | | CTAGCAACACCAAGGTGGACAAGACAGTGGAAAGAAAGTGCTGTGTGGAATG |
| | | CCCTCCTTGTCCTGCTCCTCCAGTGGCTGGACCTTCTGTGTTTCTGTTCCCT |
| | | CCAAAGCCTAAGGACACCCTGATGATCAGCAGAACCCCTGAAGTGACCTGTG |
| | | TGGTGGTGGATGTGTCCCAAGAGGACCCTGAGGTGCAGTTCAATTGGTATGT |
| | | GGATGGGGTTGAAGTGCACAATGCCAAGACCAAGCCTAGAGAGGAACAGTTC |
| | | AACAGCACCTACAGAGTGGTGTCTGTGCTGACAGTGCTGCACCAGGACTGGC |
| | | TGAATGGCAAAGAGTACAAGTGCAAGGTGTCCAACAAGGGCCTGCCTAGCAG |
| | | CATTGAGAAAACCATCAGCAAGGCCAAGGGCCAGCCAAGAGAACCCCAAGTG |
| | | TACACCCTGCCTCCAAGCCAAGAGGAAATGACCAAGAACCAGGTGTCCCTGA |
| | | CCTGCCTTGTGAAGGGCTTCTACCCCTCTGACATTGCTGTGGAATGGGAGTC |
| | | CAATGGCCAGCCTGAAAACAACTACAAGACCACACCTCCTGTGCTGGACTCT |
| | | GATGGCAGCTTCTTCCTGTACAGCAGACTGACAGTGGACAAGTCCAGATGGC |
| | | AAGAGGGCAATGTGTTCAGCTGCTCTGTGATGCATGAGGCCCTGCACAACCA |
| | | CTACACCCAGAAGTCTCTGTCTCTGAGCCTGGGCAAGAGAAAGAGAAGAGGC |
| | | TCTGGAGAAGGCAGAGGCTCCCTGCTGACATGTGGGGATGTTGAAGAGAATC |
| | | CTGGGCCTATGTATAGGATGCAACTGCTCCTCCTGATTGCTCTGAGCCTGGC |
| | | TCTTGTGACCAACTCTGACATCCAGATGACACAGAGCCCTAGCAGCCTGTCT |
| | | GCCTCTGTGGGAGACAGAGTGACAATCACCTGTGGGGCCTCTGAGAACATCT |
| | | ATGGGGCCCTGAACTGGTATCAGCAGAAGCCTGGAAAGGCCCCTAAGCTGCT |
| | | GATATATGGGGCCACCAATCTGGCTGATGGGGTGCCCTCTAGATTTTCTGGC |
| | | AGTGGCTCTGGCACAGACTTCACCCTGACCATCAGTAGCCTGCAGCCTGAGG |
| | | ACTTTGCCACCTACTACTGTCAGAATGTGCTGAACACCCCTCTGACCTTTGG |
| | | CCAGGGCACCAAGGTGGAAATCAAGAGAACAGTGGCTGCCCCTTCTGTaTTC |
| | | ATCTTCCCACCATCTGATGAGCAGCTGAAGAGTGGCACAGCCTCTGTTGTGT |
| | | GCCTGCTGAACAACTTCTACCCCAGAGAAGCCAAGGTGCAGTGGAAGGTGGA |
| | | CAATGCCCTGCAGTCTGGCAACAGCCAAGAGTCTGTGACTGAGCAGGACAGC |
| | | AAGGACTCCACCTACAGCCTGAGCAGCACCCTGACACTGAGCAAGGCTGACT |
| | | ATGAGAAGCACAAAGTGTATGCCTGTGAAGTGACCCACCAGGGGCTGAGCAG |
| | | CCCTGTGACCAAGAGCTTCAATAGAGGGGAGTGCTGATAACtcgaggacggg |
| | | gtgaactacgcctgaggatccgatctttttccctctgccaaaaattatgggg |
| | | acatcatgaagccccttgagcatctgacttctggctaataaaggaaatttat |
| | | tttcattgcaatagtgtgttggaattttttgtgtctctcactcggaagcaat |
| | | tcgttgatctgaatttcgaccacccataatacccattaccctggtagataag |
| | | tagcatggcgggttaatcattaactacaCCAGGGTAATGGGCTAGCTGCGGC |
| | | CGCaggaacccctagtgatggagttggccactccctctctgcgcgctcgctc |
| | | gctcactgaggccgggcgaccaaaggtcgcccgacgcccgggctttgcccgg |
| | | gcggcctcagtgagcgagcgagcgcgcag |
| CAG.BB5.1 | SEQ ID | ctgcgcgctcgctcgctcactgaggccgcccgggcaaagcccgggcgtcggg |
| (ITR to ITR)(5′ | NO: 48 | cgacctttggtcgcccggcctcagtgagcgagcgagcgcgcagagagggagt |
| ITR to 3′ ITR) | | ggccaactccatcactaggggttcctCATATGcagggtaatggggatcctct |
| ITR sequences | | agataggtaccctagtcgacattgattattgactagttattaatagtaatca |
| in italics | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTCAGGTTC |
| | | AGCTGCAGCAACCTGGGGCTGAGCTTGTCAGACCTGGCACATCTGTGAAGCT |
| | | GAGCTGCAAGGCCTCTGGCTACACCTTCACAAGCAGCTGGATGCACTGGGTC |
| | | AAGCAGAGGCCTGGACAGGGCCTTGAGTGGATTGGAGTGATTGACCCCTCTG |
| | | ACAGCTACACCAACTACAACCAGAAGTTCAAGGGCAAAGCCACACTGACAGT |
| | | GGACACCAGCAGCAGCACAGCCTACATGCAGCTGAGCAGCCTGACCTCTGAG |
| | | GACTCTGCTGTGTACTACTGTGCCAGAGGTGGTGGCAGCAGCTACAACAGAT |
| | | ACTTTGATGTGTGGGGCACAGGCACCACAGTGACTGTGTCCTCTGCCAAGAC |
| | | AACCCCTCCTTCTGTGTACCCTCTGGCTCCTGGATCTGCTGCCCAGACCAAC |
| | | TCTATGGTCACACTGGGCTGTCTGGTCAAGGGCTACTTCCCTGAGCCTGTGA |
| | | CAGTGACCTGGAACTCTGGATCTCTGTCCTCTGGGGTGCACACATTCCCTGC |
| | | AGTGCTGCAGTCTGATCTGTACACCCTGAGCAGCTCTGTGACTGTGCCTAGC |
| | | AGCCCCAGACCTTCTGAGACTGTGACCTGCAATGTGGCTCACCCTGCCAGCA |
| | | GCACCAAGGTGGACAAGAAAATTGTGCCCAGAGACTGTGGCTGCAAGCCCTG |
| | | CATCTGTACAGTGCCTGAGGTTTCCTCTGTGTTCATCTTCCCACCTAAGCCT |
| | | AAGGATGTGCTGACCATCACACTGACCCCTAAAGTGACCTGTGTGGTGGTGG |
| | | ACATCAGCAAGGATGACCCTGAGGTGCAGTTCTCTTGGTTTGTGGATGATGT |
| | | GGAAGTGCACACAGCCCAGACACAGCCTAGAGAGGAACAGTTCAACAGCACC |
| | | TTCAGATCAGTGTCTGAGCTGCCCATCATGCACCAGGACTGGCTGAATGGCA |
| | | AAGAGTTCAAGTGCAGAGTGAACTCTGCTGCTTTCCCTGCTCCTATTGAAAA |
| | | GACCATCTCCAAGACCAAGGGCAGACCCAAGGCTCCCCAGGTGTACACAATC |
| | | CCTCCACCTAAAGAACAGATGGCCAAGGACAAGGTGTCCCTGACCTGCATGA |
| | | TCACAGATTTCTTCCCAGAGGATATCACAGTTGAGTGGCAGTGGAATGGCCA |
| | | GCCTGCTGAGAACTACAAGAACACCCAGCCTATCATGAACACCAATGGCAGC |
| | | TACTTTGTGTACAGCAAGCTGAATGTGCAGAAGTCCAACTGGGAGGCTGGCA |
| | | ACACCTTTACCTGCTCTGTGCTGCATGAGGGCCTGCACAACCACCATACAGA |
| | | GAAGTCCCTGTCTCACAGCCCTGGCAAGAGAAAGAGAAGAGGCTCTGGAGAA |
| | | GGCAGAGGCTCCCTGCTGACATGTGGGGATGTTGAAGAGAATCCTGGGCCTA |
| | | TGTATAGGATGCAACTGCTCCTCCTGATTGCTCTGAGCCTGGCTCTTGTGAC |
| | | CAACTCTAATATCATGATGACACAGAGCCCCAGCAGCCTGGCTGTGTCTGCT |
| | | GGGGAGAAAGTGACCATGAGCTGCAAGAGCAGCCAGTCTGTGCTGTACTCCA |
| | | GCAACCAGAAGAACTACCTGGCCTGGTATCAGCAGAAGCCTGGCCAGTCTCC |
| | | TAAGCTGCTGATCTACTGGGCCAGCACCAGAGAATCTGGGGTGCCAGACAGA |
| | | TTCACAGGCTCTGGCTCTGGCACAGACTTCACCCTGACAATCAGCTCTGTGC |
| | | AGGCTGAGGACCTGGCAGTGTACTACTGTCACCAGTACCTGAGCAGCAGAAC |
| | | CTTTGGTGGTGGCACCAAGCTGGAAATCAAGAGGGCTGATGCTGCCCCTACA |
| | | GTGTCTATCTTCCCACCTAGCTCTGAGCAGCTGACCAGTGGTGGTGCCTCTG |
| | | TTGTGTGCTTCCTGAACAACTTCTACCCCAAGGACATCAATGTGAAGTGGAA |
| | | GATTGATGGCTCTGAGAGGCAGAATGGGGTGCTGAACAGCTGGACAGACCAG |
| | | GACAGCAAGGACTCCACCTACAGCATGAGCAGCACACTGACCCTGACCAAGG |
| | | ATGAGTATGAGAGGCACAACAGCTACACCTGTGAAGCCACACACAAGACCAG |
| | | CACAAGCCCCATTGTGAAGTCCTTCAACAGGGGAGAGTGCTGATAACtcgag |
| | | gacggggtgaactacgcctgaggatccgatctttttccctctgccaaaaatt |
| | | atggggacatcatgaagccccttgagcatctgacttctggctaataaaggaa |
| | | atttattttcattgcaatagtgtgttggaattttttgtgtctctcactcgga |
| | | agcaattcgttgatctgaatttcgaccacccataatacccattaccctggta |
| | | gataagtagcatggcgggttaatcattaactacaCCAGGGTAATGGGCTAGC |
| | | TGCGGCCGCaggaacccctagtgatggagttggccactccctctctgcgcgc |
| | | tcgctcgctcactgaggccgggcgaccaaaggtcgcccgacgcccgggcttt |
| | | gcccgggcggcctcagtgagcgagcgagcgcgcag |
| hCFHL.1 | SEQ ID | GAATGGAAAAATTGTCAGTAGTGCAATGGAACCAGATCGGGAATACCATTTT |
| | NO: 49 | GGACAAGCAGTACGGTTTGTATGTAACTCAGGCTACAAGATTGAAGGAGATG |
| | | AAGAAATGCATTGTTCAGACGATGGTTTTTGGAGTAAAGAGAAACCAAAGTG |
| | | TGTGGAAATTTCATGCAAATCCCCAGATGTTATAAATGGATCTCCTATATCT |
| | | CAGAAGATTATTTATAAGGAGAATGAACGATTTCAATATAAATGTAACATGG |
| | | GTTATGAATACAGTGAAAGAGGAGATGCTGTATGCACTGAATCTGGATGGCG |
| | | TCCGTTGCCTTCATGTGAAGAAAAATCATGTGATAATCCTTATATTCCAAAT |
| | | GGTGACTACTCACCTTTAAGGATTAAACACAGAACTGGAGATGAAATCACGT |
| | | ACCAGTGTAGAAATGGTTTTTATCCTGCAACCCGGGGAAATACAGCAAAATG |
| | | CACAAGTACTGGCTGGATACCTGCTCCGAGATGTACCTTGAAACCTTGTGAT |
| | | TATCCAGACATTAAACATGGAGGTCTATATCATGAGAATATGCGTAGACCAT |
| | | ACTTTCCAGTAGCTGTAGGAAAATATTACTCCTATTACTGTGATGAACATTT |
| | | TGAGACTCCGTCAGGAAGTTACTGGGATCACATTCATTGCACACAAGATGGA |
| | | TGGTCGCCAGCAGTACCATGCCTCAGAAAATGTTATTTTCCTTATTTGGAAA |
| | | ATGGATATAATCAAAATCATGGAAGAAAGTTTGTACAGGGTAAATCTATAGA |
| | | CGTTGCCTGCCATCCTGGCTACGCTCTTCCAAAAGCGCAGACCACAGTTACA |
| | | TGTATGGAGAATGGCTGGTCTCCTACTCCCAGATGCATCCGTGTCAGCTTTA |
| | | CCCTCTGA |
| CAG.hCFHL.1 | SEQ ID | ctgcgcgctcgctcgctcactgaggccgcccgggcaaagcccgggcgtcggg |
| (5′ ITR to 3′ | NO: 50 | cgacctttggtcgcccggcctcagtgagcgagcgagcgcgcagagagggagt |
| ITR) ITR | | ggccaactccatcactaggggttcctCATATGcagggtaatggggatcctct |
| sequences in | | agataggtaccctagtcgacattgattattgactagttattaatagtaatca |
| italics | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcgggggcgggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGAGACTTCTAGCA |
| | | AAGATTATTTGCCTTATGTTATGGGCTATTTGTGTAGCAGAAGATTGCAATG |
| | | AACTTCCTCCAAGAAGAAATACAGAAATTCTGACAGGTTCCTGGTCTGACCA |
| | | AACATATCCAGAAGGCACCCAGGCTATCTATAAATGCCGCCCTGGATATAGA |
| | | TCTCTTGGAAATGTAATAATGGTATGCAGGAAGGGAGAATGGGTTGCTCTTA |
| | | ATCCATTAAGGAAATGTCAGAAAAGGCCCTGTGGACATCCTGGAGATACTCC |
| | | TTTTGGTACTTTTACCCTTACAGGAGGAAATGTGTTTGAATATGGTGTAAAA |
| | | GCTGTGTATACATGTAATGAGGGGTATCAATTGCTAGGTGAGATTAATTACC |
| | | GTGAATGTGACACAGATGGATGGACCAATGATATTCCTATATGTGAAGTTGT |
| | | GAAGTGTTTACCAGTGACAGCACCAGAGAATGGAAAAATTGTCAGTAGTGCA |
| | | ATGGAACCAGATCGGGAATACCATTTTGGACAAGCAGTACGGTTTGTATGTA |
| | | ACTCAGGCTACAAGATTGAAGGAGATGAAGAAATGCATTGTTCAGACGATGG |
| | | TTTTTGGAGTAAAGAGAAACCAAAGTGTGTGGAAATTTCATGCAAATCCCCA |
| | | GATGTTATAAATGGATCTCCTATATCTCAGAAGATTATTTATAAGGAGAATG |
| | | AACGATTTCAATATAAATGTAACATGGGTTATGAATACAGTGAAAGAGGAGA |
| | | TGCTGTATGCACTGAATCTGGATGGCGTCCGTTGCCTTCATGTGAAGAAAAA |
| | | TCATGTGATAATCCTTATATTCCAAATGGTGACTACTCACCTTTAAGGATTA |
| | | AACACAGAACTGGAGATGAAATCACGTACCAGTGTAGAAATGGTTTTTATCC |
| | | TGCAACCCGGGGAAATACAGCAAAATGCACAAGTACTGGCTGGATACCTGCT |
| | | CCGAGATGTACCTTGAAACCTTGTGATTATCCAGACATTAAACATGGAGGTC |
| | | TATATCATGAGAATATGCGTAGACCATACTTTCCAGTAGCTGTAGGAAAATA |
| | | TTACTCCTATTACTGTGATGAACATTTTGAGACTCCGTCAGGAAGTTACTGG |
| | | GATCACATTCATTGCACACAAGATGGATGGTCGCCAGCAGTACCATGCCTCA |
| | | GAAAATGTTATTTTCCTTATTTGGAAAATGGATATAATCAAAATCATGGAAG |
| | | AAAGTTTGTACAGGGTAAATCTATAGACGTTGCCTGCCATCCTGGCTACGCT |
| | | CTTCCAAAAGCGCAGACCACAGTTACATGTATGGAGAATGGCTGGTCTCCTA |
| | | CTCCCAGATGCATCCGTGTCAGCTTTACCCTCTGATAACtcgaggacggggt |
| | | gaaCTGCAGctacgcctGAGCTCgaCgatccgatctttttccctctgccaaa |
| | | aattatggggacatcatgaagccccttgagcatctgacttctggctaataaa |
| | | ggaaatttattttcattgcaatagtgtgttggaattttttgtgtctctcact |
| | | cggaagcaattcgttgatctgaatttcgaccacccataatacccattaccct |
| | | ggtagataagtagcatggcgggttaatcattaactacaCCAGGGTAATGGGC |
| | | TAGCTGCGGCCGCaggaacccctagtgatggagttggccactccctctctgc |
| | | gcgctcgctcgctcactgaggccgggcgaccaaaggtcgcccgacgcccggg |
| | | ctttgcccgggcggcctcagtgagcgagcgagcgcgcag |
| Crovalimab | SEQ ID | CAGGTGCAGCTGGTTGAATCTGGTGGTGGACTGGTGCAGCCTGGCAGAAGCC |
| scFv (H-L) | NO: 267 | TGAGACTGTCTTGTGCTGCCTCTGGCTTCACAGTGCACAGCAGCTACTACAT |
| Coding | | GGCCTGGGTTAGACAGGCCCCTGGCAAAGGACTTGAGTGGGTTGGAGCCATC |
| Sequence | | TTCACAGGCTCTGGGGCTGAGTATAAGGCTGAGTGGGCCAAGGGCAGAGTGA |
| | | CCATCAGCAAGGACACCAGCAAGAACCAGGTGGTGCTGACCATGACCAACAT |
| | | GGACCCTGTGGACACAGCCACCTACTACTGTGCCTCTGATGCTGGCTATGAC |
| | | TACCCCACACATGCCATGCACTATTGGGGCCAGGGCACCCTGGTTACAGTGT |
| | | CCTCTGGAGGTGGTGGAAGTGGTGGTGGTGGAAGTGGAGGTGGTGGTTCTGA |
| | | CATCCAGATGACACAGAGCCCTAGCAGCCTGTCTGCCTCTGTGGGAGACAGA |
| | | GTGACCATCACCTGTAGAGCCAGCCAGGGCATCTCTAGCAGTCTGGCCTGGT |
| | | ATCAGCAGAAGCCTGGAAAGGCCCCTAAGCTGCTGATCTATGGGGCCTCTGA |
| | | GACAGAATCTGGGGTGCCCAGCAGATTTTCAGGCTCTGGCTCTGGCACAGAC |
| | | TTCACCCTGACCATTTCTAGCCTGCAGCCTGAGGACTTTGCCACCTACTACT |
| | | GCCAGAACACCAAAGTGGGCAGCAGCTATGGCAACACCTTTGGTGGTGGCAC |
| | | CAAGGTGGAAATCAAGAGATAA |
| Crovalimab | SEQ ID | ctgcgcgctcgctcgctcactgaggccgcccgggcaaagcccgggcggcctc |
| scFv (H-L) | NO: 268 | agtgagcgagcgagcgcgcagagagggagtggaattcacgcgtggtaccgac |
| cassette (ITR- | | tagttattaatagtaatcaattacggggtcattagttcatagcccatatatg |
| ITR) | | gagttccgcgttacataacttacggtaaatggcccgcctggctgaccgccca |
| | | acgacccccgcccattgacgtcaataatgacgtatgttcccatagtaacgcc |
| | | aatagggactttccattgacgtcaatgggtggagtatttacggtaaactgcc |
| | | cacttggcagtacatcaagtgtatcatatgccaagtacgccccctattgacg |
| | | tcaatgacggtaaatggcccgcctggcattatgcccagtacatgaccttatg |
| | | ggactttcctacttggcagtacatctacgtattagtcatcgctattaccatg |
| | | gtcgaggtgagccccacgttctgcttcactctccccatctcccccccctccc |
| | | cacccccaattttgtatttatttattttttaattattttgtgcagcgatggg |
| | | ggcggggggggggggggggcgcgcgccaggcggggcggggcggggcgagggg |
| | | cggggcggggcgaggcggagaggtgcggcggcagccaatcagagcggcgcgc |
| | | tccgaaagtttccttttatggcgaggcggcggcggcggcggccctataaaaa |
| | | gcgaagcgcgcggcgggcgggagtcgctgcgcgctgccttcgccccgtgccc |
| | | cgctccgccgccgcctcgcgccgcccgccccggctctgactgaccgcgttac |
| | | tcccacaggtgagcgggcgggacggcccttctcctccgggctgtaattagcg |
| | | cttggtttaatgacggcttgtttcttttctgtggctgcgtgaaagccttgag |
| | | gggctccgggagggccctttgtgcggggggagcggctcggggggtgcgtgcg |
| | | tgtgtgtgtgcgtggggagcgccgcgtgcggctccgaatgggcggggagggc |
| | | cttcgtgcgtcgccgcgccgccgtccccttctccctctccagcctcggggct |
| | | gtccgcggggggacggctgccttcgggggggacggggcagggcggggttcgg |
| | | cttctggcgtgtgaccggcggctctagagcctctgctaaccatgttcatgcc |
| | | ttcttctttttcctacagctcctgggcaacgtgctggttattgtgctgtctc |
| | | atcattttggcaaaggaattcGCCACCATGTACAGAATGCAGCTGCTGCTGC |
| | | TCATTGCCCTGTCTCTGGCCCTGGTCACCAATTCTCAGGTGCAGCTGGTTGA |
| | | ATCTGGTGGTGGACTGGTGCAGCCTGGCAGAAGCCTGAGACTGTCTTGTGCT |
| | | GCCTCTGGCTTCACAGTGCACAGCAGCTACTACATGGCCTGGGTTAGACAGG |
| | | CCCCTGGCAAAGGACTTGAGTGGGTTGGAGCCATCTTCACAGGCTCTGGGGC |
| | | TGAGTATAAGGCTGAGTGGGCCAAGGGCAGAGTGACCATCAGCAAGGACACC |
| | | AGCAAGAACCAGGTGGTGCTGACCATGACCAACATGGACCCTGTGGACACAG |
| | | CCACCTACTACTGTGCCTCTGATGCTGGCTATGACTACCCCACACATGCCAT |
| | | GCACTATTGGGGCCAGGGCACCCTGGTTACAGTGTCCTCTGGAGGTGGTGGA |
| | | AGTGGTGGTGGTGGAAGTGGAGGTGGTGGTTCTGACATCCAGATGACACAGA |
| | | GCCCTAGCAGCCTGTCTGCCTCTGTGGGAGACAGAGTGACCATCACCTGTAG |
| | | AGCCAGCCAGGGCATCTCTAGCAGTCTGGCCTGGTATCAGCAGAAGCCTGGA |
| | | AAGGCCCCTAAGCTGCTGATCTATGGGGCCTCTGAGACAGAATCTGGGGTGC |
| | | CCAGCAGATTTTCAGGCTCTGGCTCTGGCACAGACTTCACCCTGACCATTTC |
| | | TAGCCTGCAGCCTGAGGACTTTGCCACCTACTACTGCCAGAACACCAAAGTG |
| | | GGCAGCAGCTATGGCAACACCTTTGGTGGTGGCACCAAGGTGGAAATCAAGA |
| | | GAtaaTAAagcggccatcaagcttgatctttttccctctgccaaaaattatg |
| | | gggacatcatgaagccccttgagcatctgacttctggctaataaaggaaatt |
| | | tattttcattgcaatagtgtgttggaattttttgtgtctctcactcggctag |
| | | cgcatgctggggagagatcgatctgaggaacccctagtgatggagttggcca |
| | | ctccctctctgcgcgctcgctcgctcactgaggccgGGCGACCAAAGGTCGC |
| | | CCGACGCCCGGGCTTTgCCcGGGCGGCCTCAgTGAGcgagcgagcgcgcaga |
| | | gagggagtggcc |
| Crovalimab | SEQ ID | gactagttattaatagtaatcaattacggggtcattagttcatagcccatat |
| scFv (H-L) | NO: 269 | atggagttccgcgttacataacttacggtaaatggcccgcctggctgaccgc |
| cassette | | ccaacgacccccgcccattgacgtcaataatgacgtatgttcccatagtaac |
| (CAG-signal | | gccaatagggactttccattgacgtcaatgggtggagtatttacggtaaact |
| sequence + ScFv | | gcccacttggcagtacatcaagtgtatcatatgccaagtacgccccctattg |
| transgene- | | acgtcaatgacggtaaatggcccgcctggcattatgcccagtacatgacctt |
| polyA) | | atgggactttcctacttggcagtacatctacgtattagtcatcgctattacc |
| | | atggtcgaggtgagccccacgttctgcttcactctccccatctcccccccct |
| | | ccccacccccaattttgtatttatttattttttaattattttgtgcagcgat |
| | | gggggcggggggggggggggggcgcgcgccaggcggggcggggcggggcgag |
| | | gggcggggcggggcgaggcggagaggtgcggcggcagccaatcagagcggcg |
| | | cgctccgaaagtttccttttatggcgaggcggcggcggcggcggccctataa |
| | | aaagcgaagcgcgcggcgggcgggagtcgctgcgcgctgccttcgccccgtg |
| | | ccccgctccgccgccgcctcgcgccgcccgccccggctctgactgaccgcgt |
| | | tactcccacaggtgagcgggcgggacggcccttctcctccgggctgtaatta |
| | | gcgcttggtttaatgacggcttgtttcttttctgtggctgcgtgaaagcctt |
| | | gaggggctccgggagggccctttgtgcggggggagcggctcggggggtgcgt |
| | | gcgtgtgtgtgtgcgtggggagcgccgcgtgcggctccgaatgggcggggag |
| | | ggccttcgtgcgtcgccgcgccgccgtccccttctccctctccagcctcggg |
| | | gctgtccgcggggggacggctgccttcgggggggacggggcagggcggggtt |
| | | cggcttctggcgtgtgaccggcggctctagagcctctgctaaccatgttcat |
| | | gccttcttctttttcctacagctcctgggcaacgtgctggttattgtgctgt |
| | | ctcatcattttggcaaaggaattcGCCACCATGTACAGAATGCAGCTGCTGC |
| | | TGCTCATTGCCCTGTCTCTGGCCCTGGTCACCAATTCTCAGGTGCAGCTGGT |
| | | TGAATCTGGTGGTGGACTGGTGCAGCCTGGCAGAAGCCTGAGACTGTCTTGT |
| | | GCTGCCTCTGGCTTCACAGTGCACAGCAGCTACTACATGGCCTGGGTTAGAC |
| | | AGGCCCCTGGCAAAGGACTTGAGTGGGTTGGAGCCATCTTCACAGGCTCTGG |
| | | GGCTGAGTATAAGGCTGAGTGGGCCAAGGGCAGAGTGACCATCAGCAAGGAC |
| | | ACCAGCAAGAACCAGGTGGTGCTGACCATGACCAACATGGACCCTGTGGACA |
| | | CAGCCACCTACTACTGTGCCTCTGATGCTGGCTATGACTACCCCACACATGC |
| | | CATGCACTATTGGGGCCAGGGCACCCTGGTTACAGTGTCCTCTGGAGGTGGT |
| | | GGAAGTGGTGGTGGTGGAAGTGGAGGTGGTGGTTCTGACATCCAGATGACAC |
| | | AGAGCCCTAGCAGCCTGTCTGCCTCTGTGGGAGACAGAGTGACCATCACCTG |
| | | TAGAGCCAGCCAGGGCATCTCTAGCAGTCTGGCCTGGTATCAGCAGAAGCCT |
| | | GGAAAGGCCCCTAAGCTGCTGATCTATGGGGCCTCTGAGACAGAATCTGGGG |
| | | TGCCCAGCAGATTTTCAGGCTCTGGCTCTGGCACAGACTTCACCCTGACCAT |
| | | TTCTAGCCTGCAGCCTGAGGACTTTGCCACCTACTACTGCCAGAACACCAAA |
| | | GTGGGCAGCAGCTATGGCAACACCTTTGGTGGTGGCACCAAGGTGGAAATCA |
| | | AGAGAtaaTAAagcggccatcaagcttgatctttttccctctgccaaaaatt |
| | | atggggacatcatgaagccccttgagcatctgacttctggctaataaaggaa |
| | | atttattttcattgcaatagtgtgttggaattttttgtgtctctcactcggc |
| | | tagcgcatgctggggag |
| Crovalimab.ScFv. | SEQ ID | ATGTACAGAATGCAGCTGCTGCTGCTCATTGCCCTGTCTCTGGCCCTGGTCA |
| HL (coding | NO: 277 | CCAATTCT |
| sequence with | | CAGGTGCAGCTGGTTGAATCTGGTGGTGGACTGGTGCAGCCTGGCAGAAGCC |
| signal sequence | | TGAGACTGTCTTGTGCTGCCTCTGGCTTCACAGTGCACAGCAGCTACTACAT |
| underlined) | | GGCCTGGGTTAGACAGGCCCCTGGCAAAGGACTTGAGTGGGTTGGAGCCATC |
| | | TTCACAGGCTCTGGGGCTGAGTATAAGGCTGAGTGGGCCAAGGGCAGAGTGA |
| | | CCATCAGCAAGGACACCAGCAAGAACCAGGTGGTGCTGACCATGACCAACAT |
| | | GGACCCTGTGGACACAGCCACCTACTACTGTGCCTCTGATGCTGGCTATGAC |
| | | TACCCCACACATGCCATGCACTATTGGGGCCAGGGCACCCTGGTTACAGTGT |
| | | CCTCTGGAGGTGGTGGAAGTGGTGGTGGTGGAAGTGGAGGTGGTGGTTCTGA |
| | | CATCCAGATGACACAGAGCCCTAGCAGCCTGTCTGCCTCTGTGGGAGACAGA |
| | | GTGACCATCACCTGTAGAGCCAGCCAGGGCATCTCTAGCAGTCTGGCCTGGT |
| | | ATCAGCAGAAGCCTGGAAAGGCCCCTAAGCTGCTGATCTATGGGGCCTCTGA |
| | | GACAGAATCTGGGGTGCCCAGCAGATTTTCAGGCTCTGGCTCTGGCACAGAC |
| | | TTCACCCTGACCATTTCTAGCCTGCAGCCTGAGGACTTTGCCACCTACTACT |
| | | GCCAGAACACCAAAGTGGGCAGCAGCTATGGCAACACCTTTGGTGGTGGCAC |
| | | CAAGGTGGAAATCAAGAGATAA |
| CAG.Crovalimab. | SEQ ID | gacattgattattgactagttattaatagtaatcaattacggggtcattagt |
| scFv.HL | NO: 278 | tcatagcccatatatggagttccgcgttacataacttacggtaaatggcccg |
| | | cctggctgaccgcccaacgacccccgcccattgacgtcaataatgacgtatg |
| | | ttcccatagtaacgccaatagggactttccattgacgtcaatgggtggagta |
| | | tttacggtaaactgcccacttggcagtacatcaagtgtatcatatgccaagt |
| | | acgccccctattgacgtcaatgacggtaaatggcccgcctggcattatgccc |
| | | agtacatgaccttatgggactttcctacttggcagtacatctacgtattagt |
| | | catcgctattaccatggtcgaggtgagccccacgttctgcttcactctcccc |
| | | atctcccccccctccccacccccaattttgtatttatttattttttaattat |
| | | tttgtgcagcgatgggggcggggggggggggggggcgcgcgccaggcggggc |
| | | ggggcggggcgaggggcggggcggggcgaggcggagaggtgcggcggcagcc |
| | | aatcagagcggcgcgctccgaaagtttccttttatggcgaggcggcggcggc |
| | | ggcggccctataaaaagcgaagcgcgcggcgggcgggagtcgctgcgcgctg |
| | | ccttcgccccgtgccccgctccgccgccgcctcgcgccgcccgccccggctc |
| | | tgactgaccgcgttactcccacaggtgagcgggcgggacggcccttctcctc |
| | | cgggctgtaattagcgcttggtttaatgacggcttgtttcttttctgtggct |
| | | gcgtgaaagccttgaggggctccgggagggccctttgtgcggggggagcggc |
| | | tcggggggtgcgtgcgtgtgtgtgtgcgtggggagcgccgcgtgcggctccg |
| | | cgctgcccggcggctgtgagcgctgcgggcgcggcgcggggctttgtgcgct |
| | | ccgcagtgtgcgcgaggggagcgcggccgggggcggtgccccgcggtgcggg |
| | | gggggctgcgaggggaacaaaggctgcgtgcggggtgtgtgcgtgggggggt |
| | | gagcagggggtgtgggcgcgtcggtcgggctgcaaccccccctgcacccccc |
| | | tccccgagttgctgagcacggcccggcttcgggtgcggggctccgtacgggg |
| | | cgtggcgcggggctcgccgtgccgggcggggggtggcggcaggtgggggtgc |
| | | cgggcggggcggggccgcctcgggccggggagggctcgggggaggggcgcgg |
| | | cggcccccggagcgccggcggctgtcgaggcgcggcgagccgcagccattgc |
| | | cttttatggtaatcgtgcgagagggcgcagggacttcctttgtcccaaatct |
| | | gtgcggagccgaaatctgggaggcgccgccgcaccccctctagcgggcgcgg |
| | | ggcgaagcggtgcggcgccggcaggaaggaaatgggcggggagggccttcgt |
| | | gcgtcgccgcgccgccgtccccttctccctctccagcctcggggctgtccgc |
| | | ggggggacggctgccttcgggggggacggggcagggcggggttcggcttctg |
| | | gcgtgtgaccggcggctctagagcctctgctaaccatgttcatgccttcttc |
| | | tttttcctacagctcctgggcaacgtgctggttattgtgctgtctcatcatt |
| | | ttggcaaagAATTCGCCACCATGTACAGAATGCAGCTGCTGCTGCTCATTGC |
| | | CCTGTCTCTGGCCCTGGTCACCAATTCTCAGGTGCAGCTGGTTGAATCTGGT |
| | | GGTGGACTGGTGCAGCCTGGCAGAAGCCTGAGACTGTCTTGTGCTGCCTCTG |
| | | GCTTCACAGTGCACAGCAGCTACTACATGGCCTGGGTTAGACAGGCCCCTGG |
| | | CAAAGGACTTGAGTGGGTTGGAGCCATCTTCACAGGCTCTGGGGCTGAGTAT |
| | | AAGGCTGAGTGGGCCAAGGGCAGAGTGACCATCAGCAAGGACACCAGCAAGA |
| | | ACCAGGTGGTGCTGACCATGACCAACATGGACCCTGTGGACACAGCCACCTA |
| | | CTACTGTGCCTCTGATGCTGGCTATGACTACCCCACACATGCCATGCACTAT |
| | | TGGGGCCAGGGCACCCTGGTTACAGTGTCCTCTGGAGGTGGTGGAAGTGGTG |
| | | GTGGTGGAAGTGGAGGTGGTGGTTCTGACATCCAGATGACACAGAGCCCTAG |
| | | CAGCCTGTCTGCCTCTGTGGGAGACAGAGTGACCATCACCTGTAGAGCCAGC |
| | | CAGGGCATCTCTAGCAGTCTGGCCTGGTATCAGCAGAAGCCTGGAAAGGCCC |
| | | CTAAGCTGCTGATCTATGGGGCCTCTGAGACAGAATCTGGGGTGCCCAGCAG |
| | | ATTTTCAGGCTCTGGCTCTGGCACAGACTTCACCCTGACCATTTCTAGCCTG |
| | | CAGCCTGAGGACTTTGCCACCTACTACTGCCAGAACACCAAAGTGGGCAGCA |
| | | GCTATGGCAACACCTTTGGTGGTGGCACCAAGGTGGAAATCAAGAGAtaaTA |
| | | ACtcgaggacggggtgaactacgcctgaggatccgatctttttccctctgcc |
| | | aaaaattatggggacatcatgaagccccttgagcatctgacttctggctaat |
| | | aaaggaaatttattttcattgcaatagtgtgttggaattttttgtgtctctc |
| | | actcg |
| ss.CAG.Crovalimab. | SEQ ID | ctgcgcgctcgctcgctcactgaggccgcccgggcaaagcccgggcgtcggg |
| scFv.HL | NO: 279 | cgacctttggtcgcccggcctcagtgagcgagcgagcgcgcagagagggagt |
| | | ggccaactccatcactaggggttcctCATATGcagggtaatggggatcctct |
| | | agataggtaccctagtcgacattgattattgactagttattaatagtaatca |
| | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTCAGGTGC |
| | | AGCTGGTTGAATCTGGTGGTGGACTGGTGCAGCCTGGCAGAAGCCTGAGACT |
| | | GTCTTGTGCTGCCTCTGGCTTCACAGTGCACAGCAGCTACTACATGGCCTGG |
| | | GTTAGACAGGCCCCTGGCAAAGGACTTGAGTGGGTTGGAGCCATCTTCACAG |
| | | GCTCTGGGGCTGAGTATAAGGCTGAGTGGGCCAAGGGCAGAGTGACCATCAG |
| | | CAAGGACACCAGCAAGAACCAGGTGGTGCTGACCATGACCAACATGGACCCT |
| | | GTGGACACAGCCACCTACTACTGTGCCTCTGATGCTGGCTATGACTACCCCA |
| | | CACATGCCATGCACTATTGGGGCCAGGGCACCCTGGTTACAGTGTCCTCTGG |
| | | AGGTGGTGGAAGTGGTGGTGGTGGAAGTGGAGGTGGTGGTTCTGACATCCAG |
| | | ATGACACAGAGCCCTAGCAGCCTGTCTGCCTCTGTGGGAGACAGAGTGACCA |
| | | TCACCTGTAGAGCCAGCCAGGGCATCTCTAGCAGTCTGGCCTGGTATCAGCA |
| | | GAAGCCTGGAAAGGCCCCTAAGCTGCTGATCTATGGGGCCTCTGAGACAGAA |
| | | TCTGGGGTGCCCAGCAGATTTTCAGGCTCTGGCTCTGGCACAGACTTCACCC |
| | | TGACCATTTCTAGCCTGCAGCCTGAGGACTTTGCCACCTACTACTGCCAGAA |
| | | CACCAAAGTGGGCAGCAGCTATGGCAACACCTTTGGTGGTGGCACCAAGGTG |
| | | GAAATCAAGAGAtaaTAACtcgaggacggggtgaactacgcctgaggatccg |
| | | atctttttccctctgccaaaaattatggggacatcatgaagccccttgagca |
| | | tctgacttctggctaataaaggaaatttattttcattgcaatagtgtgttgg |
| | | aattttttgtgtctctcactcggaagcaattcgttgatctgaatttcgacca |
| | | cccataatacccattaccctgCCAGGGTAATGGGCTAGCTGCGGCCGCagga |
| | | acccctagtgatggagttggccactccctctctgcgcgctcgctcgctcact |
| | | gaggccgggcgaccaaaggtcgcccgacgcccgggctttgcccgggcggcct |
| | | cagtgagcgagcgagcgcgcag |
| Crovalimab.scFv. | SEQ ID | ATGTACAGAATGCAGCTGCTGCTGCTCATTGCCCTGTCTCTGGCCCTGGTCA |
| LH (coding | NO: 280 | CCAATTCTGACATCCAGATGACACAGAGCCCTAGCAGCCTGTCTGCCTCTGT |
| sequence - | | GGGAGACAGAGTGACCATCACCTGTAGAGCCAGCCAGGGCATCTCTAGCAGT |
| signal sequence | | CTGGCCTGGTATCAGCAGAAGCCTGGAAAGGCCCCTAAGCTGCTGATCTATG |
| underlined) | | GGGCCTCTGAGACAGAATCTGGGGTGCCCAGCAGATTTTCAGGCTCTGGCTC |
| | | TGGCACAGACTTCACCCTGACCATTTCTAGCCTGCAGCCTGAGGACTTTGCC |
| | | ACCTACTACTGCCAGAACACCAAAGTGGGCAGCAGCTATGGCAACACCTTTG |
| | | GTGGTGGCACCAAGGTGGAAATCAAGAGAGGAGGTGGTGGAAGTGGTGGTGG |
| | | TGGAAGTGGAGGTGGTGGTTCTCAGGTGCAGCTGGTTGAATCTGGTGGTGGA |
| | | CTGGTGCAGCCTGGCAGAAGCCTGAGACTGTCTTGTGCTGCCTCTGGCTTCA |
| | | CAGTGCACAGCAGCTACTACATGGCCTGGGTTAGACAGGCCCCTGGCAAAGG |
| | | ACTTGAGTGGGTTGGAGCCATCTTCACAGGCTCTGGGGCTGAGTATAAGGCT |
| | | GAGTGGGCCAAGGGCAGAGTGACCATCAGCAAGGACACCAGCAAGAACCAGG |
| | | TGGTGCTGACCATGACCAACATGGACCCTGTGGACACAGCCACCTACTACTG |
| | | TGCCTCTGATGCTGGCTATGACTACCCCACACATGCCATGCACTATTGGGGC |
| | | CAGGGCACCCTGGTTACAGTGTCCTCTtaaTAACtcgaggacggggtgaact |
| | | acgcctgaggatccgatctttttccctctgccaaaaattatggggacatcat |
| | | gaagccccttgagcatctgacttctggctaataaaggaaatttattttcatt |
| | | gcaatagtgtgttggaattttttgtgtctctcactcgtaa |
| CAG.Crovalimab. | SEQ ID | gacattgattattgactagttattaatagtaatcaattacggggtcattagt |
| scFv.LH | NO: 281 | tcatagcccatatatggagttccgcgttacataacttacggtaaatggcccg |
| (Promoter to | | cctggctgaccgcccaacgacccccgcccattgacgtcaataatgacgtatg |
| polyA) | | ttcccatagtaacgccaatagggactttccattgacgtcaatgggtggagta |
| | | tttacggtaaactgcccacttggcagtacatcaagtgtatcatatgccaagt |
| | | acgccccctattgacgtcaatgacggtaaatggcccgcctggcattatgccc |
| | | agtacatgaccttatgggactttcctacttggcagtacatctacgtattagt |
| | | catcgctattaccatggtcgaggtgagccccacgttctgcttcactctcccc |
| | | atctcccccccctccccacccccaattttgtatttatttattttttaattat |
| | | tttgtgcagcgatgggggcggggggggggggggggcgcgcgccaggcggggc |
| | | ggggcggggcgaggggcggggcggggcgaggcggagaggtgcggcggcagcc |
| | | aatcagagcggcgcgctccgaaagtttccttttatggcgaggcggcggcggc |
| | | ggcggccctataaaaagcgaagcgcgcggcgggcgggagtcgctgcgcgctg |
| | | ccttcgccccgtgccccgctccgccgccgcctcgcgccgcccgccccggctc |
| | | tgactgaccgcgttactcccacaggtgagcgggcgggacggcccttctcctc |
| | | cgggctgtaattagcgcttggtttaatgacggcttgtttcttttctgtggct |
| | | gcgtgaaagccttgaggggctccgggagggccctttgtgcggggggagcggc |
| | | tcggggggtgcgtgcgtgtgtgtgtgcgtggggagcgccgcgtgcggctccg |
| | | cgctgcccggcggctgtgagcgctgcgggcgcggcgcggggctttgtgcgct |
| | | ccgcagtgtgcgcgaggggagcgcggccgggggcggtgccccgcggtgcggg |
| | | gggggctgcgaggggaacaaaggctgcgtgcggggtgtgtgcgtgggggggt |
| | | gagcagggggtgtgggcgcgtcggtcgggctgcaaccccccctgcacccccc |
| | | tccccgagttgctgagcacggcccggcttcgggtgcggggctccgtacgggg |
| | | cgtggcgcggggctcgccgtgccgggcggggggtggcggcaggtgggggtgc |
| | | cgggcggggcggggccgcctcgggccggggagggctcgggggaggggcgcgg |
| | | cggcccccggagcgccggcggctgtcgaggcgcggcgagccgcagccattgc |
| | | cttttatggtaatcgtgcgagagggcgcagggacttcctttgtcccaaatct |
| | | gtgcggagccgaaatctgggaggcgccgccgcaccccctctagcgggcgcgg |
| | | ggcgaagcggtgcggcgccggcaggaaggaaatgggcggggagggccttcgt |
| | | gcgtcgccgcgccgccgtccccttctccctctccagcctcggggctgtccgc |
| | | ggggggacggctgccttcgggggggacggggcagggcggggttcggcttctg |
| | | gcgtgtgaccggcggctctagagcctctgctaaccatgttcatgccttcttc |
| | | tttttcctacagctcctgggcaacgtgctggttattgtgctgtctcatcatt |
| | | ttggcaaagAATTCGCCACCATGTACAGAATGCAGCTGCTGCTGCTCATTGC |
| | | CCTGTCTCTGGCCCTGGTCACCAATTCTGACATCCAGATGACACAGAGCCCT |
| | | AGCAGCCTGTCTGCCTCTGTGGGAGACAGAGTGACCATCACCTGTAGAGCCA |
| | | GCCAGGGCATCTCTAGCAGTCTGGCCTGGTATCAGCAGAAGCCTGGAAAGGC |
| | | CCCTAAGCTGCTGATCTATGGGGCCTCTGAGACAGAATCTGGGGTGCCCAGC |
| | | AGATTTTCAGGCTCTGGCTCTGGCACAGACTTCACCCTGACCATTTCTAGCC |
| | | TGCAGCCTGAGGACTTTGCCACCTACTACTGCCAGAACACCAAAGTGGGCAG |
| | | CAGCTATGGCAACACCTTTGGTGGTGGCACCAAGGTGGAAATCAAGAGAGGA |
| | | GGTGGTGGAAGTGGTGGTGGTGGAAGTGGAGGTGGTGGTTCTCAGGTGCAGC |
| | | TGGTTGAATCTGGTGGTGGACTGGTGCAGCCTGGCAGAAGCCTGAGACTGTC |
| | | TTGTGCTGCCTCTGGCTTCACAGTGCACAGCAGCTACTACATGGCCTGGGTT |
| | | AGACAGGCCCCTGGCAAAGGACTTGAGTGGGTTGGAGCCATCTTCACAGGCT |
| | | CTGGGGCTGAGTATAAGGCTGAGTGGGCCAAGGGCAGAGTGACCATCAGCAA |
| | | GGACACCAGCAAGAACCAGGTGGTGCTGACCATGACCAACATGGACCCTGTG |
| | | GACACAGCCACCTACTACTGTGCCTCTGATGCTGGCTATGACTACCCCACAC |
| | | ATGCCATGCACTATTGGGGCCAGGGCACCCTGGTTACAGTGTCCTCTtaaTA |
| | | ACtcgaggacggggtgaactacgcctgaggatccgatctttttccctctgcc |
| | | aaaaattatggggacatcatgaagccccttgagcatctgacttctggctaat |
| | | aaaggaaatttattttcattgcaatagtgtgttggaattttttgtgtctctc |
| | | actcg |
| ss.CAG.Crovalimab. | SEQ ID | ctgcgcgctcgctcgctcactgaggccgcccgggcaaagcccgggcgtcggg |
| scFv.LH (ITR | NO: 282 | cgacctttggtcgcccggcctcagtgagcgagcgagcgcgcagagagggagt |
| to ITR) | | ggccaactccatcactaggggttcctCATATGcagggtaatggggatcctct |
| | | agataggtaccctagtcgacattgattattgactagttattaatagtaatca |
| | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTGACATCC |
| | | AGATGACACAGAGCCCTAGCAGCCTGTCTGCCTCTGTGGGAGACAGAGTGAC |
| | | CATCACCTGTAGAGCCAGCCAGGGCATCTCTAGCAGTCTGGCCTGGTATCAG |
| | | CAGAAGCCTGGAAAGGCCCCTAAGCTGCTGATCTATGGGGCCTCTGAGACAG |
| | | AATCTGGGGTGCCCAGCAGATTTTCAGGCTCTGGCTCTGGCACAGACTTCAC |
| | | CCTGACCATTTCTAGCCTGCAGCCTGAGGACTTTGCCACCTACTACTGCCAG |
| | | AACACCAAAGTGGGCAGCAGCTATGGCAACACCTTTGGTGGTGGCACCAAGG |
| | | TGGAAATCAAGAGAGGAGGTGGTGGAAGTGGTGGTGGTGGAAGTGGAGGTGG |
| | | TGGTTCTCAGGTGCAGCTGGTTGAATCTGGTGGTGGACTGGTGCAGCCTGGC |
| | | AGAAGCCTGAGACTGTCTTGTGCTGCCTCTGGCTTCACAGTGCACAGCAGCT |
| | | ACTACATGGCCTGGGTTAGACAGGCCCCTGGCAAAGGACTTGAGTGGGTTGG |
| | | AGCCATCTTCACAGGCTCTGGGGCTGAGTATAAGGCTGAGTGGGCCAAGGGC |
| | | AGAGTGACCATCAGCAAGGACACCAGCAAGAACCAGGTGGTGCTGACCATGA |
| | | CCAACATGGACCCTGTGGACACAGCCACCTACTACTGTGCCTCTGATGCTGG |
| | | CTATGACTACCCCACACATGCCATGCACTATTGGGGCCAGGGCACCCTGGTT |
| | | ACAGTGTCCTCTtaaTAACtcgaggacggggtgaactacgcctgaggatccg |
| | | atctttttccctctgccaaaaattatggggacatcatgaagccccttgagca |
| | | tctgacttctggctaataaaggaaatttattttcattgcaatagtgtgttgg |
| | | aattttttgtgtctctcactcggaagcaattcgttgatctgaatttcgacca |
| | | cccataatacccattaccctgCCAGGGTAATGGGCTAGCTGCGGCCGCagga |
| | | acccctagtgatggagttggccactccctctctgcgcgctcgctcgctcact |
| | | gaggccgggcgaccaaaggtcgcccgacgcccgggctttgcccgggcggcct |
| | | cagtgagcgagcgagcgcgcag |
| VH4i.Crovalimab. | SEQ ID | ATGGAGGCGGTACTATGTAGATGAGAATTCAGGAGCAAACTGGGAAAAGCAA |
| HL.scFv.RBGpA | NO: 283 | CTGCTTCCAAATATTTGTGATTTTTACAGTGTAGTTTTGGAAAAACTCTTAG |
| (Promoter to | | CCTACCAATTCTTCTAAGTGTTTTAAAATGTGGGAGCCAGTACACATGAAGT |
| polyA) | | TATAGAGTGTTTTAATGAGGCTTAAATATTTACCGTAACTATGAAATGCTAC |
| | | GCATATCATGCTGTTCAGGCTCCGTGGCCACGCAACTCATACTCAGGTGAGT |
| | | ATCTCAGGGATCCAGACATGGGGATATGGGAGGTGCCTCTGATCCCAGGGCT |
| | | CACTGTGGGTCTCTCTGTTCACAGGTTaccggtGCCACCATGTACAGAATGC |
| | | AGCTGCTGCTGCTCATTGCCCTGTCTCTGGCCCTGGTCACCAATTCTCAGGT |
| | | GCAGCTGGTTGAATCTGGTGGTGGACTGGTGCAGCCTGGCAGAAGCCTGAGA |
| | | CTGTCTTGTGCTGCCTCTGGCTTCACAGTGCACAGCAGCTACTACATGGCCT |
| | | GGGTTAGACAGGCCCCTGGCAAAGGACTTGAGTGGGTTGGAGCCATCTTCAC |
| | | AGGCTCTGGGGCTGAGTATAAGGCTGAGTGGGCCAAGGGCAGAGTGACCATC |
| | | AGCAAGGACACCAGCAAGAACCAGGTGGTGCTGACCATGACCAACATGGACC |
| | | CTGTGGACACAGCCACCTACTACTGTGCCTCTGATGCTGGCTATGACTACCC |
| | | CACACATGCCATGCACTATTGGGGCCAGGGCACCCTGGTTACAGTGTCCTCT |
| | | GGAGGTGGTGGAAGTGGTGGTGGTGGAAGTGGAGGTGGTGGTTCTGACATCC |
| | | AGATGACACAGAGCCCTAGCAGCCTGTCTGCCTCTGTGGGAGACAGAGTGAC |
| | | CATCACCTGTAGAGCCAGCCAGGGCATCTCTAGCAGTCTGGCCTGGTATCAG |
| | | CAGAAGCCTGGAAAGGCCCCTAAGCTGCTGATCTATGGGGCCTCTGAGACAG |
| | | AATCTGGGGTGCCCAGCAGATTTTCAGGCTCTGGCTCTGGCACAGACTTCAC |
| | | CCTGACCATTTCTAGCCTGCAGCCTGAGGACTTTGCCACCTACTACTGCCAG |
| | | AACACCAAAGTGGGCAGCAGCTATGGCAACACCTTTGGTGGTGGCACCAAGG |
| | | TGGAAATCAAGAGAtaaTAACtcgaggacggggtgaactacgcctgaggatc |
| | | cgatctttttccctctgccaaaaattatggggacatcatgaagccccttgag |
| | | catctgacttctggctaataaaggaaatttattttcattgcaatagtgtgtt |
| | | ggaattttttgtgtctctcactcg |
| scAAV.mula.VH4i. | SEQ ID | ctgcgcgctcgctcgctcactgaggccgcccgggcaaagcccgggcggcctc |
| Crovalimab.scFv. | NO: 284 | agtgagcgagcgagcgcgcagagagggagtggaattcaCGCGTATGGAGGCG |
| HL.RBGpA | | GTACTATGTAGATGAGAATTCAGGAGCAAACTGGGAAAAGCAACTGCTTCCA |
| (ITR to ITR) | | AATATTTGTGATTTTTACAGTGTAGTTTTGGAAAAACTCTTAGCCTACCAAT |
| | | TCTTCTAAGTGTTTTAAAATGTGGGAGCCAGTACACATGAAGTTATAGAGTG |
| | | TTTTAATGAGGCTTAAATATTTACCGTAACTATGAAATGCTACGCATATCAT |
| | | GCTGTTCAGGCTCCGTGGCCACGCAACTCATACTCAGGTGAGTATCTCAGGG |
| | | ATCCAGACATGGGGATATGGGAGGTGCCTCTGATCCCAGGGCTCACTGTGGG |
| | | TCTCTCTGTTCACAGGTTaccggtGCCACCATGTACAGAATGCAGCTGCTGC |
| | | TGCTCATTGCCCTGTCTCTGGCCCTGGTCACCAATTCTCAGGTGCAGCTGGT |
| | | TGAATCTGGTGGTGGACTGGTGCAGCCTGGCAGAAGCCTGAGACTGTCTTGT |
| | | GCTGCCTCTGGCTTCACAGTGCACAGCAGCTACTACATGGCCTGGGTTAGAC |
| | | AGGCCCCTGGCAAAGGACTTGAGTGGGTTGGAGCCATCTTCACAGGCTCTGG |
| | | GGCTGAGTATAAGGCTGAGTGGGCCAAGGGCAGAGTGACCATCAGCAAGGAC |
| | | ACCAGCAAGAACCAGGTGGTGCTGACCATGACCAACATGGACCCTGTGGACA |
| | | CAGCCACCTACTACTGTGCCTCTGATGCTGGCTATGACTACCCCACACATGC |
| | | CATGCACTATTGGGGCCAGGGCACCCTGGTTACAGTGTCCTCTGGAGGTGGT |
| | | GGAAGTGGTGGTGGTGGAAGTGGAGGTGGTGGTTCTGACATCCAGATGACAC |
| | | AGAGCCCTAGCAGCCTGTCTGCCTCTGTGGGAGACAGAGTGACCATCACCTG |
| | | TAGAGCCAGCCAGGGCATCTCTAGCAGTCTGGCCTGGTATCAGCAGAAGCCT |
| | | GGAAAGGCCCCTAAGCTGCTGATCTATGGGGCCTCTGAGACAGAATCTGGGG |
| | | TGCCCAGCAGATTTTCAGGCTCTGGCTCTGGCACAGACTTCACCCTGACCAT |
| | | TTCTAGCCTGCAGCCTGAGGACTTTGCCACCTACTACTGCCAGAACACCAAA |
| | | GTGGGCAGCAGCTATGGCAACACCTTTGGTGGTGGCACCAAGGTGGAAATCA |
| | | AGAGAtaaTAACtcgaggacggggtgaactacgcctgaggatccgatctttt |
| | | tccctctgccaaaaattatggggacatcatgaagccccttgagcatctgact |
| | | tctggctaataaaggaaatttattttcattgcaatagtgtgttggaattttt |
| | | tgtgtctctcactcggaagcaattcgttgatctgaatttcgaccacccataa |
| | | tacccattaccctgCCAGGGTAATGGGctagcgaagcaattctagcaggcat |
| | | gctggggagagatcgatctgaggaacccctagtgatggagttggccactccc |
| | | tctctgcgcgctcgctcgctcactgaggccgGGCGACCAAAGGTCGCCCGAC |
| | | GCCCGGGCTTTgCCcGGGCGGCCTCAgTGAGcgagcgagcgcgcagagaggg |
| | | agtggcc |
| SCAAV.CB.sv40. | SEQ ID | acgcgtggtacctctggtcgttacataacttacggtaaatggcccgcctggc |
| Crovalimab.scFv. | NO: 285 | tgaccgcccaacgacccccgcccattgacgtcaataatgacgtatgttccca |
| HL (Promoter to | | tagtaacgccaatagggactttccattgacgtcaatgggtggagtatttacg |
| polyA) | | gtaaactgcccacttggcagtacatcaagtgtatcatatgccaagtacgccc |
| | | cctattgacgtcaatgacggtaaatggcccgcctggcattatgcccagtaca |
| | | tgaccttatgggactttcctacttggcagtacatctactcgaggccacgttc |
| | | tgcttcactctccccatctcccccccctccccacccccaattttgtatttat |
| | | ttattttttaattattttgtgcagcgatgggggcgggggggggggggggggg |
| | | ggcgcgcgccaggcggggcggggcggggcgaggggcggggcggggcgaggcg |
| | | gagaggtgcggcggcagccaatcagagcggcgcgctccgaaagtttcctttt |
| | | atggcgaggcggcggcggcggcggccctataaaaagcgaagcgcgcggcggg |
| | | cgggagcgggatcagccaccgcggtggcggcctagagtcgacgaggaactga |
| | | aaaaccagaaagttaactggtaagtttagtctttttgtcttttatttcaggt |
| | | cccggatccggtggtggtgcaaatcaaagaactgctcctcagtggatgttgc |
| | | ctttacttctaggcctgtacggaagtgttacttctgctctaaaagctgcgga |
| | | attgtacccgcggccgatccaccggtcGCCACCATGTACAGAATGCAGCTGC |
| | | TGCTGCTCATTGCCCTGTCTCTGGCCCTGGTCACCAATTCTCAGGTGCAGCT |
| | | GGTTGAATCTGGTGGTGGACTGGTGCAGCCTGGCAGAAGCCTGAGACTGTCT |
| | | TGTGCTGCCTCTGGCTTCACAGTGCACAGCAGCTACTACATGGCCTGGGTTA |
| | | GACAGGCCCCTGGCAAAGGACTTGAGTGGGTTGGAGCCATCTTCACAGGCTC |
| | | TGGGGCTGAGTATAAGGCTGAGTGGGCCAAGGGCAGAGTGACCATCAGCAAG |
| | | GACACCAGCAAGAACCAGGTGGTGCTGACCATGACCAACATGGACCCTGTGG |
| | | ACACAGCCACCTACTACTGTGCCTCTGATGCTGGCTATGACTACCCCACACA |
| | | TGCCATGCACTATTGGGGCCAGGGCACCCTGGTTACAGTGTCCTCTGGAGGT |
| | | GGTGGAAGTGGTGGTGGTGGAAGTGGAGGTGGTGGTTCTGACATCCAGATGA |
| | | CACAGAGCCCTAGCAGCCTGTCTGCCTCTGTGGGAGACAGAGTGACCATCAC |
| | | CTGTAGAGCCAGCCAGGGCATCTCTAGCAGTCTGGCCTGGTATCAGCAGAAG |
| | | CCTGGAAAGGCCCCTAAGCTGCTGATCTATGGGGCCTCTGAGACAGAATCTG |
| | | GGGTGCCCAGCAGATTTTCAGGCTCTGGCTCTGGCACAGACTTCACCCTGAC |
| | | CATTTCTAGCCTGCAGCCTGAGGACTTTGCCACCTACTACTGCCAGAACACC |
| | | AAAGTGGGCAGCAGCTATGGCAACACCTTTGGTGGTGGCACCAAGGTGGAAA |
| | | TCAAGAGAtaaTAAagcggccatcaagcttgatctttttccctctgccaaaa |
| | | attatggggacatcatgaagccccttgagcatctgacttctggctaataaag |
| | | gaaatttattttcattgcaatagtgtgttggaattttttgtgtctctcactc |
| | | g |
| SCAAV.CB.sv40. | SEQ ID | ctgcgcgctcgctcgctcactgaggccgcccgggcaaagcccgggcggcctc |
| Crovalimab.scFv. | NO: 286 | agtgagcgagcgagcgcgcagagagggagtggaattcacgcgtggtacctct |
| HL (ITR to ITR) | | ggtcgttacataacttacggtaaatggcccgcctggctgaccgcccaacgac |
| | | ccccgcccattgacgtcaataatgacgtatgttcccatagtaacgccaatag |
| | | ggactttccattgacgtcaatgggtggagtatttacggtaaactgcccactt |
| | | ggcagtacatcaagtgtatcatatgccaagtacgccccctattgacgtcaat |
| | | gacggtaaatggcccgcctggcattatgcccagtacatgaccttatgggact |
| | | ttcctacttggcagtacatctactcgaggccacgttctgcttcactctcccc |
| | | atctcccccccctccccacccccaattttgtatttatttattttttaattat |
| | | tttgtgcagcgatgggggcggggggggggggggggggggcgcgcgccaggcg |
| | | gggcggggcggggcgaggggcggggcggggcgaggcggagaggtgcggcggc |
| | | agccaatcagagcggcgcgctccgaaagtttccttttatggcgaggcggcgg |
| | | cggcggcggccctataaaaagcgaagcgcgcggcgggcgggagcgggatcag |
| | | ccaccgcggtggcggcctagagtcgacgaggaactgaaaaaccagaaagtta |
| | | actggtaagtttagtctttttgtcttttatttcaggtcccggatccggtggt |
| | | ggtgcaaatcaaagaactgctcctcagtggatgttgcctttacttctaggcc |
| | | tgtacggaagtgttacttctgctctaaaagctgcggaattgtacccgcggcc |
| | | gatccaccggtcGCCACCATGTACAGAATGCAGCTGCTGCTGCTCATTGCCC |
| | | TGTCTCTGGCCCTGGTCACCAATTCTCAGGTGCAGCTGGTTGAATCTGGTGG |
| | | TGGACTGGTGCAGCCTGGCAGAAGCCTGAGACTGTCTTGTGCTGCCTCTGGC |
| | | TTCACAGTGCACAGCAGCTACTACATGGCCTGGGTTAGACAGGCCCCTGGCA |
| | | AAGGACTTGAGTGGGTTGGAGCCATCTTCACAGGCTCTGGGGCTGAGTATAA |
| | | GGCTGAGTGGGCCAAGGGCAGAGTGACCATCAGCAAGGACACCAGCAAGAAC |
| | | CAGGTGGTGCTGACCATGACCAACATGGACCCTGTGGACACAGCCACCTACT |
| | | ACTGTGCCTCTGATGCTGGCTATGACTACCCCACACATGCCATGCACTATTG |
| | | GGGCCAGGGCACCCTGGTTACAGTGTCCTCTGGAGGTGGTGGAAGTGGTGGT |
| | | GGTGGAAGTGGAGGTGGTGGTTCTGACATCCAGATGACACAGAGCCCTAGCA |
| | | GCCTGTCTGCCTCTGTGGGAGACAGAGTGACCATCACCTGTAGAGCCAGCCA |
| | | GGGCATCTCTAGCAGTCTGGCCTGGTATCAGCAGAAGCCTGGAAAGGCCCCT |
| | | AAGCTGCTGATCTATGGGGCCTCTGAGACAGAATCTGGGGTGCCCAGCAGAT |
| | | TTTCAGGCTCTGGCTCTGGCACAGACTTCACCCTGACCATTTCTAGCCTGCA |
| | | GCCTGAGGACTTTGCCACCTACTACTGCCAGAACACCAAAGTGGGCAGCAGC |
| | | TATGGCAACACCTTTGGTGGTGGCACCAAGGTGGAAATCAAGAGAtaaTAAa |
| | | gcggccatcaagcttgatctttttccctctgccaaaaattatggggacatca |
| | | tgaagccccttgagcatctgacttctggctaataaaggaaatttattttcat |
| | | tgcaatagtgtgttggaattttttgtgtctctcactcggctagcgaagcaat |
| | | tctagcaggcatgctggggagagatcgatctgaggaacccctagtgatggag |
| | | ttggccactccctctctgcgcgctcgctcgctcactgaggccgGGCGACCAA |
| | | AGGTCGCCCGACGCCCGGGCTTTgCCCGGGCGGCCTCAgTGAGcgagcgagc |
| | | gcgcagagagggagtggcc |
| CAG(Del5). | SEQ ID | gactagttattaatagtaatcaattacggggtcattagttcatagcccatat |
| Crovalimab.ScFv. | NO: 287 | atggagttccgcgttacataacttacggtaaatggcccgcctggctgaccgc |
| HL | | ccaacgacccccgcccattgacgtcaataatgacgtatgttcccatagtaac |
| (Promoter to | | gccaatagggactttccattgacgtcaatgggtggagtatttacggtaaact |
| polyA) | | gcccacttggcagtacatcaagtgtatcatatgccaagtacgccccctattg |
| | | acgtcaatgacggtaaatggcccgcctggcattatgcccagtacatgacctt |
| | | atgggactttcctacttggcagtacatctacgtattagtcatcgctattacc |
| | | atggtcgaggtgagccccacgttctgcttcactctccccatctcccccccct |
| | | ccccacccccaattttgtatttatttattttttaattattttgtgcagcgat |
| | | gggggcggggggggggggggggcgcgcgccaggcggggcggggcggggcgag |
| | | gggcggggcggggcgaggcggagaggtgcggcggcagccaatcagagcggcg |
| | | cgctccgaaagtttccttttatggcgaggcggcggcggcggcggccctataa |
| | | aaagcgaagcgcgcggcgggcgggagtcgctgcgcgctgccttcgccccgtg |
| | | ccccgctccgccgccgcctcgcgccgcccgccccggctctgactgaccgcgt |
| | | tactcccacaggtgagcgggcgggacggcccttctcctccgggctgtgcgag |
| | | ccgcagccattgccttttatggtaatcgtgcgagagggcgcagggacttcct |
| | | ttgtcccaaatctgtgcggagccgaaatctgggaggcgccgccgcaccccct |
| | | ctagcgggcgcggggcgaagcggtgcggcgccggcaggaaggaaatgggcgg |
| | | ggagggccttcgtgcgtcgccgcgccgccgtccccttctccctctccagcct |
| | | cggggctgtccgcggggggacggctgccttcgggggggacggggcagggcgg |
| | | ggttcggcttctggcgtgtgaccggcggctctagagcctctgctaaccatgt |
| | | tcatgccttcttctttttcctacagctcctgggcaacgtgctggttattgtg |
| | | ctgtctcatcattttggcaaaggaattcGCCACCATGTACAGAATGCAGCTG |
| | | CTGCTGCTCATTGCCCTGTCTCTGGCCCTGGTCACCAATTCTCAGGTGCAGC |
| | | TGGTTGAATCTGGTGGTGGACTGGTGCAGCCTGGCAGAAGCCTGAGACTGTC |
| | | TTGTGCTGCCTCTGGCTTCACAGTGCACAGCAGCTACTACATGGCCTGGGTT |
| | | AGACAGGCCCCTGGCAAAGGACTTGAGTGGGTTGGAGCCATCTTCACAGGCT |
| | | CTGGGGCTGAGTATAAGGCTGAGTGGGCCAAGGGCAGAGTGACCATCAGCAA |
| | | GGACACCAGCAAGAACCAGGTGGTGCTGACCATGACCAACATGGACCCTGTG |
| | | GACACAGCCACCTACTACTGTGCCTCTGATGCTGGCTATGACTACCCCACAC |
| | | ATGCCATGCACTATTGGGGCCAGGGCACCCTGGTTACAGTGTCCTCTGGAGG |
| | | TGGTGGAAGTGGTGGTGGTGGAAGTGGAGGTGGTGGTTCTGACATCCAGATG |
| | | ACACAGAGCCCTAGCAGCCTGTCTGCCTCTGTGGGAGACAGAGTGACCATCA |
| | | CCTGTAGAGCCAGCCAGGGCATCTCTAGCAGTCTGGCCTGGTATCAGCAGAA |
| | | GCCTGGAAAGGCCCCTAAGCTGCTGATCTATGGGGCCTCTGAGACAGAATCT |
| | | GGGGTGCCCAGCAGATTTTCAGGCTCTGGCTCTGGCACAGACTTCACCCTGA |
| | | CCATTTCTAGCCTGCAGCCTGAGGACTTTGCCACCTACTACTGCCAGAACAC |
| | | CAAAGTGGGCAGCAGCTATGGCAACACCTTTGGTGGTGGCACCAAGGTGGAA |
| | | ATCAAGAGAtaaTAAagcggccatcaagcttgatctttttccctctgccaaa |
| | | aattatggggacatcatgaagccccttgagcatctgacttctggctaataaa |
| | | ggaaatttattttcattgcaatagtgtgttggaattttttgtgtctctcact |
| | | cg |
| sCAAV.CAG(Del5). | SEQ ID | ctgcgcgctcgctcgctcactgaggccgcccgggcaaagcccgggcggcctc |
| Crovalimab.ScFv. | NO: 288 | agtgagcgagcgagcgcgcagagagggagtggaattcacgcgtggtaccgac |
| HL (ITR to ITR) | | tagttattaatagtaatcaattacggggtcattagttcatagcccatatatg |
| | | gagttccgcgttacataacttacggtaaatggcccgcctggctgaccgccca |
| | | acgacccccgcccattgacgtcaataatgacgtatgttcccatagtaacgcc |
| | | aatagggactttccattgacgtcaatgggtggagtatttacggtaaactgcc |
| | | cacttggcagtacatcaagtgtatcatatgccaagtacgccccctattgacg |
| | | tcaatgacggtaaatggcccgcctggcattatgcccagtacatgaccttatg |
| | | ggactttcctacttggcagtacatctacgtattagtcatcgctattaccatg |
| | | gtcgaggtgagccccacgttctgcttcactctccccatctcccccccctccc |
| | | cacccccaattttgtatttatttattttttaattattttgtgcagcgatggg |
| | | ggcggggggggggggggggcgcgcgccaggcggggcggggcggggcgagggg |
| | | cggggcggggcgaggcggagaggtgcggcggcagccaatcagagcggcgcgc |
| | | tccgaaagtttccttttatggcgaggcggcggcggcggcggccctataaaaa |
| | | gcgaagcgcgcggcgggcgggagtcgctgcgcgctgccttcgccccgtgccc |
| | | cgctccgccgccgcctcgcgccgcccgccccggctctgactgaccgcgttac |
| | | tcccacaggtgagcgggcgggacggcccttctcctccgggctgtgcgagccg |
| | | cagccattgccttttatggtaatcgtgcgagagggcgcagggacttcctttg |
| | | tcccaaatctgtgcggagccgaaatctgggaggcgccgccgcaccccctcta |
| | | gcgggcgcggggcgaagcggtgcggcgccggcaggaaggaaatgggcgggga |
| | | gggccttcgtgcgtcgccgcgccgccgtccccttctccctctccagcctcgg |
| | | ggctgtccgcggggggacggctgccttcgggggggacggggcagggcggggt |
| | | tcggcttctggcgtgtgaccggcggctctagagcctctgctaaccatgttca |
| | | tgccttcttctttttcctacagctcctgggcaacgtgctggttattgtgctg |
| | | tctcatcattttggcaaaggaattcGCCACCATGTACAGAATGCAGCTGCTG |
| | | CTGCTCATTGCCCTGTCTCTGGCCCTGGTCACCAATTCTCAGGTGCAGCTGG |
| | | TTGAATCTGGTGGTGGACTGGTGCAGCCTGGCAGAAGCCTGAGACTGTCTTG |
| | | TGCTGCCTCTGGCTTCACAGTGCACAGCAGCTACTACATGGCCTGGGTTAGA |
| | | CAGGCCCCTGGCAAAGGACTTGAGTGGGTTGGAGCCATCTTCACAGGCTCTG |
| | | GGGCTGAGTATAAGGCTGAGTGGGCCAAGGGCAGAGTGACCATCAGCAAGGA |
| | | CACCAGCAAGAACCAGGTGGTGCTGACCATGACCAACATGGACCCTGTGGAC |
| | | ACAGCCACCTACTACTGTGCCTCTGATGCTGGCTATGACTACCCCACACATG |
| | | CCATGCACTATTGGGGCCAGGGCACCCTGGTTACAGTGTCCTCTGGAGGTGG |
| | | TGGAAGTGGTGGTGGTGGAAGTGGAGGTGGTGGTTCTGACATCCAGATGACA |
| | | CAGAGCCCTAGCAGCCTGTCTGCCTCTGTGGGAGACAGAGTGACCATCACCT |
| | | GTAGAGCCAGCCAGGGCATCTCTAGCAGTCTGGCCTGGTATCAGCAGAAGCC |
| | | TGGAAAGGCCCCTAAGCTGCTGATCTATGGGGCCTCTGAGACAGAATCTGGG |
| | | GTGCCCAGCAGATTTTCAGGCTCTGGCTCTGGCACAGACTTCACCCTGACCA |
| | | TTTCTAGCCTGCAGCCTGAGGACTTTGCCACCTACTACTGCCAGAACACCAA |
| | | AGTGGGCAGCAGCTATGGCAACACCTTTGGTGGTGGCACCAAGGTGGAAATC |
| | | AAGAGAtaaTAAagcggccatcaagcttgatctttttccctctgccaaaaat |
| | | tatggggacatcatgaagccccttgagcatctgacttctggctaataaagga |
| | | aatttattttcattgcaatagtgtgttggaattttttgtgtctctcactcgg |
| | | ctagcgcatgctggggagagatcgatctgaggaacccctagtgatggagttg |
| | | gccactccctctctgcgcgctcgctcgctcactgaggccgGGCGACCAAAGG |
| | | TCGCCCGACGCCCGGGCTTTgCCcGGGCGGCCTCAgTGAGcgagcgagcgcg |
| | | cagagagggagtggcc |
| CAG(Delm). | SEQ ID | gactagttattaatagtaatcaattacggggtcattagttcatagcccatat |
| Crovalimab.ScFv. | NO: 289 | atggagttccgcgttacataacttacggtaaatggcccgcctggctgaccgc |
| HL | | ccaacgacccccgcccattgacgtcaataatgacgtatgttcccatagtaac |
| (Promoter to | | gccaatagggactttccattgacgtcaatgggtggagtatttacggtaaact |
| polyA) | | gcccacttggcagtacatcaagtgtatcatatgccaagtacgccccctattg |
| | | acgtcaatgacggtaaatggcccgcctggcattatgcccagtacatgacctt |
| | | atgggactttcctacttggcagtacatctacgtattagtcatcgctattacc |
| | | atggtcgaggtgagccccacgttctgcttcactctccccatctcccccccct |
| | | ccccacccccaattttgtatttatttattttttaattattttgtgcagcgat |
| | | gggggcggggggggggggggggcgcgcgccaggcggggcggggcggggcgag |
| | | gggcggggcggggcgaggcggagaggtgcggcggcagccaatcagagcggcg |
| | | cgctccgaaagtttccttttatggcgaggcggcggcggcggcggccctataa |
| | | aaagcgaagcgcgcggcgggcgggagtcgctgcgcgctgccttcgccccgtg |
| | | ccccgctccgccgccgcctcgcgccgcccgccccggctctgactgaccgcgt |
| | | tactcccacaggtgagcgggcgggacggcccttctcctccgggctgtaatta |
| | | gcgcttggtttaatgacggcttgtttcttttctgtggctgcgtgaaagcctt |
| | | gaggggctccgggagggccctttgtgcggggggagcggctcggggggtgcgt |
| | | gcgtgtgtgtgtgcgtggggagcgccgcgtgcggctccgaatgggcggggag |
| | | ggccttcgtgcgtcgccgcgccgccgtccccttctccctctccagcctcggg |
| | | gctgtccgcggggggacggctgccttcgggggggacggggcagggcggggtt |
| | | cggcttctggcgtgtgaccggcggctctagagcctctgctaaccatgttcat |
| | | gccttcttctttttcctacagctcctgggcaacgtgctggttattgtgctgt |
| | | ctcatcattttggcaaaggaattcGCCACCATGTACAGAATGCAGCTGCTGC |
| | | TGCTCATTGCCCTGTCTCTGGCCCTGGTCACCAATTCTCAGGTGCAGCTGGT |
| | | TGAATCTGGTGGTGGACTGGTGCAGCCTGGCAGAAGCCTGAGACTGTCTTGT |
| | | GCTGCCTCTGGCTTCACAGTGCACAGCAGCTACTACATGGCCTGGGTTAGAC |
| | | AGGCCCCTGGCAAAGGACTTGAGTGGGTTGGAGCCATCTTCACAGGCTCTGG |
| | | GGCTGAGTATAAGGCTGAGTGGGCCAAGGGCAGAGTGACCATCAGCAAGGAC |
| | | ACCAGCAAGAACCAGGTGGTGCTGACCATGACCAACATGGACCCTGTGGACA |
| | | CAGCCACCTACTACTGTGCCTCTGATGCTGGCTATGACTACCCCACACATGC |
| | | CATGCACTATTGGGGCCAGGGCACCCTGGTTACAGTGTCCTCTGGAGGTGGT |
| | | GGAAGTGGTGGTGGTGGAAGTGGAGGTGGTGGTTCTGACATCCAGATGACAC |
| | | AGAGCCCTAGCAGCCTGTCTGCCTCTGTGGGAGACAGAGTGACCATCACCTG |
| | | TAGAGCCAGCCAGGGCATCTCTAGCAGTCTGGCCTGGTATCAGCAGAAGCCT |
| | | GGAAAGGCCCCTAAGCTGCTGATCTATGGGGCCTCTGAGACAGAATCTGGGG |
| | | TGCCCAGCAGATTTTCAGGCTCTGGCTCTGGCACAGACTTCACCCTGACCAT |
| | | TTCTAGCCTGCAGCCTGAGGACTTTGCCACCTACTACTGCCAGAACACCAAA |
| | | GTGGGCAGCAGCTATGGCAACACCTTTGGTGGTGGCACCAAGGTGGAAATCA |
| | | AGAGAtaaTAAagcggccatcaagcttgatctttttccctctgccaaaaatt |
| | | atggggacatcatgaagccccttgagcatctgacttctggctaataaaggaa |
| | | atttattttcattgcaatagtgtgttggaattttttgtgtctctcactcg |
| SCAAV.CAG(Delm). | SEQ ID | ctgcgcgctcgctcgctcactgaggccgcccgggcaaagcccgggcggcctc |
| Crovalimab.ScFv. | NO: 290 | agtgagcgagcgagcgcgcagagagggagtggaattcacgcgtggtaccgac |
| HL (ITR to | | tagttattaatagtaatcaattacggggtcattagttcatagcccatatatg |
| ITR) | | gagttccgcgttacataacttacggtaaatggcccgcctggctgaccgccca |
| | | acgacccccgcccattgacgtcaataatgacgtatgttcccatagtaacgcc |
| | | aatagggactttccattgacgtcaatgggtggagtatttacggtaaactgcc |
| | | cacttggcagtacatcaagtgtatcatatgccaagtacgccccctattgacg |
| | | tcaatgacggtaaatggcccgcctggcattatgcccagtacatgaccttatg |
| | | ggactttcctacttggcagtacatctacgtattagtcatcgctattaccatg |
| | | gtcgaggtgagccccacgttctgcttcactctccccatctcccccccctccc |
| | | cacccccaattttgtatttatttattttttaattattttgtgcagcgatggg |
| | | ggcggggggggggggggggcgcgcgccaggcggggcggggcggggcgagggg |
| | | cggggcggggcgaggcggagaggtgcggcggcagccaatcagagcggcgcgc |
| | | tccgaaagtttccttttatggcgaggcggcggcggcggcggccctataaaaa |
| | | gcgaagcgcgcggcgggcgggagtcgctgcgcgctgccttcgccccgtgccc |
| | | cgctccgccgccgcctcgcgccgcccgccccggctctgactgaccgcgttac |
| | | tcccacaggtgagcgggcgggacggcccttctcctccgggctgtaattagcg |
| | | cttggtttaatgacggcttgtttcttttctgtggctgcgtgaaagccttgag |
| | | gggctccgggagggccctttgtgcggggggagcggctcggggggtgcgtgcg |
| | | tgtgtgtgtgcgtggggagcgccgcgtgcggctccgaatgggcggggagggc |
| | | cttcgtgcgtcgccgcgccgccgtccccttctccctctccagcctcggggct |
| | | gtccgcggggggacggctgccttcgggggggacggggcagggcggggttcgg |
| | | cttctggcgtgtgaccggcggctctagagcctctgctaaccatgttcatgcc |
| | | ttcttctttttcctacagctcctgggcaacgtgctggttattgtgctgtctc |
| | | atcattttggcaaaggaattcGCCACCATGTACAGAATGCAGCTGCTGCTGC |
| | | TCATTGCCCTGTCTCTGGCCCTGGTCACCAATTCTCAGGTGCAGCTGGTTGA |
| | | ATCTGGTGGTGGACTGGTGCAGCCTGGCAGAAGCCTGAGACTGTCTTGTGCT |
| | | GCCTCTGGCTTCACAGTGCACAGCAGCTACTACATGGCCTGGGTTAGACAGG |
| | | CCCCTGGCAAAGGACTTGAGTGGGTTGGAGCCATCTTCACAGGCTCTGGGGC |
| | | TGAGTATAAGGCTGAGTGGGCCAAGGGCAGAGTGACCATCAGCAAGGACACC |
| | | AGCAAGAACCAGGTGGTGCTGACCATGACCAACATGGACCCTGTGGACACAG |
| | | CCACCTACTACTGTGCCTCTGATGCTGGCTATGACTACCCCACACATGCCAT |
| | | GCACTATTGGGGCCAGGGCACCCTGGTTACAGTGTCCTCTGGAGGTGGTGGA |
| | | AGTGGTGGTGGTGGAAGTGGAGGTGGTGGTTCTGACATCCAGATGACACAGA |
| | | GCCCTAGCAGCCTGTCTGCCTCTGTGGGAGACAGAGTGACCATCACCTGTAG |
| | | AGCCAGCCAGGGCATCTCTAGCAGTCTGGCCTGGTATCAGCAGAAGCCTGGA |
| | | AAGGCCCCTAAGCTGCTGATCTATGGGGCCTCTGAGACAGAATCTGGGGTGC |
| | | CCAGCAGATTTTCAGGCTCTGGCTCTGGCACAGACTTCACCCTGACCATTTC |
| | | TAGCCTGCAGCCTGAGGACTTTGCCACCTACTACTGCCAGAACACCAAAGTG |
| | | GGCAGCAGCTATGGCAACACCTTTGGTGGTGGCACCAAGGTGGAAATCAAGA |
| | | GAtaaTAAagcggccatcaagcttgatctttttccctctgccaaaaattatg |
| | | gggacatcatgaagccccttgagcatctgacttctggctaataaaggaaatt |
| | | tattttcattgcaatagtgtgttggaattttttgtgtctctcactcggctag |
| | | cgcatgctggggagagatcgatctgaggaacccctagtgatggagttggcca |
| | | ctccctctctgcgcgctcgctcgctcactgaggccgGGCGACCAAAGGTCGC |
| | | CCGACGCCCGGGCTTTgCCcGGGCGGCCTCAgTGAGcgagcgagcgcgcaga |
| | | gagggagtggcc |
| CAG(Del3). | SEQ ID | gactagttattaatagtaatcaattacggggtcattagttcatagcccatat |
| Crovalimab.ScFv. | NO: 291 | atggagttccgcgttacataacttacggtaaatggcccgcctggctgaccgc |
| HL | | ccaacgacccccgcccattgacgtcaataatgacgtatgttcccatagtaac |
| | | gccaatagggactttccattgacgtcaatgggtggagtatttacggtaaact |
| | | gcccacttggcagtacatcaagtgtatcatatgccaagtacgccccctattg |
| | | acgtcaatgacggtaaatggcccgcctggcattatgcccagtacatgacctt |
| | | atgggactttcctacttggcagtacatctacgtattagtcatcgctattacc |
| | | atggtcgaggtgagccccacgttctgcttcactctccccatctcccccccct |
| | | ccccacccccaattttgtatttatttattttttaattattttgtgcagcgat |
| | | gggggcggggggggggggggggcgcgcgccaggcggggcggggcggggcgag |
| | | gggcggggcggggcgaggcggagaggtgcggcggcagccaatcagagcggcg |
| | | cgctccgaaagtttccttttatggcgaggcggcggcggcggcggccctataa |
| | | aaagcgaagcgcgcggcgggcgggagtcgctgcgcgctgccttcgccccgtg |
| | | ccccgctccgccgccgcctcgcgccgcccgccccggctctgactgaccgcgt |
| | | tactcccacaggtgagcgggcgggacggcccttctcctccgggctgtaatta |
| | | gcgcttggtttaatgacggcttgtttcttttctgtggctgcgtgaaagcctt |
| | | gaggggctccgggagggccctttgtgcggggggagcggctcggggggtgcgt |
| | | gcgtgtgtgtgtgcgtggggagcgccgcgtgcggctccgcgctgcccggcgg |
| | | ctgtgagcgctgcgggcgcggcgcggggctttgtgcgctccgcagtgtgcgc |
| | | gaggggagcgcggccgggggcggtgccccgcggtgcggggggggctgcgagg |
| | | ggaacaaaggctgcgtgcgggctctagagcctctgctaaccatgttcatgcc |
| | | ttcttctttttcctacagctcctgggcaacgtgctggttattgtgctgtctc |
| | | atcattttggcaaaggaattcGCCACCATGTACAGAATGCAGCTGCTGCTGC |
| | | TCATTGCCCTGTCTCTGGCCCTGGTCACCAATTCTCAGGTGCAGCTGGTTGA |
| | | ATCTGGTGGTGGACTGGTGCAGCCTGGCAGAAGCCTGAGACTGTCTTGTGCT |
| | | GCCTCTGGCTTCACAGTGCACAGCAGCTACTACATGGCCTGGGTTAGACAGG |
| | | CCCCTGGCAAAGGACTTGAGTGGGTTGGAGCCATCTTCACAGGCTCTGGGGC |
| | | TGAGTATAAGGCTGAGTGGGCCAAGGGCAGAGTGACCATCAGCAAGGACACC |
| | | AGCAAGAACCAGGTGGTGCTGACCATGACCAACATGGACCCTGTGGACACAG |
| | | CCACCTACTACTGTGCCTCTGATGCTGGCTATGACTACCCCACACATGCCAT |
| | | GCACTATTGGGGCCAGGGCACCCTGGTTACAGTGTCCTCTGGAGGTGGTGGA |
| | | AGTGGTGGTGGTGGAAGTGGAGGTGGTGGTTCTGACATCCAGATGACACAGA |
| | | GCCCTAGCAGCCTGTCTGCCTCTGTGGGAGACAGAGTGACCATCACCTGTAG |
| | | AGCCAGCCAGGGCATCTCTAGCAGTCTGGCCTGGTATCAGCAGAAGCCTGGA |
| | | AAGGCCCCTAAGCTGCTGATCTATGGGGCCTCTGAGACAGAATCTGGGGTGC |
| | | CCAGCAGATTTTCAGGCTCTGGCTCTGGCACAGACTTCACCCTGACCATTTC |
| | | TAGCCTGCAGCCTGAGGACTTTGCCACCTACTACTGCCAGAACACCAAAGTG |
| | | GGCAGCAGCTATGGCAACACCTTTGGTGGTGGCACCAAGGTGGAAATCAAGA |
| | | GAtaaTAAagcggccatcaagcttgatctttttccctctgccaaaaattatg |
| | | gggacatcatgaagccccttgagcatctgacttctggctaataaaggaaatt |
| | | tattttcattgcaatagtgtgttggaattttttgtgtctctcactcg |
| scAAV.CAG(Del3). | SEQ ID | ctgcgcgctcgctcgctcactgaggccgcccgggcaaagcccgggcggcctc |
| Crovalimab.ScFv. | NO: 292 | agtgagcgagcgagcgcgcagagagggagtggaattcacgcgtggtaccgac |
| HL (ITR to ITR) | | tagttattaatagtaatcaattacggggtcattagttcatagcccatatatg |
| | | gagttccgcgttacataacttacggtaaatggcccgcctggctgaccgccca |
| | | acgacccccgcccattgacgtcaataatgacgtatgttcccatagtaacgcc |
| | | aatagggactttccattgacgtcaatgggtggagtatttacggtaaactgcc |
| | | cacttggcagtacatcaagtgtatcatatgccaagtacgccccctattgacg |
| | | tcaatgacggtaaatggcccgcctggcattatgcccagtacatgaccttatg |
| | | ggactttcctacttggcagtacatctacgtattagtcatcgctattaccatg |
| | | gtcgaggtgagccccacgttctgcttcactctccccatctcccccccctccc |
| | | cacccccaattttgtatttatttattttttaattattttgtgcagcgatggg |
| | | ggcggggggggggggggggcgcgcgccaggcggggcggggcggggcgagggg |
| | | cggggcggggcgaggcggagaggtgcggcggcagccaatcagagcggcgcgc |
| | | tccgaaagtttccttttatggcgaggcggcggcggcggcggccctataaaaa |
| | | gcgaagcgcgcggcgggcgggagtcgctgcgcgctgccttcgccccgtgccc |
| | | cgctccgccgccgcctcgcgccgcccgccccggctctgactgaccgcgttac |
| | | tcccacaggtgagcgggcgggacggcccttctcctccgggctgtaattagcg |
| | | cttggtttaatgacggcttgtttcttttctgtggctgcgtgaaagccttgag |
| | | gggctccgggagggccctttgtgcggggggagcggctcggggggtgcgtgcg |
| | | tgtgtgtgtgcgtggggagcgccgcgtgcggctccgcgctgcccggcggctg |
| | | tgagcgctgcgggcgcggcgcggggctttgtgcgctccgcagtgtgcgcgag |
| | | gggagcgcggccgggggcggtgccccgcggtgcggggggggctgcgagggga |
| | | acaaaggctgcgtgcgggctctagagcctctgctaaccatgttcatgccttc |
| | | ttctttttcctacagctcctgggcaacgtgctggttattgtgctgtctcatc |
| | | attttggcaaaggaattcGCCACCATGTACAGAATGCAGCTGCTGCTGCTCA |
| | | TTGCCCTGTCTCTGGCCCTGGTCACCAATTCTCAGGTGCAGCTGGTTGAATC |
| | | TGGTGGTGGACTGGTGCAGCCTGGCAGAAGCCTGAGACTGTCTTGTGCTGCC |
| | | TCTGGCTTCACAGTGCACAGCAGCTACTACATGGCCTGGGTTAGACAGGCCC |
| | | CTGGCAAAGGACTTGAGTGGGTTGGAGCCATCTTCACAGGCTCTGGGGCTGA |
| | | GTATAAGGCTGAGTGGGCCAAGGGCAGAGTGACCATCAGCAAGGACACCAGC |
| | | AAGAACCAGGTGGTGCTGACCATGACCAACATGGACCCTGTGGACACAGCCA |
| | | CCTACTACTGTGCCTCTGATGCTGGCTATGACTACCCCACACATGCCATGCA |
| | | CTATTGGGGCCAGGGCACCCTGGTTACAGTGTCCTCTGGAGGTGGTGGAAGT |
| | | GGTGGTGGTGGAAGTGGAGGTGGTGGTTCTGACATCCAGATGACACAGAGCC |
| | | CTAGCAGCCTGTCTGCCTCTGTGGGAGACAGAGTGACCATCACCTGTAGAGC |
| | | CAGCCAGGGCATCTCTAGCAGTCTGGCCTGGTATCAGCAGAAGCCTGGAAAG |
| | | GCCCCTAAGCTGCTGATCTATGGGGCCTCTGAGACAGAATCTGGGGTGCCCA |
| | | GCAGATTTTCAGGCTCTGGCTCTGGCACAGACTTCACCCTGACCATTTCTAG |
| | | CCTGCAGCCTGAGGACTTTGCCACCTACTACTGCCAGAACACCAAAGTGGGC |
| | | AGCAGCTATGGCAACACCTTTGGTGGTGGCACCAAGGTGGAAATCAAGAGAt |
| | | aaTAAagcggccatcaagcttgatctttttccctctgccaaaaattatgggg |
| | | acatcatgaagccccttgagcatctgacttctggctaataaaggaaatttat |
| | | tttcattgcaatagtgtgttggaattttttgtgtctctcactcggctagcgc |
| | | atgctggggagagatcgatctgaggaacccctagtgatggagttggccactc |
| | | cctctctgcgcgctcgctcgctcactgaggccgGGCGACCAAAGGTCGCCCG |
| | | ACGCCCGGGCTTTgCCcGGGCGGCCTCAgTGAGcgagcgagcgcgcagagag |
| | | ggagtggcc |
| BB5.1.scFv.HL | SEQ ID | ATGTACAGAATGCAGCTGCTGCTGCTCATTGCCCTGTCTCTGGCCCTGGTCA |
| (coding | NO: 293 | CCAATTCT |
| sequence- signal | | CAGGTTCAGCTGCAGCAACCTGGGGCTGAGCTTGTCAGACCTGGCACATCTG |
| sequence | | TGAAGCTGAGCTGCAAGGCCTCTGGCTACACCTTCACAAGCAGCTGGATGCA |
| underlined) | | CTGGGTCAAGCAGAGGCCTGGACAGGGCCTTGAGTGGATTGGAGTGATTGAC |
| | | CCCTCTGACAGCTACACCAACTACAACCAGAAGTTCAAGGGCAAAGCCACAC |
| | | TGACAGTGGACACCAGCAGCAGCACAGCCTACATGCAGCTGAGCAGCCTGAC |
| | | CTCTGAGGACTCTGCTGTGTACTACTGTGCCAGAGGTGGTGGCAGCAGCTAC |
| | | AACAGATACTTTGATGTGTGGGGCACAGGCACCACAGTGACTGTGTCCTCTG |
| | | GAGGTGGTGGAAGTGGTGGTGGTGGAAGTGGAGGTGGTGGTTCTAATATCAT |
| | | GATGACACAGAGCCCCAGCAGCCTGGCTGTGTCTGCTGGGGAGAAAGTGACC |
| | | ATGAGCTGCAAGAGCAGCCAGTCTGTGCTGTACTCCAGCAACCAGAAGAACT |
| | | ACCTGGCCTGGTATCAGCAGAAGCCTGGCCAGTCTCCTAAGCTGCTGATCTA |
| | | CTGGGCCAGCACCAGAGAATCTGGGGTGCCAGACAGATTCACAGGCTCTGGC |
| | | TCTGGCACAGACTTCACCCTGACAATCAGCTCTGTGCAGGCTGAGGACCTGG |
| | | CAGTGTACTACTGTCACCAGTACCTGAGCAGCAGAACCTTTGGTGGTGGCAC |
| | | CAAGCTGGAAATCAAGAGGTAA |
| CAG.BB5.1.scFv. | SEQ ID | gacattgattattgactagttattaatagtaatcaattacggggtcattagt |
| HL (Promoter to | NO: 294 | tcatagcccatatatggagttccgcgttacataacttacggtaaatggcccg |
| polyA) | | cctggctgaccgcccaacgacccccgcccattgacgtcaataatgacgtatg |
| | | ttcccatagtaacgccaatagggactttccattgacgtcaatgggtggagta |
| | | tttacggtaaactgcccacttggcagtacatcaagtgtatcatatgccaagt |
| | | acgccccctattgacgtcaatgacggtaaatggcccgcctggcattatgccc |
| | | agtacatgaccttatgggactttcctacttggcagtacatctacgtattagt |
| | | catcgctattaccatggtcgaggtgagccccacgttctgcttcactctcccc |
| | | atctcccccccctccccacccccaattttgtatttatttattttttaattat |
| | | tttgtgcagcgatgggggcggggggggggggggggcgcgcgccaggcggggc |
| | | ggggcggggcgaggggcggggcggggcgaggcggagaggtgcggcggcagcc |
| | | aatcagagcggcgcgctccgaaagtttccttttatggcgaggcggcggcggc |
| | | ggcggccctataaaaagcgaagcgcgcggcgggcgggagtcgctgcgcgctg |
| | | ccttcgccccgtgccccgctccgccgccgcctcgcgccgcccgccccggctc |
| | | tgactgaccgcgttactcccacaggtgagcgggcgggacggcccttctcctc |
| | | cgggctgtaattagcgcttggtttaatgacggcttgtttcttttctgtggct |
| | | gcgtgaaagccttgaggggctccgggagggccctttgtgcggggggagcggc |
| | | tcggggggtgcgtgcgtgtgtgtgtgcgtggggagcgccgcgtgcggctccg |
| | | cgctgcccggcggctgtgagcgctgcgggcgcggcgcggggctttgtgcgct |
| | | ccgcagtgtgcgcgaggggagcgcggccgggggcggtgccccgcggtgcggg |
| | | gggggctgcgaggggaacaaaggctgcgtgcggggtgtgtgcgtgggggggt |
| | | gagcagggggtgtgggcgcgtcggtcgggctgcaaccccccctgcacccccc |
| | | tccccgagttgctgagcacggcccggcttcgggtgcggggctccgtacgggg |
| | | cgtggcgcggggctcgccgtgccgggcggggggtggcggcaggtgggggtgc |
| | | cgggcggggcggggccgcctcgggccggggagggctcgggggaggggcgcgg |
| | | cggcccccggagcgccggcggctgtcgaggcgcggcgagccgcagccattgc |
| | | cttttatggtaatcgtgcgagagggcgcagggacttcctttgtcccaaatct |
| | | gtgcggagccgaaatctgggaggcgccgccgcaccccctctagcgggcgcgg |
| | | ggcgaagcggtgcggcgccggcaggaaggaaatgggcggggagggccttcgt |
| | | gcgtcgccgcgccgccgtccccttctccctctccagcctcggggctgtccgc |
| | | ggggggacggctgccttcgggggggacggggcagggcggggttcggcttctg |
| | | gcgtgtgaccggcggctctagagcctctgctaaccatgttcatgccttcttc |
| | | tttttcctacagctcctgggcaacgtgctggttattgtgctgtctcatcatt |
| | | ttggcaaagAATTCGCCACCATGTACAGAATGCAGCTGCTGCTGCTCATTGC |
| | | CCTGTCTCTGGCCCTGGTCACCAATTCTCAGGTTCAGCTGCAGCAACCTGGG |
| | | GCTGAGCTTGTCAGACCTGGCACATCTGTGAAGCTGAGCTGCAAGGCCTCTG |
| | | GCTACACCTTCACAAGCAGCTGGATGCACTGGGTCAAGCAGAGGCCTGGACA |
| | | GGGCCTTGAGTGGATTGGAGTGATTGACCCCTCTGACAGCTACACCAACTAC |
| | | AACCAGAAGTTCAAGGGCAAAGCCACACTGACAGTGGACACCAGCAGCAGCA |
| | | CAGCCTACATGCAGCTGAGCAGCCTGACCTCTGAGGACTCTGCTGTGTACTA |
| | | CTGTGCCAGAGGTGGTGGCAGCAGCTACAACAGATACTTTGATGTGTGGGGC |
| | | ACAGGCACCACAGTGACTGTGTCCTCTGGAGGTGGTGGAAGTGGTGGTGGTG |
| | | GAAGTGGAGGTGGTGGTTCTAATATCATGATGACACAGAGCCCCAGCAGCCT |
| | | GGCTGTGTCTGCTGGGGAGAAAGTGACCATGAGCTGCAAGAGCAGCCAGTCT |
| | | GTGCTGTACTCCAGCAACCAGAAGAACTACCTGGCCTGGTATCAGCAGAAGC |
| | | CTGGCCAGTCTCCTAAGCTGCTGATCTACTGGGCCAGCACCAGAGAATCTGG |
| | | GGTGCCAGACAGATTCACAGGCTCTGGCTCTGGCACAGACTTCACCCTGACA |
| | | ATCAGCTCTGTGCAGGCTGAGGACCTGGCAGTGTACTACTGTCACCAGTACC |
| | | TGAGCAGCAGAACCTTTGGTGGTGGCACCAAGCTGGAAATCAAGAGGtaaTA |
| | | ACtcgaggacggggtgaactacgcctgaggatccgatctttttccctctgcc |
| | | aaaaattatggggacatcatgaagccccttgagcatctgacttctggctaat |
| | | aaaggaaatttattttcattgcaatagtgtgttggaattttttgtgtctctc |
| | | a |
| ss.CAG.BB5.1.scFv. | SEQ ID | ctgcgcgctcgctcgctcactgaggccgcccgggcaaagcccgggcgtcggg |
| HL (ITR to | NO: 295 | cgacctttggtcgcccggcctcagtgagcgagcgagcgcgcagagagggagt |
| ITR) | | ggccaactccatcactaggggttcctCATATGcagggtaatggggatcctct |
| | | agataggtaccctagtcgacattgattattgactagttattaatagtaatca |
| | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTCAGGTTC |
| | | AGCTGCAGCAACCTGGGGCTGAGCTTGTCAGACCTGGCACATCTGTGAAGCT |
| | | GAGCTGCAAGGCCTCTGGCTACACCTTCACAAGCAGCTGGATGCACTGGGTC |
| | | AAGCAGAGGCCTGGACAGGGCCTTGAGTGGATTGGAGTGATTGACCCCTCTG |
| | | ACAGCTACACCAACTACAACCAGAAGTTCAAGGGCAAAGCCACACTGACAGT |
| | | GGACACCAGCAGCAGCACAGCCTACATGCAGCTGAGCAGCCTGACCTCTGAG |
| | | GACTCTGCTGTGTACTACTGTGCCAGAGGTGGTGGCAGCAGCTACAACAGAT |
| | | ACTTTGATGTGTGGGGCACAGGCACCACAGTGACTGTGTCCTCTGGAGGTGG |
| | | TGGAAGTGGTGGTGGTGGAAGTGGAGGTGGTGGTTCTAATATCATGATGACA |
| | | CAGAGCCCCAGCAGCCTGGCTGTGTCTGCTGGGGAGAAAGTGACCATGAGCT |
| | | GCAAGAGCAGCCAGTCTGTGCTGTACTCCAGCAACCAGAAGAACTACCTGGC |
| | | CTGGTATCAGCAGAAGCCTGGCCAGTCTCCTAAGCTGCTGATCTACTGGGCC |
| | | AGCACCAGAGAATCTGGGGTGCCAGACAGATTCACAGGCTCTGGCTCTGGCA |
| | | CAGACTTCACCCTGACAATCAGCTCTGTGCAGGCTGAGGACCTGGCAGTGTA |
| | | CTACTGTCACCAGTACCTGAGCAGCAGAACCTTTGGTGGTGGCACCAAGCTG |
| | | GAAATCAAGAGGtaaTAACtcgaggacggggtgaactacgcctgaggatccg |
| | | atctttttccctctgccaaaaattatggggacatcatgaagccccttgagca |
| | | tctgacttctggctaataaaggaaatttattttcattgcaatagtgtgttgg |
| | | aattttttgtgtctctcactcggaagcaattcgttgatctgaatttcgacca |
| | | cccataatacccattaccctgCCAGGGTAATGGGCTAGCTGCGGCCGCagga |
| | | acccctagtgatggagttggccactccctctctgcgcgctcgctcgctcact |
| | | gaggccgggcgaccaaaggtcgcccgacgcccgggctttgcccgggcggcct |
| | | cagtgagcgagcgagcgcgcag |
| CAG.BB5.1.scFv. | SEQ ID | gacattgattattgactagttattaatagtaatcaattacggggtcattagt |
| LH (promoter to | NO: 296 | tcatagcccatatatggagttccgcgttacataacttacggtaaatggcccg |
| polyA) | | cctggctgaccgcccaacgacccccgcccattgacgtcaataatgacgtatg |
| | | ttcccatagtaacgccaatagggactttccattgacgtcaatgggtggagta |
| | | tttacggtaaactgcccacttggcagtacatcaagtgtatcatatgccaagt |
| | | acgccccctattgacgtcaatgacggtaaatggcccgcctggcattatgccc |
| | | agtacatgaccttatgggactttcctacttggcagtacatctacgtattagt |
| | | catcgctattaccatggtcgaggtgagccccacgttctgcttcactctcccc |
| | | atctcccccccctccccacccccaattttgtatttatttattttttaattat |
| | | tttgtgcagcgatgggggcggggggggggggggggcgcgcgccaggcggggc |
| | | ggggcggggcgaggggcggggcggggcgaggcggagaggtgcggcggcagcc |
| | | aatcagagcggcgcgctccgaaagtttccttttatggcgaggcggcggcggc |
| | | ggcggccctataaaaagcgaagcgcgcggcgggcgggagtcgctgcgcgctg |
| | | ccttcgccccgtgccccgctccgccgccgcctcgcgccgcccgccccggctc |
| | | tgactgaccgcgttactcccacaggtgagcgggcgggacggcccttctcctc |
| | | cgggctgtaattagcgcttggtttaatgacggcttgtttcttttctgtggct |
| | | gcgtgaaagccttgaggggctccgggagggccctttgtgcggggggagcggc |
| | | tcggggggtgcgtgcgtgtgtgtgtgcgtggggagcgccgcgtgcggctccg |
| | | cgctgcccggcggctgtgagcgctgcgggcgcggcgcggggctttgtgcgct |
| | | ccgcagtgtgcgcgaggggagcgcggccgggggcggtgccccgcggtgcggg |
| | | gggggctgcgaggggaacaaaggctgcgtgcggggtgtgtgcgtgggggggt |
| | | gagcagggggtgtgggcgcgtcggtcgggctgcaaccccccctgcacccccc |
| | | tccccgagttgctgagcacggcccggcttcgggtgcggggctccgtacgggg |
| | | cgtggcgcggggctcgccgtgccgggcggggggtggcggcaggtgggggtgc |
| | | cgggcggggcggggccgcctcgggccggggagggctcgggggaggggcgcgg |
| | | cggcccccggagcgccggcggctgtcgaggcgcggcgagccgcagccattgc |
| | | cttttatggtaatcgtgcgagagggcgcagggacttcctttgtcccaaatct |
| | | gtgcggagccgaaatctgggaggcgccgccgcaccccctctagcgggcgcgg |
| | | ggcgaagcggtgcggcgccggcaggaaggaaatgggcggggagggccttcgt |
| | | gcgtcgccgcgccgccgtccccttctccctctccagcctcggggctgtccgc |
| | | ggggggacggctgccttcgggggggacggggcagggcggggttcggcttctg |
| | | gcgtgtgaccggcggctctagagcctctgctaaccatgttcatgccttcttc |
| | | tttttcctacagctcctgggcaacgtgctggttattgtgctgtctcatcatt |
| | | ttggcaaagAATTCGCCACCATGTACAGAATGCAGCTGCTGCTGCTCATTGC |
| | | CCTGTCTCTGGCCCTGGTCACCAATTCTAATATCATGATGACACAGAGCCCC |
| | | AGCAGCCTGGCTGTGTCTGCTGGGGAGAAAGTGACCATGAGCTGCAAGAGCA |
| | | GCCAGTCTGTGCTGTACTCCAGCAACCAGAAGAACTACCTGGCCTGGTATCA |
| | | GCAGAAGCCTGGCCAGTCTCCTAAGCTGCTGATCTACTGGGCCAGCACCAGA |
| | | GAATCTGGGGTGCCAGACAGATTCACAGGCTCTGGCTCTGGCACAGACTTCA |
| | | CCCTGACAATCAGCTCTGTGCAGGCTGAGGACCTGGCAGTGTACTACTGTCA |
| | | CCAGTACCTGAGCAGCAGAACCTTTGGTGGTGGCACCAAGCTGGAAATCAAG |
| | | AGGGGAGGTGGTGGAAGTGGTGGTGGTGGAAGTGGAGGTGGTGGTTCTCAGG |
| | | TTCAGCTGCAGCAACCTGGGGCTGAGCTTGTCAGACCTGGCACATCTGTGAA |
| | | GCTGAGCTGCAAGGCCTCTGGCTACACCTTCACAAGCAGCTGGATGCACTGG |
| | | GTCAAGCAGAGGCCTGGACAGGGCCTTGAGTGGATTGGAGTGATTGACCCCT |
| | | CTGACAGCTACACCAACTACAACCAGAAGTTCAAGGGCAAAGCCACACTGAC |
| | | AGTGGACACCAGCAGCAGCACAGCCTACATGCAGCTGAGCAGCCTGACCTCT |
| | | GAGGACTCTGCTGTGTACTACTGTGCCAGAGGTGGTGGCAGCAGCTACAACA |
| | | GATACTTTGATGTGTGGGGCACAGGCACCACAGTGACTGTGTCCTCTtaaTA |
| | | ACtcgaggacggggtgaactacgcctgaggatccgatctttttccctctgcc |
| | | aaaaattatggggacatcatgaagccccttgagcatctgacttctggctaat |
| | | aaaggaaatttattttcattgcaatagtgtgttggaattttttgtgtctctc |
| | | a |
| ss.CAG.BB5.1.scFv. | SEQ ID | ctgcgcgctcgctcgctcactgaggccgcccgggcaaagcccgggcgtcggg |
| LH (ITR to | NO: 297 | cgacctttggtcgcccggcctcagtgagcgagcgagcgcgcagagagggagt |
| ITR) | | ggccaactccatcactaggggttcctCATATGcagggtaatggggatcctct |
| | | agataggtaccctagtcgacattgattattgactagttattaatagtaatca |
| | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcgggggcgggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTAATATCA |
| | | TGATGACACAGAGCCCCAGCAGCCTGGCTGTGTCTGCTGGGGAGAAAGTGAC |
| | | CATGAGCTGCAAGAGCAGCCAGTCTGTGCTGTACTCCAGCAACCAGAAGAAC |
| | | TACCTGGCCTGGTATCAGCAGAAGCCTGGCCAGTCTCCTAAGCTGCTGATCT |
| | | ACTGGGCCAGCACCAGAGAATCTGGGGTGCCAGACAGATTCACAGGCTCTGG |
| | | CTCTGGCACAGACTTCACCCTGACAATCAGCTCTGTGCAGGCTGAGGACCTG |
| | | GCAGTGTACTACTGTCACCAGTACCTGAGCAGCAGAACCTTTGGTGGTGGCA |
| | | CCAAGCTGGAAATCAAGAGGGGAGGTGGTGGAAGTGGTGGTGGTGGAAGTGG |
| | | AGGTGGTGGTTCTCAGGTTCAGCTGCAGCAACCTGGGGCTGAGCTTGTCAGA |
| | | CCTGGCACATCTGTGAAGCTGAGCTGCAAGGCCTCTGGCTACACCTTCACAA |
| | | GCAGCTGGATGCACTGGGTCAAGCAGAGGCCTGGACAGGGCCTTGAGTGGAT |
| | | TGGAGTGATTGACCCCTCTGACAGCTACACCAACTACAACCAGAAGTTCAAG |
| | | GGCAAAGCCACACTGACAGTGGACACCAGCAGCAGCACAGCCTACATGCAGC |
| | | TGAGCAGCCTGACCTCTGAGGACTCTGCTGTGTACTACTGTGCCAGAGGTGG |
| | | TGGCAGCAGCTACAACAGATACTTTGATGTGTGGGGCACAGGCACCACAGTG |
| | | ACTGTGTCCTCTtaaTAACtcgaggacggggtgaactacgcctgaggatccg |
| | | atctttttccctctgccaaaaattatggggacatcatgaagccccttgagca |
| | | tctgacttctggctaataaaggaaatttattttcattgcaatagtgtgttgg |
| | | aattttttgtgtctctcactcggaagcaattcgttgatctgaatttcgacca |
| | | cccataatacccattaccctgCCAGGGTAATGGGCTAGCTGCGGCCGCagga |
| | | acccctagtgatggagttggccactccctctctgcgcgctcgctcgctcact |
| | | gaggccgggcgaccaaaggtcgcccgacgcccgggctttgcccgggcggcct |
| | | cagtgagcgagcgagcgcgcag |
| BB5.1.scFv.LH | SEQ ID | ATGTACAGAATGCAGCTGCTGCTGCTCATTGCCCTGTCTCTGGCCCTGGTCA |
| (coding | NO: 298 | CCAATTCT |
| sequence- signal | | AATATCATGATGACACAGAGCCCCAGCAGCCTGGCTGTGTCTGCTGGGGAGA |
| peptide (leader) | | AAGTGACCATGAGCTGCAAGAGCAGCCAGTCTGTGCTGTACTCCAGCAACCA |
| sequence | | GAAGAACTACCTGGCCTGGTATCAGCAGAAGCCTGGCCAGTCTCCTAAGCTG |
| underlined) | | CTGATCTACTGGGCCAGCACCAGAGAATCTGGGGTGCCAGACAGATTCACAG |
| | | GCTCTGGCTCTGGCACAGACTTCACCCTGACAATCAGCTCTGTGCAGGCTGA |
| | | GGACCTGGCAGTGTACTACTGTCACCAGTACCTGAGCAGCAGAACCTTTGGT |
| | | GGTGGCACCAAGCTGGAAATCAAGAGGGGAGGTGGTGGAAGTGGTGGTGGTG |
| | | GAAGTGGAGGTGGTGGTTCTCAGGTTCAGCTGCAGCAACCTGGGGCTGAGCT |
| | | TGTCAGACCTGGCACATCTGTGAAGCTGAGCTGCAAGGCCTCTGGCTACACC |
| | | TTCACAAGCAGCTGGATGCACTGGGTCAAGCAGAGGCCTGGACAGGGCCTTG |
| | | AGTGGATTGGAGTGATTGACCCCTCTGACAGCTACACCAACTACAACCAGAA |
| | | GTTCAAGGGCAAAGCCACACTGACAGTGGACACCAGCAGCAGCACAGCCTAC |
| | | ATGCAGCTGAGCAGCCTGACCTCTGAGGACTCTGCTGTGTACTACTGTGCCA |
| | | GAGGTGGTGGCAGCAGCTACAACAGATACTTTGATGTGTGGGGCACAGGCAC |
| | | CACAGTGACTGTGTCCTCTTAA |
| CB.sv40.BB5.1. | SEQ ID | acgcgtggtacctctggtcgttacataacttacggtaaatggcccgcctggc |
| scFv.LH | NO: 299 | tgaccgcccaacgacccccgcccattgacgtcaataatgacgtatgttccca |
| (Promoter to | | tagtaacgccaatagggactttccattgacgtcaatgggtggagtatttacg |
| polyA) | | gtaaactgcccacttggcagtacatcaagtgtatcatatgccaagtacgccc |
| | | cctattgacgtcaatgacggtaaatggcccgcctggcattatgcccagtaca |
| | | tgaccttatgggactttcctacttggcagtacatctactcgaggccacgttc |
| | | tgcttcactctccccatctcccccccctccccacccccaattttgtatttat |
| | | ttattttttaattattttgtgcagcgatgggggcgggggggggggggggggg |
| | | ggcgcgcgccaggcggggcggggcggggcgaggggcggggcggggcgaggcg |
| | | gagaggtgcggcggcagccaatcagagcggcgcgctccgaaagtttcctttt |
| | | atggcgaggcggcggcggcggcggccctataaaaagcgaagcgcgcggcggg |
| | | cgggagcgggatcagccaccgcggtggcggcctagagtcgacgaggaactga |
| | | aaaaccagaaagttaactggtaagtttagtctttttgtcttttatttcaggt |
| | | cccggatccggtggtggtgcaaatcaaagaactgctcctcagtggatgttgc |
| | | ctttacttctaggcctgtacggaagtgttacttctgctctaaaagctgcgga |
| | | attgtacccgcggccgatccaccggtcGCCACCATGTACAGAATGCAGCTGC |
| | | TGCTGCTCATTGCCCTGTCTCTGGCCCTGGTCACCAATTCTAATATCATGAT |
| | | GACACAGAGCCCCAGCAGCCTGGCTGTGTCTGCTGGGGAGAAAGTGACCATG |
| | | AGCTGCAAGAGCAGCCAGTCTGTGCTGTACTCCAGCAACCAGAAGAACTACC |
| | | TGGCCTGGTATCAGCAGAAGCCTGGCCAGTCTCCTAAGCTGCTGATCTACTG |
| | | GGCCAGCACCAGAGAATCTGGGGTGCCAGACAGATTCACAGGCTCTGGCTCT |
| | | GGCACAGACTTCACCCTGACAATCAGCTCTGTGCAGGCTGAGGACCTGGCAG |
| | | TGTACTACTGTCACCAGTACCTGAGCAGCAGAACCTTTGGTGGTGGCACCAA |
| | | GCTGGAAATCAAGAGGGGAGGTGGTGGAAGTGGTGGTGGTGGAAGTGGAGGT |
| | | GGTGGTTCTCAGGTTCAGCTGCAGCAACCTGGGGCTGAGCTTGTCAGACCTG |
| | | GCACATCTGTGAAGCTGAGCTGCAAGGCCTCTGGCTACACCTTCACAAGCAG |
| | | CTGGATGCACTGGGTCAAGCAGAGGCCTGGACAGGGCCTTGAGTGGATTGGA |
| | | GTGATTGACCCCTCTGACAGCTACACCAACTACAACCAGAAGTTCAAGGGCA |
| | | AAGCCACACTGACAGTGGACACCAGCAGCAGCACAGCCTACATGCAGCTGAG |
| | | CAGCCTGACCTCTGAGGACTCTGCTGTGTACTACTGTGCCAGAGGTGGTGGC |
| | | AGCAGCTACAACAGATACTTTGATGTGTGGGGCACAGGCACCACAGTGACTG |
| | | TGTCCTCTtaaTAAagcggccatcaagcttgatctttttccctctgccaaaa |
| | | attatggggacatcatgaagccccttgagcatctgacttctggctaataaag |
| | | gaaatttattttcattgcaatagtgtgttggaattttttgtgtctctcactc |
| | | ggctagcgaagcaattctagcaggcatgctggggag |
| SCAAV.CB.sv40. | SEQ ID | ctgcgcgctcgctcgctcactgaggccgcccgggcaaagcccgggcggcctc |
| BB5.1.scFv.LH | NO: 300 | agtgagcgagcgagcgcgcagagagggagtggaattcacgcgtggtacctct |
| (ITR to ITR) | | ggtcgttacataacttacggtaaatggcccgcctggctgaccgcccaacgac |
| | | ccccgcccattgacgtcaataatgacgtatgttcccatagtaacgccaatag |
| | | ggactttccattgacgtcaatgggtggagtatttacggtaaactgcccactt |
| | | ggcagtacatcaagtgtatcatatgccaagtacgccccctattgacgtcaat |
| | | gacggtaaatggcccgcctggcattatgcccagtacatgaccttatgggact |
| | | ttcctacttggcagtacatctactcgaggccacgttctgcttcactctcccc |
| | | atctcccccccctccccacccccaattttgtatttatttattttttaattat |
| | | tttgtgcagcgatgggggcggggggggggggggggggggcgcgcgccaggcg |
| | | gggcggggcggggcgaggggcggggcggggcgaggcggagaggtgcggcggc |
| | | agccaatcagagcggcgcgctccgaaagtttccttttatggcgaggcggcgg |
| | | cggcggcggccctataaaaagcgaagcgcgcggcgggcgggagcgggatcag |
| | | ccaccgcggtggcggcctagagtcgacgaggaactgaaaaaccagaaagtta |
| | | actggtaagtttagtctttttgtcttttatttcaggtcccggatccggtggt |
| | | ggtgcaaatcaaagaactgctcctcagtggatgttgcctttacttctaggcc |
| | | tgtacggaagtgttacttctgctctaaaagctgcggaattgtacccgcggcc |
| | | gatccaccggtcGCCACCATGTACAGAATGCAGCTGCTGCTGCTCATTGCCC |
| | | TGTCTCTGGCCCTGGTCACCAATTCTAATATCATGATGACACAGAGCCCCAG |
| | | CAGCCTGGCTGTGTCTGCTGGGGAGAAAGTGACCATGAGCTGCAAGAGCAGC |
| | | CAGTCTGTGCTGTACTCCAGCAACCAGAAGAACTACCTGGCCTGGTATCAGC |
| | | AGAAGCCTGGCCAGTCTCCTAAGCTGCTGATCTACTGGGCCAGCACCAGAGA |
| | | ATCTGGGGTGCCAGACAGATTCACAGGCTCTGGCTCTGGCACAGACTTCACC |
| | | CTGACAATCAGCTCTGTGCAGGCTGAGGACCTGGCAGTGTACTACTGTCACC |
| | | AGTACCTGAGCAGCAGAACCTTTGGTGGTGGCACCAAGCTGGAAATCAAGAG |
| | | GGGAGGTGGTGGAAGTGGTGGTGGTGGAAGTGGAGGTGGTGGTTCTCAGGTT |
| | | CAGCTGCAGCAACCTGGGGCTGAGCTTGTCAGACCTGGCACATCTGTGAAGC |
| | | TGAGCTGCAAGGCCTCTGGCTACACCTTCACAAGCAGCTGGATGCACTGGGT |
| | | CAAGCAGAGGCCTGGACAGGGCCTTGAGTGGATTGGAGTGATTGACCCCTCT |
| | | GACAGCTACACCAACTACAACCAGAAGTTCAAGGGCAAAGCCACACTGACAG |
| | | TGGACACCAGCAGCAGCACAGCCTACATGCAGCTGAGCAGCCTGACCTCTGA |
| | | GGACTCTGCTGTGTACTACTGTGCCAGAGGTGGTGGCAGCAGCTACAACAGA |
| | | TACTTTGATGTGTGGGGCACAGGCACCACAGTGACTGTGTCCTCTtaaTAAa |
| | | gcggccatcaagcttgatctttttccctctgccaaaaattatggggacatca |
| | | tgaagccccttgagcatctgacttctggctaataaaggaaatttattttcat |
| | | tgcaatagtgtgttggaattttttgtgtctctcactcggctagcgaagcaat |
| | | tctagcaggcatgctggggagagatcgatctgaggaacccctagtgatggag |
| | | ttggccactccctctctgcgcgctcgctcgctcactgaggccgGGCGACCAA |
| | | AGGTCGCCCGACGCCCGGGCTTTgCCCGGGCGGCCTCAgTGAGcgagcgagc |
| | | gcgcagagagggagtggcc |
| BB5.1.scFv.LH- | SEQ ID | AATATCATGATGACACAGAGCCCCAGCAGCCTGGCTGTGTCTGCTGGGGAGA |
| FLAG-Tag | NO: 301 | AAGTGACCATGAGCTGCAAGAGCAGCCAGTCTGTGCTGTACTCCAGCAACCA |
| (coding | | GAAGAACTACCTGGCCTGGTATCAGCAGAAGCCTGGCCAGTCTCCTAAGCTG |
| sequence) | | CTGATCTACTGGGCCAGCACCAGAGAATCTGGGGTGCCAGACAGATTCACAG |
| | | GCTCTGGCTCTGGCACAGACTTCACCCTGACAATCAGCTCTGTGCAGGCTGA |
| | | GGACCTGGCAGTGTACTACTGTCACCAGTACCTGAGCAGCAGAACCTTTGGT |
| | | GGTGGCACCAAGCTGGAAATCAAGAGGGGAGGTGGTGGAAGTGGTGGTGGTG |
| | | GAAGTGGAGGTGGTGGTTCTCAGGTTCAGCTGCAGCAACCTGGGGCTGAGCT |
| | | TGTCAGACCTGGCACATCTGTGAAGCTGAGCTGCAAGGCCTCTGGCTACACC |
| | | TTCACAAGCAGCTGGATGCACTGGGTCAAGCAGAGGCCTGGACAGGGCCTTG |
| | | AGTGGATTGGAGTGATTGACCCCTCTGACAGCTACACCAACTACAACCAGAA |
| | | GTTCAAGGGCAAAGCCACACTGACAGTGGACACCAGCAGCAGCACAGCCTAC |
| | | ATGCAGCTGAGCAGCCTGACCTCTGAGGACTCTGCTGTGTACTACTGTGCCA |
| | | GAGGTGGTGGCAGCAGCTACAACAGATACTTTGATGTGTGGGGCACAGGCAC |
| | | CACAGTGACTGTGTCCTCT |
| CAG.BB5.1.scFv. | SEQ ID | gacattgattattgactagttattaatagtaatcaattacggggtcattagt |
| LH-FLAG-Tag | NO: 302 | tcatagcccatatatggagttccgcgttacataacttacggtaaatggcccg |
| (Promoter to | | cctggctgaccgcccaacgacccccgcccattgacgtcaataatgacgtatg |
| polyA) | | ttcccatagtaacgccaatagggactttccattgacgtcaatgggtggagta |
| | | tttacggtaaactgcccacttggcagtacatcaagtgtatcatatgccaagt |
| | | acgccccctattgacgtcaatgacggtaaatggcccgcctggcattatgccc |
| | | agtacatgaccttatgggactttcctacttggcagtacatctacgtattagt |
| | | catcgctattaccatggtcgaggtgagccccacgttctgcttcactctcccc |
| | | atctcccccccctccccacccccaattttgtatttatttattttttaattat |
| | | tttgtgcagcgatgggggcggggggggggggggggcgcgcgccaggcggggc |
| | | ggggcggggcgaggggcggggcggggcgaggcggagaggtgcggcggcagcc |
| | | aatcagagcggcgcgctccgaaagtttccttttatggcgaggcggcggcggc |
| | | ggcggccctataaaaagcgaagcgcgcggcgggcgggagtcgctgcgcgctg |
| | | ccttcgccccgtgccccgctccgccgccgcctcgcgccgcccgccccggctc |
| | | tgactgaccgcgttactcccacaggtgagcgggcgggacggcccttctcctc |
| | | cgggctgtaattagcgcttggtttaatgacggcttgtttcttttctgtggct |
| | | gcgtgaaagccttgaggggctccgggagggccctttgtgcggggggagcggc |
| | | tcggggggtgcgtgcgtgtgtgtgtgcgtggggagcgccgcgtgcggctccg |
| | | cgctgcccggcggctgtgagcgctgcgggcgcggcgcggggctttgtgcgct |
| | | ccgcagtgtgcgcgaggggagcgcggccgggggcggtgccccgcggtgcggg |
| | | gggggctgcgaggggaacaaaggctgcgtgcggggtgtgtgcgtgggggggt |
| | | gagcagggggtgtgggcgcgtcggtcgggctgcaaccccccctgcacccccc |
| | | tccccgagttgctgagcacggcccggcttcgggtgcggggctccgtacgggg |
| | | cgtggcgcggggctcgccgtgccgggcggggggtggcggcaggtgggggtgc |
| | | cgggcggggcggggccgcctcgggccggggagggctcgggggaggggcgcgg |
| | | cggcccccggagcgccggcggctgtcgaggcgcggcgagccgcagccattgc |
| | | cttttatggtaatcgtgcgagagggcgcagggacttcctttgtcccaaatct |
| | | gtgcggagccgaaatctgggaggcgccgccgcaccccctctagcgggcgcgg |
| | | ggcgaagcggtgcggcgccggcaggaaggaaatgggcggggagggccttcgt |
| | | gcgtcgccgcgccgccgtccccttctccctctccagcctcggggctgtccgc |
| | | ggggggacggctgccttcgggggggacggggcagggcggggttcggcttctg |
| | | gcgtgtgaccggcggctctagagcctctgctaaccatgttcatgccttcttc |
| | | tttttcctacagctcctgggcaacgtgctggttattgtgctgtctcatcatt |
| | | ttggcaaagAATTCGCCACCATGTACAGAATGCAGCTGCTGCTGCTCATTGC |
| | | CCTGTCTCTGGCCCTGGTCACCAATTCTAATATCATGATGACACAGAGCCCC |
| | | AGCAGCCTGGCTGTGTCTGCTGGGGAGAAAGTGACCATGAGCTGCAAGAGCA |
| | | GCCAGTCTGTGCTGTACTCCAGCAACCAGAAGAACTACCTGGCCTGGTATCA |
| | | GCAGAAGCCTGGCCAGTCTCCTAAGCTGCTGATCTACTGGGCCAGCACCAGA |
| | | GAATCTGGGGTGCCAGACAGATTCACAGGCTCTGGCTCTGGCACAGACTTCA |
| | | CCCTGACAATCAGCTCTGTGCAGGCTGAGGACCTGGCAGTGTACTACTGTCA |
| | | CCAGTACCTGAGCAGCAGAACCTTTGGTGGTGGCACCAAGCTGGAAATCAAG |
| | | AGGGGAGGTGGTGGAAGTGGTGGTGGTGGAAGTGGAGGTGGTGGTTCTCAGG |
| | | TTCAGCTGCAGCAACCTGGGGCTGAGCTTGTCAGACCTGGCACATCTGTGAA |
| | | GCTGAGCTGCAAGGCCTCTGGCTACACCTTCACAAGCAGCTGGATGCACTGG |
| | | GTCAAGCAGAGGCCTGGACAGGGCCTTGAGTGGATTGGAGTGATTGACCCCT |
| | | CTGACAGCTACACCAACTACAACCAGAAGTTCAAGGGCAAAGCCACACTGAC |
| | | AGTGGACACCAGCAGCAGCACAGCCTACATGCAGCTGAGCAGCCTGACCTCT |
| | | GAGGACTCTGCTGTGTACTACTGTGCCAGAGGTGGTGGCAGCAGCTACAACA |
| | | GATACTTTGATGTGTGGGGCACAGGCACCACAGTGACTGTGTCCTCTGACTA |
| | | CAAGGACGACGACGACAAGtaaTAACtcgaggacggggtgaactacgcctga |
| | | ggatccgatctttttccctctgccaaaaattatggggacatcatgaagcccc |
| | | ttgagcatctgacttctggctaataaaggaaatttattttcattgcaatagt |
| | | gtgttggaattttttgtgtctctcactcg |
| CAG.BB5.1.scFv. | SEQ ID | ctgcgcgctcgctcgctcactgaggccgcccgggcaaagcccgggcgtcggg |
| LH-FLAG-Tag | NO: 303 | cgacctttggtcgcccggcctcagtgagcgagcgagcgcgcagagagggagt |
| (ITR to ITR) | | ggccaactccatcactaggggttcctCATATGcagggtaatggggatcctct |
| | | agataggtaccctagtcgacattgattattgactagttattaatagtaatca |
| | | attacggggtcattagttcatagcccatatatggagttccgcgttacataac |
| | | ttacggtaaatggcccgcctggctgaccgcccaacgacccccgcccattgac |
| | | gtcaataatgacgtatgttcccatagtaacgccaatagggactttccattga |
| | | cgtcaatgggtggagtatttacggtaaactgcccacttggcagtacatcaag |
| | | tgtatcatatgccaagtacgccccctattgacgtcaatgacggtaaatggcc |
| | | cgcctggcattatgcccagtacatgaccttatgggactttcctacttggcag |
| | | tacatctacgtattagtcatcgctattaccatggtcgaggtgagccccacgt |
| | | tctgcttcactctccccatctcccccccctccccacccccaattttgtattt |
| | | atttattttttaattattttgtgcagcgatgggggcgggggggggggggggg |
| | | cgcgcgccaggcggggcggggcggggcgaggggcggggcggggcgaggcgga |
| | | gaggtgcggcggcagccaatcagagcggcgcgctccgaaagtttccttttat |
| | | ggcgaggcggcggcggcggcggccctataaaaagcgaagcgcgcggcgggcg |
| | | ggagtcgctgcgcgctgccttcgccccgtgccccgctccgccgccgcctcgc |
| | | gccgcccgccccggctctgactgaccgcgttactcccacaggtgagcgggcg |
| | | ggacggcccttctcctccgggctgtaattagcgcttggtttaatgacggctt |
| | | gtttcttttctgtggctgcgtgaaagccttgaggggctccgggagggccctt |
| | | tgtgcggggggagcggctcggggggtgcgtgcgtgtgtgtgtgcgtggggag |
| | | cgccgcgtgcggctccgcgctgcccggcggctgtgagcgctgcgggcgcggc |
| | | gcggggctttgtgcgctccgcagtgtgcgcgaggggagcgcggccgggggcg |
| | | gtgccccgcggtgcggggggggctgcgaggggaacaaaggctgcgtgcgggg |
| | | tgtgtgcgtgggggggtgagcagggggtgtgggcgcgtcggtcgggctgcaa |
| | | ccccccctgcacccccctccccgagttgctgagcacggcccggcttcgggtg |
| | | cggggctccgtacggggcgtggcgcggggctcgccgtgccgggcggggggtg |
| | | gcggcaggtgggggtgccgggcggggcggggccgcctcgggccggggagggc |
| | | tcgggggaggggcgcggcggcccccggagcgccggcggctgtcgaggcgcgg |
| | | cgagccgcagccattgccttttatggtaatcgtgcgagagggcgcagggact |
| | | tcctttgtcccaaatctgtgcggagccgaaatctgggaggcgccgccgcacc |
| | | ccctctagcgggcgcggggcgaagcggtgcggcgccggcaggaaggaaatgg |
| | | gcggggagggccttcgtgcgtcgccgcgccgccgtccccttctccctctcca |
| | | gcctcggggctgtccgcggggggacggctgccttcgggggggacggggcagg |
| | | gcggggttcggcttctggcgtgtgaccggcggctctagagcctctgctaacc |
| | | atgttcatgccttcttctttttcctacagctcctgggcaacgtgctggttat |
| | | tgtgctgtctcatcattttggcaaagAATTCGCCACCATGTACAGAATGCAG |
| | | CTGCTGCTGCTCATTGCCCTGTCTCTGGCCCTGGTCACCAATTCTAATATCA |
| | | TGATGACACAGAGCCCCAGCAGCCTGGCTGTGTCTGCTGGGGAGAAAGTGAC |
| | | CATGAGCTGCAAGAGCAGCCAGTCTGTGCTGTACTCCAGCAACCAGAAGAAC |
| | | TACCTGGCCTGGTATCAGCAGAAGCCTGGCCAGTCTCCTAAGCTGCTGATCT |
| | | ACTGGGCCAGCACCAGAGAATCTGGGGTGCCAGACAGATTCACAGGCTCTGG |
| | | CTCTGGCACAGACTTCACCCTGACAATCAGCTCTGTGCAGGCTGAGGACCTG |
| | | GCAGTGTACTACTGTCACCAGTACCTGAGCAGCAGAACCTTTGGTGGTGGCA |
| | | CCAAGCTGGAAATCAAGAGGGGAGGTGGTGGAAGTGGTGGTGGTGGAAGTGG |
| | | AGGTGGTGGTTCTCAGGTTCAGCTGCAGCAACCTGGGGCTGAGCTTGTCAGA |
| | | CCTGGCACATCTGTGAAGCTGAGCTGCAAGGCCTCTGGCTACACCTTCACAA |
| | | GCAGCTGGATGCACTGGGTCAAGCAGAGGCCTGGACAGGGCCTTGAGTGGAT |
| | | TGGAGTGATTGACCCCTCTGACAGCTACACCAACTACAACCAGAAGTTCAAG |
| | | GGCAAAGCCACACTGACAGTGGACACCAGCAGCAGCACAGCCTACATGCAGC |
| | | TGAGCAGCCTGACCTCTGAGGACTCTGCTGTGTACTACTGTGCCAGAGGTGG |
| | | TGGCAGCAGCTACAACAGATACTTTGATGTGTGGGGCACAGGCACCACAGTG |
| | | ACTGTGTCCTCTGACTACAAGGACGACGACGACAAGtaaTAACtcgaggacg |
| | | gggtgaactacgcctgaggatccgatctttttccctctgccaaaaattatgg |
| | | ggacatcatgaagccccttgagcatctgacttctggctaataaaggaaattt |
| | | attttcattgcaatagtgtgttggaattttttgtgtctctcactcggaagca |
| | | attcgttgatctgaatttcgaccacccataatacccattaccctgCCAGGGT |
| | | AATGGGCTAGCTGCGGCCGCaggaacccctagtgatggagttggccactccc |
| | | tctctgcgcgctcgctcgctcactgaggccgggcgaccaaaggtcgcccgac |
| | | gcccgggctttgcccgggcggcctcagtgagcgagcgagcgcgcag |
| Eculizumab | | ITR - CAG - signal sequence + Eculizumab |
| scFv (H-L) | | ScFv (H-linker-L) - polyA -ITR |
| cassette (ITR- | | |
| ITR) | | |
| Eculizumab | | ITR - CAG - signal sequence + Eculizumab |
| scFv (L-H) | | ScFv (L-linker-H) - polyA -ITR |
| cassette (ITR- | | |
| ITR) | | |
| Eculizumab | | CAG - signal sequence + Eculizumab scFv (H- |
| scFv (H-L) | | linker-L) - polyA |
| cassette | | |
| (CAG-signal | | |
| sequence + ScFv | | |
| transgene- | | |
| polyA) | | |
| Eculizumab | | CAG - signal sequence + Eculizumab scFv (L- |
| scFv (L-H) | | linker-H) - polyA |
| cassette | | |
| (CAG-signal | | |
| sequence + ScFv | | |
| transgene- | | |
| polyA) | | |

### Gene Therapy Methods

Provided are methods of treating human subjects for dry AMD by administration of a viral vector containing a transgene encoding an anti-C5 or anti-C3 antibody, or antigen binding fragment thereof, or CFHL-1 protein. The antibody may be crovalimab, eculizumab, ravulizumab, tesidolumab, or NGM621 and is, e.g., a full length or substantially full length antibody or Fab fragment thereof, or other antigen-binding fragment thereof. The viral vector has an AAV capsid with tropism for human ocular tissues and may be an AAV8, AAV9, AAV3B, or AAVrh73 (or a variant thereof, for example having 90%, 95% or 99% sequence identity to the capsid sequence of AAV8, AAV9, AAV3B, or AAVrh73). The transgene is operably linked by regulatory sequences that promote expression of the transgene in human ocular tissue cells (including in retinal cells, RPE, choroid, BrM, choriocapillaris, photoreceptor cells, retinal ganglion cells), for example a CAG (SEQ ID NO: 74) promoter or a mutated CAG promoter (SEQ ID NO: 304, SEQ ID NO: 305 or SEQ ID NO: 306), or ocular specific promoter, such as a human rhodopsin kinase (GRK1) promoter (SEQ ID NOS: 77 or 217), a mouse cone arresting (CAR) promoter (SEQ ID NOS: 214-216), a human red opsin (RedO) promoter (SEQ ID NO: 212) or a Best1/GRK1 tandem promoter (SEQ ID NO: 224). Regulatory sequences may also include polyadenylation signal sequences. The expression cassette comprising the transgene and operably linked regulatory sequences are flanked by ITR sequences, as an artificial AAV genome. The flanking ITR sequences may be configured to provide a self-complementary AAV (scAAV) genome. The recombinant vectors, including those as shown in FIGS. 2A-2G, can be administered in any manner such that the recombinant vector enters one or more ocular tissue cells. In particular embodiments, the recombinant AAV comprises an artificial genome of (or is produced using a cis plasmid or construct comprising) CAG Crovalimab.scFv (SEQ ID NO: 269), CAG.Crovalimab.Fab (SEQ ID NO: 43), CAG.Crovalimab.full (SEQ ID NO: 44), CAG.Eculizumab.Fab.IgG1 (SEQ ID NO: 45). CAG.Eculizumab.Fab.IgG2 (SEQ ID NO: 46), CAG.Eculizumab.Full (SEQ ID NO: 47). CAG.hCFHL-1 (SEQ ID NO: 50). In particular embodiments, the recombinant AAV comprises an artificial genome of (or is produced using a cis plasmid or construct comprising) Crovalimab.scFv (SEQ ID NO: 267 or SEQ ID NO: 268 or SEQ ID NO: 269 or SEQ ID NO: 277 or SEQ ID NO: 278 or SEQ ID NO: 279 or SEQ ID NO: 280 or SEQ ID NO: 281 or SEQ ID NO: 282 or SEQ ID NO: 283 or SEQ ID NO: 284 or SEQ ID NO: 285 or SEQ ID NO: 286 or SEQ ID NO: 287 or SEQ ID NO: 288 or SEQ ID NO: 289 or SEQ ID NO: 290 or SEQ ID NO: 291 or SEQ ID NO: 292). In certain embodiments, the artificial genome is self complementary. In other embodiments, the recombinant AAV comprises a construct comprising a transgene encoding a surrogate anti-C5 antibody, or antigen binding fragment thereof, including BB5.1, for use in animal model, such as non-human primate, testing of anti-C5 antibodies. Constructs encoding a BB5.1 antibody include CAG.BB5.1 (SEQ ID NO: 48).

Provided also are method of administering recombinant AAV vectors comprising a transgene which is a scFv. In some embodiments, the transgene encodes an scFv with the structure: signal sequence-VH-linker-VL-polyA. In some embodiments, the transgene encodes an scFv with the structure: signal sequence-VL-linker-VH-poly A. In some embodiments, the linker is GGGGS (SEQ ID NO: 51), GGGGSGGGGS (SEQ ID NO: 52), GGGGSGGGGSGGGGS (SEQ ID NO: 53), GGGGSGGGGSGGGGSGGGGS (SEQ ID NO: 54) or GGGGSGGGGSGGGGSGGGGSGGGGS (SEQ ID NO: 55). In some embodiments, the signal sequence is MYRMQLLLLIALSLALVTNS (SEQ ID NO: 85) or a signal sequence from Table 2. In some embodiments, the VH is SEQ ID NO: 251 and VL is SEQ ID NO: 252, wherein VH is SEQ ID NO: 253 and VL is SEQ ID NO: 254, wherein VH is SEQ ID NO: 255 and the VL is SEQ ID NO: 256, wherein VH is SEQ ID NO: 257 and VL is SEQ ID NO: 258, or wherein VH is SEQ ID NO: 259 and VL is SEQ ID NO: 260.

Therapeutically effective doses of any of these recombinant vectors should be administered in any manner such that the recombinant vector enters ocular tissue cells (e.g., retinal cells), e.g., via subretinal, intravitreal, intracameral, or suprachoroidal injection or intranasal administration. Alternatively, the vector is administered peripherally (for example, intravenously, intramuscularly or subcutaneously) such that the recombinant vector transduces liver and/or muscle cells, creating a depot in liver and/or muscle tissue which express the transgene product into the bloodstream, delivering the therapeutic to ocular tissues. Alternatively, subretinal, intravitreal, intracameral, suprachoroidal administration should result in expression of the transgene product in cells of the eye, creating a depot in one or more ocular tissue cells of the patient that continuously supplies the anti-C3 or C5 HuPTM mAb, or antigen binding fragment of the anti-C3 or C5mAb (or CFHL-1) to ocular tissues of the subject. The transgene expression results in therapeutically effective levels of the anti-C5 or anti-C3 antibody or antigen-binding fragment thereof of CFHL-1 protein in the aqueous humor, the vitreous humor, retinal tissue, the RPE, the BrM or choriocapillaris.

Subjects to whom such gene therapy is administered can be those responsive to anti-complement therapy. In certain embodiments, the methods encompass treating patients who have been diagnosed with dry AMD, or have one or more symptoms associated therewith, and identified as responsive to treatment with an anti-cC3 or C5 antibody, or considered a good candidate for therapy with an anti-C3 or C5 antibody or CFHL-1 protein. In specific embodiments, the patients have previously been treated with crovalimab, eculizumab, ravulizumab, tesidolumab or other complement activation inhibitor, and have been found to be responsive to thereto. To determine responsiveness, the anti-C3 or C5 transgene product (e.g., produced in cell culture, bioreactors, etc.) may be administered directly to the subject.

In embodiments, administration of the recombinant AAV comprising a construct for expressing a transgene encoding an anti-C3 or anti-C5 antibody or antigen-binding fragment thereof, or CFHL-1 protein in ocular tissues results in reduction or slowing the progression of one or more symptoms of dry AMD within 10 days, 20 days, 30 days, 40 days, 6 months, 9 months or 1 year after administration of the AAV. In embodiments, the administration results in a slowing or reduction in the rate of the progression of geographic atrophy, including of the fovea, in the subject relative to an untreated subject or as expected in the subject based upon natural history of dry AMD, for example as measured by fundus autofluorescence (FAF). In embodiments, the administration results in an improvement or reduction in the rate of loss of visual acuity or best corrected visual acuity (BCVA), for example, as measured by a standard ETDRS chart or to improve visual function as measured by dark adaptation methodology; to improve contrast sensitivity by the Pelli-Robson test or to reduce the drusen area or accumulation of drusen. In other embodiments, the dose of therapeutic gene delivered by gene therapy is sufficient to inhibit complement activation without exacerbating choroidal neovascularization (CNV).

However, in all cases because the transgene product is continuously produced, maintenance of lower concentrations can be effective. Notwithstanding, because the transgene product is continuously produced, maintenance of lower concentrations can be effective. The concentration of the transgene product can be measured in patient blood serum samples.

Pharmaceutical compositions suitable for subretinal, intravitreal, intranasal, intracameral, suprachoroidal, or systemic (intravenous, intramuscular or subcutaneous) administration comprise a suspension of the recombinant vector comprising the transgene encoding the anti-C3 or C5 antibody, or antigen-binding fragment thereof, or CFHL-1 protein in a formulation buffer comprising a physiologically compatible aqueous buffer. The formulation buffer can comprise one or more of a polysaccharide, a surfactant, polymer, or oil.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of or relieve one or more symptoms of dry AMD, such as to reduce the rate of geographic atrophy or improve visual acuity (or reduce the rate of loss of visual acuity).

Combinations of delivery of the anti-C3 or C5 HuPTM mAb or antigen-binding fragment thereof or CFHL-1, to the eye, liver and/or muscles accompanied by delivery of other available treatments are encompassed by the methods provided herein. The additional treatments may be administered before, concurrently, or subsequent to the gene therapy treatment. Available treatments for a subject with dry AMD that could be combined with the gene therapy provided herein include but are not limited to, elamipretide, risuteganib, photobiomodulation, brimonidine tartrate, kamuvudine, Xiflam, or doxycycline, and others and administration with anti-C3 antibody or C5 antibody or CFHL-1 protein.

### 5.6. Monitoring of Efficacy

The compositions and methods described herein may be assessed for efficacy using any method for assessing efficacy in treating, preventing, or ameliorating dry AMD. The assessment may be determined in animal models or in human subjects. The efficacy on visual deficits may be measured by best corrected visual acuity (BCVA), for example, assessing the increase in numbers of letters or lines and where efficacy may be assessed as an increase in greater than or equal to 2 ETDRS lines or reduction in geographic atrophy, including of the fovea, to be assessed by visual inspection.

The compositions and methods described herein may be assessed for efficacy using any method for assessing efficacy in treating, preventing, or ameliorating dry AMD. The assessment may be determined in animal models or in human subjects. The efficacy on visual deficits may be measured by best corrected visual acuity (BCVA), for example, assessing the increase in numbers of letters or lines and where efficacy may be assessed as an increase in greater than or equal to 2 ETDRS lines or an increase in log MAR. Physical changes to the eye, including changes in geographic atrophy may be measured Optical Coherence Tomography, using methods known in the art.

The compositions and methods described herein may be assessed for efficacy using in vitro complement inhibition assays, such as membrane attack complex (“MAC”) formation, C5a generation and hemolysis. Complement inhibition assays can be performed in any appropriate cell type, such as ARPE19 cells (MAC and C5a assays), iPSC-derived RPE cells (MAC and C5a assays) or sheep/rabbit erythrocytes (hemolysis assay). MAC formation assays measure the deposition of MAC on the surface of RPE cells (% relative inhibition of MAC formation). C5a generation assays measure the ability of the C5 antibody to prevent C5 cleavage (less C5 cleavage=less C5a) Hemolysis assays allow the comparison of complement inhibition among different complement inhibitors (50% complement inhibition dose (ng/ml) (CH₅₀; AH₅₀).

Animal models may be used to assess the recombinant vectors encoding the anti-C3 antibodies, anti-C5 antibodies of CFHL-1 proteins for expression, therapeutic effect and adverse effects. Animal models may include a humanized C3-/C5-rodent model (Example 9) or a NaIO3 induction rat or mouse model (also see Example 9), or a CFH−/− mouse model. Animals may be administered vectors described herein, for example, subretinally or suprachoroidally, and then assessed for geographic atrophy (or change therein) by OCT, retinal pathology (damage to RPE), and other assessments of dry AMD pathology, as well as reduction in C3a or C5a, cleavage of C3 or C5 or other markers of complement activation.

Endpoints may include, but are not limited to, mean change in geographic atrophy in the study eye from baseline to 12, 16, 20, 24, or 28 weeks or at time of administration, if earlier, proportion of responders in the study eye at 12, 16, 20, 24, or 28 weeks, mean change in best corrected visual acuity from baseline to 12, 16, 20, 24, or 28 weeks, change from baseline in quality of life/patient reported outcome assessments, mean change in visual acuity from baseline to 12, 16, 20, 24, or 28 weeks.

### 6 EXAMPLES

### 6.1 Example 1: Crovalimab Fab cDNA-Based Vector

A Crovalimab Fab cDNA-based vector was constructed comprising a transgene comprising nucleotide sequences encoding the Fab portion of the heavy and light chain sequences of craovalimab (amino acid sequences being SEQ ID NOs. 1 and 2, respectively). The nucleotide sequence coding for the Fab portion of the heavy and light chain is the nucleotide sequence of SEQ ID NOs. 26 and 27, respectively. The transgene also comprises nucleotide sequences that encodes a signal peptide, e.g., MYRMQLLLLIALSLALVINS (SEQ ID NO:85). The nucleotide sequences encoding the light chain and heavy chain are separated by IRES elements or 2A cleavage sites (See Table 4, particularly, SEQ ID NO: 142 or 144) to create a bicistronic vector. The vector additionally includes the constitutive promoter CAG (SEQ ID NO: 74). Alternatively, other constitutive promoters, such as mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 224), or an inducible promoter, such as a hypoxia-inducible promoter, may be used. The artificial genome from the 5′ ITR to the 3′ ITR is shown in SEQ ID NO: 43.

### 6.2 Example 2: Eculizumab IgG1 Fab cDNA-Based Vector

An eculizumab Fab IgG1 cDNA-based vector was constructed comprising a transgene comprising nucleotide sequences encoding the Fab portion of the heavy and light chain sequences of eculizumab (amino acid sequences being SEQ ID NOs. 3 and 5, respectively). The nucleotide sequence coding for the Fab portion of the heavy and light chain is the nucleotide sequence of SEQ ID NOs. 28 and 30, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes the constitutive promoter CAG (SEQ ID NO: 74). Alternatively, other constitutive promoters, such as mU1a, EF1a, a CB or CB long promoter, a tissue-specific promoter, such as a ocular tissue-specific promoter, particularly GRK1 promoter (SEQ ID NO:77). or a BEST1/GRK1 tandem promoter (SEQ ID NO: 224), or an inducible promoter, such as a hypoxia-inducible promoter, may be used. The sequence of the vector from the 5′ ITR to the 3′ ITR is shown in SEQ ID NO: 45.

### 6.3 Example 3: Eculizumab IgG2 Fab cDNA-Based Vector

An eculizumab Fab cDNA-based vector was constructed comprising a transgene comprising nucleotide sequences encoding the Fab portion of the heavy and light chain sequences of eculizumab (amino acid sequences being SEQ ID NOs. 4 and 5, respectively). The nucleotide sequence coding for the Fab portion of the heavy and light chain is the nucleotide sequence of SEQ ID NOs. 29 and 30, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes the constitutive promoter CAG (SEQ ID NO: 74). Alternatively, other constitutive promoters, such as mU1a, EF1a, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 224), or an inducible promoter, such as a hypoxia-inducible promoter, may be used. The sequence of the vector from the 5′ ITR to the 3′ ITR is shown in SEQ ID NO: 46.

### 6.4 Example 4: Ravulizumab Fab cDNA-Based Vector

A ravulizumab Fab cDNA-based vector is constructed comprising a transgene comprising nucleotide sequences encoding the Fab portion of the heavy and light chain sequences of ravulizumab (amino acid sequences may be SEQ ID NOs. 6 and 7, respectively). The nucleotide sequence coding for the Fab portion of the heavy and light chain may be the nucleotide sequence of SEQ ID NOs. 31 and 32, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG (SEQ ID NO: 74), mU1a, EF1a, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 224), or an inducible promoter, such as a hypoxia-inducible promoter.

### 6.5 Example 5: Tesidolumab Fab cDNA-Based Vector

A tesidolumab Fab cDNA-based vector is constructed comprising a transgene comprising nucleotide sequences encoding the Fab portion of the heavy and light chain sequences of tesidolumab (amino acid sequences being SEQ ID NOs. 8 and 9, respectively). The nucleotide sequence coding for the Fab portion of the heavy and light chain may be the nucleotide sequence of SEQ ID NOs. 33 and 34, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG (SEQ ID NO: 74), mU1a, EF1a, a CB or CB long promoter, such as a ocular tissue-specific promoter, particularly GRK1 promoter (SEQ ID NO: 77), or a BEST1/GRK1 tandem promoter (SEQ ID NO: 224), or an inducible promoter, such as a hypoxia-inducible promoter.

### 6.6 Example 6: NGM621 Fab cDNA-Based Vector

A NGM621 Fab cDNA-based vector is constructed comprising a transgene comprising nucleotide sequences encoding the Fab portion of the heavy and light chain sequences of NGM621 (amino acid sequences being SEQ ID NOs. 10 and 13, respectively). Alternatively, the vector is constructed comprising a transgene comprising nucleotide sequences encoding the Fab portion of the heavy and light chain sequences of NGM621 (amino acid sequences may be SEQ ID NOs. 11 and 13, respectively). The nucleotide sequence coding for the Fab portion of the heavy and light chain may be codon-optimized nucleotide sequences that encode SEQ ID NOs. 10 and 13, respectively or, alternatively, codon-optimized nucleotide sequence that encode SEQ ID NOs. 11 and 13, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO: 142 or 144) to create a bicistronic vector. The vector additionally includes a constitutive promoter, such as CAG (SEQ ID NO: 74), mU1a, EF1a, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 224), or an inducible promoter, such as a hypoxia-inducible promoter.

### 6.7 Example 7: Expression of Eculizumab, Crovalimab and hCFHL.1 in HEK293 Cells

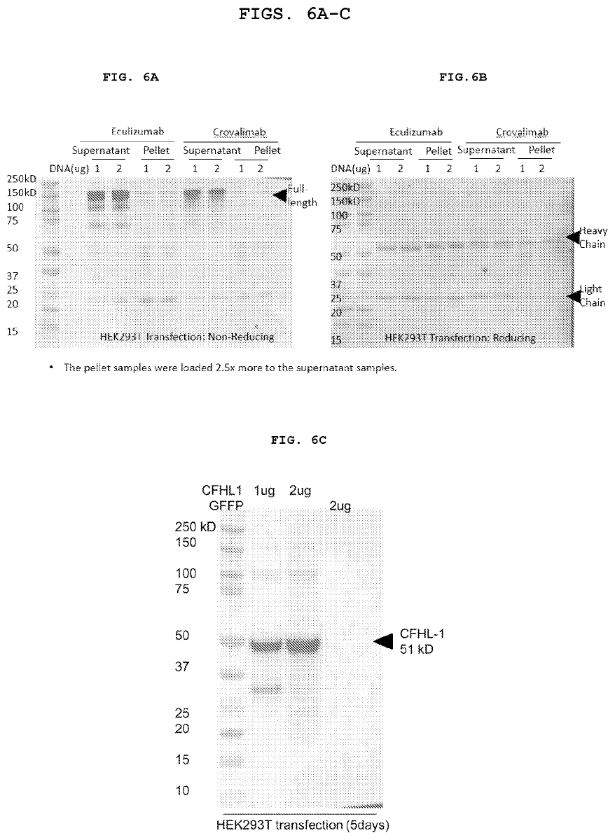

Cis plasmids pITR-CAG-Eculizumab.IgG1 (SEQ ID NO: 45) or pITR-CAG-Crovalimab (SEQ ID NO: 43) were transfected into HEK293 cells. Supernatant and pellet samples were collected and run on a non-reducing gel (FIG. 6A), with a band representing full length antibodies observed in the supernatant of the cell culture, or run on a reducing gel (FIG. 6B), indicating heavy and light chains of the antibodies seen in both cell pellet and supernatant. Plasmids pITR-CAG-hCFHL.1 or control (GFFP) were transfected into HEK293 cells and expression was detected (FIG. 6C).

### 6.8 Example 8: Self-Complementary hCFHL1 Transgene Cassette

A CFHL-1 cDNA-based vector was constructed comprising a transgene comprising nucleotide sequences encoding CFHL-1 (SEQ ID NO: 23) including the native complement factor H signal sequence, which are the first 18 amino acids of SEQ ID NO: 23 (also SEQ ID NO: 90, Table 2). The nucleotide sequence coding for the CFHL-1 may be a codon-optimized nucleotide sequence of SEQ ID NO: 49. The vector additionally includes a constitutive promoter, such as CAG (SEQ ID NO: 74), mU1a, EF1a, a CB or CB long promoter, a tissue-specific promoter, such as a ocular tissue-specific promoter, particularly GRK1 promoter (SEQ ID NO: 77), or a BEST1/GRK1 tandem promoter (SEQ ID NO: 224), or an inducible promoter, such as a hypoxia-inducible promoter and a polyadenylation signal. The expression cassette is flanked by ITR sequences, where one ITR is mutated to generate a self-complementary artificial genome (for example a 3′ ITR sequence of SEQ ID NO: 84, Table 1), and the artificial genome may have a nucleotide sequence of SEQ ID NO: 50.

### 6.9 Example 9: NaIO3 Induction Mouse Model

A NaIO3 induced model of dry AMD in rodents (inducing RPE damage) will be used to assess the anti-C3, anti-C5 antibodies and CFHL-1 protein AAV constructs. AAV8 constructs, AAV8.CAG.crovalimab.Fab (SEQ ID NO: 43), AAV8.CAG.crovalimab.full (SEQ ID NO:NO: 44), AAV8.eculizumab.Fab.IgG1 (SEQ ID NO: 45), AAV8.eculizumab.Fab.IgG2 (SEQ ID NO: 46), AAV8.eculizumab.full (SEQ ID NOL 47), AAV8.CAG.BB5.1 (SEQ ID NO: 48) and AAV8.CAG.hCFHL.1f (SEQ ID NO: 5) will be administered to humanized C3-/C5-mice at a dose of 1E7, 1E8 or 1E9 subretinally or suprachoroidally. 28 days later, NaIO3 will be administered to the mouse to induce geographic atrophy. One week later, the eyes of the mice will be assessed by fundus and for visual function deficits and then will be sacrificed and eyes assessed for inhibition of RPE damage and photoreceptor loss and for transgene, C3 and C5 levels.

### 6.10 Example 10: Hemolysis Assay

All C5 inhibitor expression cassettes utilized in this study were constructed with a CAG promoter and rabbit beta-globin polyA. All transgenes were codon-optimized and CpG depleted. Cis-plasmids were initially screened in the assay following transfection in 293T cells and then subsequently packaged as AAV8 viral vectors (including scAAV8 vectors) for further study.

The classical pathway of complement activation (CP) is initiated primarily by immune complexes. The standard assay for the overall functional activity of this pathway is the CH50. This assay uses sheep erythrocytes coated with rabbit antibodies (referred to as EA) to activate the complement system.

Such classical complement pathway-related hemolysis inhibition assay was employed using supernatant collected from plasmids (encoding complement inhibitors as described herein) transfected into HEK293T cells. The supernatants (containing the complement inhibitor, or negative controls containing media without inhibitor or containing a vectorized antibody to a non-complement related target) were collected and applied to sheep erythrocytes coated with optimum levels of rabbit anti-sheep erythrocyte IgM antibodies suspended at 5×10⁸ cells/ml in Gelatin Veronal Buffered saline (GVB++ Buffer) in the wells of an assay plate. Percent hemolysis was compared to a positive test hemolytic solution containing normal human serum that has been titrated up to 50% hemolysis. Percent hemolysis was calculated as such: % hemolysis-(test sample hemolysis (OD405)-background hemolysis (OD405))/(maximal hemolysis (OD405)-background hemolysis (OD405))×100.

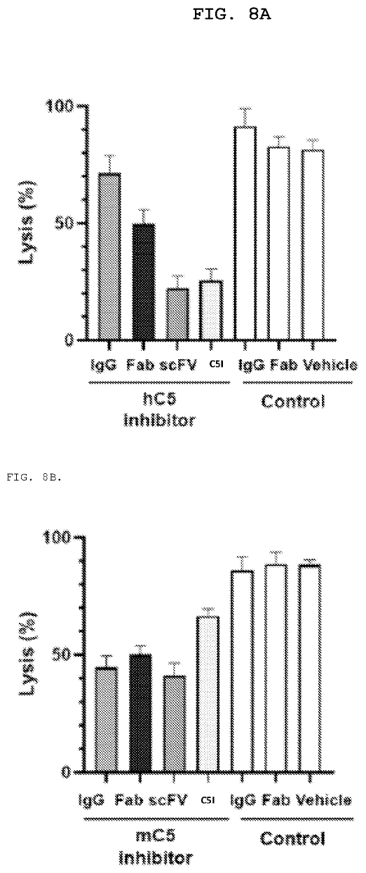

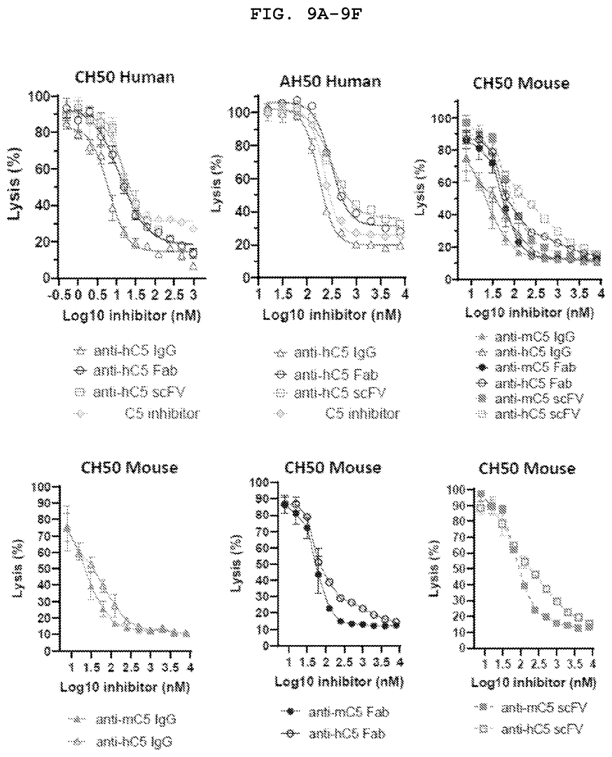

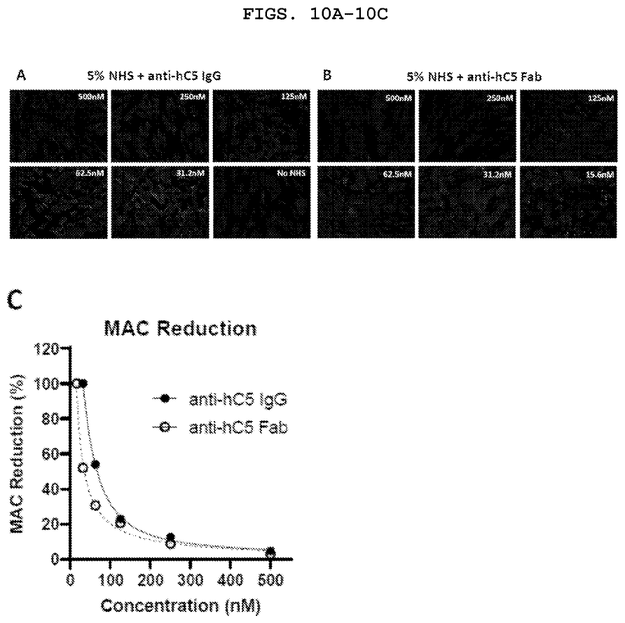

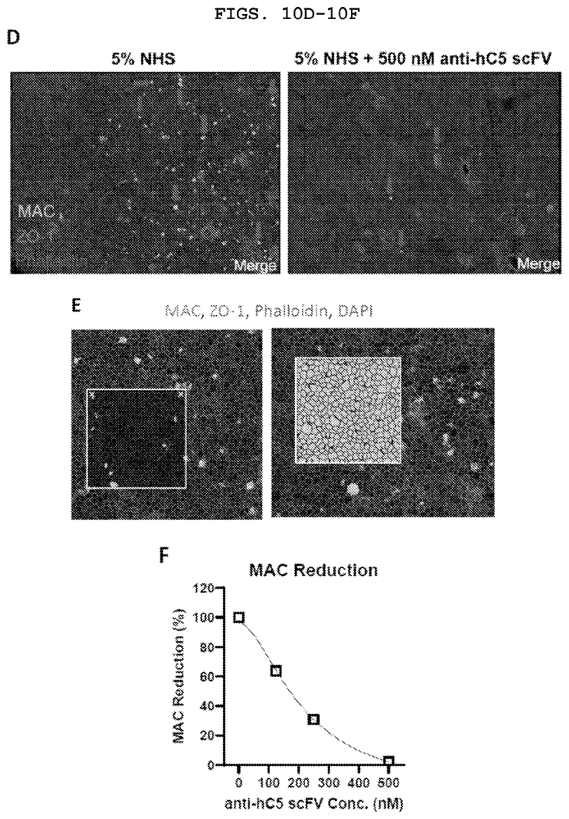

The neat supernatant collected from HEK293 cells transfected with vectorized Crovalimab or BB5.1 plasmids (different vectorized forms IgG, Fab or ScFv) or a recombinant protein C5 inhibitor were tested in the assay for the % of lysis of the EA as an indication of inhibition of complement activation (FIGS. 8A-8B). Purified proteins of C5 inhibitors were tested at series concentrations (nM) to determine the minimum concentration necessary to lyse 50% of the cells (1 CH50 Unit) (FIGS. 9A-9F). Activation of the classical pathway requires calcium and magnesium ions. The initial reactions include binding of C1 and activation of C2 and C4 to form a C3 convertase. This enzyme cleaves C3 which promotes cleavage of C5 and activation of the membrane attack pathway (proteins C5, C6, C7, C8 and C9). These five components assemble in the membrane of the sheep erythrocyte and lyse the cell. The release of hemoglobin is subsequently quantitated to measure the total complement activity present in the sample.

C5 inhibitors expressed in HEK293 cells suppress complement pathway activation in hemolysis inhibition assays at varying degrees. The scFv formats displayed strong inhibition of complement (FIGS. 8A-B). Recombinant purified forms of each C5 inhibitor displayed potent inhibition of complement activation in classical and alternative hemolysis assays (FIGS. 9A-F).

### 6.11 Example 11 Binding Kinetics and Affinity of Recombinant Purified Forms of Each C5 Inhibitor

The generated recombinant purified proteins of each C5 inhibitor (expressed in HEK293 cells as above) were compared by their IC50 values in both the classical and alternative complement pathways. The binding kinetics and affinity of each C5 inhibitor to human, cynomolgous macaque, and mouse C5 were measured with the OctetRED384 system, as follows.

a. Affinity and Kinetics for Human Complement C5:

The assay was performed at 30° C. and at 1000 rpm. Biotinylated Human Complement C5 Protein was firstly immobilized onto SA biosensor. Crovalimab IgG and Ab fragments and Coversin was applied as analyte for association and dissociation steps.

TABLE 9
Assay conditions for human C5 assay after optimization

| | | Ligand | Immobilization | | Analyte | | |
|---|---|---|---|---|---|---|---|
| | | Conc. | Level | | Conc. | Association | Dissociation |
| Method | Ligand | (μg/ml) | (nm) | Analyte | (nM) | Time | Time |
| SA | Biotinlyated | 2.5 | −0.8 | Crovalimab | 20, 10, | 100 | 300 |
| | Human | | | IgG | 5, 2.5 | | |
| | Complement | 2.5 | −1.2 | Crovalimab- | 30, 15, | 110 | 300 |
| | C5 | | | Fab-His6 | 7.5, | | |
| | Protein | | | | 3.75, | | |
| | | | | | 1.88 | | |
| | | 2.5 | −1.8 | Crovalimab | 20, 10, | 90 | 300 |
| | | | | scFv.HL | 5, 2.5 | | |
| | | 3 | −2.0 | Protein C5 | 200, | 130 | 300 |
| | | | | inhibitor | 100, 50 | | |

b. Affinity and Kinetics for Cynomolgous Complement C5:

The assay was performed at 30° C. and at 1000 rpm. Biotinylated cyno C5 antigen was firstly immobilized onto SA biosensor. Crovalimab IgG and Ab fragments and a recombinant C5 inhibitor protein were applied as analyte for association and dissociation steps.

TABLE 10
Assay conditions for cyno C5 assay after optimization

| | | Ligand | Immobilization | | Analyte | | |
|---|---|---|---|---|---|---|---|
| | | Conc. | Level | | Conc. | Association | Dissociation |
| Method | Ligand | (μg/ml) | (nm) | Analyte | (nM) | Time | Time |
| SA | Biotinlyated | 2.5 | −0.8 | Crovalimab | 20, 10, | 100 | 600 |
| | Cyno | | | IgG | 5, 2.5 | | |
| | Complement | 2.5 | −1.2 | Crovalimab- | 30, 15, | 101 | 600 |
| | C5 | | | Fab-His6 | 7.5, | | |
| | Protein | | | | 3.75, | | |
| | | | | | 1.88 | | |
| | | 2.5 | −1.8 | Crovalimab | 20, 10, | 120 | 600 |
| | | | | scFv.HL | 5, 2.5, | | |
| | | | | | 1.25 | | |
| | | 2.5 | −2.0 | Protein C5 | 100, 50, | 100 | 200 |
| | | | | inhibitor | 25, 12.5 | | |

c. Affinity and Kinetics for Mouse Complement C5:

The assay was performed at 30° C. and at 1000 rpm. Biotinylated mouse C5 antigen was firstly immobilized onto SA biosensor. BB5.1 IgG and Ab fragments were applied as analyte for association and dissociation steps.

TABLE 11
Assay conditions for mouse C5 assay after optimization (surrogate anti-mouse C5 mAb)

| | | Ligand | Immobilization | | Analyte | | |
|---|---|---|---|---|---|---|---|
| | | Conc. | Level | | Conc. | Association | Dissociation |
| Method | Ligand | (μg/ml) | (nm) | Analyte | (nM) | Time | Time |
| SA | Biotinlyated | 1.0 | −0.6 | BB5.1 IgG | 2.5, 1.5, | 100 | 600 |
| | Mouse | | | | 0.0625, | | |
| | Complement | | | | 0.3125 | | |
| | C5 | 2.0 | −1.6 | BB5.1-Fab- | 20, 10, | 120 | 180 |
| | Protein | | | His6 | 5, 2.5, | | |
| | | | | | 1.25 | | |
| | | 2.0 | −2.0 | BB5.1 | 10, 5, | 100 | 180 |
| | | | | scFv.LH | 2.5, | | |
| | | | | | 1.25 | | |

The assay was performed at 30° C. and at 1000 rpm. Biotinylated mouse C5 antigen was firstly immobilized onto SA biosensor. Crovalimab IgG and Ab fragments and Coversin were applied as analyte for association and dissociation steps.

TABLE 12
Assay conditions for mouse C5 assay after optimization (cross-species C5 binders)

| | | Ligand | Immobilization | | Analyte | | |
|---|---|---|---|---|---|---|---|
| | | Conc. | Level | | Conc. | Association | Dissociation |
| Method | Ligand | (μg/ml) | (nm) | Analyte | (nM) | Time | Time |
| SA | Biotinlyated | 1.7 | −0.6 | Crovalimab | 10, 5, | 110 | 500 |
| | Mouse | | | IgG | 2.5 | | |
| | Complement | 1.7 | −1.2 | Crovalimab- | 30, 15, | 102 | 181 |
| | C5 | | | Fab-His6 | 7.5, | | |
| | Protein | | | | 3.75, | | |
| | | | | | 1.88 | | |
| | | 2.5 | −1.2 | Crovalimab | 20, 10, | 120 | 180 |
| | | | | scFv.HL | 5, 2.5 | | |
| | | 2.5 | −3.0 | Protein C5 | 200, | 100 | 200 |
| | | | | inhibitor | 100, 50 | | |

All three formats of anti-human C5 inhibitor (Crovalimab) demonstrated KD values for human and cyno C5 in the low to high picomolar range, whereas the C5 inhibitor bound less strongly with a low nanomolar affinity constant. Anti-Mouse (BB5.1) and Anti-human (Crovalimab) C5 inhibitors with the same vectorized antibody format demonstrated comparable affinity to mouse C5. See Table 13.

TABLE 13

| | Ligand | | |
|---|---|---|---|
| | Human C5 | Cyno C5 | Mouse C5 |
| Analyte | K_{D} (M) | K_{D} (M) | K_{D} (M) |
| Anti-hC5 IgG | 3.136E−11 | 1.310E−11 | 2.291E−11 |
| Anti-hC5 Fab | 4.001E−10 | 6.023E−11 | 4.142E−09 |
| Anti-hC5 scFV | 2.533E−10 | 8.454E−11 | 4.539E−09 |
| Anti-mC5 IgG | N/A | N/A | 1.214E−11 |
| Anti-mC5 Fab | N/A | N/A | 3.469E−09 |
| Anti-mC5 scFV | N/A | N/A | 3.692E−09 |
| recombinant C5 | 1.017E−08 | 5.092E−09 | 6.278E−09 |
| inhibitor (C5I) protein | | | |

### 6.12 Example 12: Evaluation of AAV Expressed C5 Inhibitors in iPSC-Derived RPE

Membrane attack complex (MAC) formation was measured. Briefly, ARPE19 cells or 4-5 weeks old fully matured, polarized iPSC-RPE (iRPE cells, Cellular Dynamics International Catalog, #: R1102) monolayers were used in the studies. ARPE19 or iRPE cells were treated for 24 h with purified recombinant C5 inhibitors in the culture media followed by 48 h with a second treatment of purified recombinant C5 inhibitors in the culture media along with 5% CC-HS (NHS, Complement technology), or 5% CI-HS (heat-inactivated CC-HS). Cells were fixed in 4% paraformaldehyde for 20 min, at RT and immunostained with antibodies or cell stain against C5b-9 (Invitrogen, MA5-28502), ZO-1 (Invitrogen, REF 40220), Phalloidin (AF-568 Phalloidin: Invitrogen A12380) and DAPI.

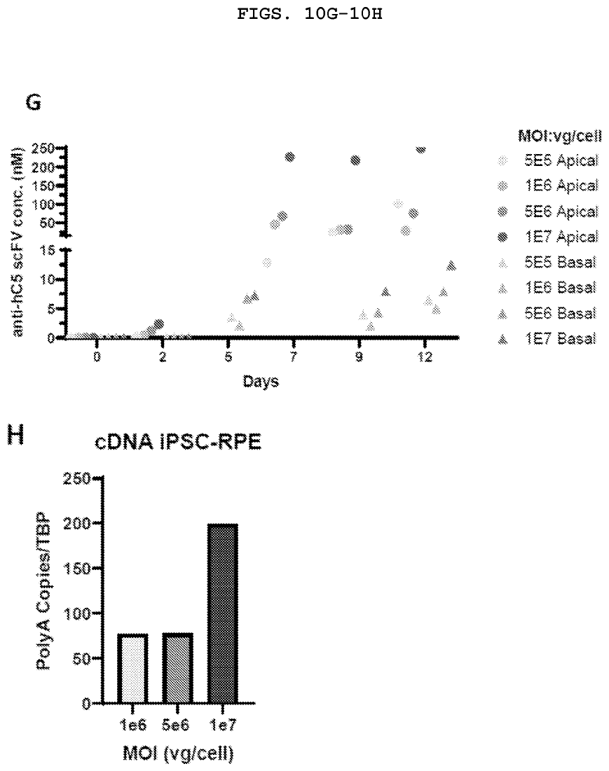

C5 inhibitors prevented C5 cleavage and reduced membrane attack complex (MAC) formation (FIGS. 10A-C, ARPE19; FIGS. 10D-10H, iPSC-derived RPE). iPSC-derived RPE transduced with AAV.anti-hC5 scFV (Crovalimab scFv) at increasing MOIs demonstrate a dose-dependent increase in transgene product (TP) level in apical and basal compartments (FIG. 10G). The TP level (FIG. 10G) was consistent with mRNA/cDNA of AAV measured by ddPCR (FIG. 10H).

### 6.13 Example 13: Evaluation of AAV Expressed C5 Inhibitors In Vivo

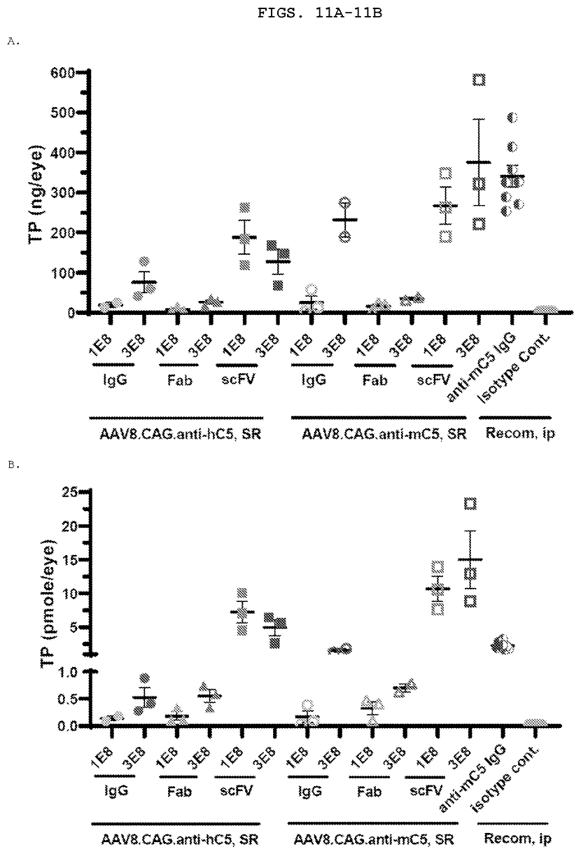

AAV8-encoding C5 inhibitors were injected into wild-type mouse eyes via subretinal (SR) administration at 1E8 and 3E8 vg/eye. Anti-C5 scFV (anti-hC5: Crovalimab or anti-mC5: BB5.1) delivered via AAV administered subretinally demonstrated a >10-fold higher TP level (FIG. 11A=ng/eye; FIG. 11B=pmol/eye) than IgG (full-length antibody) and Fab forms and similar levels to purified anti-mC5 IgG delivered intraperitoneal (IP). Also Anti-C5 scFv forms showed improved distribution to the outer ocular layers compared to IgG and Fab forms.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1.-119. (canceled)

120. A recombinant adeno-associated virus (rAAV) vector having:
a) a viral AAV capsid; and
b) an expression cassette comprising a transgene encoding a full-length anti-C5 antibody, or an antigen-binding fragment thereof,
wherein the transgene encodes i) a heavy chain comprising a CDR1 having the amino acid sequence of SSYYMAW (SEQ ID NO: 309), a CDR 2 having the amino acid sequence of AIFTGSGAEYKAEWAKG (SEQ ID NO: 310), and a CDR 3 having the amino acid sequence of AGYDYPTHAMHYW (SEQ ID NO: 311), and a light chain comprising a CDR4 having the amino acid sequence of ASQGISSSLA (SEQ ID NO: 312), a CDR5 having the amino acid sequence of YGASETES (SEQ ID NO: 313), and a CDR6 having the amino acid sequence of QNTKVGSSTGNT (SEQ ID NO: 314), or ii) a heavy chain comprising a CDR1 having the amino acid sequence of GYIFSNYWIQ (SEQ ID NO: 315), a CDR 2 having the amino acid sequence of EILPGSGSTEYTENFKD (SEQ ID NO: 316), and a CDR 3 having the amino acid sequence of YFFGSSPNWYFDV (SEQ ID NO: 317), and a light chain comprising a CDR4 having the amino acid sequence of GASENIYGALN (SEQ ID NO: 318), a CDR5 having the amino acid sequence of GATNLAD (SEQ ID NO: 319), and a CDR6 having the amino acid sequence of QNVLNTPLT (SEQ ID NO: 320),
wherein the transgene encodes a signal sequence at the N-terminus of the heavy chain, and wherein the transgene is operably linked to one or more regulatory sequences that promote expression of the transgene in human ocular tissue cells, and wherein the expression cassette is flanked by AAV inverted terminal repeats (ITRs).

121. The rAAV of claim 120, wherein the capsid comprises a capsid protein that is at least 95% identical to the amino acid sequence of AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).

122. The rAAV of claim 121, wherein the capsid comprises a capsid protein that is at least 95% identical to the amino acid sequence of AAV3B or AAV8.

123. The rAAV of claim 120, wherein the transgene encodes a heavy chain comprising the amino acid sequence of SEQ ID NO: 251 and a light chain comprising the amino acid sequence of SEQ ID NO: 252 or ii) a heavy chain comprising the amino acid sequence of SEQ ID NO: 253 and a light chain comprising the amino acid sequence of SEQ ID NO: 254.

124. The rAAV of claim 123, wherein the transgene comprises i) the nucleotide sequence of SEQ ID NO: 26 encoding the heavy chain and the nucleotide sequence of SEQ ID NO: 27 encoding the light chain or ii) the nucleotide sequence of SEQ ID NO: 28 or SEQ ID NO: 29 encoding the heavy chain and the nucleotide sequence of SEQ ID NO: 30 encoding the light chain.

125. The rAAV of claim 120, wherein the signal sequence comprises the amino acid sequence of SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 95.

126. The rAAV of claim 120, wherein the antigen binding fragment is a single chain variable fragment (scFv), wherein the transgene further encodes a linker between the heavy chain and the light chain, and wherein the linker comprises the amino acid sequence of SEQ ID NO: 51, SEQ ID NO: 52, SEQ ID NO: 53, SEQ ID NO: 54, or SEQ ID NO: 55.

127. The rAAV of claim 126, wherein the transgene encodes a polypeptide having the amino acid sequence of SEQ ID NO: 261, SEQ ID NO: 262, SEQ ID NO: 263, SEQ ID NO: 264, SEQ ID NO: 272, or SEQ ID NO: 273.

128. The rAAV of claim 126, wherein the expression cassette comprises the nucleotide sequence of SEQ ID NO: 269, SEQ ID NO: 278, SEQ ID NO: 281, SEQ ID NO: 283, SEQ ID NO: 287, SEQ ID NO: 289, or SEQ ID NO: 291.

129. The rAAV of claim 120, wherein the transgene comprises the nucleotide sequence of SEQ ID NO: 37, SEQ ID NO: 38, SEQ ID NO: 39, SEQ ID NO: 40, SEQ ID NO: 41, SEQ ID NO: 267, or SEQ ID NO: 277.

130. The rAAV of claim 120, wherein the rAAV comprises an artificial genome comprising the nucleotide sequence of SEQ ID NO: 43, SEQ ID NO: 44, SEQ ID NO: 45, SEQ ID NO: 46, SEQ ID NO: 47, SEQ ID NO: 268, SEQ ID NO: 279, SEQ ID NO: 282, SEQ ID NO: 284, SEQ ID NO: 285, SEQ ID NO: 286, SEQ ID NO: 288, SEQ ID NO: 290, or SEQ ID NO: 292.

131. A method for treating Age Related Macular Degeneration (AMD) in a human subject in need thereof comprising administering a therapeutically effective amount of a composition comprising a recombinant adeno-associated viral (rAAV) vector comprising
a) a viral capsid; and
b) an expression cassette comprising a transgene encoding a full-length anti-C5 antibody, or an antigen-binding fragment thereof,
wherein the transgene encodes i) a heavy chain comprising a CDR1 having the amino acid sequence of SSYYMAW (SEQ ID NO: 309), a CDR 2 having the amino acid sequence of AIFTGSGAEYKAEWAKG (SEQ ID NO: 310), and a CDR 3 having the amino acid sequence of AGYDYPTHAMHYW (SEQ ID NO: 311), and a light chain comprising a CDR4 having the amino acid sequence of ASQGISSSLA (SEQ ID NO: 312), a CDR5 having the amino acid sequence of YGASETES (SEQ ID NO: 313), and a CDR6 having the amino acid sequence of QNTKVGSSTGNT (SEQ ID NO: 314), or ii) a heavy chain comprising a CDR1 having the amino acid sequence of GYIFSNYWIQ (SEQ ID NO: 315), a CDR 2 having the amino acid sequence of EILPGSGSTEYTENFKD (SEQ ID NO: 316), and a CDR 3 having the amino acid sequence of YFFGSSPNWYFDV (SEQ ID NO: 317), and a light chain comprising a CDR4 having the amino acid sequence of GASENIYGALN (SEQ ID NO: 318), a CDR5 having the amino acid sequence of GATNLAD (SEQ ID NO: 319), and a CDR6 having the amino acid sequence of QNVLNTPLT (SEQ ID NO: 320), wherein the transgene encodes a signal sequence at the N-terminus of the heavy chain, and
wherein the transgene is operably linked to one or more regulatory sequences that promote expression of the transgene in human ocular tissue cells, and
wherein the expression cassette is flanked by AAV inverted terminal repeats (ITRs), and
wherein the administering is subretinal, intravitreal, intranasal, intracameral, suprachoroidal, or systemic to the subject.

132. The method of claim 131, wherein the capsid comprises a capsid protein that is at least 95% identical to the amino acid sequence of AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).

133. The method of claim 132, wherein the capsid comprises a capsid protein that is at least 95% identical to the amino acid sequence of AAV3B or AAV8

134. The method of claim 131, wherein the transgene encodes a heavy chain comprising the amino acid sequence of SEQ ID NO: 251 and a light chain comprising the amino acid sequence of SEQ ID NO: 252 or ii) a heavy chain comprising the amino acid sequence of SEQ ID NO: 253 and a light chain comprising the amino acid sequence of SEQ ID NO: 254.

135. The method of claim 134, wherein the transgene comprises i) the nucleotide sequence of SEQ ID NO: 26 encoding the heavy chain and the nucleotide sequence of SEQ ID NO: 27 encoding the light chain or ii) the nucleotide sequence of SEQ ID NO: 28 or SEQ ID NO: 29 encoding the heavy chain and the nucleotide sequence of SEQ ID NO: 30 encoding the light chain.

136. The method of claim 131, wherein the signal sequence comprises the amino acid sequence of SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, or SEQ ID NO: 95.

137. The method of claim 131, wherein the antigen binding fragment is a single chain variable fragment (scFv), wherein the transgene further encodes a linker between the heavy chain and the light chain, and wherein the linker comprises the amino acid sequence of SEQ ID NO: 51, SEQ ID NO: 52, SEQ ID NO: 53, SEQ ID NO: 54, or SEQ ID NO: 55.

138. The method of claim 137, wherein the transgene encodes a polypeptide having the amino acid sequence of SEQ ID NO: 261, SEQ ID NO: 262, SEQ ID NO: 263, SEQ ID NO: 264, SEQ ID NO: 272, or SEQ ID NO: 273

139. The method of claim 138, wherein the expression cassette comprises the nucleotide sequence of SEQ ID NO: 269, SEQ ID NO: 278, SEQ ID NO: 283, SEQ ID NO: 287, SEQ ID NO: 289, or SEQ ID NO: 291.

140. The method of claim 131, wherein the transgene comprises the nucleotide sequence of SEQ ID NO: 37, SEQ ID NO: 38, SEQ ID NO: 39, SEQ ID NO: 40, SEQ ID NO: 41, SEQ ID NO: 267, or SEQ ID NO: 277.

141. The method of claim 131, wherein the rAAV comprises an artificial genome comprising the nucleotide sequence of SEQ ID NO: 43, SEQ ID NO: 44, SEQ ID NO: 45, SEQ ID NO: 46, SEQ ID NO: 47, SEQ ID NO: 268, SEQ ID NO: 279, SEQ ID NO: 284, SEQ ID NO: 285, SEQ ID NO: 286, SEQ ID NO: 288, SEQ ID NO: 290, or SEQ ID NO: 292.

142. The method of claim 131, wherein the therapeutically effective amount is sufficient to maintain a concentration of at least 10 ng/ml of the antibody or antigen binding fragment in retinal pigment epithelium (RPE), retina, aqueous humor or vitreous humor.

143. The method of claim 131, wherein the therapeutically effective amount is sufficient to improve best corrected visual acuity (BCVA) by >=2 Early Treatment Diabetic Retinopathy Study (ETDRS) lines; to decrease the mean rate of change in geographic atrophy as measured by fundus autofluorescence (FAF); to improve visual function as measured by dark adaptation methodology; to improve contrast sensitivity by the Pelli-Robson test; or reduce the drusen area within 10 weeks, 20 weeks, 6 months or 1 year of administration.

144. The method of claim 131, wherein the anti-C5 antibody, or antigen-binding fragment thereof, inhibits both classical and alternative complement pathways and/or inhibits membrane attack complex (MAC) formation.

145. A method of producing recombinant AAVs comprising:
(a) culturing a host cell containing:
(a) an artificial genome comprising a cis expression cassette flanked by AAV ITRs, wherein the cis expression cassette comprises comprising a transgene encoding a full-length anti-C5 antibody or antigen-binding fragment thereof, operably linked to one or more regulatory sequences that promote expression of the transgene in human ocular tissue cells, wherein the anti-C5 antibody comprises i) a heavy chain comprising the amino acid sequence of SEQ ID NO: 1 and a light chain comprising the amino acid sequence of SEQ ID NO: 2 or ii) a heavy chain comprising the amino acid sequence of SEQ ID NO: 3 or SEQ ID NO: 4 and a light chain comprising the amino acid sequence of SEQ ID NO: 5;
(b) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has ocular tissue cell tropism;
(c) sufficient adenovirus helper functions to permit replication and packaging of the artificial genome by the AAV capsid protein; and
(b) recovering recombinant AAV encapsidating the artificial genome from the cell culture.

146. A host cell comprising:
a plasmid comprising a cis expression cassette flanked by AAV ITRs, wherein the cis expression cassette comprises a transgene encoding a full-length anti-C5 antibody, or antigen-binding fragment thereof, operably linked to one or more regulatory sequences that promote expression of the transgene in human ocular tissue cells, wherein the anti-C5 antibody comprises i) a heavy chain comprising the amino acid sequence of SEQ ID NO: 1 and a light chain comprising the amino acid sequence of SEQ ID NO: 2 or ii) a heavy chain comprising the amino acid sequence of SEQ ID NO: 3 or SEQ ID NO: 4 and a light chain comprising the amino acid sequence of SEQ ID NO: 5.


SEQUENCE LISTING (308 sequences)

SEQ ID NO: 1 - protein, 230 aa, synthetic construct
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 2 - protein, 217 aa, synthetic construct
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 230 aa, synthetic construct [sequence omitted]

SEQ ID NO: 4 - protein, 220 aa, synthetic construct
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 5 - protein, 214 aa, synthetic construct [sequence omitted]

SEQ ID NO: 6 - protein, 238 aa, synthetic construct
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAG

SEQ ID NO: 7 - protein, 214 aa, synthetic construct [sequence omitted]

SEQ ID NO: 8 - protein, 247 aa, synthetic construct
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTORKTHLCPPCPAPEAAGGPSVFL

SEQ ID NO: 9 - protein, 214 aa, synthetic construct
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 248 aa, synthetic construct
QVQLVQSGAEVKKPGASVKVSCKASGYTFTDFYMDWVRQAPGQRLEWMGYIYPHNAGTTYNQQFTGRVTITVDKSASTAYMELSSLRSEDTAVYYCARRGGFDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTORKTHLCPPCPAPALAGGPSVFL

SEQ ID NO: 11 - protein, 248 aa, synthetic construct
QVQLVQSGAEVKKPGASVKVSCKASGYTFTDFYMDWVRQAPGQRLEWMGYIYPHNTGTTYNQQFTGRVTITVDKSASTAYMELSSLRSEDTAVYYCARRGGFDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTORKTHLCPPCPAPALAGGPSVFL

SEQ ID NO: 12 - protein, 447 aa, synthetic construct
QVQLVQSGAEVKKPGASVKVSCKASGYTFTDFYMDWVRQAPGQRLEWMGYIYPHNAGTTYNQQFTGRVTITVDKSASTAYMELSSLRSEDTAVYYCARRGGFDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRIPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 107 aa, synthetic construct
DIQMTQSPSSLSASVGDRVTITCKASENVDTYVSWYQQKPGKAPKLLIYGASNRYTGVPSRFSGSGSGTDFTFTISSLQPEDIATYHCGQSHSYPLTFGQGTKLEIK

SEQ ID NO: 14 - protein, 214 aa, synthetic construct
DIQMTQSPSSLSASVGDRVTITCKASENVDTYVSWYQQKPGKAPKLLIYGASNRYTGVPSRFSGSGSGTDFTFTISSLQPEDIATYHCGQSHSYPLTFGQGTKLEIKRTVAAPSVFIFPPSDEQLKSGTASVVCLLNNFYPREAKVQWKVDNALQSGNSQESVTEQDSKDSTYSLSSILTLSKADYEKHKVYACEVTHQGLSSPVTKSFNRGEC

SEQ ID NO: 15 - protein, 445 aa, synthetic construct
QVQLQQPGAELVRPGTSVKLSCKASGYTFTSSWMHWVKQRPGQGLEWIGVIDPSDSYTNYNQKFKGKATLTVDTSSSTAYMQLSSLTSEDSAVYYCARGGGSSYNRYFDVWGTGTTVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 16 - protein, 219 aa, synthetic construct
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 17 - protein, 512 aa, synthetic construct
MYRMQLLLLIALSLALVTNSQVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHRKRRGSGEGRGSLLTCGDVEENPGPMYRMQLLLLIALSLALVTNSDIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 733 aa, synthetic construct
MYRMQLLLLIALSLALVTNSQVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSPRKRRGSGEGRGSLLTCGDVEENPGPMYRMQLLLLIALSLALVTNSDIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 509 aa, synthetic construct
MYRMQLLLLIALSLALVTNSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTRKRRGSGEGRGSLLTCGDVEENPGPMYRMQLLLLIALSLALVTNSDIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 499 aa, synthetic construct
MYRMQLLLLIALSLALVTNSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVRKRRGSGEGRGSLLTCGDVEENPGPMYRMQLLLLIALSLALVTNSDIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 727 aa, synthetic construct
MYRMQLLLLIALSLALVTNSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKRKRRGSGEGRGSLLTCGDVEENPGPMYRMQLLLLIALSLALVTNSDIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 729 aa, synthetic construct
MYRMQLLLLIALSLALVTNSQVQLQQPGAELVRPGTSVKLSCKASGYTFTSSWMHWVKQRPGQGLEWIGVIDPSDSYTNYNQKFKGKATLTVDTSSSTAYMQLSSLTSEDSAVYYCARGGGSSYNRYFDVWGTGTTVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGKRKRRGSGEGRGSLLTCGDVEENPGPMYRMQLLLLIALSLALVTNSNIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 23 - protein, 449 aa, synthetic construct
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVSFTL

SEQ ID NO: 24 - protein, 1231 aa, synthetic construct
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 25 - skipped

SEQ ID NO: 26 - DNA, 690 bp, synthetic construct
  source 1..690: mol_type = other DNA
caggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctgcctctacaaagggcccctctgtgtttcctctggctcctagcagcaagagcaccagtggtggaacagctgccctgggctgtctggtcaaggattacttccctgagccagtgactgtgtcctggaactctggtgcactgacctctggggtgcacacctttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccac

SEQ ID NO: 27 - DNA, 651 bp, synthetic construct
  source 1..651: mol_type = other DNA
gacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagaacagtggctgccccttctgtgttcatcttcccaccatctgatgagcagctgaagagtggcacagcctctgttgtgtgcctgctgaacaacttctaccccagagaagccaaggtgcagtggaaggtggacaatgccctgcagtcaggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgc

SEQ ID NO: 28 - DNA, 690 bp, synthetic construct
  source 1..690: mol_type = other DNA
caggtgcagctggttcagtctggggctgaagtgaagaaacctggggcctctgtgaaggtgtcctgcaaggcctctggctacatcttcagcaactattggatccagtgggtcagacaggcccctggccaaggacttgagtggatgggagagatcctgcctggctctggcagcacagagtacacagagaacttcaaggacagagtgaccatgaccagagacaccagcacctccacagtgtacatggaactgagcagcctgagatctgaggacacagcagtgtactattgtgccagatacttctttggcagcagccccaactggtactttgatgtgtggggccagggcaccctggttacagtgtcctctgcctctacaaagggcccctctgtgtttcctctggctcctagcagcaagagcaccagtggtggaacagctgccctgggctgtctggtcaaggattacttccctgagcctgtgactgtgtcctggaactctggtgcactgacctctggggtgcacacctttccagctgtgctgcagtcctctggcctgtactctctgtcctctgtggtcacagtgcctagctctagcctgggcacccagacctacatctgcaatgtgaaccacaagcctagcaacaccaaggtggacaagagagtggaacccaagagctgtgacaagacccacacc

SEQ ID NO: 29 - DNA, 660 bp, synthetic construct
  source 1..660: mol_type = other DNA
caggtgcagctggttcagtctggggctgaagtgaagaaacctggggcctctgtgaaggtgtcctgcaaggcctctggctacatcttcagcaactactggattcagtgggtcagacaggcccctggccaaggacttgagtggatgggagagatcctgcctggctctggcagcacagagtacacagagaacttcaaggacagagtgaccatgaccagagacaccagcacctccacagtgtacatggaactgagcagcctgagatctgaggacacagcagtgtactattgtgccagatacttctttggcagcagccccaactggtactttgatgtgtggggccagggcaccctggttacagtgtcctctgcctctacaaagggcccctctgtgttccctctggctccttgtagcagaagcacctctgagtctacagctgccctgggctgcctggtcaaggattactttcctgagcctgtgactgtgtcctggaactctggtgctctgacctctggggtgcacacctttccagctgtgctgcagtcctctggcctgtactctctgtcctctgtggtcacagtgcccagcagcaattttggcacccagacctacacctgtaatgtggaccacaagcctagcaacaccaaggtggacaagacagtg

SEQ ID NO: 30 - DNA, 642 bp, synthetic construct
  source 1..642: mol_type = other DNA
gacatccagatgacacagagccctagcagcctgtctgcctctgtgggagacagagtgacaatcacctgtggggcctctgagaacatctatggggccctgaactggtatcagcagaagcctggaaaggcccctaagctgctgatatatggggccaccaatctggctgatggggtgccctctagattttctggcagtggctctggcacagacttcaccctgaccatcagtagcctgcagcctgaggactttgccacctactactgtcagaatgtgctgaacacccctctgacctttggccagggcaccaaggtggaaatcaagagaacagtggctgccccttctgtattcatcttcccaccatctgatgagcagctgaagagtggcacagcctctgttgtgtgcctgctgaacaacttctaccccagagaagccaaggtgcagtggaaggtggacaatgccctgcagtctggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgc

SEQ ID NO: 31 - DNA, 714 bp, synthetic construct
  source 1..714: mol_type = other DNA
caggtgcagctggtgcagagcggcgcggaagtgaaaaaaccgggcgcgagcgtgaaagtgagctgcaaagcgagcggccatatttttagcaactattggattcagtgggtgcgccaggcgccgggccagggcctggaatggatgggcgaaattctgccgggcagcggccataccgaatataccgaaaactttaaagatcgcgtgaccatgacccgcgataccagcaccagcaccgtgtatatggaactgagcagcctgcgcagcgaagataccgcggtgtattattgcgcgcgctatttttttggcagcagcccgaactggtattttgatgtgtggggccagggcaccctggtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcaactttggcacccagacctatacctgcaacgtggatcataaaccgagcaacaccaaagtggataaaaccgtggaacgcaaatgctgcgtggaatgccccccctgccccgccccccccgtggccggc

SEQ ID NO: 32 - DNA, 642 bp, synthetic construct
  source 1..642: mol_type = other DNA
gatattcagatgacccagagcccgagcagcctgagcgcgagcgtgggcgatcgcgtgaccattacctgcggcgcgagcgaaaacatttatggcgcgctgaactggtatcagcagaaaccgggcaaagcgccgaaactgctgatttatggcgcgaccaacctggcggatggcgtgccgagccgctttagcggcagcggcagcggcaccgattttaccctgaccattagcagcctgcagccggaagattttgcgacctattattgccagaacgtgctgaacaccccgctgacctttggccagggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgc

SEQ ID NO: 33 - DNA, 735 bp, synthetic construct
  source 1..735: mol_type = other DNA
gaggtgcagctggtgcagagcggcgccgaggtgaagaagcccggcagcagcgtgaaggtgagctgcaaggccagcggcggcaccttcagcagctacgccatcagctgggtgaggcaggcccccggccagggcctggagtggatgggcggcatcggccccttcttcggcaccgccaactacgcccagaagttccagggcagggtgaccatcaccgccgacgagagcaccagcaccgcctacatggagctgagcagcctgaggagcgaggacaccgccgtgtactactgcgccagggacaccccctacttcgactactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagggtggagcccaagagctgcgacaagacccacaccaagacccacctgtgccccccctgccccgcccccgaggccgccggcggccccagcgtgttcctg

SEQ ID NO: 34 - DNA, 642 bp, synthetic construct
  source 1..642: mol_type = other DNA
agctacgagctgacccagcccctgagcgtgagcgtggccctgggccagaccgccaggatcacctgcagcggcgacagcatccccaactactacgtgtactggtaccagcagaagcccggccaggcccccgtgctggtgatctacgacgacagcaacaggcccagcggcatccccgagaggttcagcggcagcaacagcggcaacaccgccaccctgaccatcagcagggcccaggccggcgacgaggccgactactactgccagagcttcgacagcagcctgaacgccgaggtgttcggcggcggcaccaagctgaccgtgctgggccagcccaaggccgcccccagcgtgaccctgttcccccccagcagcgaggagctgcaggccaacaaggccaccctggtgtgcctgatcagcgacttctaccccggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggagctacagctgccaggtgacccacgagggcagcaccgtggagaagaccgtggcccccaccgagtgcagc

SEQ ID NO: 35 - DNA, 1335 bp, synthetic construct
  source 1..1335: mol_type = other DNA
caggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctctgccaagacaacccctccttctgtgtaccctctggctcctggatctgctgcccagaccaactctatggtcacactgggctgtctggtcaagggctacttccctgagcctgtgacagtgacctggaactctggatctctgtcctctggggtgcacacattccctgcagtgctgcagtctgatctgtacaccctgagcagctctgtgactgtgcctagcagccccagaccttctgagactgtgacctgcaatgtggctcaccctgccagcagcaccaaggtggacaagaaaattgtgcccagagactgtggctgcaagccctgcatctgtacagtgcctgaggtttcctctgtgttcatcttcccacctaagcctaaggatgtgctgaccatcacactgacccctaaagtgacctgtgtggtggtggacatcagcaaggatgaccctgaggtgcagttctcttggtttgtggatgatgtggaagtgcacacagcccagacacagcctagagaggaacagttcaacagcaccttcagatcagtgtctgagctgcccatcatgcaccaggactggctgaatggcaaagagttcaagtgcagagtgaactctgctgctttccctgctcctattgaaaagaccatctccaagaccaagggcagacccaaggctccccaggtgtacacaatccctccacctaaagaacagatggccaaggacaaggtgtccctgacctgcatgatcacagatttcttcccagaggatatcacagttgagtggcagtggaatggccagcctgctgagaactacaagaacacccagcctatcatgaacaccaatggcagctactttgtgtacagcaagctgaatgtgcagaagtccaactgggaggctggcaacacctttacctgctctgtgctgcatgagggcctgcacaaccaccatacagagaagtccctgtctcacagccctggcaag

SEQ ID NO: 36 - DNA, 657 bp, synthetic construct
  source 1..657: mol_type = other DNA
aatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagagggctgatgctgcccctacagtgtctatcttcccacctagctctgagcagctgaccagtggtggtgcctctgttgtgtgcttcctgaacaacttctaccccaaggacatcaatgtgaagtggaagattgatggctctgagaggcagaatggggtgctgaacagctggacagaccaggacagcaaggactccacctacagcatgagcagcacactgaccctgaccaaggatgagtatgagaggcacaacagctacacctgtgaagccacacacaagaccagcacaagccccattgtgaagtccttcaacaggggagagtgc

SEQ ID NO: 37 - DNA, 1539 bp, synthetic construct
  source 1..1539: mol_type = other DNA
atgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctgcctctacaaagggcccctctgtgtttcctctggctcctagcagcaagagcaccagtggtggaacagctgccctgggctgtctggtcaaggattacttccctgagccagtgactgtgtcctggaactctggtgcactgacctctggggtgcacacctttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagaacagtggctgccccttctgtgttcatcttcccaccatctgatgagcagctgaagagtggcacagcctctgttgtgtgcctgctgaacaacttctaccccagagaagccaaggtgcagtggaaggtggacaatgccctgcagtcaggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgctga

SEQ ID NO: 38 - DNA, 2202 bp, synthetic construct
  source 1..2202: mol_type = other DNA
atgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctgcctctacaaagggcccctctgtgtttcctctggctcctagcagcaagagcaccagtggtggaacagctgccctgggctgtctggtcaaggattacttccctgagccagtgactgtgtcctggaactctggtgcactgacctctggggtgcacacctttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacacctgtcctccatgtcctgctccagagctgagaagaggccccaaggtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtgtctgtgctgacagtgctgcatcaggactggctgaatggcaaagagtacaagtgcaaggtgtccaacaagggcctgccaagcagcattgagaaaaccatctccaaggccaaggggcagcccagagaacctcaggtttacaccctgcctccaagcagggaagagatgaccaagaatcaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcagggcaatgtgttcagctgcagtgtgctgcatgaagccctgcatgcccactacaccagaaaagagctgtctctgagccccagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagaacagtggctgccccttctgtgttcatcttcccaccatctgatgagcagctgaagagtggcacagcctctgttgtgtgcctgctgaacaacttctaccccagagaagccaaggtgcagtggaaggtggacaatgccctgcagtcaggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgctga

SEQ ID NO: 39 - DNA, 1530 bp, synthetic construct
  source 1..1530: mol_type = other DNA
atgtacagaatgcagctgctgctgctcattgccctgtctctggccctggtcaccaattctcaggtgcagctggttcagtctggggctgaagtgaagaaacctggggcctctgtgaaggtgtcctgcaaggcctctggctacatcttcagcaactattggatccagtgggtcagacaggcccctggccaaggacttgagtggatgggagagatcctgcctggctctggcagcacagagtacacagagaacttcaaggacagagtgaccatgaccagagacaccagcacctccacagtgtacatggaactgagcagcctgagatctgaggacacagcagtgtactattgtgccagatacttctttggcagcagccccaactggtactttgatgtgtggggccagggcaccctggttacagtgtcctctgcctctacaaagggcccctctgtgtttcctctggctcctagcagcaagagcaccagtggtggaacagctgccctgggctgtctggtcaaggattacttccctgagcctgtgactgtgtcctggaactctggtgcactgacctctggggtgcacacctttccagctgtgctgcagtcctctggcctgtactctctgtcctctgtggtcacagtgcctagctctagcctgggcacccagacctacatctgcaatgtgaaccacaagcctagcaacaccaaggtggacaagagagtggaacccaagagctgtgacaagacccacaccagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcctctgtgggagacagagtgacaatcacctgtggggcctctgagaacatctatggggccctgaactggtatcagcagaagcctggaaaggcccctaagctgctgatatatggggccaccaatctggctgatggggtgccctctagattttctggcagtggctctggcacagacttcaccctgaccatcagtagcctgcagcctgaggactttgccacctactactgtcagaatgtgctgaacacccctctgacctttggccagggcaccaaggtggaaatcaagagaacagtggctgccccttctgtattcatcttcccaccatctgatgagcagctgaagagtggcacagcctctgttgtgtgcctgctgaacaacttctaccccagagaagccaaggtgcagtggaaggtggacaatgccctgcagtctggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgctga

SEQ ID NO: 40 - DNA, 1500 bp, synthetic construct
  source 1..1500: mol_type = other DNA
atgtacagaatgcagctgctgctgctcattgccctgtctctggccctggtcaccaattctcaggtgcagctggttcagtctggggctgaagtgaagaaacctggggcctctgtgaaggtgtcctgcaaggcctctggctacatcttcagcaactactggattcagtgggtcagacaggcccctggccaaggacttgagtggatgggagagatcctgcctggctctggcagcacagagtacacagagaacttcaaggacagagtgaccatgaccagagacaccagcacctccacagtgtacatggaactgagcagcctgagatctgaggacacagcagtgtactattgtgccagatacttctttggcagcagccccaactggtactttgatgtgtggggccagggcaccctggttacagtgtcctctgcctctacaaagggcccctctgtgttccctctggctccttgtagcagaagcacctctgagtctacagctgccctgggctgcctggtcaaggattactttcctgagcctgtgactgtgtcctggaactctggtgctctgacctctggggtgcacacctttccagctgtgctgcagtcctctggcctgtactctctgtcctctgtggtcacagtgcccagcagcaattttggcacccagacctacacctgtaatgtggaccacaagcctagcaacaccaaggtggacaagacagtgagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcctctgtgggagacagagtgacaatcacctgtggggcctctgagaacatctatggggccctgaactggtatcagcagaagcctggaaaggcccctaagctgctgatatatggggccaccaatctggctgatggggtgccctctagattttctggcagtggctctggcacagacttcaccctgaccatcagtagcctgcagcctgaggactttgccacctactactgtcagaatgtgctgaacacccctctgacctttggccagggcaccaaggtggaaatcaagagaacagtggctgccccttctgtattcatcttcccaccatctgatgagcagctgaagagtggcacagcctctgttgtgtgcctgctgaacaacttctaccccagagaagccaaggtgcagtggaaggtggacaatgccctgcagtctggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgctga

SEQ ID NO: 41 - DNA, 2184 bp, synthetic construct
  source 1..2184: mol_type = other DNA
atgtacagaatgcagctgctgctgctcattgccctgtctctggccctggtcaccaattctcaggtgcagctggttcagtctggggctgaagtgaagaaacctggggcctctgtgaaggtgtcctgcaaggcctctggctacatcttcagcaactactggattcagtgggtcagacaggcccctggccaaggacttgagtggatgggagagatcctgcctggctctggcagcacagagtacacagagaacttcaaggacagagtgaccatgaccagagacaccagcacctccacagtgtacatggaactgagcagcctgagatctgaggacacagcagtgtactattgtgccagatacttctttggcagcagccccaactggtactttgatgtgtggggccagggcaccctggttacagtgtcctctgcctctacaaagggcccctctgtgttccctctggctccttgtagcagaagcacctctgagtctacagctgccctgggctgcctggtcaaggattactttcctgagcctgtgactgtgtcctggaactctggtgctctgacctctggggtgcacacctttccagctgtgctgcagtcctctggcctgtactctctgtcctctgtggtcacagtgcccagcagcaattttggcacccagacctacacctgtaatgtggaccacaagcctagcaacaccaaggtggacaagacagtggaaagaaagtgctgtgtggaatgccctccttgtcctgctcctccagtggctggaccttctgtgtttctgttccctccaaagcctaaggacaccctgatgatcagcagaacccctgaagtgacctgtgtggtggtggatgtgtcccaagaggaccctgaggtgcagttcaattggtatgtggatggggttgaagtgcacaatgccaagaccaagcctagagaggaacagttcaacagcacctacagagtggtgtctgtgctgacagtgctgcaccaggactggctgaatggcaaagagtacaagtgcaaggtgtccaacaagggcctgcctagcagcattgagaaaaccatcagcaaggccaagggccagccaagagaaccccaagtgtacaccctgcctccaagccaagaggaaatgaccaagaaccaggtgtccctgacctgccttgtgaagggcttctacccctctgacattgctgtggaatgggagtccaatggccagcctgaaaacaactacaagaccacacctcctgtgctggactctgatggcagcttcttcctgtacagcagactgacagtggacaagtccagatggcaagagggcaatgtgttcagctgctctgtgatgcatgaggccctgcacaaccactacacccagaagtctctgtctctgagcctgggcaagagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcctctgtgggagacagagtgacaatcacctgtggggcctctgagaacatctatggggccctgaactggtatcagcagaagcctggaaaggcccctaagctgctgatatatggggccaccaatctggctgatggggtgccctctagattttctggcagtggctctggcacagacttcaccctgaccatcagtagcctgcagcctgaggactttgccacctactactgtcagaatgtgctgaacacccctctgacctttggccagggcaccaaggtggaaatcaagagaacagtggctgccccttctgtattcatcttcccaccatctgatgagcagctgaagagtggcacagcctctgttgtgtgcctgctgaacaacttctaccccagagaagccaaggtgcagtggaaggtggacaatgccctgcagtctggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgctga

SEQ ID NO: 42 - DNA, 2190 bp, synthetic construct
  source 1..2190: mol_type = other DNA
atgtacagaatgcagctgctgctgctcattgccctgtctctggccctggtcaccaattctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctctgccaagacaacccctccttctgtgtaccctctggctcctggatctgctgcccagaccaactctatggtcacactgggctgtctggtcaagggctacttccctgagcctgtgacagtgacctggaactctggatctctgtcctctggggtgcacacattccctgcagtgctgcagtctgatctgtacaccctgagcagctctgtgactgtgcctagcagccccagaccttctgagactgtgacctgcaatgtggctcaccctgccagcagcaccaaggtggacaagaaaattgtgcccagagactgtggctgcaagccctgcatctgtacagtgcctgaggtttcctctgtgttcatcttcccacctaagcctaaggatgtgctgaccatcacactgacccctaaagtgacctgtgtggtggtggacatcagcaaggatgaccctgaggtgcagttctcttggtttgtggatgatgtggaagtgcacacagcccagacacagcctagagaggaacagttcaacagcaccttcagatcagtgtctgagctgcccatcatgcaccaggactggctgaatggcaaagagttcaagtgcagagtgaactctgctgctttccctgctcctattgaaaagaccatctccaagaccaagggcagacccaaggctccccaggtgtacacaatccctccacctaaagaacagatggccaaggacaaggtgtccctgacctgcatgatcacagatttcttcccagaggatatcacagttgagtggcagtggaatggccagcctgctgagaactacaagaacacccagcctatcatgaacaccaatggcagctactttgtgtacagcaagctgaatgtgcagaagtccaactgggaggctggcaacacctttacctgctctgtgctgcatgagggcctgcacaaccaccatacagagaagtccctgtctcacagccctggcaagagaaagagaagaggctctggagaaggcagaggctccctgctgacatgtggggatgttgaagagaatcctgggcctatgtataggatgcaactgctcctcctgattgctctgagcctggctcttgtgaccaactctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagagggctgatgctgcccctacagtgtctatcttcccacctagctctgagcagctgaccagtggtggtgcctctgttgtgtgcttcctgaacaacttctaccccaaggacatcaatgtgaagtggaagattgatggctctgagaggcagaatggggtgctgaacagctggacagaccaggacagcaaggactccacctacagcatgagcagcacactgaccctgaccaaggatgagtatgagaggcacaacagctacacctgtgaagccacacacaagaccagcacaagccccattgtgaagtccttcaacaggggagagtgctga

SEQ ID NO: 43 - DNA, 3856 bp, synthetic construct
  source 1..3856: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctgcctctacaaagggcccctctgtgtttcctctggctcctagcagcaagagcaccagtggtggaacagctgccctgggctgtctggtcaaggattacttccctgagccagtgactgtgtcctggaactctggtgcactgacctctggggtgcacacctttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagaacagtggctgccccttctgtgttcatcttcccaccatctgatgagcagctgaagagtggcacagcctctgttgtgtgcctgctgaacaacttctaccccagagaagccaaggtgcagtggaaggtggacaatgccctgcagtcaggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 44 - DNA, 4519 bp, synthetic construct
  source 1..4519: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctgcctctacaaagggcccctctgtgtttcctctggctcctagcagcaagagcaccagtggtggaacagctgccctgggctgtctggtcaaggattacttccctgagccagtgactgtgtcctggaactctggtgcactgacctctggggtgcacacctttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacacctgtcctccatgtcctgctccagagctgagaagaggccccaaggtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtgtctgtgctgacagtgctgcatcaggactggctgaatggcaaagagtacaagtgcaaggtgtccaacaagggcctgccaagcagcattgagaaaaccatctccaaggccaaggggcagcccagagaacctcaggtttacaccctgcctccaagcagggaagagatgaccaagaatcaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcagggcaatgtgttcagctgcagtgtgctgcatgaagccctgcatgcccactacaccagaaaagagctgtctctgagccccagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagaacagtggctgccccttctgtgttcatcttcccaccatctgatgagcagctgaagagtggcacagcctctgttgtgtgcctgctgaacaacttctaccccagagaagccaaggtgcagtggaaggtggacaatgccctgcagtcaggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 45 - DNA, 3847 bp, synthetic construct
  source 1..3847: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctcaggtgcagctggttcagtctggggctgaagtgaagaaacctggggcctctgtgaaggtgtcctgcaaggcctctggctacatcttcagcaactattggatccagtgggtcagacaggcccctggccaaggacttgagtggatgggagagatcctgcctggctctggcagcacagagtacacagagaacttcaaggacagagtgaccatgaccagagacaccagcacctccacagtgtacatggaactgagcagcctgagatctgaggacacagcagtgtactattgtgccagatacttctttggcagcagccccaactggtactttgatgtgtggggccagggcaccctggttacagtgtcctctgcctctacaaagggcccctctgtgtttcctctggctcctagcagcaagagcaccagtggtggaacagctgccctgggctgtctggtcaaggattacttccctgagcctgtgactgtgtcctggaactctggtgcactgacctctggggtgcacacctttccagctgtgctgcagtcctctggcctgtactctctgtcctctgtggtcacagtgcctagctctagcctgggcacccagacctacatctgcaatgtgaaccacaagcctagcaacaccaaggtggacaagagagtggaacccaagagctgtgacaagacccacaccagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcctctgtgggagacagagtgacaatcacctgtggggcctctgagaacatctatggggccctgaactggtatcagcagaagcctggaaaggcccctaagctgctgatatatggggccaccaatctggctgatggggtgccctctagattttctggcagtggctctggcacagacttcaccctgaccatcagtagcctgcagcctgaggactttgccacctactactgtcagaatgtgctgaacacccctctgacctttggccagggcaccaaggtggaaatcaagagaacagtggctgccccttctgtattcatcttcccaccatctgatgagcagctgaagagtggcacagcctctgttgtgtgcctgctgaacaacttctaccccagagaagccaaggtgcagtggaaggtggacaatgccctgcagtctggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 46 - DNA, 3817 bp, synthetic construct
  source 1..3817: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctcaggtgcagctggttcagtctggggctgaagtgaagaaacctggggcctctgtgaaggtgtcctgcaaggcctctggctacatcttcagcaactactggattcagtgggtcagacaggcccctggccaaggacttgagtggatgggagagatcctgcctggctctggcagcacagagtacacagagaacttcaaggacagagtgaccatgaccagagacaccagcacctccacagtgtacatggaactgagcagcctgagatctgaggacacagcagtgtactattgtgccagatacttctttggcagcagccccaactggtactttgatgtgtggggccagggcaccctggttacagtgtcctctgcctctacaaagggcccctctgtgttccctctggctccttgtagcagaagcacctctgagtctacagctgccctgggctgcctggtcaaggattactttcctgagcctgtgactgtgtcctggaactctggtgctctgacctctggggtgcacacctttccagctgtgctgcagtcctctggcctgtactctctgtcctctgtggtcacagtgcccagcagcaattttggcacccagacctacacctgtaatgtggaccacaagcctagcaacaccaaggtggacaagacagtgagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcctctgtgggagacagagtgacaatcacctgtggggcctctgagaacatctatggggccctgaactggtatcagcagaagcctggaaaggcccctaagctgctgatatatggggccaccaatctggctgatggggtgccctctagattttctggcagtggctctggcacagacttcaccctgaccatcagtagcctgcagcctgaggactttgccacctactactgtcagaatgtgctgaacacccctctgacctttggccagggcaccaaggtggaaatcaagagaacagtggctgccccttctgtattcatcttcccaccatctgatgagcagctgaagagtggcacagcctctgttgtgtgcctgctgaacaacttctaccccagagaagccaaggtgcagtggaaggtggacaatgccctgcagtctggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 47 - DNA, 4501 bp, synthetic construct
  source 1..4501: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctcaggtgcagctggttcagtctggggctgaagtgaagaaacctggggcctctgtgaaggtgtcctgcaaggcctctggctacatcttcagcaactactggattcagtgggtcagacaggcccctggccaaggacttgagtggatgggagagatcctgcctggctctggcagcacagagtacacagagaacttcaaggacagagtgaccatgaccagagacaccagcacctccacagtgtacatggaactgagcagcctgagatctgaggacacagcagtgtactattgtgccagatacttctttggcagcagccccaactggtactttgatgtgtggggccagggcaccctggttacagtgtcctctgcctctacaaagggcccctctgtgttccctctggctccttgtagcagaagcacctctgagtctacagctgccctgggctgcctggtcaaggattactttcctgagcctgtgactgtgtcctggaactctggtgctctgacctctggggtgcacacctttccagctgtgctgcagtcctctggcctgtactctctgtcctctgtggtcacagtgcccagcagcaattttggcacccagacctacacctgtaatgtggaccacaagcctagcaacaccaaggtggacaagacagtggaaagaaagtgctgtgtggaatgccctccttgtcctgctcctccagtggctggaccttctgtgtttctgttccctccaaagcctaaggacaccctgatgatcagcagaacccctgaagtgacctgtgtggtggtggatgtgtcccaagaggaccctgaggtgcagttcaattggtatgtggatggggttgaagtgcacaatgccaagaccaagcctagagaggaacagttcaacagcacctacagagtggtgtctgtgctgacagtgctgcaccaggactggctgaatggcaaagagtacaagtgcaaggtgtccaacaagggcctgcctagcagcattgagaaaaccatcagcaaggccaagggccagccaagagaaccccaagtgtacaccctgcctccaagccaagaggaaatgaccaagaaccaggtgtccctgacctgccttgtgaagggcttctacccctctgacattgctgtggaatgggagtccaatggccagcctgaaaacaactacaagaccacacctcctgtgctggactctgatggcagcttcttcctgtacagcagactgacagtggacaagtccagatggcaagagggcaatgtgttcagctgctctgtgatgcatgaggccctgcacaaccactacacccagaagtctctgtctctgagcctgggcaagagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcctctgtgggagacagagtgacaatcacctgtggggcctctgagaacatctatggggccctgaactggtatcagcagaagcctggaaaggcccctaagctgctgatatatggggccaccaatctggctgatggggtgccctctagattttctggcagtggctctggcacagacttcaccctgaccatcagtagcctgcagcctgaggactttgccacctactactgtcagaatgtgctgaacacccctctgacctttggccagggcaccaaggtggaaatcaagagaacagtggctgccccttctgtattcatcttcccaccatctgatgagcagctgaagagtggcacagcctctgttgtgtgcctgctgaacaacttctaccccagagaagccaaggtgcagtggaaggtggacaatgccctgcagtctggcaacagccaagagtctgtgactgagcaggacagcaaggactccacctacagcctgagcagcaccctgacactgagcaaggctgactatgagaagcacaaagtgtatgcctgtgaagtgacccaccaggggctgagcagccctgtgaccaagagcttcaatagaggggagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 48 - DNA, 4507 bp, synthetic construct
  source 1..4507: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctctgccaagacaacccctccttctgtgtaccctctggctcctggatctgctgcccagaccaactctatggtcacactgggctgtctggtcaagggctacttccctgagcctgtgacagtgacctggaactctggatctctgtcctctggggtgcacacattccctgcagtgctgcagtctgatctgtacaccctgagcagctctgtgactgtgcctagcagccccagaccttctgagactgtgacctgcaatgtggctcaccctgccagcagcaccaaggtggacaagaaaattgtgcccagagactgtggctgcaagccctgcatctgtacagtgcctgaggtttcctctgtgttcatcttcccacctaagcctaaggatgtgctgaccatcacactgacccctaaagtgacctgtgtggtggtggacatcagcaaggatgaccctgaggtgcagttctcttggtttgtggatgatgtggaagtgcacacagcccagacacagcctagagaggaacagttcaacagcaccttcagatcagtgtctgagctgcccatcatgcaccaggactggctgaatggcaaagagttcaagtgcagagtgaactctgctgctttccctgctcctattgaaaagaccatctccaagaccaagggcagacccaaggctccccaggtgtacacaatccctccacctaaagaacagatggccaaggacaaggtgtccctgacctgcatgatcacagatttcttcccagaggatatcacagttgagtggcagtggaatggccagcctgctgagaactacaagaacacccagcctatcatgaacaccaatggcagctactttgtgtacagcaagctgaatgtgcagaagtccaactgggaggctggcaacacctttacctgctctgtgctgcatgagggcctgcacaaccaccatacagagaagtccctgtctcacagccctggcaagagaaagagaagaggctctggagaaggcagaggctccctgctgacatgtggggatgttgaagagaatcctgggcctatgtataggatgcaactgctcctcctgattgctctgagcctggctcttgtgaccaactctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagagggctgatgctgcccctacagtgtctatcttcccacctagctctgagcagctgaccagtggtggtgcctctgttgtgtgcttcctgaacaacttctaccccaaggacatcaatgtgaagtggaagattgatggctctgagaggcagaatggggtgctgaacagctggacagaccaggacagcaaggactccacctacagcatgagcagcacactgaccctgaccaaggatgagtatgagaggcacaacagctacacctgtgaagccacacacaagaccagcacaagccccattgtgaagtccttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 49 - DNA, 892 bp, synthetic construct
  source 1..892: mol_type = other DNA
gaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctga

SEQ ID NO: 50 - DNA, 3679 bp, synthetic construct
  source 1..3679: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgccgccctggatatagatctcttggaaatgtaataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattaccgtgaatgtgacacagatggatggaccaatgatattcctatatgtgaagttgtgaagtgtttaccagtgacagcaccagagaatggaaaaattgtcagtagtgcaatggaaccagatcgggaataccattttggacaagcagtacggtttgtatgtaactcaggctacaagattgaaggagatgaagaaatgcattgttcagacgatggtttttggagtaaagagaaaccaaagtgtgtggaaatttcatgcaaatccccagatgttataaatggatctcctatatctcagaagattatttataaggagaatgaacgatttcaatataaatgtaacatgggttatgaatacagtgaaagaggagatgctgtatgcactgaatctggatggcgtccgttgccttcatgtgaagaaaaatcatgtgataatccttatattccaaatggtgactactcacctttaaggattaaacacagaactggagatgaaatcacgtaccagtgtagaaatggtttttatcctgcaacccggggaaatacagcaaaatgcacaagtactggctggatacctgctccgagatgtaccttgaaaccttgtgattatccagacattaaacatggaggtctatatcatgagaatatgcgtagaccatactttccagtagctgtaggaaaatattactcctattactgtgatgaacattttgagactccgtcaggaagttactgggatcacattcattgcacacaagatggatggtcgccagcagtaccatgcctcagaaaatgttattttccttatttggaaaatggatataatcaaaatcatggaagaaagtttgtacagggtaaatctatagacgttgcctgccatcctggctacgctcttccaaaagcgcagaccacagttacatgtatggagaatggctggtctcctactcccagatgcatccgtgtcagctttaccctctgataactcgaggacggggtgaactgcagctacgcctgagctcgacgatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 51 - protein, 5 aa, synthetic construct
GGGGS

SEQ ID NO: 52 - protein, 10 aa, synthetic construct
GGGGSGGGGS

SEQ ID NO: 53 - protein, 15 aa, synthetic construct
GGGGSGGGGSGGGGS

SEQ ID NO: 54 - protein, 20 aa, synthetic construct
GGGGSGGGGSGGGGSGGGGS

SEQ ID NO: 55 - protein, 25 aa, synthetic construct
GGGGSGGGGSGGGGSGGGGSGGGGS

SEQ ID NO: 56-60 - skipped (5 entries)

SEQ ID NO: 61 - protein, 205 aa, synthetic construct
FPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 205 aa, synthetic construct
FPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 63 - protein, 205 aa, synthetic construct
FPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 203 aa, synthetic construct
FPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 65 - protein, 205 aa, synthetic construct; identical to SEQ ID NO: 63

SEQ ID NO: 66 - protein, 205 aa, synthetic construct
FPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 67 - protein, 205 aa, synthetic construct
FPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 205 aa, synthetic construct
FPPKPKDTLMISRIPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69-72 - skipped (4 entries)

SEQ ID NO: 73 - DNA, 1725 bp, synthetic construct
  source 1..1725: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 74 - DNA, 1676 bp, synthetic construct
  source 1..1676: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 75 - DNA, 251 bp, synthetic construct
  source 1..251: mol_type = other DNA
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatact

SEQ ID NO: 76 - DNA, 212 bp, synthetic construct
  source 1..212: mol_type = other DNA
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 77 - DNA, 295 bp, synthetic construct
  source 1..295: mol_type = other DNA
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggc

SEQ ID NO: 78 - DNA, 127 bp, synthetic construct
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 79 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 80 - DNA, 82 bp, synthetic construct
  source 1..82: mol_type = other DNA
gtgagtatctcagggatccagacatggggatatgggaggtgcctctgatcccagggctcactgtgggtctctctgttcacag

SEQ ID NO: 81 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 82 - DNA, 106 bp, synthetic construct
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 83 - DNA, 143 bp, synthetic construct
  source 1..143: mol_type = other DNA
gaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 84 - DNA, 110 bp, synthetic construct
  source 1..110: mol_type = other DNA
ttggccactccctctctgcgcgctcgctcgctcactgaggccgggcgaccaaaggtcgcccgacgcccgggctttgcccgggcggcctcagtgagcgagcgagcgcgcag

SEQ ID NO: 85 - protein, 20 aa, synthetic construct
MYRMQLLLLIALSLALVTNS

SEQ ID NO: 86 - DNA, 60 bp, synthetic construct
  source 1..60: mol_type = other DNA
atgtataggatgcaactgctcctcctgattgctctgagcctggctcttgtgaccaactct

SEQ ID NO: 87 - protein, 26 aa, synthetic construct
MNFLLSWVHWSLALLLYLHHAKWSQA

SEQ ID NO: 88 - protein, 29 aa, synthetic construct
MERAAPSRRVPLPLLLLGGLALLAAGVDA

SEQ ID NO: 89 - protein, 19 aa, synthetic construct
MAPLRPLLILALLAWVALA

SEQ ID NO: 90 - protein, 18 aa, synthetic construct
MRLLAKIICLMLWAICVA

SEQ ID NO: 91 - protein, 19 aa, synthetic construct
MRLLAFLSLLALVLQETGT

SEQ ID NO: 92 - protein, 18 aa, synthetic construct
MKWVTFISLLFLFSSAYS

SEQ ID NO: 93 - protein, 18 aa, synthetic construct
MAFLWLLSCWALLGTTFG

SEQ ID NO: 94 - protein, 20 aa, synthetic construct
MYRMQLLSCIALILALVTNS

SEQ ID NO: 95 - protein, 15 aa, synthetic construct
MNLLLILTFVAAAVA

SEQ ID NO: 96 - protein, 18 aa, synthetic construct
MKWVTFISLLFLFSSAYS

SEQ ID NO: 97 - protein, 24 aa, synthetic construct
MPSSVSWGILLLAGLCCLVPVSLA

SEQ ID NO: 98 - protein, 18 aa, synthetic construct
MKAAVLTLAVLFLTGSQA

SEQ ID NO: 99 - protein, 18 aa, synthetic construct
MKLLAATVLLLTICSLEG

SEQ ID NO: 100 - protein, 27 aa, synthetic construct
MDPPRPALLALLALPALLLLLLAGARA

SEQ ID NO: 101 - protein, 28 aa, synthetic construct
MQRVNMIMAESPGLITICLLGYLLSAEC

SEQ ID NO: 102 - protein, 20 aa, synthetic construct
MGPLMVLFCLLFLYPGLADS

SEQ ID NO: 103 - protein, 18 aa, synthetic construct
MWLLVSVILISRISSVGG

SEQ ID NO: 104 - protein, 18 aa, synthetic construct
MLLLFSVILISWVSTVGG

SEQ ID NO: 105 - protein, 19 aa, synthetic construct
MFSMRIVCLVLSVVGTAWT

SEQ ID NO: 106 - protein, 30 aa, synthetic construct
MKRMVSWSFHKLKTMKHLLLLLLCVFLVKS

SEQ ID NO: 107 - protein, 26 aa, synthetic construct
MSWSLHPRNLILYFYALLFLSSTCVA

SEQ ID NO: 108 - protein, 18 aa, synthetic construct
MKSLVLLLCLAQLWGCHS

SEQ ID NO: 109 - protein, 23 aa, synthetic construct
MARVLGAPVALGLWSLCWSLAIA

SEQ ID NO: 110 - protein, 18 aa, synthetic construct
MKLITILFLCSRLLLSLT

SEQ ID NO: 111 - protein, 20 aa, synthetic construct
MSLFPSLPLLLLSMVAASYS

SEQ ID NO: 112 - protein, 19 aa, synthetic construct
MEHKEVVLLLLLFLKSGQG

SEQ ID NO: 113 - protein, 24 aa, synthetic construct
MAHVRGLQLPGCLALAALCSLVHS

SEQ ID NO: 114 - protein, 29 aa, synthetic construct
MISRMEKMTMMMKILIMFALGMNYWSCSG

SEQ ID NO: 115 - protein, 32 aa, synthetic construct
MYSNVIGTVTSGKRKVYLLSLLLIGFWDCVTC

SEQ ID NO: 116 - protein, 19 aa, synthetic construct
MRLAVGALLVCAVLGLCLA

SEQ ID NO: 117 - protein, 17 aa, synthetic construct
MRAWIFFLLCLAGRALA

SEQ ID NO: 118 - protein, 22 aa, synthetic construct
MFSFVDLRLLLLLAATALLTHG

SEQ ID NO: 119 - protein, 19 aa, synthetic construct
MKLVFLVLLFLGALGLCLA

SEQ ID NO: 120 - protein, 22 aa, synthetic construct
MGPTSGPSLLLLLLTHLPLALG

SEQ ID NO: 121 - protein, 18 aa, synthetic construct
MSLSAFTLFLALIGGTSG

SEQ ID NO: 122 - protein, 27 aa, synthetic construct
MAPHRPAPALLCALSLALCALSLPVRA

SEQ ID NO: 123 - protein, 22 aa, synthetic construct
MWATLPLLCAGAWLLGVPVCGA

SEQ ID NO: 124 - protein, 19 aa, synthetic construct
MQALVLLLCIGALLGHSSC

SEQ ID NO: 125 - skipped

SEQ ID NO: 126 - protein, 20 aa, synthetic construct
MQPSSLLPLALCLLAAPASA

SEQ ID NO: 127 - protein, 23 aa, synthetic construct
MAPFEPLASGILLLLWLIAPSRA

SEQ ID NO: 128 - skipped

SEQ ID NO: 129 - protein, 4 aa, synthetic construct
RKRR

SEQ ID NO: 130 - protein, 4 aa, synthetic construct
RRRR

SEQ ID NO: 131 - protein, 4 aa, synthetic construct
RRKR

SEQ ID NO: 132 - protein, 4 aa, synthetic construct
RKKR

SEQ ID NO: 133 - protein, 18 aa, synthetic construct
EGRGSLLTCGDVEENPGP

SEQ ID NO: 134 - protein, 21 aa, synthetic construct
GSGEGRGSLLTCGDVEENPGP

SEQ ID NO: 135 - protein, 19 aa, synthetic construct
ATNFSLLKQAGDVEENPGP

SEQ ID NO: 136 - protein, 22 aa, synthetic construct
GSGATNFSLLKQAGDVEENPGP

SEQ ID NO: 137 - protein, 20 aa, synthetic construct
QCTNYALLKLAGDVESNPGP

SEQ ID NO: 138 - protein, 23 aa, synthetic construct
GSGQCTNYALLKLAGDVESNPGP

SEQ ID NO: 139 - protein, 24 aa, synthetic construct
APVKQTLNFDLLKLAGDVESNPGP

SEQ ID NO: 140 - protein, 27 aa, synthetic construct
GSGAPVKQTLNFDLLKLAGDVESNPGP

SEQ ID NO: 141 - protein, 22 aa, synthetic construct
RKRREGRGSLLTCGDVEENPGP

SEQ ID NO: 142 - protein, 25 aa, synthetic construct
RKRRGSGEGRGSLLTCGDVEENPGP

SEQ ID NO: 143 - protein, 28 aa, synthetic construct
RKRRAPVKQTLNFDLLKLAGDVESNPGP

SEQ ID NO: 144 - protein, 31 aa, synthetic construct
RKRRGSGAPVKQTLNFDLLKLAGDVESNPGP

SEQ ID NO: 145 - DNA, 75 bp, synthetic construct
  source 1..75: mol_type = other DNA
agaaagagaagaggctctggagaaggcagaggctccctgctgacatgtggggatgttgaagagaatcctgggcct

SEQ ID NO: 146 - DNA, 12 bp, synthetic construct
  source 1..12: mol_type = other DNA
agaaagagaaga

SEQ ID NO: 147 - DNA, 21 bp, synthetic construct
  source 1..21: mol_type = other DNA
agaaagagaagaggctctgga

SEQ ID NO: 148 - skipped

SEQ ID NO: 149 - protein, 9 aa, synthetic construct
EPKSCDKTH

SEQ ID NO: 150 - protein, 5 aa, synthetic construct
CPPCP

SEQ ID NO: 151 - protein, 6 aa, synthetic construct
CPPCPA

SEQ ID NO: 152 - protein, 5 aa, synthetic construct
CPPCA

SEQ ID NO: 153 - protein, 22 aa, synthetic construct
EPKSCDKTHTCPPCPAPELLGG

SEQ ID NO: 154 - protein, 22 aa, synthetic construct
EPKSCDKTHLCPPCPAPELLGG

SEQ ID NO: 155 - protein, 10 aa, synthetic construct
EPKSCDKTHL

SEQ ID NO: 156 - protein, 10 aa, synthetic construct
EPKSCDKTHT

SEQ ID NO: 157 - protein, 16 aa, synthetic construct
EPKSCDKTHTCPPCPA

SEQ ID NO: 158 - protein, 16 aa, synthetic construct
EPKSCDKTHLCPPCPA

SEQ ID NO: 159 - protein, 27 aa, synthetic construct
EPKSCDKTHTCPPCPAPELLGGPSVFL

SEQ ID NO: 160 - protein, 27 aa, synthetic construct
EPKSCDKTHLCPPCPAPELLGGPSVFL

SEQ ID NO: 161 - protein, 22 aa, synthetic construct
EPKSCDKTHTCPPCPAPEAAGG

SEQ ID NO: 162 - protein, 27 aa, synthetic construct
EPKSCDKTHTCPPCPAPEAAGGPSVFL

SEQ ID NO: 163 - protein, 27 aa, synthetic construct
EPKSCDKTHLCPPCPAPEAAGGPSVFL

SEQ ID NO: 164 - protein, 18 aa, synthetic construct
ERKSCVECPPCPAPPVAG

SEQ ID NO: 165 - protein, 13 aa, synthetic construct
ERKSCVECPPCPA

SEQ ID NO: 166 - protein, 19 aa, synthetic construct
ESKYGPPCPPCPAPEFLGG

SEQ ID NO: 167 - protein, 13 aa, synthetic construct
ESKYGPPCPPCPA

SEQ ID NO: 168 - protein, 13 aa, synthetic construct
ESKYGPPCPSCPA

SEQ ID NO: 169 - protein, 24 aa, synthetic construct
ESKYGPPCPPCPAPEFLGGPSVFL

SEQ ID NO: 170 - protein, 18 aa, synthetic construct
ERKCCVECPPCPAPPVAG

SEQ ID NO: 171 - protein, 13 aa, unidentified
ERKCCVECPPCPA

SEQ ID NO: 172 - protein, 22 aa, synthetic construct
EPKSCDKTHTCPPCPAPELAGA

SEQ ID NO: 173 - protein, 27 aa, synthetic construct
EPKSCDKTHTCPPCPAPELAGAPSVFL

SEQ ID NO: 174 - protein, 27 aa, synthetic construct
EPKSCDKTHLCPPCPAPELAGAPSVFL

SEQ ID NO: 175 - protein, 22 aa, synthetic construct
EPKSCDKTHTCPPCPAPEFEGG

SEQ ID NO: 176 - protein, 27 aa, synthetic construct
EPKSCDKTHTCPPCPAPEFEGGPSVFL

SEQ ID NO: 177 - protein, 27 aa, synthetic construct
EPKSCDKTHLCPPCPAPEFEGGPSVFL

SEQ ID NO: 178 - protein, 15 aa, synthetic construct
MWCIVLFSLLAWVYA

SEQ ID NO: 179 - protein, 17 aa, synthetic construct
MNPTLILAAFCLGIASA

SEQ ID NO: 180 - protein, 17 aa, synthetic construct
MWQLWASLCCLLVLANA

SEQ ID NO: 181 - protein, 16 aa, synthetic construct
MLLILLSVALLAFSSA

SEQ ID NO: 182 - protein, 20 aa, synthetic construct
MWKRWLALALALVAVAWVRA

SEQ ID NO: 183 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 228 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 185 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 186 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 187 - protein, 735 aa, synthetic construct [sequence omitted]

SEQ ID NO: 188 - protein, 736 aa, synthetic construct
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 189 - protein, 736 aa, synthetic construct; identical to SEQ ID NO: 188

SEQ ID NO: 190 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAAPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 191 - protein, 734 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 192 - protein, 734 aa, synthetic construct; identical to SEQ ID NO: 191

SEQ ID NO: 193 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 194 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 195 - protein, 737 aa, synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 196 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 197 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 198 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 199 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 200 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 201 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 202 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSTLAAPFKSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 203 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 204 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 205 - protein, 735 aa, synthetic construct
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGHQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRPSFYCLEYFPSQMLRTGNNFTFSYTFEDVPFHSSYAHSQSLDRLMNPLIDQYLYYLNRTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLEHVMITDEEEIRTTNPVATEQYGNVSNNLQNSNTGPTTENVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 206 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPRPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 207 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 208 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 209 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSTLAAPFKGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 210 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLRQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYYLNGRDSLVNPGPAMASHKDDEEKFFPMHGTLIFGKEGTNATNAELENVMITDEEEIRTTNPVATEQYGYVSNNLQNSNTAASTETVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 211 - DNA, 1212 bp, synthetic construct
  source 1..1212: mol_type = other DNA
gtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgagggcgcgcgctcccaaaggtacgggtgcactgcccaacggcaccgccataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggcc

SEQ ID NO: 212 - DNA, 1724 bp, synthetic construct
  source 1..1724: mol_type = other DNA
ggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagcc

SEQ ID NO: 213 - DNA, 806 bp, synthetic construct
  source 1..806: mol_type = other DNA
agatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatggatcctgagtaccttctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtc

SEQ ID NO: 214 - DNA, 1003 bp, synthetic construct
  source 1..1003: mol_type = other DNA
tggccctggcattcccctatactgggacatagaaccttcacaggaccaagggcctctcctcccattgatgactgactaggccatcctctagctacataggtggtggagcctagagtccctccttgtgtactctttggtggtggttactctgggggtactaggttagttcgtattgttgttcctcctaggggactgcaaaccccttcagctccttgggtcctttctctagttccttctttggggaccctgtgctcagttcaatggatggccaatttccttcttaaatgcccctagcagtaactgttaggtctcaatcccaagacaaatgtctgaggtgcctatttaacagatcaaagcggacctgtcctcaggttaacccagtcactccctgtacctcagtccctacccatcacaattctccagcccatgagcttcgggctgtacttccccaacgggttctcccattttgggtacatggccttttttttttacctttttggttcctttggccttttggcttttggcttccagggcttctggatcccccccaacccctcccatacacatacacatgtgcactcgtgcactcaacccagcacaggataatgttcattcttgacctttccacatacatctggctatgttctctctcttatctacaataaatctcctccactatacttaggagcagttatgttcttcttctttctttcttttttttttttttttcattcagtaacatcatcagaatcccctagctctggcctacctcctcagtaacaatcagctgatccctggccactaatctgtactcactaatctgttttccaaactcttggcccctgagctaattatagcagtgcttcatgccacccaccccaaccctatccttgttctctgactcccactaatctacacattcagaggattgtggatataagaggctgggaggccagcttagcaaccagagctcgaggctgatgcgagcttcatctcttccctc

SEQ ID NO: 215 - DNA, 523 bp, synthetic construct
  source 1..523: mol_type = other DNA
ttttttttttacctttttggttcctttggccttttggcttttggcttccagggcttctggatcccccccaacccctcccatacacatacacatgtgcactcgtgcactcaacccagcacaggataatgttcattcttgacctttccacatacatctggctatgttctctctcttatctacaataaatctcctccactatacttaggagcagttatgttcttcttctttctttcttttttttttttttttcattcagtaacatcatcagaatcccctagctctggcctacctcctcagtaacaatcagctgatccctggccactaatctgtactcactaatctgttttccaaactcttggcccctgagctaattatagcagtgcttcatgccacccaccccaaccctatccttgttctctgactcccactaatctacacattcagaggattgtggatataagaggctgggaggccagcttagcaaccagagctcgaggctgatgcgagcttcatctcttccctc

SEQ ID NO: 216 - DNA, 215 bp, synthetic construct
  source 1..215: mol_type = other DNA
tgatccctggccactaatctgtactcactaatctgttttccaaactcttggcccctgagctaattatagcagtgcttcatgccacccaccccaaccctatccttgttctctgactcccactaatctacacattcagaggattgtggatataagaggctgggaggccagcttagcaaccagagctcgaggctgatgcgagcttcatctcttccctc

SEQ ID NO: 217 - DNA, 198 bp, synthetic construct
  source 1..198: mol_type = other DNA
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgg

SEQ ID NO: 218 - DNA, 142 bp, synthetic construct
  source 1..142: mol_type = other DNA
aaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 219 - DNA, 109 bp, synthetic construct
  source 1..109: mol_type = other DNA
gtcaagtgacggcggctcagcactcacgtgggcagtgccagcctctaagagtgggcaggggcactggccacagagtcccagggagtcccaccagcctagtcgccagacc

SEQ ID NO: 220 - DNA, 93 bp, synthetic construct
  source 1..93: mol_type = other DNA
ctcagcactcacgtgggcagtgccagcctctaagagtgggcaggggcactggccacagagtcccagggagtcccaccagcctagtcgccagac

SEQ ID NO: 221 - DNA, 1080 bp, synthetic construct
  source 1..1080: mol_type = other DNA
ttaataaacatttgggcgattcttacggcctctaaagaccaagaaccactgctgcctagagctctgctctcttcattgaacaatacaagaggagtgtgtaggtagacacccaccacttccaacagcttaggagagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcaccccgctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggaccaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggaccca

SEQ ID NO: 222 - DNA, 663 bp, synthetic construct
  source 1..663: mol_type = other DNA
gacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 223 - DNA, 762 bp, synthetic construct
  source 1..762: mol_type = other DNA
gacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcg

SEQ ID NO: 224 - DNA, 935 bp, synthetic construct
  source 1..935: mol_type = other DNA
gtcgacaaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagatctagagggccccagaagcctggtggttgtttgtccttctcaggggaaaagtgaggcggccccttggaggaaggggccgggcagaagatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggccttaagc

SEQ ID NO: 225 - DNA, 97 bp, synthetic construct
  source 1..97: mol_type = other DNA
gtaagtttagtctttttgtcttttatttcaggtcccggatccggtggtggtgcaaatcaaagaactgctcctcagtggatgttgcctttacttctag

SEQ ID NO: 226 - protein, 31 aa, synthetic construct
MLRGPGPGLLLLAVQCLGTAVPSTGASKSKR

SEQ ID NO: 227 - protein, 5 aa, synthetic construct
RCGTG

SEQ ID NO: 228 - DNA, 969 bp, synthetic construct
  source 1..969: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 229 - DNA, 1050 bp, synthetic construct
  source 1..1050: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 230 - DNA, 1089 bp, synthetic construct
  source 1..1089: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 231 - DNA, 1430 bp, synthetic construct
  source 1..1430: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 232 - DNA, 1318 bp, synthetic construct
  source 1..1318: mol_type = other DNA
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtaaaacaggtaagtccgctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctggcctctactaaccatgttcatgttttctttttttttctacaggtcctgggtgacgaacag

SEQ ID NO: 233 - DNA, 1579 bp, synthetic construct
  source 1..1579: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 234 - DNA, 1080 bp, synthetic construct
  source 1..1080: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgtgagtatcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 235 - DNA, 568 bp, synthetic construct
  source 1..568: mol_type = other DNA
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 236 - DNA, 895 bp, synthetic construct
  source 1..895: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 237 - DNA, 1291 bp, synthetic construct
  source 1..1291: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 238 - DNA, 1147 bp, synthetic construct
  source 1..1147: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 239 - DNA, 1490 bp, synthetic construct
  source 1..1490: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 240 - DNA, 723 bp, synthetic construct
  source 1..723: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 241 - DNA, 100 bp, synthetic construct
  source 1..100: mol_type = other DNA
aggttaatttttaaaaagcagtcaaaagtccaagtggcccttggcagcatttactctctctgtttgctctggttaataatctcaggagcacaaacattcc

SEQ ID NO: 242 - DNA, 200 bp, synthetic construct
  source 1..200: mol_type = other DNA
aggttaatttttaaaaagcagtcaaaagtccaagtggcccttggcagcatttactctctctgtttgctctggttaataatctcaggagcacaaacattccaggttaatttttaaaaagcagtcaaaagtccaagtggcccttggcagcatttactctctctgtttgctctggttaataatctcaggagcacaaacattcc

SEQ ID NO: 243 - DNA, 321 bp, synthetic construct
  source 1..321: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 244 - DNA, 554 bp, synthetic construct
  source 1..554: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctg

SEQ ID NO: 245 - DNA, 393 bp, synthetic construct
  source 1..393: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 246 - DNA, 393 bp, synthetic construct
  source 1..393: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 247 - DNA, 144 bp, synthetic construct
  source 1..144: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 248 - DNA, 288 bp, synthetic construct
  source 1..288: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 249 - DNA, 432 bp, synthetic construct
  source 1..432: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 250 - DNA, 199 bp, synthetic construct
  source 1..199: mol_type = other DNA
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggc

SEQ ID NO: 251 - protein, 124 aa, synthetic construct
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSA

SEQ ID NO: 252 - protein, 111 aa, synthetic construct
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKR

SEQ ID NO: 253 - protein, 230 aa, synthetic construct [sequence omitted]

SEQ ID NO: 254 - protein, 108 aa, synthetic construct
DIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKR

SEQ ID NO: 255 - protein, 122 aa, synthetic construct [sequence omitted]

SEQ ID NO: 256 - protein, 108 aa, synthetic construct; identical to SEQ ID NO: 254

SEQ ID NO: 257 - protein, 116 aa, synthetic construct
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSS

SEQ ID NO: 258 - protein, 108 aa, synthetic construct
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVL

SEQ ID NO: 259 - protein, 117 aa, synthetic construct
QVQLVQSGAEVKKPGASVKVSCKASGYTFTDFYMDWVRQAPGQRLEWMGYIYPHNAGTTYNQQFTGRVTITVDKSASTAYMELSSLRSEDTAVYYCARRGGFDFDYWGQGTLVTVSS

SEQ ID NO: 260 - protein, 107 aa, synthetic construct; identical to SEQ ID NO: 13

SEQ ID NO: 261 - protein, 249 aa, synthetic construct
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSGGGGSGGGGSGGGGSDIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKR

SEQ ID NO: 262 - protein, 249 aa, synthetic construct
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRGGGGSGGGGSGGGGSQVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSS

SEQ ID NO: 263 - protein, 246 aa, synthetic construct
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSAGGGGSGGGGSGGGGSDIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKR

SEQ ID NO: 264 - protein, 246 aa, synthetic construct
DIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRGGGGSGGGGSGGGGSQVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSA

SEQ ID NO: 265 - protein, 245 aa, synthetic construct
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSGGGGSGGGGSGGGGSDIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKR

SEQ ID NO: 266 - protein, 245 aa, synthetic construct
DIQMTQSPSSLSASVGDRVTITCGASENIYGALNWYQQKPGKAPKLLIYGATNLADGVPSRFSGSGSGTDFTLTISSLQPEDFATYYCQNVLNTPLTFGQGTKVEIKRGGGGSGGGGSGGGGSQVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSS

SEQ ID NO: 267 - DNA, 750 bp, synthetic construct
  source 1..750: mol_type = other DNA
caggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataa

SEQ ID NO: 268 - DNA, 2404 bp, synthetic construct
  source 1..2404: mol_type = other DNA
ctgcgcgctcgctcgctcactgaggccgcccgggcaaagcccgggcggcctcagtgagcgagcgagcgcgcagagagggagtggaattcacgcgtggtaccgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaggaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataaagcggccatcaagcttgatctttttccctctgccaaaaattatggggacatcatgaagccccttgagcatctgacttctggctaataaaggaaatttattttcattgcaatagtgtgttggaattttttgtgtctctcactcggctagcgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 269 - DNA, 2149 bp, synthetic construct
  source 1..2149: mol_type = other DNA
gactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaggaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataaagcggccatcaagcttgatctttttccctctgccaaaaattatggggacatcatgaagccccttgagcatctgacttctggctaataaaggaaatttattttcattgcaatagtgtgttggaattttttgtgtctctcactcggctagcgcatgctggggag

SEQ ID NO: 270 - protein, 239 aa, synthetic construct
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSSGGGGSGGGGSGGGGSSYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVL

SEQ ID NO: 271 - protein, 239 aa, synthetic construct
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVLGGGGSGGGGSGGGGSEVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSS

SEQ ID NO: 272 - protein, 269 aa, synthetic construct
MYRMQLLLLIALSLALVTNSQVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSGGGGSGGGGSGGGGSDIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKR

SEQ ID NO: 273 - protein, 269 aa, synthetic construct
MYRMQLLLLIALSLALVTNSDIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRGGGGSGGGGSGGGGSQVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSS

SEQ ID NO: 274 - protein, 269 aa, synthetic construct
MYRMQLLLLIALSLALVTNSQVQLQQPGAELVRPGTSVKLSCKASGYTFTSSWMHWVKQRPGQGLEWIGVIDPSDSYTNYNQKFKGKATLTVDTSSSTAYMQLSSLTSEDSAVYYCARGGGSSYNRYFDVWGTGTTVTVSSGGGGSGGGGSGGGGSNIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSRTFGGGTKLEIKR

SEQ ID NO: 275 - protein, 269 aa, synthetic construct
MYRMQLLLLIALSLALVTNSNIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSRTFGGGTKLEIKRGGGGSGGGGSGGGGSQVQLQQPGAELVRPGTSVKLSCKASGYTFTSSWMHWVKQRPGQGLEWIGVIDPSDSYTNYNQKFKGKATLTVDTSSSTAYMQLSSLTSEDSAVYYCARGGGSSYNRYFDVWGTGTTVTVSS

SEQ ID NO: 276 - protein, 249 aa, synthetic construct
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSRTFGGGTKLEIKRGGGGSGGGGSGGGGSQVQLQQPGAELVRPGTSVKLSCKASGYTFTSSWMHWVKQRPGQGLEWIGVIDPSDSYTNYNQKFKGKATLTVDTSSSTAYMQLSSLTSEDSAVYYCARGGGSSYNRYFDVWGTGTTVTVSS

SEQ ID NO: 277 - DNA, 810 bp, synthetic construct
  source 1..810: mol_type = other DNA
atgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataa

SEQ ID NO: 278 - DNA, 2709 bp, synthetic construct
  source 1..2709: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 279 - DNA, 3090 bp, synthetic construct
  source 1..3090: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctgccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 280 - DNA, 976 bp, synthetic construct
  source 1..976: mol_type = other DNA
atgtacagaatgcagctgctgctgctcattgccctgtctctggccctggtcaccaattctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagaggaggtggtggaagtggtggtggtggaagtggaggtggtggttctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctcttaataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcgtaa

SEQ ID NO: 281 - DNA, 2709 bp, synthetic construct
  source 1..2709: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagaggaggtggtggaagtggtggtggtggaagtggaggtggtggttctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctcttaataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 282 - DNA, 3090 bp, synthetic construct
  source 1..3090: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagaggaggtggtggaagtggtggtggtggaagtggaggtggtggttctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctcttaataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctgccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 283 - DNA, 1324 bp, synthetic construct
  source 1..1324: mol_type = other DNA
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatactcaggtgagtatctcagggatccagacatggggatatgggaggtgcctctgatcccagggctcactgtgggtctctctgttcacaggttaccggtgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 284 - DNA, 1671 bp, synthetic construct
  source 1..1671: mol_type = other DNA
ctgcgcgctcgctcgctcactgaggccgcccgggcaaagcccgggcggcctcagtgagcgagcgagcgcgcagagagggagtggaattcacgcgtatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatactcaggtgagtatctcagggatccagacatggggatatgggaggtgcctctgatcccagggctcactgtgggtctctctgttcacaggttaccggtgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctgccagggtaatgggctagcgaagcaattctagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 285 - DNA, 1769 bp, synthetic construct
  source 1..1769: mol_type = other DNA
acgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataaagcggccatcaagcttgatctttttccctctgccaaaaattatggggacatcatgaagccccttgagcatctgacttctggctaataaaggaaatttattttcattgcaatagtgtgttggaattttttgtgtctctcactcg

SEQ ID NO: 286 - DNA, 2047 bp, synthetic construct
  source 1..2047: mol_type = other DNA
ctgcgcgctcgctcgctcactgaggccgcccgggcaaagcccgggcggcctcagtgagcgagcgagcgcgcagagagggagtggaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataaagcggccatcaagcttgatctttttccctctgccaaaaattatggggacatcatgaagccccttgagcatctgacttctggctaataaaggaaatttattttcattgcaatagtgtgttggaattttttgtgtctctcactcggctagcgaagcaattctagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 287 - DNA, 2134 bp, synthetic construct
  source 1..2134: mol_type = other DNA
gactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtgcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaggaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataaagcggccatcaagcttgatctttttccctctgccaaaaattatggggacatcatgaagccccttgagcatctgacttctggctaataaaggaaatttattttcattgcaatagtgtgttggaattttttgtgtctctcactcg

SEQ ID NO: 288 - DNA, 2408 bp, synthetic construct
  source 1..2408: mol_type = other DNA
ctgcgcgctcgctcgctcactgaggccgcccgggcaaagcccgggcggcctcagtgagcgagcgagcgcgcagagagggagtggaattcacgcgtggtaccgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtgcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaggaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataaagcggccatcaagcttgatctttttccctctgccaaaaattatggggacatcatgaagccccttgagcatctgacttctggctaataaaggaaatttattttcattgcaatagtgtgttggaattttttgtgtctctcactcggctagcgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 289 - DNA, 2130 bp, synthetic construct
  source 1..2130: mol_type = other DNA
gactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaggaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataaagcggccatcaagcttgatctttttccctctgccaaaaattatggggacatcatgaagccccttgagcatctgacttctggctaataaaggaaatttattttcattgcaatagtgtgttggaattttttgtgtctctcactcg

SEQ ID NO: 290 - DNA, 2404 bp, synthetic construct; identical to SEQ ID NO: 268
  source 1..2404: mol_type = other DNA

SEQ ID NO: 291 - DNA, 2127 bp, synthetic construct
  source 1..2127: mol_type = other DNA
gactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcgggctctagagcctctgctaaccatgttcatgccttcttctttttcctacagctcctgggcaacgtgctggttattgtgctgtctcatcattttggcaaaggaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataaagcggccatcaagcttgatctttttccctctgccaaaaattatggggacatcatgaagccccttgagcatctgacttctggctaataaaggaaatttattttcattgcaatagtgtgttggaattttttgtgtctctcactcg

SEQ ID NO: 292 - DNA, 2401 bp, synthetic construct
  source 1..2401: mol_type = other DNA
ctgcgcgctcgctcgctcactgaggccgcccgggcaaagcccgggcggcctcagtgagcgagcgagcgcgcagagagggagtggaattcacgcgtggtaccgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcgggctctagagcctctgctaaccatgttcatgccttcttctttttcctacagctcctgggcaacgtgctggttattgtgctgtctcatcattttggcaaaggaattcgccaccatgtacagaatgcagctgctgctgctcattgccctgtctctggccctggtcaccaattctcaggtgcagctggttgaatctggtggtggactggtgcagcctggcagaagcctgagactgtcttgtgctgcctctggcttcacagtgcacagcagctactacatggcctgggttagacaggcccctggcaaaggacttgagtgggttggagccatcttcacaggctctggggctgagtataaggctgagtgggccaagggcagagtgaccatcagcaaggacaccagcaagaaccaggtggtgctgaccatgaccaacatggaccctgtggacacagccacctactactgtgcctctgatgctggctatgactaccccacacatgccatgcactattggggccagggcaccctggttacagtgtcctctggaggtggtggaagtggtggtggtggaagtggaggtggtggttctgacatccagatgacacagagccctagcagcctgtctgcctctgtgggagacagagtgaccatcacctgtagagccagccagggcatctctagcagtctggcctggtatcagcagaagcctggaaaggcccctaagctgctgatctatggggcctctgagacagaatctggggtgcccagcagattttcaggctctggctctggcacagacttcaccctgaccatttctagcctgcagcctgaggactttgccacctactactgccagaacaccaaagtgggcagcagctatggcaacacctttggtggtggcaccaaggtggaaatcaagagataataaagcggccatcaagcttgatctttttccctctgccaaaaattatggggacatcatgaagccccttgagcatctgacttctggctaataaaggaaatttattttcattgcaatagtgtgttggaattttttgtgtctctcactcggctagcgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 293 - DNA, 810 bp, synthetic construct
  source 1..810: mol_type = other DNA
atgtacagaatgcagctgctgctgctcattgccctgtctctggccctggtcaccaattctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctctggaggtggtggaagtggtggtggtggaagtggaggtggtggttctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggtaa

SEQ ID NO: 294 - DNA, 2705 bp, synthetic construct
  source 1..2705: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctctggaggtggtggaagtggtggtggtggaagtggaggtggtggttctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggtaataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 295 - DNA, 3090 bp, synthetic construct
  source 1..3090: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctctggaggtggtggaagtggtggtggtggaagtggaggtggtggttctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggtaataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctgccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 296 - DNA, 2705 bp, synthetic construct
  source 1..2705: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggggaggtggtggaagtggtggtggtggaagtggaggtggtggttctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctcttaataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 297 - DNA, 3090 bp, synthetic construct
  source 1..3090: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggggaggtggtggaagtggtggtggtggaagtggaggtggtggttctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctcttaataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctgccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 298 - DNA, 810 bp, synthetic construct
  source 1..810: mol_type = other DNA
atgtacagaatgcagctgctgctgctcattgccctgtctctggccctggtcaccaattctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggggaggtggtggaagtggtggtggtggaagtggaggtggtggttctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctcttaa

SEQ ID NO: 299 - DNA, 1804 bp, synthetic construct
  source 1..1804: mol_type = other DNA
acgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatgtacagaatgcagctgctgctgctcattgccctgtctctggccctggtcaccaattctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggggaggtggtggaagtggtggtggtggaagtggaggtggtggttctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctcttaataaagcggccatcaagcttgatctttttccctctgccaaaaattatggggacatcatgaagccccttgagcatctgacttctggctaataaaggaaatttattttcattgcaatagtgtgttggaattttttgtgtctctcactcggctagcgaagcaattctagcaggcatgctggggag

SEQ ID NO: 300 - DNA, 2047 bp, synthetic construct
  source 1..2047: mol_type = other DNA
ctgcgcgctcgctcgctcactgaggccgcccgggcaaagcccgggcggcctcagtgagcgagcgagcgcgcagagagggagtggaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatgtacagaatgcagctgctgctgctcattgccctgtctctggccctggtcaccaattctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggggaggtggtggaagtggtggtggtggaagtggaggtggtggttctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctcttaataaagcggccatcaagcttgatctttttccctctgccaaaaattatggggacatcatgaagccccttgagcatctgacttctggctaataaaggaaatttattttcattgcaatagtgtgttggaattttttgtgtctctcactcggctagcgaagcaattctagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 301 - DNA, 747 bp, synthetic construct
  source 1..747: mol_type = other DNA
aatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggggaggtggtggaagtggtggtggtggaagtggaggtggtggttctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctct

SEQ ID NO: 302 - DNA, 2733 bp, synthetic construct
  source 1..2733: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggggaggtggtggaagtggtggtggtggaagtggaggtggtggttctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctctgactacaaggacgacgacgacaagtaataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 303 - DNA, 3114 bp, synthetic construct
  source 1..3114: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagataggtaccctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctaatatcatgatgacacagagccccagcagcctggctgtgtctgctggggagaaagtgaccatgagctgcaagagcagccagtctgtgctgtactccagcaaccagaagaactacctggcctggtatcagcagaagcctggccagtctcctaagctgctgatctactgggccagcaccagagaatctggggtgccagacagattcacaggctctggctctggcacagacttcaccctgacaatcagctctgtgcaggctgaggacctggcagtgtactactgtcaccagtacctgagcagcagaacctttggtggtggcaccaagctggaaatcaagaggggaggtggtggaagtggtggtggtggaagtggaggtggtggttctcaggttcagctgcagcaacctggggctgagcttgtcagacctggcacatctgtgaagctgagctgcaaggcctctggctacaccttcacaagcagctggatgcactgggtcaagcagaggcctggacagggccttgagtggattggagtgattgacccctctgacagctacaccaactacaaccagaagttcaagggcaaagccacactgacagtggacaccagcagcagcacagcctacatgcagctgagcagcctgacctctgaggactctgctgtgtactactgtgccagaggtggtggcagcagctacaacagatactttgatgtgtggggcacaggcaccacagtgactgtgtcctctgactacaaggacgacgacgacaagtaataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctgccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 304 - DNA, 1166 bp, synthetic construct
  source 1..1166: mol_type = other DNA
gactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtgcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 305 - DNA, 1162 bp, synthetic construct
  source 1..1162: mol_type = other DNA
gactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 306 - DNA, 1159 bp, synthetic construct
  source 1..1159: mol_type = other DNA
gactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcgggctctagagcctctgctaaccatgttcatgccttcttctttttcctacagctcctgggcaacgtgctggttattgtgctgtctcatcattttggcaaag

SEQ ID NO: 307 - protein, 9 aa, synthetic construct
EPKSCDKTH

SEQ ID NO: 308 - protein, 24 aa, synthetic construct
ESKYGPPCPSCPAPEFLGGPSVFL